(19) United States
(12) Patent Application Publication
(10) Pub. No.: US 2012/0230913 A1
(43) Pub. Date: Sep. 13, 2012

(54) PROTEIN NANOPARTICLE DISPERSIONS

(75) Inventors: Keith P. Johnston, Austin, TX (US); Jennifer A. Maynard, Austin, TX (US); Andrea Miller, Alexandria, VA (US); Brian Wilson, Austin, TX (US); Thomas M. Truskett, Austin, TX (US); Ameya Borwankar, Austin, TX (US); Aileen Dinin, Austin, TX (US)

(73) Assignee: Board of Regents, The University of Texas System, Austin, TX (US)

(21) Appl. No.: 13/417,158

(22) Filed: Mar. 9, 2012

Related U.S. Application Data
(60) Provisional application No. 61/587,648, filed on Jan. 17, 2012.
(60) Provisional application No. 61/451,571, filed on Mar. 10, 2011.

(51) Int. Cl. A61K 39/395; A61K 39/44; A61B 5/055; A61K 39/40; A61K 49/00; A61K 51/10

(52) U.S. Cl. 424/1.49; 424/9.1; 424/9.34; 424/130.1; 424/150.1; 424/178.1; 424/183.1

(57) ABSTRACT

Provided herein, inter alia, are protein dispersions comprising dense protein nanoclusters and methods of making the. Upon dilution, the clusters may reversibly dissociate into native protein molecules with high biological activity. The viscosities of the nanocluster dispersions may be sufficiently low to allow small-volume subcutaneous injections.

### RELATED APPLICATIONS

This application claims priority to U.S. Provisional Patent Application No. 61/587,648 filed, Jan. 17, 2012, entitled “HIGH PROTEIN CONCENTRATION NANOPARTICLE DISPERSIONS” and U.S. Provisional Patent Application No. 61/451,571 filed, Mar. 10, 2011, entitled “LOW VISCOSITY HIGH CONCENTRATION NANOPARTICLE ANTIBODY DISPERSIONS”. The disclosure of each of the above-referenced applications is incorporated by reference herein in their entirety.

### STATEMENT AS TO RIGHTS TO INVENTIONS MADE UNDER FEDERALLY SPONSORED RESEARCH OR DEVELOPMENT

This invention was made with government support under grants NSFSTC-CHE-9876674, CBET-0968038, CBET-1065357 awarded by the National Science Foundation. The Government has certain rights in the invention.

### BACKGROUND OF THE INVENTION

The present invention relates in general to the field of high concentration protein dispersion, and methods of making dispersions of protein nanoparticles. There is a need in the art for highly concentrated protein dispersion for a variety of applications including, for example, pharmaceutical formulations of subcutaneous administration. The present inventions addresses these and other needs in the art.

### BRIEF SUMMARY OF THE INVENTION

In a first aspect, a transparent, low viscosity, high protein concentration dispersion is provided. The dispersion includes a plurality of nanoclusters. Each of the plurality of nanoclusters includes a plurality of proteins and each of the plurality of proteins shares amino acid sequence identity.

In a second aspect a pharmaceutical composition is provided, including any of the dispersions as described herein (including embodiments), wherein the plurality of proteins is a plurality of pharmaceutically active proteins.

In a third aspect a kit is provided, wherein the kit includes a dispersion or pharmaceutical composition described herein (including embodiments).

In a fourth aspect, a method of making a transparent, low viscosity, high protein dispersion of protein nanoclusters is provided, including concentrating a protein-crowder liquid combination and thereby forming the dispersion. The dispersion includes a plurality of nanoclusters, each of the plurality of nanoclusters includes a plurality of proteins, and each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL), and wherein the dispersion includes a plurality of a crowder.

In a fifth aspect, a method of making a transparent, low viscosity, high protein dispersion of protein nanoclusters is provided, including the step of combining a protein in powder form with a crowder and a dispersion liquid thereby forming a dispersion, the dispersion including a plurality of nanoclusters, the nanoclusters including a plurality of the protein. Each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL).

In a sixth aspect, a method of making a transparent, low viscosity, high protein dispersion of protein nanoclusters is provided, including the step of combining a protein in powder form with a dispersion liquid thereby forming a dispersion, the dispersion including a plurality of nanoclusters, the nanoclusters including a plurality of the protein. Each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL).

In a seventh aspect, a method is provided for treating a disease in a patient in need of such treatment, the method including administering an effective amount of any one of the dispersions described herein (including embodiments) to the patient.

In an eighth aspect, a method is provided for modifying the average protein nanocluster diameter of a transparent, low viscosity, high protein dispersion of protein nanoclusters including increasing or decreasing the concentration of a crowder or the protein in the dispersion. The dispersion includes a plurality of nanoclusters and each of the plurality of nanoclusters includes a plurality of proteins. Each of the plurality of proteins shares amino acid sequence identity. The dispersion is a transparent, low viscosity, dispersion; and the dispersion includes a concentration of the protein of greater than about 200 mg/mL (e.g. greater than 200 mg/mL).

In a further aspect a kit is provided, wherein the kit includes protein in powder form or a protein-crowder mixture in powder form, and a dispersion liquid.

### BRIEF DESCRIPTION OF THE DRAWINGS

FIG. 1. Digital image of transparent dispersion of the present invention: FIG. 1A 157 mg/ml—0.08 φ_{P}/0.16 φ_{N}, FIG. 1B 275 mg/ml. All of the dispersions in Table 1 looked very similar.

FIG. 2. Dynamic light scattering (DLS) hydrodynamic diameters of nanoclusters:

FIG. 2A: Trehalose is only extrinsic crowder and mass ratio of trehalose to protein is 1:1 (At 142 mg/ml, φ_{T}=0.09), FIG. 2B: 157 mg/ml IgG dispersion with φ_{P}=0.16 or 0.24. Additional sample information can be found in Table 1.

FIG. 3. DLS hydrodynamic diameters at constant extrinsic crowder concentrations versus protein to determine protein solubilities: FIG. 3A: Initial 250 mg/ml IgG in pH 6.4 buffer with 250 mg/ml trehalose (φ_{T}=0.15). The protein monomer solubility is between ˜31 and 50 mg/ml, FIG. 3B: Initial 200 mg/ml IgG in pH 6.4 buffer with 0.16 φ_{N}/0.08 φ_{P} and 200 mg/ml trehalose (φ_{E}=0.34). The protein monomer solubility is between ˜1.5 and 2.5 mg/ml.

FIG. 4. Representative cryo-SEMs and STEM images of the 157 mg/ml—0.08 φ_{P}/0.16 φ_{N} IgG dispersion in Table 1.

FIG. 5. FIG. 5A shows the hydrodynamic diameter of protein nanoclusters at a constant IgG concentration of 50 mg/ml. In path 1, trehalose concentration was increased with 500 mg/ml trehalose in pH 6.4 phosphate buffer along with small amounts of dispersion of 200 mg/ml IgG with IgG:trehaolose (1:1 w/w) to maintain constant IgG concentration. For decreasing sugar conc. set, pure buffer was added while maintaining const. IgG conc. in the same way. In path 2, solid sugar crystals were added to a 50 mg/ml IgG solution to increase the sugar concentration. In path 3, trehalose concentration was decreased in a way similar to the decrease in path 1 using pure pH 6.4 phosphate buffer and 200 mg/mL IgG dispersion with 1:1 IgG:trehalose by weight. The values for cluster diameters obtained from theory are also superimposed on the plot. FIG. 5B shows IgG and trehalose concentration both constant at 30 mg/ml. Volume fractions of PEG300 and NMP were increased by adding a 1:2 volume solution of PEG300:NMP along with lyophilized powder with 1:1 weight ratio of IgG and trehalose to maintain constant IgG and trehalose concentrations.

FIG. 6. Plotted distribution of the hydrodynamic diameter from DLS for selected samples from FIG. 5A at different concentrations of trehalose for different paths of preparing the solution from FIG. 5A.

FIG. 7. Universal scaling of hydrodynamic diameter measured by DLS for data in FIG. 5A with increasing trehalose concentrations, (plus signs) pure sugar crowder. (diamonds) const. φ_{T}=0.018 with increasing NMP/PEG300 at conditions in FIG. 5B.

FIG. 8. Static light scattering (SLS) data on dilutions of the protein/trehalose nanocluster dispersions with constant 0.08 φ_{P}/0.16 φ_{N}.

FIG. 9. Total potential (Vₜₒₜ(r)), attractive potentials from van der Waals, V_{vwd}(r), specific short-range attraction, Vₛᵣ(r), and depletion-attraction, V_{dep}(r) for a 0.5 nm radius crowder and electrostatic repulsive potential, V_{electrostatic}, for: FIG. 9A Electrostatically stabilized protein monomer with added crowders, FIG. 9B Unstable protein monomer near the pI with added crowders, FIG. 9C an electrostatically stabilized protein nanocluster near the pI (assuming 1 charge/protein molecule) with added crowders, and FIG. 9D An electrostatically stabilized protein nanocluster near the pI (assuming 2 charges/protein molecule) with added crowders.

FIG. 10. Phase diagram for a protein dispersion based on the theory described herein. The steep solid line is the gel line above which the solution forms a gel phase. The lines indicate clusters of the same size or aggregation number. The number in the legend is the diameter of the cluster in nanometers for that particular curve.

FIG. 11. FIG. 11A shows a digital image of transparent dispersion of BSA at 200 mg/ml with 300 mg/ml of trehalose according to the present invention. FIGS. 11B and 11C show SEM images of a 1B7 nanocluster (11B) and a sheep IgG nanocluster (11C). Spherical protein monomer with a halo of trehalose molecules around them can be seen in the figure. FIG. 11D shows the distribution of hydrodynamic diameter by DLS of a concentrated nanocluster dispersion and protein dilution at a constant crowder (trehalose) concentration of 270 mg/ml. The size of the nanocluster is seen to be nearly constant until the concentration drops to 50 mg/ml of protein. FIG. 11E shows the distribution of hydrodynamic diameters from DLS for high concentration dispersions of Sheep IgG with a mass ratio of 1:0.5 of IgG to trehalose which demonstrates the concept at higher concentrations.

FIG. 12. FIG. 12A shows the distribution of hydrodynamic diameters of 1B7 clusters from DLS for a range of sugar concentrations. The concentration of the 1B7 is maintained constant at 70 mg/ml for all these dispersions. FIG. 12B shows average cluster size versus crowder concentration from theoretical predictions based upon the theory described herein and the actual experimentally observed size. FIG. 12C is a plot similar to the plot in FIG. 10, it is a theoretical prediction for cluster sizes giving a phase diagram for mAb 1B7. It shows protein volume fraction against the volume fraction of extrinsic crowder. The gel line indicates the locus of points above which the dispersion is predicted to gel up while the other curves on the plot are curves indicating constant cluster size. FIG. 12D is a plot showing the potential between the protein nanoclusters. The electrostatic repulsion and the attractive forces namely the specific short ranged forces, the depletion forces and the Van der Waals forces together create a potential barrier of about 19 kT. This barrier serves to prevent the protein nanoclusters from aggregating together.

FIG. 13. Pharmacokinetics for 1B7 administered to mice by different administration methods. The concentration of the antibody was monitored at different timepoints by ELISA. The dispersion was 235 mg/ml 1B7 with 235 mg/ml trehalose in the solution.

FIG. 14. Fraction of protein folded as a function of the volume fraction of the protein based on calculations from the coarse grained model. At high volume fractions, the protein gets self-crowded causing the protein molecules to favor being in the folded form.

FIG. 15. SEM micrographs of dried IgG powders frozen at 20 mg/ml with a 1:1 by weight ratio of protein to trehalose after lyophilization of the slow frozen lyophilized sheep IgG.

FIG. 16. Calibration curve for small conical vials for viscosity measurements using various solution standards. DI water (ηₒ=1 cP), PEG200 (ηₒ=50 cP), PEG300 (ηₒ=70 cP), PEG400 (ηₒ=90 cP), and benzyl benzoate (ηₒ=8.8 cP). The time for the liquid level to be drawn from 0.4″ to 0.1″ in small conical vial was measured from a video of the solution converted to a stack of images with 30 images per second.

FIG. 17. IEF analysis of sheep IgG solution, from left to right lanes are IEF markers (Bio-Rad), 2 μg sheep IgG and 1 μg sheep IgG. B) Zeta potential measurements on sheep IgG solution.

FIG. 18. Hydrodynamic diameter distribution. FIG. 18A is the hydrodynamic diameter distribution from DLS on a concentrated (10% solids weight) polystyrene standard of 298 nm spheres while FIG. 18B is the correlation function for sample in A, raw data (G2(Raw)), and fit using CONTIN algorithm (G2(Rec)).

FIG. 19. Plots showing the static light scattering measurement at various angles to determine the fractal dimension of the nanoclusters. FIG. 19A is for nanoclusters at 50 mg/ml with 250 mg/ml trehalose and FIG. 19B is for nanoclusters at 10 mg/ml with 8% PEG300/16% NMP.

FIG. 20. Plot of the maximum emission wavelength measured from an IgG sample at various concentrations of urea.

FIG. 21. Plot of the hydrodynamic diameter distributions from DLS of a Sheep IgG dispersion with a 1:1 by weight ratio of IgG to trehalose as it is serially diluted using a solution of phosphate buffer at pH 6.4. The size can be seen to decrease as the solution becomes dilute.

FIG. 22. SEM images of 1B7 clusters showing the morphology of the clusters.

FIG. 23. Plot of the potential between two monomeric protein molecules as a function of the inter-monomer distance for a protein near its pI.

FIG. 24. Distribution of hydrodynamic diameter from DLS for nanoclusters of BSA at high concentrations of 400 mg/ml and 350 mg/ml.

FIG. 25. Schematic of the SWIFT freezing process. The unfrozen protein solution in a cylindrical vial is placed on its side and rolled while exposed to liquid nitrogen. This causes a thin film of the protein solution to freeze on the inside edge of the vial followed by subsequent films towards the center of the vial resulting in a frozen annulus of protein solution which is placed in the lyophilizer to remove water.

FIG. 26. Image of an iso-electric focusing (IEF) gel to determine the isoelectric point (pI) of mAb 1B7. Lane 1: IEF standards, ranging from 4.45 to 9.6 (BioRad); 2: 1 mg/ml 1B7; 3: 2 mg/ml 1B7; 4: 5 mg/ml 1B7.

FIG. 27. FIGS. 27A and 27B are calibration data for the anti-pertussis toxin activity ELISA: FIG. 27A shows sample spiked serum pertussis ELISA assay analyzed using parallel line fit to a 100 μg/ml spiked serum standard to determine EC₅₀ in SpectraMax Pro software, FIG. 27B shows a measurement of the correlation between standards: natural log of the sample EC₅₀ divided by the EC₅₀ of the 100 μg/ml spiked serum standard versus the spiked serum concentration. For each sample, the natural log of the EC₅₀/EC₅₀ of the 100 μg/ml standard and used to determine the serum mAb 1B7 concentration.

FIG. 28. Schematic of SWIFT freezing process and dry powder SEM. A) The unfrozen protein solution in a cylindrical vial is placed on its side and rolled while exposed to liquid nitrogen. This causes a thin film of the protein solution to freeze on the inside edge of the vial followed by subsequent films towards the center of the vial resulting in a frozen annulus of protein solution which is placed in the lyophilizer to remove water. B) Morphology of SWIFT powder after lyophilization by SEM.

FIG. 29. Comparison of unprocessed, lyophilized and dispersed 1B7 by DLS. All samples were diluted to 5 mg/ml in PBS.

FIG. 30. Comparison of unprocessed, lyophilized and dispersed 1B7 by PTx ELISA to monitor antibody activity.

FIG. 31. SWIFT freezing temperature profiles of lysozyme solutions (10 mg/ml) inside vials. The solutions were frozen in different film thicknesses 2.6 mm and 0.6 mm corresponding to the total liquid volume of 4 ml and 2.6 ml in vials with 15 mm diameter. The coolant temperature was 80 K and the vial rotation speed was 30 rpm.

FIG. 32. Effect of antibody concentration on particle size in dispersion buffer. At high concentration (200 mg/ml) in dispersion buffer, dynamic light scattering (DLS) detects only large particles of ˜200 nm. Upon dilution to 5 mg/ml in dispersion buffer, the nanoparticles equilibrate between the large 200 nm and smaller 50 nm nanoclusters. Further dilution with dispersion buffer to below the solubility limit (2.5 and 5 mg/ml), detects only particles of ˜10 nm size, the expected size for monomeric IgG.

FIG. 33. Visual appearance of dispersion: FIG. 33A is a digital image of suspended particles, FIG. 33B is a SEM image of the mAb1B7 dispersion (200 mg/ml) when diluted to 100 mg/ml in the dispersion buffer, rapidly frozen with the water removed by lyophilization.

FIG. 34. Characterization of antibody recovered from dispersion: FIG. 34A is an image of a SDS-PAGE gel comparing unprocessed, purified mAb1B7 (lane 1) and dispersion diluted from 200 to 1 mg/ml in PBS (lane 2) and FIG. 34B shows a comparison of unprocessed, lyophilized and dispersed 1B7 by PTx ELISA to monitor antibody activity.

FIG. 35. Non-reducing western blot to detect biotinylated 1B7 in the terminal serum samples. 4 μg of 1B7 from serum samples were combined with non-reducing SDS-PAGE loading buffer, boiled and applied to a 4-20% SDS-PAGE gel. After separation and transfer to a PVDF membrane, the blot was blocked with 5% BSA and probed with SA-HRP to detect intact and fragments of mAb 1B7. Lanes contain the following mouse samples: 1: IV solution, mouse #2; 2: IV solution 145; 3: SQ solution #7; 4: SQ solution #10; 5: SQ low dose dispersion #13; 6: SQ low dose dispersion #17; 7: SQ high dose dispersion #20; 8: SQ high dose dispersion #24; 9: SQ dispersion buffer only #18. The amount of serum used for lane 9 corresponded to amount of serum used in the most dilute sample (SQ low dose dispersion #13).

FIG. 36. Nanocluster morphology for 1B7 antibody with trehalose as extrinsic crowder. A. Schematic of protein cluster where large circles represent proteins, small dots, counterions and medium circles, extrinsic crowders. Similar clusters are observed for colloids in organic solvent. B. Transparent dispersion at c=c_{E}=220 mg/ml. C. SEM image of 36B indicating closely-spaced, self-crowded protein. (The “halo” on the component particles is an artifact of trehalose deposition during sample preparation). D. Schematic of dispersion of nanoclusters drawn to scale.

FIG. 37. Hydrodynamic diameter by DLS for 1B7 antibody and polyclonal sheep IgG with trehalose as extrinsic crowder. A. 1B7: serial dilutions in buffer such that c/c_{E}=1. B. 1B7: dilution in pH 7.2 phosphate buffer with starting c=c_{E}=220 mg/ml as in FIG. 36a (squares) and decreasing c_{E} with a constant c of 70 mg/ml with a starting c_{E} of 270 mg/ml (diamonds). Error bars indicate ±s.d. in peak width. The predictions of Eq. 14 are in qualitative agreement. C. 1B7: constant c of 70 mg/ml for decreasing c_{E} of trehalose from 270 to 150 mg/ml as shown in legend and then a final point where c_{E} is raised back to 270 mg/ml, labeled as 270 mg/ml—2. D. polyclonal sheep IgG: constant c of 50 mg/ml for increasing (diamonds) followed by decreasing (squares) trehalose concentration. The reversibility suggests equilibrium cluster behavior. The theoretical predictions of Eq. 14 are in qualitative agreement with the data.

FIG. 38. BSA nanocluster size for high protein concentrations. A high concentration BSA dispersion formulated at c=400 mg/ml and c_{E}=240 mg/ml forms nanoclusters with hydrodynamic diameter of 40 nm. Dispersions formulated with lower concentrations of BSA and/or trehalose yield progressively smaller nanoclusters. Also shown is BSA monomer which is 3-4 nm diameter.

FIG. 39. Antibody conformation and activity. A. Circular dichroism spectra of monoclonal antibody 1B7 control and 267 mg/ml dispersion. All samples were diluted to 0.1 mg/ml in PBS and analyzed on a Jasco J-815 CD Spectrometer. B. Theoretical prediction of the fraction of folded protein suggesting that the native state would be favored at high φᵢₙₜ=0.6 found in antibody nanocluster (Shen, Cheung et al. 2006).

FIG. 40. Protein-protein, protein-cluster and cluster-cluster hierarchical interactions in nanocluster dispersions. The potential of mean force includes specific short-ranged (ssr), depletion attraction (dep) and electrostatic (el) components: V(r)=Vₛₛᵣ(r)+V_{dep}(r)+Vₑₗ(r). A. Components of V(r) for protein monomers at pI and 3 pH units away from pI. B. Predicted cluster diameter contours. The triangle denotes the conditions of the injected dispersion into mice at c=235 mg/ml for 1B7 as given in Table 16. The diagonal pathway represents dilution of the dispersion (FIG. 37a). C. V(r) for two 50 nm nanoclusters based on experimental zeta potential for polyclonal IgG. Inset, arc depicts range of long-ranged repulsion at the edges of two clusters and ring around circles indicates short-ranged inter-cluster attraction.

FIG. 41. Pharmacokinetics of concentrated 1B7 dispersion and solution controls. Time course of serum antibody concentration normalized by dose after administration of intravenous solution, subcutaneous solution or subcutaneous dispersion. Serum samples were recovered from the tail vein and the 1B7 concentration determined by ELISA.

FIG. 42. Schematic for the depletion attraction between two protein particles (large gray circles) induced by the presence of crowders (small circles) in solution. The attractive force reflects the entropic preference for configurations such as this where the volume excluded to the centers of the crowders is reduced by the size of the overlap region.

FIG. 43. SEM images of antibody nanoclusters with trehalose as extrinsic crowder. A, Reproducibility of multiple SEM images of 1B7 antibody nanoclusters at c=c_{E}=220 mg/ml (identical conditions as in FIG. 36c). The SEM micrographs clearly show good reproducibility in the size of the ˜300 nm clusters in the dispersion for four clusters, consistent with the DLS results in FIG. 37a. The images were obtained from regular carbon film copper TEM grids where the nanoclusters were resting on the copper mesh. The individual protein monomers, on the order of 10 nm, appear to have a halo around them. This halo is a layer of trehalose deposited during freezing and lyophilization in sample preparation for SEM. B, Polyclonal IgG nanocluster at c=c_{E}=260 mg/ml. The imaging was done on a lacey carbon TEM grid and the nanocluster is resting on a strand of lacey carbon.

FIG. 44. Static light scattering to determine fractal dimension. The 80 nm sheep IgG nanoclusters were formed at c=70 mg/ml IgG and c_{E}=270 mg/ml trehalose. The intensity which scales as the measured count rate was plotted versus the scattering vector 4π sin(θ/2)/λ at various angles from 45° to 90°. The slope of the line fit through the data multiplied by −1, i.e., 2.6 is the fractal dimension. (Hiemenz and Rajagopalan 1997) In static light scattering, we assume that the structure factor is not a function of the scattering vector and therefore, the intensity is related to the scattering vector through the fractal dimension.

FIG. 45. Hydrodynamic diameter by DLS of polyclonal IgG nanoclusters upon dilution in buffer (c/c_{E}=1). The protein concentrations are shown in the legend. Sequential dilution with phosphate buffer at constant c/c_{E} yields progressively smaller nanoclusters until monomeric protein with a hydrodynamic diameter of ˜10 nm is observed at c=c_{E}=47 mg/ml. The behavior and mechanism for nanocluster dissociation is similar as observed for monoclonal antibody 1B7 in FIG. 37a and b.

FIG. 46. Polyclonal IgG nanocluster size at high concentration. Polyclonal sheep IgG dispersions were formulated with 300 and 350 mg/ml protein with c/c_{E}=1:0.5 with trehalose and the resulting nanocluster hydrodynamic diameter measured by DLS

FIG. 47. HPLC SEC of monomer concentration after dilution of the dispersion. All samples were diluted to 1 mg/ml in PBS and analyzed with Waters Breeze HPLC with TOSOH Biosciences TSKgel G2000SW and G3000SW_{XL} columns. The mobile phase comprised 100 mM sodium phosphate and 300 mM sodium chloride buffer (pH 7.0), and the eluate was monitored by absorbance at 214 nm. A. Chromatographs are shown for (1) solution control 1B7, (2) lyophilized, reconstituted 1B7, and dispersion formulated with (3) 260 mg/ml 1B7 and 260 mg/ml trehalose. No increase was seen in aggregate concentration throughout formation of the dispersion, dilution of the clusters, and reformation of the clusters with trehalose. B. The % monomer values are given here for a wide range of indicated experiments, shown in FIGS. 37a and 37b. Error indicated is ±s.d.

FIG. 48. SDS-PAGE gel. Absence of higher molecular weight aggregates as assessed by non-reducing SDS-PAGE. All dispersions were diluted to 1 mg/ml with PBS prior to analysis. 5 μg of each sample was combined with non-reducing loading buffer and loaded on to a precast 4-20% SDS-PAGE gel (Bio Rad). Lane (I) molecular weight markers (Spectra BR); (2) solution control 1B7; (3) & (4) 1B7 post-lyophilization; (5) molecular weight markers (Spectra BR); (6) & (7) diluted 260 mg/ml 1B7 dispersion; (8) & (9) 260 mg/ml dispersion diluted to 75 mg/ml that was further diluted. None of the samples showed any change in molecular weight, or formation of any higher molecular weight aggregates.

FIG. 49. Viscosity calibration curve for measurements with small conical vials. The calibration curve was created using the following solution standards: DI water (ηₒ=1 cP), benzyl benzoate (ηₒ=8.8 cP), PEG200 (ηₒ=50 cP), PEG300 (ηₒ=70 cP), and PEG400 (ηₒ=90 cP). The time for the liquid level to be drawn from 0.4″ to 0.1″ in small conical vial (0.1 mL V-Vial, Wheaton) was measured from a video of the solution (taken with a Kodak EasyShare Z812 IS), converted using Image J software to a stack of images with 30 images per second. The time was measured to within 0.05 seconds at least 3 times and averaged, while maintaining the end of the plunger at the 1 ml mark. A maximum volume of 10% of the cavity in the syringe was filled with dispersion to minimize variation in the pressure drop.

FIG. 50. Dispersion characteristics before and after using a centrifugal filtration-concentration method—pre and post-freezing. The dispersions were formulated with 217 mg/mL IgG and 70 mg/mL trehalose and frozen for 1 month.

FIG. 51. Dispersion turbidity at varying wavelengths. Turbidity was measured on a Cary 3E UV/Vis spectrophotometer and is given for pre-filtrated dispersions.

FIG. 52. SEM images of antibody nanoclusters with arginine as extrinsic crowder. The dispersion was diluted 4 fold at a constant crowder volume fraction of 0.077 using NMP as a crowder before dropping on a copper TEM grid with lacey carbon film. Each image contains a single nanoparticle on top of a lacey carbon grid and is between 50-100 nm in diameter.

FIG. 53. Schematic for forming dispersions through centrifugal filtration-concentration. Protein is added to form a protein solution. To the protein solution is added crowder. The solution is transferred to a tube for centrifugal filtration-concentration. Concentration is achieved after centrifugation with some loss of the crowder through the filter.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

### I. DEFINITIONS

To facilitate the understanding of this invention, a number of terms are defined below.

As used herein, the term “nanocluster” refers to 10 or more proteins or peptides that are not irreversibly aggregated, having a diameter between 20 and 1,000 nanometers, which may optionally be physically associated with additional compounds, components, or compositions. In some embodiments, the diameter is a hydrodynamic diameter. In some embodiments, the nanocluster may include subclusters of proteins or peptides that form a larger cluster. In some embodiments, the nanocluster may be self-crowding, wherein the crowding is caused by the proteins or peptides. In some embodiments, the nanoclusters may form in the presence of an extrinsic crowder. In some embodiments, the nanoclusters may be mostly self-crowding. The term nanocluster does not include protein or peptide crystals.

As used herein, the terms “syringable” and “syringeable” are used interchangeably and refer to a final composition for delivery to a subject that is sufficiently fluid to be flowable through a syringe (e.g. a syringe with a needle that is 21 to 27 gauge). For example, a composition that is “syringable” has a low enough viscosity to load the syringe and inject a subject from the syringe without undue force, wherein undue force is an amount in excess of the force exerted by a skilled practitioner in the medical field (e.g. doctor, nurse) to deliver compositions to a patient (e.g. through iv injection, SQ injection) through a syringe (e.g. a syringe with a needle that is 21 to 27 gauge) without adverse effects to the patient solely due to the force applied in the delivery.

As used herein, the term “non-settling” or “redispersible” refers to a composition that remains in solution phase (i.e., does not sediment) after an extended period of time, e.g., 1 hour, 2 hours, 1 day, 3 days, 5 days, 1 week, 1 month, 3 months, 6 months, 1 year or more). For example, a composition is “re-dispersible” if upon re-dispersion it does not flocculate so quickly as to prevent reproducible dosing of a drug.

As used herein, the term “additive(s)” refers to salts, sugars, organics, buffers, polymers and other compositions that include: Disodium edetate, Sodium chloride, Sodium citrate, Sodium succinate, Sodium hydroxide, Sodium glucoheptonate, Sodium acetyltryptophanate, Sodium bicarbonate, Sodium caprylate, Sodium pertechnetate, sodium acetate, sodium dodecyl sulfate, aluminum hydroxide, aluminum phosphate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, triton n101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, Polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol (antiseptic), glyclyglycine, thimerosal (antiseptic, antifungal), Parabens (preservative), Gelatin, Formaldehyde. Dulbecco's modified eagles medium, Hydrocortisone, Neomycin, Von Willebrand factor, Gluteraldehyde, Benzethonium chloride, White petroleum, p-aminopheyl-p-anisate, monosodium glutamate, beta-propiolactone, Acetate, Citrate, Glutamate, Glycinate, Histidine, Lactate, Maleate, Phosphate, Succinate, Tartrate, Tris, Carbomer 1342 (copolymer of acrylic acid and a long chain alkyl methacrylate cross-linked with allyl ethers of pentaerythritol), Glucose star polymer, Silicone polymer, Polydimethylsiloxane, Polyethylene glycol, carboxymethylcellulose, Poly(glycolic acid), Poly(lactic-co-glycolic acid). Polylactic acid. Dextran 40, Poloxamers (triblock copolymers of ethylene oxide and propylene oxide).

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

An “effective amount” is an amount sufficient to accomplish a stated purpose (e.g. achieve the effect for which it is administered, treat a disease, reduce enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of a composition (e.g. antagonist, protein, low molecular weight compound) required to decrease the activity of an enzyme relative to the absence of the composition (e.g. antagonist). A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. compounds including biomolecules, proteins, antibodies, or cells) to become sufficiently proximal to react, interact or physically touch. It should be appreciated, however, the resulting reaction product can be produced directly from a reaction between the added reagents or from an intermediate from one or more of the added reagents which can be produced in the reaction mixture.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the presence of a disease-related protein. Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound that inhibits the activity of a protein or production of a protein, e.g., by binding, partially or totally blocking stimulation (e.g. production), decreasing, preventing, or delaying activation, or inactivating, desensitizing, or down-regulating signal transduction or enzymatic activity. Inhibition may also reduce the amount of a protein by increasing clearance or degradation of the protein. In some embodiments, an inhibitor is an antibody.

The term “modulator” refers to a composition that increases or decreases the level of a target molecule or the function of a target molecule.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions (e.g. proteins, crowders, nanoclusters, dispersions) of the invention. One of skill in the art will recognize that other pharmaceutical excipients are useful in the present invention.

The term “preparation” is intended to include the formulation of the active composition (e.g. protein nanoclusters, protein-crowder nanoclusters, dispersions) with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

The terms “isolated” “purified” or “biologically pure” refer to material that is substantially or essentially free from components which normally accompany it as found in its native state. Purity and homogeneity of biological molecules (e.g. nucleic acids or proteins) are typically determined using analytical chemistry techniques such as polyacrylamide gel electrophoresis or high performance liquid chromatography. A protein that is the predominant species present in a preparation is substantially purified. The term “purified” may denote that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure. As an example, an isolated cell or isolated sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

The term “hydrodynamic diameter” has its plain ordinary meaning within Chemistry and refers to the apparent diameter of a hypothetical hard sphere that diffuses through a medium at the same speed as the molecule under observation (e.g. as measured by dynamic light scattering).

As used herein, the term “transparent” refers to the physical property of allowing light to pass through a material. In some embodiments, transparent refers to the property of allowing a majority of the incident light, at a given wavelength(s), to pass through the material. In some embodiments, transparent refers to the property of allowing greater than about 75% of the incident light at specified wavelengths (e.g. visible light, 600 nm, 400-700 nm) to pass through the material. In some embodiments, transparent refers to the property of allowing greater than about 80% of the incident light at specified wavelengths to pass through the material. In some embodiments, transparent refers to the property of allowing greater than about 85% of the incident light at specified wavelengths to pass through the material. In some embodiments, transparent refers to the property of allowing greater than about 90% of the incident light at specified wavelengths to pass through the material. In some embodiments, transparent refers to the property of allowing greater than about 95% of the incident light at specified wavelengths to pass through the material. In some embodiments, transparent refers to the property of allowing greater than about 96% of the incident light at specified wavelengths to pass through the material. In some embodiments, transparent refers to the property of allowing greater than about 97% of the incident light at specified wavelengths to pass through the material. In some embodiments, transparent refers to the property of allowing greater than about 98% of the incident light at specified wavelengths to pass through the material. In some embodiments, transparent refers to the property of allowing greater than about 99% of the incident light at specified wavelengths to pass through the material. In some embodiments, transparent refers to the property of allowing greater than about 99.5% of the incident light at specified wavelengths to pass through the material. In some embodiments, transparent refers to the property of allowing greater than about 99.6% of the incident light at specified wavelengths to pass through the material. In some embodiments, transparent refers to the property of allowing greater than about 99.7% of the incident light at specified wavelengths to pass through the material. In some embodiments, transparent refers to the property of allowing greater than about 99.8% of the incident light at specified wavelengths to pass through the material. In some embodiments, transparent refers to the property of allowing greater than about 99.9% of the incident light at specified wavelengths to pass through the material In some embodiments, when referring to the transparency of a dispersion of protein nanoclusters, the percentages above (e.g. percentage value of any one of 75, 80, 85, 90, 95, 96, 97, 98, 99, 99.5, 99.6, 99.7, 99.8, 99.9), is in comparison to a control sample lacking the protein, which would be assigned the value of 100% incident light at specified wavelengths passing through the material. In some embodiments, transparency is measured by light extinction, wherein the term “light extinction” as used herein refers to the combined absorption and scattering of incident light at zero degrees from the angle of the incident light. In some embodiments, transparent means having a light extinction of less than about 0.05 cm⁻¹. In some embodiments, transparent means having a light extinction of less than about 0.1 cm⁻¹. In some embodiments, transparent means having a light extinction of less than about 0.25 cm⁻¹. In some embodiments, transparent means having a light extinction of less than about 0.5 cm⁻¹. In some embodiments, transparent means having a light extinction of less than about 1%. In some embodiments, transparent means having a light extinction of less than about 2%. In some embodiments, transparent means having a light extinction of less than about 3%. In some embodiments, transparent means having a light extinction of less than about 4%. In some embodiments, transparent means having a light extinction of less than about 5%. In some embodiments, transparent means having a light extinction of less than about 10%. In some embodiments, transparent means having a light extinction of less than about 15%. In some embodiments, transparent means having a light extinction of less than about 20%. In some embodiments, transparent means having a light extinction of less than about 25%. In some embodiments, when referring to the transparency of a dispersion of protein nanoclusters in terms of light extinction, the percentages above (e.g. percentage value of any one of 1, 2, 3, 4, 5, 10, 15, 20, or 25), is in comparison to a control sample lacking the protein, which would be assigned the value of 0% light extinction. A “light extinction measurement” refers to a light extinction value physically measured by a person of ordinary skill.

The term “viscosity” has its plain ordinary meaning within Chemistry, as applied to liquids and fluids.

As used herein, the term “low viscosity” refers to a viscosity that is less than about 100 centipoise. In some embodiments, “low viscosity” refers to a viscosity of less than about 90 centipoise. In some embodiments, “low viscosity” refers to a viscosity of less than about 80 centipoise. In some embodiments, “low viscosity” refers to a viscosity of less than about 70 centipoise. In some embodiments, “low viscosity” refers to a viscosity of less than about 60 centipoise. In some embodiments, “low viscosity” refers to a viscosity of less than about 50 centipoise. In some embodiments, “low viscosity” refers to a viscosity of less than about 40 centipoise. In some embodiments, “low viscosity” refers to a viscosity of less than about 30 centipoise. In some embodiments, “low viscosity” refers to a viscosity of less than about 20 centipoise. In some embodiments, “low viscosity” refers to a viscosity of less than about 10 centipoise. In some embodiments, a low viscosity is measured with a viscometer, rheometer, or syringe loading method as described herein. In some embodiments, a low viscosity is measured with a shear rate that is about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 second⁻¹. In some embodiments, an average shear rate may be determined from the flow rate and geometric properties with a syringe loading method as described herein. In some embodiments, “low viscosity” refers to any one of the combinations of viscosity and shear rate shown in the table/matrix below having number 1 to 280, wherein each cell corresponds to the viscosity for that column and the shear rate for that row:

| shear rate about | Viscosity less than about(centipoise) | | | | | | | | | |
|---|---|---|---|---|---|---|---|---|---|---|
| (second⁻¹ | 100 | 90 | 80 | 70 | 60 | 50 | 40 | 30 | 20 | 10 |
| 100 | 1 | 2 | 3 | 4 | 5 | 6 | 7 | 8 | 9 | 10 |
| 200 | 11 | 12 | 13 | 14 | 15 | 16 | 17 | 18 | 19 | 20 |
| 300 | 21 | 22 | 23 | 24 | 25 | 26 | 27 | 28 | 29 | 30 |
| 400 | 31 | 32 | 33 | 34 | 35 | 36 | 37 | 38 | 39 | 40 |
| 500 | 41 | 42 | 43 | 44 | 45 | 46 | 47 | 48 | 49 | 50 |
| 600 | 51 | 52 | 53 | 54 | 55 | 56 | 57 | 58 | 59 | 60 |
| 700 | 61 | 62 | 63 | 64 | 65 | 66 | 67 | 68 | 69 | 70 |
| 800 | 71 | 72 | 73 | 74 | 75 | 76 | 77 | 78 | 79 | 80 |
| 900 | 81 | 82 | 83 | 84 | 85 | 86 | 87 | 88 | 89 | 90 |
| 1000 | 91 | 92 | 93 | 94 | 95 | 96 | 97 | 98 | 99 | 100 |
| 2000 | 101 | 102 | 103 | 104 | 105 | 106 | 107 | 108 | 109 | 110 |
| 3000 | 111 | 112 | 113 | 114 | 115 | 116 | 117 | 118 | 119 | 120 |
| 4000 | 121 | 122 | 123 | 124 | 125 | 126 | 127 | 128 | 129 | 130 |
| 5000 | 131 | 132 | 133 | 134 | 135 | 136 | 137 | 138 | 139 | 140 |
| 6000 | 141 | 142 | 143 | 144 | 145 | 146 | 147 | 148 | 149 | 150 |
| 7000 | 151 | 152 | 153 | 154 | 155 | 156 | 157 | 158 | 159 | 160 |
| 8000 | 161 | 162 | 163 | 164 | 165 | 166 | 167 | 168 | 169 | 170 |
| 9000 | 171 | 172 | 173 | 174 | 175 | 176 | 177 | 178 | 179 | 180 |
| 10000 | 181 | 182 | 183 | 184 | 185 | 186 | 187 | 188 | 189 | 190 |
| 20000 | 191 | 192 | 193 | 194 | 195 | 196 | 197 | 198 | 199 | 200 |
| 30000 | 201 | 202 | 203 | 204 | 205 | 206 | 207 | 208 | 209 | 210 |
| 40000 | 211 | 212 | 213 | 214 | 215 | 216 | 217 | 218 | 219 | 220 |
| 50000 | 221 | 222 | 223 | 224 | 225 | 226 | 227 | 228 | 229 | 230 |
| 60000 | 231 | 232 | 233 | 234 | 235 | 236 | 237 | 238 | 239 | 240 |
| 70000 | 241 | 242 | 243 | 244 | 245 | 246 | 247 | 248 | 249 | 250 |
| 80000 | 251 | 252 | 253 | 254 | 255 | 256 | 257 | 258 | 259 | 260 |
| 90000 | 261 | 262 | 263 | 264 | 265 | 266 | 267 | 268 | 269 | 270 |
| 100000 | 271 | 272 | 273 | 274 | 275 | 276 | 277 | 278 | 279 | 280 |

In some embodiments, the viscosity is the value in the column heading and the shear rate is the value in the row heading that together correspond to any one of the cells number 1 to 280 in the table immediately above. In some embodiments, the viscosity and shear rate combinations in the table above are measured by a syringe loading method as described herein.

As used herein, the term “high protein concentration” or “high protein” refers to a protein concentration of greater than about 200 mg/mL. In some embodiments, the protein concentration is greater than about 300 mg/mL. In some embodiments, the protein concentration is greater than about 400 mg/mL. In some embodiments, the protein concentration is greater than about 500 mg/mL. In some embodiments, the protein concentration is greater than about 600 mg/mL. In some embodiments, the protein concentration is greater than about 700 mg/mL. In some embodiments, the protein concentration is greater than about 800 mg/mL. In some embodiments, the protein concentration is greater than about 900 mg/mL. In some embodiments, the protein concentration is greater than about 1000 mg/mL. In some embodiments, the protein concentration is the concentration of one protein species (proteins substantially identical). In some embodiments, the protein concentration is the concentration of all proteins in a mixture. In some embodiments, “high protein concentration” or “high protein” refers to a protein concentration that is greater than about 200, 300, 400, 500, 600, 700, 800, 900, or 1000 mg/mL. In some embodiments “high protein concentration” or “high protein” refers to a protein concentration range, wherein the range is entirely greater than about 200, 300, 400, 500, 600, 700, 800, 900, or 1000 mg/mL.

As used herein, the term “dispersion” has its plain ordinary meaning within the field of Chemistry and refers to a system containing particles dispersed in a continuous phase of a different composition (e.g. nanoparticles or nanoclusters dispersed in a liquid phase). In some embodiments, a dispersion may be a suspension, wherein a suspension has its plain ordinary meaning within Chemistry and refers to a dispersion of solid particles in a continuous liquid phase, wherein the solid particles are large enough for sedimentation. In some embodiments, a dispersion may be a colloid, wherein a colloid has its plain ordinary meaning as used within Chemistry. In some embodiments, a dispersion comprises nanoparticles dispersed in a continuous liquid phase. In some embodiments the dispersed particles are protein nanoclusters. In some embodiments, the continuous phase of a different composition comprises protein in solution. In some embodiments, the protein in solution is less than about 50%, 40%, 30%, 20%, 10%, 9%, 8%, 7%, 6%, 5%, 4%, 3%, 2%, 1%, 0.5%, 0.4%, 0.3%, 0.2%, 0.1%, of the total protein in the dispersion (i.e. particles and continuous phase combined).

The terms “polypeptide,” “peptide” and “protein” are used interchangeably herein to refer to a polymer of amino acid residues, wherein the polymer may optionally be conjugated to a moiety that does not consist of amino acids (e.g. small molecular weight compounds). The terms apply to amino acid polymers in which one or more amino acid residue is an artificial chemical mimetic of a corresponding naturally occurring amino acid, as well as to naturally occurring amino acid polymers and non-naturally occurring amino acid polymer. In some embodiments, a protein comprises a non-protein composition (e.g. low molecular weight compound) conjugated (e.g. bonded) to the polymer of amino acid residues (collectively a “conjugate” or “conjugated protein”). In some embodiments, a protein consists of a polymer of amino acids (a “non-conjugated protein”). In some embodiments, a protein is a polymer of about 10, 20, 30, 40, 50, 60, 70, 80, 90, 100; 200, 300, 400, 500, 600, 700, 800, 900, 1000, 2000, 3000, 4000, or 5000 amino acid residues. In some embodiments, a protein is a polymer of 2, 3, 4, 5, 6, 7, 8, 9, 10, 11, 12, 13, 14, 15, 16, 17, 18, 19, or 20 amino acid residues.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A). Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or percent sequence “identity,” or “shares amino acid sequence identity” in the context of two or more nucleic acids or polypeptide sequences, refer to two or more sequences or subsequences that are the same or have a percentage of amino acide residues or nucleotides that are the same over a specified region, or have a specified percentage of amino acid residues or nucleotides that are the same (i.e., about 60% identity, preferably about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

Aptamers are nucleic acids that are designed to bind to a wide variety of targets in a non-Watson Crick manner. An aptamer can thus be used to detect or otherwise target nearly any molecule of interest, including an autoimmune, inflammatory autoimmune, cancer, infectious disease, or other disease associated protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer.

As used herein, the term “crowder” refers to a compound that, when present in a solvent (e.g. a dispersion liquid) with concentrated proteins, aids formation of a stable colloidal dispersion containing nanoclusters of non-irreversibly aggregated proteins. In some embodiments, a crowder may be the protein itself (e.g. self-crowding protein). In some embodiments, the crowder may be an amino acid. In some embodiments, the crowder may be a second protein species (e.g. a dipeptide, tripeptide, oligopeptide, conjugated protein, non-conjugated protein). In some embodiments, the crowder may be a non-protein crowder such as a polysaccharide, polyelectrolyte, polyacid, dextran, polaxamer, surfactant, a glycerol, an erythritol, an arabinose, a xylose, a ribose, an inositol, a fructose, a galactose, a maltose, a glucose, a mannose, a trehalose, a sucrose, a poly(ethylene glycol), a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt, a buffer or a combination thereof.

The terms “polysaccharide”, “polyelectrolyte”, “polyacid”, “polaxamer”, “surfactant”, “buffer” have their plain ordinary meaning within the field of Chemistry.

As used herein, the term “dextran” refers to a branched polysaccharide comprising glucose molecules. In some embodiments, the dextran has a molecular weight between about one to 2000 kilodaltons. In some embodiments, the dextran is one kilodalton. In some embodiments, the molecular weight is between about one and 10 kilodaltons. In some embodiments, the molecular weight is between about one and 100 kilodaltons. In some embodiments, the molecular weight is between about one and 1000 kilodaltons. In some embodiments, the molecular weight is between about 10 and 100 kilodaltons. In some embodiments, the molecular weight is between about 10 and 50 kilodaltons. In some embodiments, the molecular weight is between about 10 and 2000 kilodaltons. In some embodiments, the molecular weight is between about 100 and 2000 kilodaltons. In some embodiments, the molecular weight is between about 100 and 500 kilodaltons.

As used herein, the term “about”, when modifying a number (e.g. an amount, measurement, size, viscosity, diameter, concentration), refers to a range of values, including the number, and values greater and/or less than the number, wherein the range is an amount that would not affect the function or use of a composition or method, as described herein (including embodiments thereof) when compared to the function applied with exactly the number. In some embodiments the range would not significantly affect the function. In some embodiments, the range would not substantially affect the function. In some embodiments, the composition is a nanocluster as described herein (including embodiments thereof). In some embodiments, the composition is a dispersion of nanoclusters as described herein (including embodiments thereof). In some embodiments, the composition is a pharmaceutical composition, as described herein (including embodiments thereof). In some embodiments, the composition is a kit as described herein (including embodiments thereof). In some embodiments, the method is a method of making a dispersion as described herein (including embodiments thereof). In some embodiments, the method is a method of treating a disease, as described herein (including embodiments thereof). In some embodiments, the method is a method of modifying a dispersion of nanoclusters, as described herein (including embodiments thereof). In some embodiments, about includes 0.1, 0.2, 0.3, 0.4, 0.5, 0.6, 0.7, 0.8, or 0.9 times an increase, decrease, or both, of the number. In some embodiments, about is exactly the number. In some embodiments, about is the standard deviation of the number when measured by a person of ordinary skill in measuring the number, using a common technique or apparatus for taking such measurement. In some embodiments, about includes 0.1, 1.0, or 10 times the number. In some embodiments, about includes plus and minus 0.1 times the number (e.g. about 200 mg/mL is 180-220 mg/mL).

As used herein, the term “average diameter”, when applied to nanoclusters, refers to the average diameter of the nanoclusters in a sample. In some embodiments, the average diameter is an average hydrodynamic diameter. In some embodiments, the average diameter is the average length of the longest axis of the nanocluster. In some embodiments the average diameter is measured by dynamic light scattering. In some embodiments the average diameter is measured by static light scattering. In some embodiments the average diameter is measured by size exclusion chromatography. In some embodiments the average diameter is measured by microscopy. In some embodiments the average diameter is measured by scanning electron microscopy. In some embodiments the average diameter is measured by cryoelectron microscopy. In some embodiments the average diameter is measured by transmission electron microscopy. In some embodiments the average diameter is measured by x-ray scattering (e.g. small angle x-ray scattering).

As used herein, the term “plurality” refers to more than one.

As used herein, the term “irreversibly aggregated” refers to proteins physically associated together in a mixture, comprising a liquid medium, wherein upon dilution of the concentration of the protein or concentration of crowder if the mixture contains crowder, the proteins do not dissociate from the aggregates to form functional protein possessing the secondary, tertiary, and quaternary structure appropriate for the medium and concentration of protein if the protein had not previously been aggregated. An irreversibly aggregated protein may also be termed an “unstable” protein. A “stable” protein (e.g. antibody) is a protein that dissociates from a protein aggregate or protein cluster upon dilution of either the protein concentration or crowder concentration, if a crowder is present and promoted the formation of the protein aggregate or protein cluster, to form functional (e.g. active, enzymatically active) proteins possessing the secondary, tertiary, and quaternary structure appropriate for the medium and concentration of protein if the protein had not previously been aggregated.

As used herein, the term “low molecular weight compound” refers to a composition having a molecular weight less than 1 kilodalton. In some embodiments, the low molecular weight compound may be a diagnostic agent, pharmaceutical agent, contrast agent, fluorophore, paramagnetic agent, peptide, or toxin. In some embodiments, the low molecular weight compound may be conjugated (e.g. bonded) to another composition (e.g. protein, antibody).

As used herein, the term “diagnostic agent” refers to a composition that is useful for detecting the presence of a disease state or a symptom of a disease state. In some embodiments, a diagnostic agent may be a label or detectable moiety.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

As used herein, a “pharmaceutical” refers to a composition that is useful in the treatment of a disease or a symptom of a disease.

As used herein, a “pharmaceutically active protein” refers to a protein that is useful in the treatment of a disease or a symptom of a disease.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein could successfully treat cancer by decreasing the incidence of cancer and or causing remission of cancer. The term “treating,” and conjugations thereof, include prevention of an injury, pathology, condition, or disease.

“Disease” or “condition” refer to a state of being or health status of a patient or subject capable of being treated with the compositions, dispersions, or methods provided herein.

As used herein, the term “contrast agent” refers to a composition that, when administered to a subject, improves the detection limit or detection capability of a method, technique, or apparatus for medical imaging (e.g. radiographic instrument, X-ray, CT, PET, MRI, ultrasound). A contrast agent may enhance the contrast of signals related to different structures or fluids within a subject.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “fluorophore” has its plain ordinary meaning within Chemistry and refers to a chromophore used in fluorescent imaging of spectroscopy. A fluorophore absorbs light within a first range of wavelengths and emits light within a second range of wavelengths.

As used herein, the term “shear rate” has its plain ordinary meaning within Chemistry, and fluid mechanics and refers to the rate (e.g. seconds⁻¹) of application of a shear, wherein shear refers to a force or pressure applied to an object (e.g. deformable object, liquid, solid object) perpendicular to a given axis with greater value (i.e. greater force or pressure) on one side of the axis compared to the other. For flow through a cylinder, shear rate at the wall is proportional to the flow rate divided by the cube of the radius.

As used herein, the term “syringe loading method” refers to a method of measuring the viscosity of a liquid (e.g. dispersion, solution, suspension, mixture) by using the same pressure drop in a needle attached to a syringe wherein the piston of the syringe is displaced by a set amount, causing flow through the needle, wherein the needle has a known diameter and length. The unknown viscosity of liquid being measured is compared to a plurality of measurements conducted in exactly the same way as the current measurement, wherein the plurality of measurements is conducted on liquids with known viscosities. In some embodiments, the needle has a gauge between 21 and 27. In some embodiments, the needle is a 25 gauge needle. In some embodiments, the syringe is a 1 mL syringe. In some embodiments, the needle is 1.5 inches long. In some embodiments, the time to draw the liquid (e.g. dispersion) from a height in a conical vial, wherein the distance from the liquid meniscus to the bottom of the cone is at 0.4 inches, to a height, wherein the distance from the liquid meniscus to the bottom of the cone is at 0.1 inches, corresponding to a volume of 48 microliters, is measured. In some embodiments, a syringe loading method is a method described herein above using the parameters described in Example VI of the present application.

As used herein, the term “packing fraction” refers to a ratio of the volume occupied by a first object or plurality of first objects to the volume of a defined space containing the first object or plurality of objects and a second object or plurality of objects. In some embodiments, a packing fraction is the ratio of the volume of protein within a nanocluster to the volume of the nanocluster. In some embodiments, the packing fraction is the average of a plurality of ratios of the volume of protein within a nanocluster to the volume of the nanocluster.

As used herein, the term “controlled release component” refers to a compound that when combined with a composition as described herein (including embodiments) releases the composition at a controlled rate into a patient. Such compounds include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. In some embodiments, the control release component may be a sustained release, sustained action, extended release, time release, timed release, controlled release, modified release, or continuous release compound. In some embodiments, the compound is degraded by the patient at the site of administration (e.g. subcutaneous, intravenous) or within the digestive tract (e.g. stomach, intestines) if the compound and composition are administered orally. In some embodiments, the controlled release component is a polymer and may be called a “controlled release polymer”.

As used herein, the term “paramagnetic agent” refers to a paramagnetic compound useful in diagnostic imaging methods (e.g. magnetic resonance imaging) as a contrast agent. In some embodiments, the paramagnetic agent comprises gadolinium, iron oxide, iron platinum, or manganese.

As used herein, the term “isotonic” refers to two liquids having the same osmotic pressure. A liquid is isotonic with another if it has the same effective osmotic pressure as the liquid inside the cell across the membrane of a given type of cell. In some embodiments, a dispersion is isotonic with blood. In some embodiments, a dispersion is isotonic with the site of administration of the dispersion in a patient. In some embodiments, a dispersion is isotonic with a subcutaneous site of administration of the dispersion.

As used herein, the term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of a protein. Such effector moieties include, but are not limited to, an anti-tumor drug, a toxin, a radioactive agent, a cytokine, a second antibody or an enzyme. In some embodiments, the antibody of the invention is linked to an enzyme that converts a prodrug into a cytotoxic agent.

The immunoconjugate can be used for targeting the effector moiety to a target molecule or target molecule positive cell. Such differences can be readily apparent when viewing the bands of gels with approximately similarly loaded with test and controls samples. Examples of cytotoxic agents (e.g. toxins) include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Protein levels can be detected using antibodies or antibody fragments specific for that protein, natural ligands, small molecules, aptamers, etc.

Antibody based techniques are known in the art, and described, e.g., in Harlow & Lane (1988) Antibodies: A Laboratory Manual and Harlow (1998) Using Antibodies: A Laboratory Manual; Wild, The Immunoassay Handbook, 3d edition (2005) and Law, Immunoassay: A Practical Guide (1996). The assay can be directed to detection of a molecular target (e.g., protein or antigen), or a cell, tissue, biological sample, liquid sample or surface suspected of carrying an antibody or antibody target.

A non-exhaustive list of immunoassays includes: competitive and non-competitive formats, enzyme linked immunosorption assays (ELISA), microspot assays, Western blots, gel filtration and chromatography, immunochromatography, immunohistochemistry, flow cytometry or fluorescence activated cell sorting (FACS), microarrays, and more. Such techniques can also be used in situ, ex vivo, in vitro, or in vivo, e.g., for diagnostic imaging.

As used herein, the term “protein-crowder liquid combination” refers to a liquid mixture including a plurality of a protein and a plurality of a crowder. In some embodiments, the protein-crowder liquid combination is a dispersion of protein nanoclusters. In some embodiments, the protein-crowder liquid combination is a dispersion of nanoclusters comprising a plurality of proteins and a plurality of crowder. In some embodiments, the protein-crowder liquid combination is a suspension of nanoclusters comprising a plurality of protein. In some embodiments, the protein-crowder liquid combination is a solution comprising a plurality of protein and a plurality of crowder.

As used herein, the term “protein-crowder mixture” refers to a mixture of protein and crowder, which may optionally include additional components or compounds. In some embodiments, a “protein-crowder mixture” is a “protein-crowder liquid combination”.

As used herein, the term “dispersion liquid” refers to the continuous liquid mixture of a dispersion. In some embodiments, a dispersion liquid is a liquid solution in which protein nanoclusters are dispersed. In some embodiments, a dispersion liquid is a non-aqueous liquid in which protein nanoclusters are dispersed. In some embodiments, a dispersion liquid is an aqueous liquid in which protein nanoclusters are dispersed.

As used herein, the term “cryogenic agent” refers to a composition having a temperature below −150 degrees Celsius. In some embodiments, a cryogenic agent is liquid nitrogen. In some embodiments, a cryogenic agent is liquid helium.

As used herein, the term “centrifugal filtration” refers to the process of filtering or separating components in a mixture by flowing one or more, but not all, components of the mixture through a filter, wherein the components are moved through the filter by centrifugal force. In some embodiments, the mixture and filter are spun in a centrifuge. In some embodiments the filtration is carried out in an Amicon, Microcon, or Centricon device (available from Millipore).

As used herein, the term “tangential flow filtration” refers to a method of filtration wherein the majority of movement of the liquid mixture, prior to passing through the filter, is tangential to the surface of the filter. The term “crossflow filtration” may be used interchangeably with “tangential flow filtration”.

As used herein, the term “protein solution” refers to a mixture of a plurality of protein in a liquid medium (e.g. water, buffer), wherein the protein does not form nanoclusters having an average diameter of 20 to 1000 nm. In some embodiments, a protein solution includes proteins having a quaternary state appropriate for the dissociate constant of the protein and concentration of protein mixed in the liquid, without forming clusters of 10 or more proteins. In some embodiments, a protein dispersion may comprise a dispersion of protein nanoclusters in a protein solution.

As used herein, the term “thin film freezing” refers to a method comprising freezing a liquid on a cooled solid surface, wherein the liquid forms a thin film on the surface of thickness less than 500 micrometers and a surface area to volume ratio between 25 and 500 cm⁻¹. In some embodiments, the liquid and surface have temperatures differing by about 30 degrees Celsius or more. The liquid may be delivered to the cooled solid surface as droplets. In some embodiments, the droplets freeze within 50, 75, 100, 125, 150, 175, 200, 250, 500, 1,000, or 2,000 milliseconds of contacting the cooled solid surface. In some embodiments, the droplets may have an average diameter of 0.1 mm to 5 mm at room temperature. In some embodiments, the droplets will have a cooling rate of between 50 and 250 K/second. The cooled solid surface may be the interior surface of a vial, a belt, platen, plate, roller, platter, or converyor surface.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

### II. COMPOSITIONS

In a first aspect, a transparent, low viscosity, high protein concentration dispersion is provided. The dispersion includes a plurality of nanoclusters. Each of the plurality of nanoclusters includes a plurality of proteins and each of the plurality of proteins shares amino acid sequence identity.

In some embodiments, the plurality of proteins shares complete amino acid sequence identity. In some embodiments, the plurality of proteins are substantially identical. In some embodiments, the plurality of proteins are about 75% identical. In some embodiments, the plurality of proteins are about 80% identical. In some embodiments, the plurality of proteins are about 85% identical. In some embodiments, the plurality of proteins are about 90% identical. In some embodiments, the plurality of proteins are about 95% identical. In some embodiments, the plurality of proteins are about 96% identical. In some embodiments, the plurality of proteins are about 75% identical. In some embodiments, the plurality of proteins are about 97% identical. In some embodiments, the plurality of proteins are about 98% identical. In some embodiments, the plurality of proteins are about 99% identical. In some embodiments, the plurality of proteins are about 99.5% identical. In some embodiments, the plurality of proteins are about 99.6% identical. In some embodiments, the plurality of proteins are about 99.7% identical. In some embodiments, 0.5 the plurality of proteins are about 99.8% identical. In some embodiments, the plurality of proteins are about 99.9% identical. In some embodiments, the plurality of proteins are the identical except for drift in the sequence attributable to mistakes in transcription or translation. In some embodiments, the plurality of nanoclusters includes a mixture of proteins with different amino acid sequences.

In some embodiments of the dispersion, each of the plurality of nanoclusters has an average diameter between about 20 and about 1,000 nanometers. In some embodiments of the dispersion, the average diameter is an average hydrodynamic diameter. In some embodiments of the dispersion, the average diameter is an average of the longest dimension of the plurality of nanoclusters. In some embodiments of the dispersion, less than 5% of the plurality of proteins in the plurality of nanoclusters are irreversibly aggregated. In some embodiments of the dispersion, less than 2% of the plurality of proteins in the plurality of nanoclusters are irreversibly aggregated. In some embodiments of the dispersion, less than 1% of the plurality of proteins in the plurality of nanoclusters are irreversibly aggregated.

In some embodiments, the viscosity of the dispersion is between about 1 centipoise and about 1000 centipoise (e.g. between 1 centipoise and 1000 centipoise). In some embodiments, the viscosity of the dispersion is between about 1 centipoise and about 500 centipoise (e.g. between 1 centipoise and 500 centipoise). In some embodiments, the viscosity of the dispersion is between about 1 centipoise and about 250 centipoise (e.g. between 1 centipoise and 250 centipoise). In some embodiments, the viscosity of the dispersion is between about 1 centipoise and about 100 centipoise. In some embodiments, the viscosity of the dispersion is between about 1 centipoise and about 90 centipoise (e.g. between 1 centipoise and 90 centipoise). In some embodiments, the viscosity of the dispersion is between about 1 centipoise and about 80 centipoise (e.g. between 1 centipoise and 80 centipoise). In some embodiments, the viscosity of the dispersion is between about 1 centipoise and about 70 centipoise (e.g. between 1 centipoise and 70 centipoise). In some embodiments, the viscosity of the dispersion is between about 1 centipoise and about 60 centipoise (e.g. between 1 centipoise and 60 centipoise). In some embodiments, the viscosity of the dispersion is between about 1 centipoise and about 50 centipoise (e.g. between 1 centipoise and 50 centipoise). In some embodiments, the viscosity of the dispersion is between about 1 centipoise and about 40 centipoise (e.g. between 1 centipoise and 40 centipoise). In some embodiments, viscosity (e.g. of a dispersion) is measured by a syringe loading method. In some embodiments, viscosity (e.g. of a dispersion) is measured with a viscometer (e.g. Stormer viscometer, vibrating viscometer, rotating viscometer, Marsh funnel viscometer, U-tube viscometer, falling sphere viscometer, falling piston viscometer, oscillating piston viscometer, Stabinger viscometer, bubble viscometer, or Cannon-Fenske viscometer). In some embodiments, viscosity (e.g. of a dispersion) is measured with a rheometer. In some embodiments, viscosity (e.g. of a dispersion) is measured with a Zahn cup. In some embodiments, viscosity (e.g. of a dispersion) is measured with a Ford viscosity cup. In some embodiments, viscosity (e.g. of a dispersion) is measured with a syringe (e.g. a syringe equipped with a needle having a size between 21 gauge and 27 gauge, or a 25 gauge needle, or a 1.5 inch long needle, or a 25 gauge 1.5 inch long needle). In some embodiments, viscosity (e.g. of a dispersion) is measured with a plastometer. In some embodiments, the viscosity of the dispersion is about 50 centipoise and the shear rate of the dispersion is about 1000 second⁻¹ (e.g. 50 centipoise and 1000 second⁻¹). In some embodiments, the viscosity of the dispersion is between about 25 centipoise and about 75 centipoise and the shear rate of the dispersion is about 1000 second⁻¹ (e.g. between 25 centipoise and 75 centipoise and 1000 second⁻¹). In some embodiments, the viscosity of the dispersion is between about 10 centipoise and about 90 centipoise and the shear rate of the dispersion is about 1000 second⁻¹ (e.g. between 10 centipoise and 90 centipoise and 1000 second⁻¹). In some embodiments, the viscosity of the dispersion is about 50 centipoise and the shear rate of the dispersion is between about 100 second⁻¹ and about 50000 second⁻¹ (e.g. 50 centipoise and between 100 second⁻¹ and 50000 second⁻¹). In some embodiments, the viscosity of the dispersion is between about 25 centipoise and 75 centipoise and the shear rate of the dispersion is between about 100 second⁻¹ and about 50000 second⁻¹ (e.g. between 25 centipoise and 75 centipoise and between 100 second⁻¹ and 50000 second⁻¹). In some embodiments, the viscosity of the dispersion is between about 25 centipoise and 75 centipoise and the shear rate of the dispersion is between about 1000 second⁻¹ and about 10000 second⁻¹ (e.g. between 25 centipoise and 75 centipoise and between 1000 second⁻¹ and 10000 second⁻¹). In some embodiments, the dispersion is syringeable and wherein an aqueous solution of the plurality of proteins at an identical concentration is not syringeable. In some embodiments, the dispersion has a viscosity about two fold lower than the viscosity of an aqueous solution of the plurality of proteins at an identical concentration (e.g. 1.6 fold, 1.7 fold, 1.8 fold, 1.9 fold, 2 fold, 2.1 fold, 2.2 fold, 2.3 fold, 2.4 fold). In some embodiments, the dispersion has a viscosity about five fold lower than the viscosity of an aqueous solution of the plurality of proteins at an identical concentration (e.g. 4.6 fold, 4.7 fold, 4.8 fold, 4.9 fold, 5 fold, 5.1 fold, 5.2 fold, 5.3 fold, 5.4 fold). In some embodiments, the dispersion has a viscosity about ten fold lower than the viscosity of an aqueous solution of the plurality of proteins at an identical concentration (e.g. 9.6 fold, 9.7 fold, 9.8 fold, 9.9 fold, 10 fold, 10.1 fold, 10.2 fold, 10.3 fold, 10.4 fold).

In some embodiments, the viscosity of the dispersion is between about the two viscosity values corresponding to any one of the cells in the table/matrix immediately below having number 1 to 240 (i.e. one viscosity for column and one viscosity for row), wherein between includes either of the two viscosity values):

| Viscosity | viscosity (cP) | | | | | | | | | |
|---|---|---|---|---|---|---|---|---|---|---|
| (cP) | 90 | 80 | 70 | 60 | 50 | 40 | 30 | 20 | 10 | 1 |
| 100 | 1 | 2 | 3 | 4 | 5 | 6 | 7 | 8 | 9 | 10 |
| 95 | 11 | 12 | 13 | 14 | 15 | 16 | 17 | 18 | 19 | 20 |
| 90 | 21 | 22 | 23 | 24 | 25 | 26 | 27 | 28 | 29 | 30 |
| 85 | 31 | 32 | 33 | 34 | 35 | 36 | 37 | 38 | 39 | 40 |
| 80 | 41 | 42 | 43 | 44 | 45 | 46 | 47 | 48 | 49 | 50 |
| 75 | 51 | 52 | 53 | 54 | 55 | 56 | 57 | 58 | 59 | 60 |
| 70 | 61 | 62 | 63 | 64 | 65 | 66 | 67 | 68 | 69 | 70 |
| 65 | 71 | 72 | 73 | 74 | 75 | 76 | 77 | 78 | 79 | 80 |
| 60 | 81 | 82 | 83 | 84 | 85 | 86 | 87 | 88 | 89 | 90 |
| 55 | 91 | 92 | 93 | 94 | 95 | 96 | 97 | 98 | 99 | 100 |
| 50 | 101 | 102 | 103 | 104 | 105 | 106 | 107 | 108 | 109 | 110 |
| 45 | 111 | 112 | 113 | 114 | 115 | 116 | 117 | 118 | 119 | 120 |
| 40 | 121 | 122 | 123 | 124 | 125 | 126 | 127 | 128 | 129 | 130 |
| 35 | 131 | 132 | 133 | 134 | 135 | 136 | 137 | 138 | 139 | 140 |
| 30 | 141 | 142 | 143 | 144 | 145 | 146 | 147 | 148 | 149 | 150 |
| 25 | 151 | 152 | 153 | 154 | 155 | 156 | 157 | 158 | 159 | 160 |
| 20 | 161 | 162 | 163 | 164 | 165 | 166 | 167 | 168 | 169 | 170 |
| 15 | 171 | 172 | 173 | 174 | 175 | 176 | 177 | 178 | 179 | 180 |
| 10 | 181 | 182 | 183 | 184 | 185 | 186 | 187 | 188 | 189 | 190 |
| 9 | 191 | 192 | 193 | 194 | 195 | 196 | 197 | 198 | 199 | 200 |
| 8 | 201 | 202 | 203 | 204 | 205 | 206 | 207 | 208 | 209 | 210 |
| 7 | 211 | 212 | 213 | 214 | 215 | 216 | 217 | 218 | 219 | 220 |
| 6 | 221 | 222 | 223 | 224 | 225 | 226 | 227 | 228 | 229 | 230 |
| 5 | 231 | 232 | 233 | 234 | 235 | 236 | 237 | 238 | 239 | 240 |

In some embodiments, the dispersion includes between about 200 mg/mL and about 600 mg/mL of the protein (e.g. between 200 mg/mL and 600 mg/mL). In some embodiments, the dispersion includes between about 200 mg/mL and about 400 mg/mL of the protein (e.g. between 200 mg/mL and 400 mg/mL). In some embodiments, the dispersion includes between about 200 mg/mL and about 300 mg/mL of the protein (e.g. between 200 mg/mL and 300 mg/mL). In some embodiments, the dispersion includes between about 200 mg/mL and about 250 mg/mL of the protein (e.g. between 200 mg/mL and 250 mg/mL). In some embodiments, the dispersion includes greater than about 200 mg/mL of the protein (e.g. greater than 200 mg/mL). In some embodiments, the dispersion includes greater than about 300 mg/mL of the protein (e.g. greater than 300 mg/mL). In some embodiments, the dispersion includes greater than about 400 mg/mL of the protein (e.g. greater than 400 mg/mL). In some embodiments, the dispersion includes greater than about 500 mg/mL of the protein (e.g. greater than 500 mg/mL). In some embodiments, the dispersion includes greater than about 600 mg/mL of the protein (e.g. greater than 600 mg/mL).

In some embodiments, the dispersion includes a light extinction measurement less than about 0.05, about 0.1, about 0.25, or about 0.5 cm⁻¹, wherein the light extinction measurement includes an average light extinction over wavelengths between 400 nm and 700 nm (e.g. less than 0.05, 0.1, 0.25, or 0.5 cm⁻¹). In some embodiments, the dispersion includes a light extinction measurement less than about 0.05, about 0.1, about 0.25, or about 0.5 cm⁻¹, wherein the light extinction measurement is made at a wavelength of 600 nm (e.g. less than 0.05, 0.1, 0.25, or 0.5 cm⁻¹). In some embodiments, the dispersion includes a light extinction measurement less than about 0.05, about 0.1, about 0.25, or about 0.5 cm⁻¹, wherein the light extinction measurement is made at a wavelength of between 400 nm and 700 nm (e.g. less than 0.05, 0.1, 0.25, or 0.5 cm⁻¹, and at a wavelength of 400, 450, 500, 550, 600, 650, or 700 nm or any other intervening wavelength).

In some embodiments of the dispersion, the plurality of nanoclusters have an average diameter between about 20 nanometers and about 800 nanometers (e.g. between 20 nanometers and 800 nanometers). In some embodiments of the dispersion, the plurality of nanoclusters have an average diameter between about 20 nanometers and about 600 nanometers (e.g. between 20 nanometers and 600 nanometers). In some embodiments of the dispersion, the plurality of nanoclusters have an average diameter between about 20 nanometers and about 400 nanometers (e.g. between 20 nanometers and 400 nanometers). In some embodiments of the dispersion, the plurality of nanoclusters have an average diameter between about 20 nanometers and about 200 nanometers (e.g. between 20 nanometers and 200 nanometers). In some embodiments of the dispersion, the plurality of nanoclusters have an average diameter between about 20 nanometers and about 100 nanometers (e.g. between 20 nanometers and 100 nanometers). In some embodiments of the dispersion, the plurality of nanoclusters have an average diameter between about 20 nanometers and about 75 nanometers (e.g. between 20 nanometers and 75 nanometers). In some embodiments of the dispersion, the plurality of nanoclusters have an average diameter between about 20 nanometers and about 50 nanometers (e.g. between 20 nanometers and 50 nanometers).

In some embodiments of the dispersion, the plurality of nanoclusters have an average packing fraction between about 30% and about 80% (e.g. between 30% and 80%). In some embodiments of the dispersion, the plurality of nanoclusters have an average packing fraction between about 30% and about 70% (e.g. between 30% and 70%). In some embodiments of the dispersion, the plurality of nanoclusters have an average packing fraction between about 30% and about 60% (e.g. between 30% and 60%). In some embodiments of the dispersion, the plurality of nanoclusters have an average packing fraction between about 30% and about 50% (e.g. between 30% and 50%). In some embodiments of the dispersion, the plurality of nanoclusters have an average packing fraction between about 50% and about 60% (e.g. between 50% and 60%). In some embodiments of the dispersion, the plurality of nanoclusters have an average packing fraction between about 60% and about 74% (e.g. between 60% and 74%).

In some embodiments, the dispersion includes a crowder. In some embodiments, the crowder is a monosaccharide. In some embodiments, the crowder is a monosaccharide selected from glucose, mannose, fructose, arabinose, xylose, ribose, and galactose. In some embodiments, the crowder is a disaccharide. In some embodiments, the crowder is a disaccharide selected from trehalose, lactulose, lactose, cellobiose, maltose, or sucrose. In some embodiments, the crowder is a polysaccharide. In some embodiments, the crowder is a polyelectrolyte. In some embodiments, the crowder is a polyacid. In some embodiments, the crowder is a poly(ethylene glycol). In some embodiments, the crowder is a poly(ethylene glycol) with a molecular weight between PEG 200 and PEG 5000. In some embodiments, the crowder is a salt. In some embodiments, the crowder is a dextran. In some embodiments, the crowder is a polaxamer. In some embodiments, the crowder is an alcohol. In some embodiments, the crowder is an amino acid or protein. In some embodiments, the crowder is a dipeptide, tripeptide, four amino acid peptide, five amino acid peptide, or oligopeptide. In some embodiments, the crowder is a conjugated protein. In some embodiments, the crowder is a non-conjugated protein. In some embodiments, the crowder is a non-protein crowder. In some embodiments, the crowder is a surfactant. In some embodiments, the dispersion includes a crowder selected from the group consisting of a trehalose, a poly(ethylene glycol), ethanol, N-methyl-2-pyrrolidone (NMP), a buffer, or a combination thereof. In some embodiments, the dispersion includes about a 1:1 weight ratio of protein to a crowder (e.g. a 1:1 weight ratio). In some embodiments, the dispersion includes about a 2:1 weight ratio of protein to a crowder (e.g. a 2:1 weight ratio). In some embodiments, the dispersion includes about a 3:1 weight ratio of protein to a crowder (e.g. a 3:1 weight ratio). In some embodiments, the dispersion includes about a 4:1 weight ratio of protein to a crowder (e.g. a 4:1 weight ratio). In some embodiments, the dispersion includes about a 5:1 weight ratio of protein to a crowder (e.g. a 5:1 weight ratio). In some embodiments, the dispersion includes about a 6:1 weight ratio of protein to a crowder (e.g. a 6:1 weight ratio). In some embodiments, the dispersion includes about a 10:1 weight ratio of protein to a crowder (e.g. a 10:1 weight ratio). In some embodiments, the dispersion includes about a 1:2 weight ratio of protein to a crowder (e.g. a 1:2 weight ratio). In some embodiments, the dispersion includes about a 1:3 weight ratio of protein to a crowder (e.g. a 1:3 weight ratio). In some embodiments, the dispersion includes about a 1:4 weight ratio of protein to a crowder (e.g. a 1:4 weight ratio). In some embodiments, the dispersion includes about a 1:5 weight ratio of protein to a crowder (e.g. a 1:5 weight ratio). In some embodiments, the dispersion includes about a 1:10 weight ratio of protein to a crowder (e.g. a 1:10 weight ratio).

In some embodiments of the dispersion, the pH of the dispersion is at about the isoelectric point of the plurality of proteins (e.g. is at the isoelectric point). In some embodiments of the dispersion, the pH of the dispersion is less than about 2.5, 2.0, 1.5, 1.0, 0.8, 0.75, 0.5, 0.3, 0.2, 0.1, or 0.05 pH units different from the isoelectric point of the plurality of proteins (e.g. less than 2.5, 2.4, 2.3, 2.2, 2.1, 2.0, 1.9, 1.8, 1.7, 1.6, 1.5, 1.4, 1.3, 1.2, 1.1, 1.0, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02, or 0.01 pH units). In some embodiments, the pH of the dispersion is about 4, 4.5, 5, 5.5, 6, 6.5, 7, 7.5, 8, 8.5, 9, or 9.5. In some embodiments, the dispersion is isotonic with human blood. In some embodiments, the dispersion is hypotonic with human blood. In some embodiments, the dispersion has an osmolarity of about 300 mOsmo/L (e.g. 300 mOsmo/L). In some embodiments, the dispersion has an osmolarity of between about 250 mOsmo/L and about 350 mOsmol/L (e.g. 250 mOsmo/L and 350 mOsmol/L). In some embodiments, the dispersion has an osmolarity of between about 150 mOsmo/L and about 450 mOsmol/L (e.g. 150 mOsmo/L and 450 mOsmol/L). In some embodiments, the dispersion has an osmolarity of between about 150 mOsmo/L and about 600 mOsmol/L (e.g. between 150 mOsmo/L and 600 mOsmol/L). In some embodiments of the dispersion, each of the plurality of proteins is an antibody, an antibody fragment, a pegylated protein, a lipidated protein, a growth factor or growth factor antagonist, a cytokine or cytokine antagonist, a receptor or receptor antagonist, an antigen, a vaccine, or an anti-inflammatory agent. In some embodiments of the dispersion, the plurality of proteins is a plurality of conjugates, wherein each of the conjugates is a protein bonded to low molecular weight compound, wherein the low molecular weight compound is a diagnostic agent, a pharmaceutical agent, a contrast agent, a fluorophore, a radioisotope, a toxin, a paramagnetic agent, or an aptamer. In some embodiments of the dispersion, the plurality of proteins is self-crowding. In some embodiments of the dispersion, the plurality of proteins is not a plurality of conjugates and each of the proteins consists of amino acids (i.e. non-conjugated protein).

In some embodiments, the plurality of nanoclusters include multiple different protein species. In some embodiments of the dispersion, the plurality of nanoclusters is a first plurality of nanoclusters and the plurality of proteins is a first plurality of proteins, the dispersion further includes a second plurality of nanoclusters wherein each of the second plurality of nanoclusters includes a second plurality of proteins and each of the second plurality of proteins shares amino acid sequence identity, and the second plurality of proteins is different from the first plurality of proteins. In some embodiments of the dispersion, the plurality of nanoclusters further includes a controlled release polymer. In some embodiments of the dispersion, the plurality of nanoclusters further includes a controlled release component. In some embodiments of the dispersion, each of the plurality of nanoclusters further includes a low molecular weight compound and the low molecular weight compound is a diagnostic agent, a pharmaceutical agent, a contrast agent, a fluorophore, a radioisotope, a toxin, a paramagnetic agent, a metal, a metal oxide, or an aptamer. In some embodiments of the dispersion, the dispersion further includes a plurality of nanoparticles. In some embodiments of the dispersion, the plurality of nanoparticles include a plurality of a compound selected from Au, a magnetic agent, an optical agent, a diagnostic agent, a pharmaceutical agent, a contrast agent, a fluorophore, a radioisotope, a toxin, a paramagnetic agent, a metal, a metal oxide, or an aptamer.

In a second aspect a pharmaceutical composition is provided, including any of the dispersions as described herein (including embodiments), wherein the plurality of proteins is a plurality of pharmaceutically active proteins. In some embodiments, the pharmaceutical composition is within a syringe attached to a 21 to 27 gauge needle. In some embodiments, pharmaceutical composition is within an osmotic pump. In some embodiments, the pharmaceutical composition is within a controlled release component, liposome, or microsphere.

In a third aspect a kit is provided, wherein the kit includes a dispersion or pharmaceutical composition described herein (including embodiments). In some embodiments, the kit includes instructions for using the included dispersion or pharmaceutical composition. In some embodiments, the kit includes a vessel containing a dispersion or pharmaceutical composition as described herein (including embodiments).

In a further aspect a kit is provided, wherein the kit includes protein in powder form or a protein-crowder mixture in powder form, and a dispersion liquid. In some embodiments, the kit may be used in a method of making a dispersion or pharmaceutical composition described herein (including embodiments). In some embodiments, the kit includes instructions for making a dispersion as described herein (including embodiments). In some embodiments, the kit includes protein in powder form and a dispersion liquid. In some embodiments, the kit includes a protein-crowder mixture in powder form and a dispersion liquid. In some embodiments, the kit includes a syringe and a needle. In some embodiments, the kit includes instructions for mixing the protein in powder form or protein-crowder mixture in powder form with the dispersion liquid. In some embodiments, the kit includes instructions for mixing the protein in powder form or protein-crowder mixture in powder form with the dispersion liquid and self-administering the resulting dispersion.

The present invention discloses a novel composition including a dispersion of submicron antibody particles and a method of making the same. A composition described herein is substantially transparent and allows for subcutaneous injection of highly concentrated antibody (˜200 mg/ml). A solution of monoclonal antibody (for example, 1B7) was rapidly frozen and lyophilized using a novel spiral-wound in situ freezing technology technique (SWIFT) to generate amorphous particles. Upon gentle stirring a transparent dispersion of protein formed rapidly in buffer containing one or more pharmaceutically acceptable crowding agents, trehalose, polyethylene glycol and n-methyl-2-pyrrolidone (NMP). Formulation near an antibody isoelectric point minimizes the charge per molecule, such that the attractive forces were sufficient to form large particles, specifically clusters composed of protein molecules (˜200 nm diameter), with a low apparent viscosity (˜24 cp).

In some embodiments, within each particle, there are no detectable changes in antibody tertiary structure, as the protein native state is stabilized by self-crowding of the protein, limiting unfolding and aggregation. In some embodiments, upon in vitro dilution of the dispersion, the particles revert to monomeric protein with full activity, as monitored by dynamic light scattering and ELISA. In some embodiments, when administered to mice as an intravenous solution, subcutaneous solution or subcutaneous dispersion at similar doses (4.6-7.3 mg/kg), the distribution and elimination kinetics were similar. In some embodiments, a dispersion formulation makes ultra-high dosages possible (51.6 mg/kg); this also exhibited a similar pharmacokinetic profile. Moreover, analysis of the terminal serum samples by in vitro binding and cellular neutralization assays indicates antibody delivered as a sub-cutaneous dispersion retains full activity over the 14-day study period.

A method of generating high-concentration, low-viscosity dispersions of submicron antibody particles as described herein is readily generalizable and could lead to improved administration and patient compliance, providing new opportunities for the biotechnology industry.

Monoclonal antibodies continue to command a large market share with numerous entities in clinical trials for a variety of therapeutic indications. These monoclonal antibodies have generated considerable interest as therapeutics because they specifically target distinct antigens with favorable pharmacokinetic, production, and safety profiles. Currently, 28 monoclonal antibodies have received FDA-approval for treatment of a wide variety of diseases, commanding an annual market size of over $20 billion dollars. In many cases, the doses required for therapeutic efficacy are large, limiting options for antibody delivery and administration. Despite advances in protein drug development which allow tailoring of key biophysical properties, such as solubility, stability, and binding affinity (Maynard, Maassen et al.) via recombinant DNA techniques, few options have been developed to deliver these macromolecules at desired dosages (>2 mg antibody/kg body weight). Typically, large volumes of dilute protein solutions are delivered intravenously to avoid the chemical and physical destabilization and resulting loss in protein activity associated with high concentration formulations. Self-administered subcutaneous injections offer several major advantages over intravenous infusion, including increased accessibility and patient compliance, along with reduced pain and cost. However, the required therapeutic dosages would indicate protein concentrations in excess of 100 mg/ml, given the maximum subcutaneous injection volume of 1.5 ml.

Formulation of therapeutic proteins at these high concentrations is intrinsically difficult, demanding solutions customized for each new product. Frequently, formulation at high concentrations is not possible due to low protein solubility, protein instability and high solution viscosity resulting from short-range attractive protein-protein interactions. These interactions, which include hydrophobic interactions, hydrogen bonds and fluctuating charge dipoles, act over distances up to ˜1 nm. At high protein concentrations (over 150 mg/ml), the average separation distance between individual antibody molecules is reduced to less than 10 nm. (Miller, 2011) Thus, the probability that two protein molecules will be less than 1 nm apart is high and the effect of the short-range attractive interactions between protein molecules becomes significant. This leads to the concentration-dependent formation of reversible and irreversible aggregates with potential adverse effects on protein activity, pharmacokinetics and immunogenicity. Most troubling are irreversible aggregates, high molecular weight aggregates comprised of monomers with altered native structure and reduced activity, which can result in a turbid solution or precipitation. The formation of these aggregates is highly protein specific and can be formed through physical mechanisms, via partially unfolded monomers with exposed hydrophobic residues or through chemical mechanisms, via formation of intermolecular bonds mediated by reactive thiols on cysteine or methionine residues.

Protein structure and activity in low viscosity formulations can be preserved at high protein concentrations by minimizing the effects of these short-range interactions. For example, concentrated suspensions of protein microparticles in water-insoluble organic solvents and aqueous suspensions of protein crystals with low viscosity have been reported. These formulations succeed by using micron-sized (5-20 μm) particles of proteins as opposed to protein monomers, thus increasing the average distance between protein particles for a given protein concentration. However, formulations of proteins in organic solvents may not be patient-friendly as they require large-bore needles and can result in additional side effects such as redness and swelling at the injection site. In addition, while highly concentrated aqueous suspensions of crystalline insulin have a history of clinical use, it is challenging to routinely crystallize large protein molecules such as immunoglobulins due to their high molecular weight, surface oligosaccharides, and high degree of segmental flexibility. Similarly, controlled release formulations in which proteins are encapsulated in polymeric matrices with non-aqueous or aqueous media have also been explored. In these cases, the low loadings of protein within the particle (˜15-20 mass %) often result in a low deliverable dose even at high particle volume fractions. Moreover, most polymeric delivery systems suffer from challenges with sterility, protein stability, incomplete protein release, and increased immunogenicity.

The present inventors have previously reported a novel approach to preserve protein activity at high concentrations while achieving a low viscosity, in the form of concentrated dispersions of amorphous protein nanoclusters. (Miller 2011) The addition of trehalose as a “crowder” molecule occupies a large volume and increases the short-range protein-protein attractive interactions. (Miller, 2011) Consequently, most of the protein molecules are concentrated into densely packed equilibrium nanoclusters. (Miller, 2011) The mechanisms of nanocluster formation and stabilization were explained in terms of the specific short-ranged attraction, van der Waals and depletion attraction balanced against weak electrostatic repulsion. The weak electrostatic repulsion was accomplished by, formulation near the protein isoelectric point (pI) where the protein was only slightly charged. Simultaneously, the nanoclusters do not aggregate, since their large size reduces the impact of the short range attractive interactions between nanoclusters. Furthermore, the electrostatic repulsion increases from the cumulative effect of hundreds to thousands of slightly charged protein monomers. (Miller, 2011) This hierarchy of attractive and repulsive interactions results in a colloidally stable protein nanocluster dispersion with low viscosity. In addition, the high volume fraction of the protein within the nanocluster, much higher than is possible with a protein solution, maintains the protein native structure due to a self-crowding, entropic stabilizing mechanism. (Shen, Cheung et al. 2006) To date, only a single extrinisic crowder, trehalose, has been reported for formation of nanoclusters of a therapeutic protein and the pharmacokinetics of that formulation. (Miller, 2011)

The present invention includes description of a multicomponent mixture of three crowding agents that may be used to create stable dispersions of highly concentrated, active monoclonal antibody particles, which retain high activity and bioavailability upon subcutaneous administration in mice. Multicomponent crowding agent mixtures provide flexibility in formulation in response to specific biochemical characteristics of a particular protein such as high protein solubility.

The present invention discloses a novel composition comprising a dispersion of submicron antibody particles and a method of making the same. A composition described herein is substantially transparent and allows for subcutaneous injection of highly concentrated antibody (˜200 mg/ml). A solution of monoclonal antibody (for example, 1B7) was rapidly frozen and lyophilized using a novel spiral-wound in situ freezing technology technique (SWIFT) to generate amorphous particles. Upon gentle stirring, a transparent dispersion of protein formed rapidly in buffer containing one or more pharmaceutically acceptable crowding agents, trehalose, polyethylene glycol and n-methyl-2-pyrrolidone (NMP). Formulation near the antibody isoelectric point minimizes the charge per molecule, such that the attractive forces were sufficient to form large particles, specifically clusters composed of protein molecules (˜200 nm diameter), with a low apparent viscosity (˜24 cp).

Two submicron antibody particle formulations were prepared as examples of the novel low viscosity high concentration protein dispersions of the present invention: (i) a polyclonal sheep IgG dispersion comprising amorphous protein particles generated by traditional tray freezing lyophilization and (ii) a murine IgG2a monoclonal antibody 1B7 comprising amorphous protein particles generated via a new freezing method, spiral-wound in situ freezing technique (SWIFT).

Combined Diagnostics and Therapy (Theranostics)

In some embodiments, the compositions as described herein (including embodiments) (e.g. protein dispersions) may comprise a pharmaceutical and a diagnostic agent. The term theranostics is commonly used to describe a single composition comprising both a therapeutic and diagnostic agent. The synergy between treatment and monitoring or diagnostics may be useful for targeting the treatment more effectively and for selecting the proper dosage. In some embodiments, the composition may comprise a high dosage of a protein therapeutic and a high but non-toxic amount of a diagnostic agent (e.g. imaging agent, contrast agent). The imaging agent may be chemically attached to the protein (e.g. a conjugate) or it may be dispersed with the protein. In some embodiments, the imaging agent may itself be a nanoparticle, for example Au for optical imaging or iron oxide for magnetic imaging. In some embodiments, the nanoparticle may be chemically attached to the protein in the nanocluster, or the nanocluster may comprise a non-conjugated protein and a diagnostic agent or a nanoparticle comprising the diagnostic agent. In some embodiments of the compositions described herein, a dispersion comprises a plurality of protein nanoclusters and a plurality of diagnostic agent (e.g. Au, contrast agent, paramagnetic agent, magnetic, optical agents) nanoparticles.

With the use of magnetic nanoparticles, magnetic imaging methods like MRI may be used in conjugation with the therapeutic functionality of the protein. With the use of nanoparticles useful in optical techniques, methods such as photoacoustic imaging, fluorescence imaging, or optical coherence tomography, may be used in conjugation with the therapeutic functionality of the protein. In some embodiments of the compositions or methods described herein, multiple functionalities including optical and magnetic imaging functionalities may be combined to create not only a bi-functional but a multi-functional formulation from the protein dispersion.

In some embodiments of the compositions described herein, conjugate-protein (i.e. conjugated protein) dispersions may comprise an aptamer crosslinked with a protein (e.g. aptamer-gelonin treatment for prostate cancer (Chu et al (2006))). In some embodiments, an aptamer provides a targeting capability (e.g. binding to the prostate-specific membrane antigen), while the protein (e.g. gelonin) has significant toxicity. In some embodiments of the compositions described herein, a mAb (monoclonal antibody) conjugated to a chemotherapeutic drug (as described for example in both Hamblett et al (2004) and Krop et al (2010)) can be used for treatment of various cancers. In some embodiments of conjugates, the mAb is a targeting agent for proteins that are either only- or over-expressed on the surfaces of tumor cells, for the conjugated cytotoxic agent. Abraxane is a clinical cancer parenteral nanoparticle therapy where paclitaxel is complexed with serum albumin, whereby the albumin helps deliver the abraxane.

In some embodiments of the transparent, low viscosity, high protein concentration dispersions as described herein (including embodiments), wherein the dispersions have been frozen, stored, and thawed, the average diameter of the plurality of nanoclusters is about the same (e.g. is the same) post-thawing as pre-freezing. In some embodiments of the dispersions, wherein the dispersions have been frozen, stored, and thawed, the post-thawing average diameter of the plurality of nanoclusters is within about 1% (e.g. within 1%) of the pre-freezing average diameter of the plurality of nanoclusters. In some embodiments of the dispersions, wherein the dispersions have been frozen, stored, and thawed, the post-thawing average diameter of the plurality of nanoclusters is within about 5% (e.g. within 5%) of the pre-freezing average diameter of the plurality of nanoclusters. In some embodiments of the dispersions, wherein the dispersions have been frozen, stored, and thawed, the post-thawing average diameter of the plurality of nanoclusters is within about 10% (e.g. within 10%) of the pre-freezing average diameter of the plurality of nanoclusters. In some embodiments of the transparent, low viscosity, high protein concentration dispersions as described herein (including embodiments), wherein the dispersions have been frozen and thawed, the average diameter of the plurality of nanoclusters is about the same (e.g. is the same) post-thawing as pre-freezing. In some embodiments of the dispersions, wherein the dispersions have been frozen and thawed, the post-thawing average diameter of the plurality of nanoclusters is within about 1% (e.g. within 1%) of the pre-freezing average diameter of the plurality of nanoclusters. In some embodiments of the dispersions, wherein the dispersions have been frozen and thawed, the post-thawing average diameter of the plurality of nanoclusters is within about 5% (e.g. within 5%) of the pre-freezing average diameter of the plurality of nanoclusters. In some embodiments of the dispersions, wherein the dispersions have been frozen and thawed, the post-thawing average diameter of the plurality of nanoclusters is within about 10% (e.g. within 10%) of the pre-freezing average diameter of the plurality of nanoclusters. As used herein, the term “store” or “storing”, as applied to a frozen dispersion, refers to maintaining the dispersion in a frozen state. In some embodiments, “store” or “storing” refers to maintaining the dispersion at a temperature of about −40 degrees Celsius. In some embodiments, “store” or “storing” refers to maintaining the dispersion at a temperature of less than about −40 degrees Celsius. In some embodiments, “store” or “storing” refers to maintaining the dispersion at a temperature of about −80 degrees Celsius. In some embodiments, “store” or “storing” refers to maintaining the dispersion at a temperature of about −20 degrees Celsius.

In some embodiments of the transparent, low viscosity, high protein concentration dispersions as described herein (including embodiments), wherein the dispersions have been frozen, stored, and thawed, the viscosity of the dispersion is about the same (e.g. is the same) post-thawing as pre-freezing. In some embodiments of the dispersions, wherein the dispersions have been frozen, stored, and thawed, the post-thawing viscosity of the dispersion is within about 1% (e.g. within 1%) of the pre-freezing viscosity of the dispersion. In some embodiments of the dispersions, wherein the dispersions have been frozen, stored, and thawed, the post-thawing viscosity of the dispersion is within about 5% (e.g. within 5%) of the pre-freezing viscosity of the dispersion. In some embodiments of the dispersions, wherein the dispersions have been frozen, stored, and thawed, the post-thawing viscosity of the dispersion is within about 10% (e.g. within 10%) of the pre-freezing viscosity of the dispersion. In some embodiments of the transparent, low viscosity, high protein concentration dispersions as described herein (including embodiments), wherein the dispersions have been frozen and thawed, the viscosity of the dispersion is about the same (e.g. is the same) post-thawing as pre-freezing. In some embodiments of the dispersions, wherein the dispersions have been frozen and thawed, the post-thawing viscosity of the dispersion is within about 1% (e.g. within 1%) of the pre-freezing viscosity of the dispersion. In some embodiments of the dispersions, wherein the dispersions have been frozen and thawed, the post-thawing viscosity of the dispersion is within about 5% (e.g. within 5%) of the pre-freezing viscosity of the dispersion. In some embodiments of the dispersions, wherein the dispersions have been frozen and thawed, the post-thawing viscosity of the dispersion is within about 10% (e.g. within 10%) of the pre-freezing viscosity of the dispersion.

In some embodiments of the transparent, low viscosity, high protein concentration dispersions as described herein (including embodiments), the dispersions are frozen for about one day (e.g. one day) and the average diameter of the plurality of nanoclusters is about the same (e.g. the same) post-thawing as pre-freezing. In some embodiments of the transparent, low viscosity, high protein concentration dispersions as described herein (including embodiments), the dispersions are frozen for about three days (e.g. three days) and the average diameter of the plurality of nanoclusters is about the same (e.g. the same) post-thawing as pre-freezing. In some embodiments of the transparent, low viscosity, high protein concentration dispersions as described herein (including embodiments), the dispersions are frozen for about one week (e.g. one week) and the average diameter of the plurality of nanoclusters is about the same (e.g. the same) post-thawing as pre-freezing. In some embodiments of the transparent, low viscosity, high protein concentration dispersions as described herein (including embodiments), the dispersions are frozen for about one month (e.g. one month) and the average diameter of the plurality of nanoclusters is about the same (e.g. the same) post-thawing as pre-freezing. In some embodiments of the transparent, low viscosity, high protein concentration dispersions as described herein (including embodiments), the dispersions are frozen for about one year (e.g. one year) and the average diameter of the plurality of nanoclusters is about the same (e.g. the same) post-thawing as pre-freezing.

In some embodiments of the transparent, low viscosity, high protein concentration dispersions as described herein (including embodiments), the dispersions are maintained (e.g. stored) as a frozen solid (e.g. at −40 degrees Celsius) for about one day (e.g. one day) and the average diameter of the plurality of nanoclusters is about the same (e.g. the same) post-thawing as pre-freezing. In some embodiments of the transparent, low viscosity, high protein concentration dispersions as described herein (including embodiments), the dispersions are maintained (e.g. stored) as a frozen solid (e.g. at −40 degrees Celsius) for about three days (e.g. three days) and the average diameter of the plurality of nanoclusters is about the same (e.g. the same) post-thawing as pre-freezing. In some embodiments of the transparent, low viscosity, high protein concentration dispersions as described herein (including embodiments), the dispersions are maintained (e.g. stored) as a frozen solid (e.g. at −40 degrees Celsius) for about one week (e.g. one week) and the average diameter of the plurality of nanoclusters is about the same (e.g. the same) post-thawing as pre-freezing. In some embodiments of the transparent, low viscosity, high protein concentration dispersions as described herein (including embodiments), the dispersions are maintained (e.g. stored) as a frozen solid (e.g. at −40 degrees Celsius) for about one month (e.g. one month) and the average diameter of the plurality of nanoclusters is about the same (e.g. the same) post-thawing as pre-freezing. In some embodiments of the transparent, low viscosity, high protein concentration dispersions as described herein (including embodiments), the dispersions are maintained (e.g. stored) as a frozen solid (e.g. at −40 degrees Celsius) for about one year (e.g. one year) and the average diameter of the plurality of nanoclusters is about the same (e.g. the same) post-thawing as pre-freezing.

### III. METHODS OF MAKING A DISPERSION

In a fourth aspect, a method of making a transparent, low viscosity, high protein dispersion of protein nanoclusters is provided, including concentrating a protein-crowder liquid combination and thereby forming the dispersion. The dispersion includes a plurality of nanoclusters, each of the plurality of nanoclusters includes a plurality of proteins, and each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL), and wherein the dispersion includes a plurality of a crowder. In some embodiments, the method includes, prior to the concentrating, combining a solution of the protein with a crowder in a vessel to form a protein-crowder liquid combination. In some embodiments of the method, the protein-crowder liquid combination includes a dispersion of protein nanoclusters with an average protein nanocluster diameter different from the average diameter of the plurality of protein nanoclusters formed by the concentrating. In some embodiments of the method of making a transparent, low viscosity, high protein dispersion of protein nanoclusters, the dispersion is selected from the dispersions described herein (including embodiments).

In a fifth aspect, a method of making a transparent, low viscosity, high protein dispersion of protein nanoclusters is provided, including the step of combining a protein in powder form with a crowder and a dispersion liquid thereby forming a dispersion including a plurality of nanoclusters including a plurality of the protein. Each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL). In some embodiments, the method includes, prior to the combining, removing a solvent from a protein mixture thereby forming the protein in powder form. In some embodiments of the method, the protein mixture is a protein dispersion or a protein solution. In some embodiments of the method, the removing includes milling, precipitating, dialyzing, sieving, spray drying, lyophilizing, or spray freeze drying, spray freezing the protein mixture; or the removing includes applying spiral wound in situ freezing technology (SWIFT) to the protein mixture. In some embodiments of the method, the removing includes thin film freezing. In some embodiments of the method, the solvent is water. In some embodiments of the method of making a transparent, low viscosity, high protein dispersion of protein nanoclusters, the dispersion is selected from the dispersions described herein (including embodiments).

In a sixth aspect, a method of making a transparent, low viscosity, high protein dispersion of protein nanoclusters is provided, including the step of combining a protein in powder form with a dispersion liquid thereby forming a dispersion including a plurality of nanoclusters including a plurality of the protein. Each of the plurality of proteins shares amino acid sequence identity. The dispersion is a transparent, low viscosity, dispersion; wherein the dispersion includes a concentration of the protein of greater than about 200 mg/mL (e.g. greater than 200 mg/mL). In some embodiments, the method includes prior to the combining, removing a solvent from a protein-crowder mixture thereby forming the protein in powder form, which may optionally contain a crowder. In some embodiments of the method, the protein-crowder mixture is a protein dispersion or a protein solution. In some embodiments of the method, the removing includes milling, precipitating, dialyzing, sieving, spray drying, lyophilizing, or spray freeze drying, spray freezing the protein-crowder mixture; or the removing includes applying spiral wound in situ freezing technology (SWIFT) to the protein-crowder mixture. In some embodiments of the method, the solvent is water. In some embodiments of the method of making a transparent, low viscosity, high protein dispersion of protein nanoclusters, the dispersion is selected from the dispersions described herein (including embodiments).

In some embodiments of the methods of making a transparent, low viscosity, high protein dispersion of protein nanoclusters, as described herein (including embodiments), the dispersion liquid is water, an aqueous liquid, or a non-aqueous liquid. In some embodiments of the methods of making a transparent, low viscosity, high protein dispersion of protein nanoclusters, as described herein (including embodiments), the dispersion liquid is benzyl benzoate or benzyl benzoate plus one or more oils selected from safflower, sesame, castor, cottonseed, canola, saffron, olive, peanut, sunflower seed, a-tocopherol, Miglyol 812, and ethyl oleate.

In some embodiments of the methods of making a transparent, low viscosity, high protein dispersion of protein nanoclusters, as described herein (including embodiments), the removing includes applying spiral wound in situ freezing technology (SWIFT) to the mixture. In some embodiments of the methods of making a transparent, low viscosity, high protein dispersion of protein nanoclusters, as described herein (including embodiments), applying SWIFT includes the steps of: (1) rotating a vial, containing the mixture, while contacting the vial with a cryogenic agent; (2) freezing all of the mixture, wherein the freezing results in a thin film of the frozen mixture on the inner side of the vial and one or more subsequent films in a spiral orientation towards the center of the vial; and (3) lyophilizing the frozen mixture. In some embodiments, SWIFT may include contacting the vial with a cold substance (e.g. dry ice) instead of a cryogenic agent.

In some embodiments of the methods of making a transparent, low viscosity, high protein dispersion of protein nanoclusters, as described herein (including embodiments), the concentration of the protein in the dispersion is greater than about 300 mg/mL (e.g. greater than 300 mg/mL). In some embodiments of the methods, the concentration of the protein in the dispersion is greater than about 400 mg/mL (e.g. greater than 400 mg/mL). In some embodiments of the methods, the concentration of the protein in the dispersion is greater than about 500 mg/mL (e.g. greater than 500 mg/mL). In some embodiments of the methods, the concentration of the protein in the dispersion is greater than about 600 mg/mL (e.g. greater than 600 mg/mL). In some embodiments of the methods, the concentration of the protein in the dispersion is between about 200 mg/mL and about 300 mg/mL (e.g. between 200 mg/mL and 300 mg/mL). In some embodiments of the methods, the concentration of the protein in the dispersion is between about 300 mg/mL and about 400 mg/mL (e.g. between 300 mg/mL and 400 mg/mL). In some embodiments of the methods, the concentration of the protein in the dispersion is between about 400 mg/mL and about 500 mg/mL (e.g. between 400 mg/mL and 500 mg/mL).

In some embodiments of the methods of making a transparent, low viscosity, high protein dispersion of protein nanoclusters, as described herein (including embodiments), the crowder is a glycerol, an erythritol, an arabinose, a xylose, a ribose, an inositol, a fructose, a galactose, a maltose, a glucose, a mannose, a trehalose, a sucrose, a poly(ethylene glycol), a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt, a buffer, protein, peptide, amino acid, or a combination thereof. In some embodiments of the methods, the crowder is a polysaccharide. In some embodiments of the methods, the crowder is a poly (ethylene glycol). In some embodiments of the methods, the crowder is NMP or an alcohol. In some embodiments of the methods, the crowder is an amino acid. In some embodiments of the methods, the crowder is a peptide. In some embodiments of the methods, the crowder is a peptide consisting of between two and 100 amino acids. In some embodiments of the methods, the crowder is a peptide consisting of between two and 75 amino acids. In some embodiments of the methods, the crowder is a peptide consisting of between two and 50 amino acids. In some embodiments of the methods, the crowder is a peptide consisting of between two and 25 amino acids. In some embodiments of the methods, the crowder is a peptide consisting of between two and 10 amino acids. In some embodiments of the methods, the crowder is a peptide consisting of between two and 5 amino acids. In some embodiments of the methods, the crowder is a peptide consisting of two amino acids. In some embodiments of the methods, the crowder is a peptide consisting of three amino acids. In some embodiments of the methods, the crowder is a peptide consisting of four amino acids.

In some embodiments of the methods of making a transparent, low viscosity, high protein dispersion of protein nanoclusters, as described herein (including embodiments), the concentrating is performed using filtration. In some embodiments of the methods of making a transparent, low viscosity, high protein dispersion of protein nanoclusters, as described herein (including embodiments), the concentrating is performed using centrifugal filtration. In some embodiments of the methods, the concentrating is performed using positive gas pressure or mechanical pressure. In some embodiments of the methods, the concentrating is performed using tangential flow filtration, dialysis, or absorption of buffer. In some embodiments of the methods, the concentrating is performed using a compound capable of absorbing liquid (e.g. a molecular sieve). In some embodiments of the methods, the concentrating includes adding a compound. (e.g. a molecular sieve) to the protein-crowder mixture, wherein the added compound absorbs liquid. In some embodiments of the methods, the concentrating includes adding a compound (e.g. a molecular sieve) to the protein-crowder mixture, wherein the added compound reduces the water in the protein-crowder mixture by removing it from the bulk solution. In some embodiments of the methods, a crowder or the protein is added to the protein-crowder liquid combination during the concentrating.

In some embodiments, the methods of making a transparent, low viscosity, high protein dispersion of protein nanoclusters, as described herein (including embodiments), further include sterilizing the dispersion. In some embodiments, the methods of making a transparent, low viscosity, high protein dispersion of protein nanoclusters, as described herein (including embodiments), further include sterilizing the dispersion by filtration. In some embodiments, the methods of making a transparent, low viscosity, high protein dispersion of protein nanoclusters, as described herein (including embodiments), further include sterilizing the dispersion by filtration through a filter having pores of about 200 nm diameter (e.g. 200 nm diameter).

In some embodiments, the methods of making a transparent, low viscosity, high protein dispersion of protein nanoclusters, as described herein (including embodiments), further include freezing, storing, and thawing the dispersion, and the average diameter of the plurality of nanoclusters is about the same (e.g. is the same) post-thawing as pre-freezing. In some embodiments of the methods, further including freezing, storing, and thawing the dispersion, the post-thawing average diameter of the plurality of nanoclusters is within about 1% (e.g. within 1%) of the pre-freezing average diameter of the plurality of nanoclusters. In some embodiments of the methods, further including freezing, storing, and thawing the dispersion, the post-thawing average diameter of the plurality of nanoclusters is within about 5% (e.g. within 5%) of the pre-freezing average diameter of the plurality of nanoclusters. In some embodiments of the methods, further including freezing, storing, and thawing the dispersion, the post-thawing average diameter of the plurality of nanoclusters is within about 10% (e.g. within 10%) of the pre-freezing average diameter of the plurality of nanoclusters. In some embodiments, the methods of making a transparent, low viscosity, high protein dispersion of protein nanoclusters, as described herein (including embodiments), further include freezing and thawing the dispersion, and the average diameter of the plurality of nanoclusters is about the same (e.g. is the same) post-thawing as pre-freezing. In some embodiments of the methods, further including freezing and thawing the dispersion, the post-thawing average diameter of the plurality of nanoclusters is within about 1% (e.g. within 1%) of the pre-freezing average diameter of the plurality of nanoclusters. In some embodiments of the methods, further including freezing and thawing the dispersion, the post-thawing average diameter of the plurality of nanoclusters is within about 5% (e.g. within 5%) of the pre-freezing average diameter of the plurality of nanoclusters. In some embodiments of the methods, further including freezing and thawing the dispersion, the post-thawing average diameter of the plurality of nanoclusters is within about 10% (e.g. within 10%) of the pre-freezing average diameter of the plurality of nanoclusters. As used herein, the term “store” or “storing”, as applied to a frozen dispersion, refers to maintaining the dispersion in a frozen state. In some embodiments, “store” or “storing” refers to maintaining the dispersion at a temperature of about −40 degrees Celsius. In some embodiments, “store” or “storing” refers to maintaining the dispersion at a temperature of less than about −40 degrees Celsius. In some embodiments, “store” or “storing” refers to maintaining the dispersion at a temperature of about −80 degrees Celsius. In some embodiments, “store” or “storing” refers to maintaining the dispersion at a temperature of about −20 degrees Celsius.

In some embodiments, the methods of making a transparent, low viscosity, high protein dispersion of protein nanoclusters, as described herein (including embodiments), further include freezing, storing, and thawing the dispersion, and the viscosity of the dispersion is about the same (e.g. is the same) post-thawing as pre-freezing. In some embodiments of the methods, further including freezing, storing, and thawing the dispersion, the post-thawing viscosity of the dispersion is within about 1% (e.g. within 1%) of the pre-freezing viscosity of the dispersion. In some embodiments of the methods, further including freezing, storing, and thawing the dispersion, the post-thawing viscosity of the dispersion is within about 5% (e.g. within 5%) of the pre-freezing viscosity of the dispersion. In some embodiments of the methods, further including freezing, storing, and thawing the dispersion, the post-thawing viscosity of the dispersion is within about 10% (e.g. within 10%) of the pre-freezing viscosity of the dispersion. In some embodiments, the methods of making a transparent, low viscosity, high protein dispersion of protein nanoclusters, as described herein (including embodiments), further include freezing and thawing the dispersion, and the viscosity of the dispersion is about the same (e.g. is the same) post-thawing as pre-freezing. In some embodiments of the methods, further including freezing and thawing the dispersion, the post-thawing viscosity of the dispersion is within about 1% (e.g. within 1%) of the pre-freezing viscosity of the dispersion. In some embodiments of the methods, further including freezing and thawing the dispersion, the post-thawing viscosity of the dispersion is within about 5% (e.g. within 5%) of the pre-freezing viscosity of the dispersion. In some embodiments of the methods, further including freezing and thawing the dispersion, the post-thawing viscosity of the dispersion is within about 10% (e.g. within 10%) of the pre-freezing viscosity of the dispersion.

In some embodiments, the methods of making a transparent, low viscosity, high protein dispersion of protein nanoclusters, as described herein (including embodiments), further includes freezing the dispersion for about one day (e.g. one day) and thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes freezing the dispersion for about three days (e.g. three days) and thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes freezing the dispersion for about one week (e.g. one week) and thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further include freezing the dispersion for about one month (e.g. one month) and thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes freezing the dispersion for about one year (e.g. one year) and thawing the dispersion, wherein the average diameter of the plurality of nanoclusters is about the same (e.g. the same) post-thawing as pre-freezing.

In some embodiments, the methods of making a transparent, low viscosity, high protein dispersion of protein nanoclusters, as described herein (including embodiments), further includes maintaining (e.g. storing) the dispersion as a frozen solid (e.g. at −40 degrees Celsius) for about one day (e.g. one day) and then thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes maintaining (e.g. storing) the dispersion as a frozen solid (e.g. at −40 degrees Celsius) for about three days (e.g. three days) and then thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes maintaining (e.g. storing) the dispersion as a frozen solid (e.g. at −40 degrees Celsius) for about one week (e.g. one week) and then thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further include maintaining (e.g. storing) the dispersion as a frozen solid (e.g. at −40 degrees Celsius) for about one month (e.g. one month) and then thawing the dispersion, wherein the average diameter of the plurality of nanoclusters is about the same (e.g. the same) post-thawing as pre-freezing. In some embodiments, the methods of making a transparent, low viscosity, high protein dispersion of protein nanoclusters, as described herein (including embodiments), further includes maintaining (e.g. storing) the dispersion as a frozen solid (e.g. at −40 degrees Celsius) for about one year (e.g. one year) and then thawing the dispersion, wherein the average diameter of the plurality of nanoclusters is about the same (e.g. the same) post-thawing as pre-freezing.

### IV. METHODS OF TREATING DISEASES

In a seventh aspect, a method is provided for treating a disease in a patient in need of such treatment, the method including administering an effective amount of any one of the dispersions described herein (including embodiments) to the patient. In some embodiments of the method of treating a disease, the administered dispersion includes about 0.5, 1, 2, 4, 6, 8, 10 mg of protein for each kg of body weight of the patient (e.g. 0.5, 1, 2, 4, 6, 8, 10 mg of protein for each kg of body weight).

The compositions (e.g. protein nanoclusters, protein-crowder nanoclusters, dispersions) of the invention can be administered alone or can be coadministered to the patient. Coadministration is meant to include simultaneous or sequential administration of the compositions individually or in combination (more than one composition). Thus, the preparations can also be combined, when desired, with other active substances (e.g. to reduce metabolic degradation). The compositions described herein can be used in combination with one another, with other active agents known to be useful in treating a disease, or with adjunctive agents that may not be effective alone, but may contribute to the efficacy of the active agent, or with diagnostic agents.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compositions (e.g. protein nanoclusters, protein-crowder nanoclusters, dispersions) of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. It is also envisioned that multiple routes of administration (e.g., intramuscular, oral, transdermal) can be used to administer the compositions described herein (including embodiments). Accordingly, the present invention also provides pharmaceutical compositions including a pharmaceutically acceptable excipient and one or more compositions of the invention. The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to the site of disease (e.g. tumor in the case of cancer) or systemic.

For preparing pharmaceutical compositions from the compositions as described herein (including embodiments), pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

In powders, the carrier is a finely divided solid in a mixture with the finely divided active component (e.g. a compositions provided herein). In tablets, the active composition is mixed with the carrier having the necessary binding properties in suitable proportions and compacted in the shape and size desired. The powders and tablets may contain from about 5% to about 70% of the active compositions.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active composition is dispersed homogeneously therein, as by stirring. The molten homogeneous mixture is then poured into convenient sized molds, allowed to cool, and thereby to solidify.

When parenteral application is needed or desired, particularly suitable admixtures for the compositions are injectable, sterile solutions, preferably oily or aqueous solutions, as well as dispersions, suspensions, emulsions, or implants, including suppositories. In particular, carriers for parenteral administration include aqueous solutions of dextrose, saline, pure water, buffers, ethanol, glycerol, propylene glycol, peanut oil, sesame oil, polyoxyethylene-block polymers, and the like. Ampules are convenient unit dosages. The compositions can also be incorporated into liposomes or administered via transdermal pumps or patches. Pharmaceutical admixtures suitable for use are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity. The aqueous suspension or dispersion can be made in water with a crowder or with a non-aqueous solvent with or without a crowder.

Also included are solid form preparations that are intended to be converted, shortly before use, to liquid form preparations for oral administration (e.g. protein in powder form or protein-crowder mixtures in powder form, or another solid form). Such liquid forms include dispersions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil ora mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions as described herein (including embodiments) may additionally include components to provide sustained release and/or comfort. Such components include high molecular weight, anionic mucomimetic polymers, gelling polysaccharides and finely-divided drug carrier substrates. These components may serve multiple functions as they may also acts as a crowder to aid nanocluster formation. These components are discussed in greater detail in U.S. Pat. Nos. 4,911,920; 5,403,841; 5,212,162; and 4,861,760. The entire contents of these patents are incorporated herein by reference in their entirety for all purposes. The nanocluster dispersions may be loaded into entities known to those in the field of drug delivery to further enable controlled (e.g. sustained) release including liposomes, microspheres, capsules, osmotic pumps, coating of polymer shells, matrices and implantable devices. In another embodiment, the nanocluster dispersions may be dried and then loaded into these entities.

The compositions as described herein (including embodiments) can be delivered by transdermally, by a topical route, formulated as applicator sticks, dispersions, suspensions, emulsions, gels, creams, ointments, pastes, jellies, paints, powders, and aerosols.

The compositions as described herein (including embodiments) can also be delivered as microspheres for slow release in the body. For example, microspheres can be administered via intradermal injection of drug-containing microspheres, which slowly release subcutaneously (see Rao, J. Biomater Sci. Polyin. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical compositions can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

In another embodiment, the compositions as described herein (including embodiments) are useful for parenteral administration, such as intravenous (IV) administration or administration into a body cavity or lumen of an organ. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques (e.g. filtration). For IV administration, the formulation can be a sterile injectable preparation, such as a sterile injectable aqueous dispersion.

In another embodiment, the formulations of the compositions as described herein (including embodiments) can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Pharmaceutical compositions include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein (including embodiments). Adjustment and manipulation of established dosages (e.g., frequency and duration) are well within the ability of those skilled in the art.

For any composition described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

### V. METHODS OF MODIFYING NANOCLUSTER SIZE

In an eighth aspect, a method is provided for modifying the average protein nanocluster diameter of a transparent, low viscosity, high protein dispersion of protein nanoclusters including increasing or decreasing the concentration of a crowder, or protein in the dispersion. The dispersion includes a plurality of nanoclusters and each of the plurality of nanoclusters includes a plurality of proteins. Each of the plurality of proteins shares amino acid sequence identity. The dispersion is a transparent, low viscosity, dispersion; and the dispersion includes a concentration of the protein of greater than about 200 mg/mL (e.g. greater than 200 mg/mL).

### VI. ADDITIONAL COMPOSITIONS AND METHODS

In another aspect, low viscosity high concentration antibody dispersions is provided as well as methods of making the same. In another aspect is provided a composition having substantially transparent conformationally stabilized protein nanoclusters that retain therapeutic activity both in vivo and in vitro. In some embodiments, upon dilution, the clusters reversibly dissociate into native monomeric protein molecules with high biological activity having low viscosities. In some embodiments, the approach is broadly applicable to wide classes of proteins, without the need to modify the amino acid sequence.

In another aspect is provided a composition having a substantially transparent, low viscosity, high concentration dispersion of nanoclusters in a dispersion medium, wherein the nanoclusters include proteins or peptides and have an average diameter between 20 and 1,000 nanometers, wherein the proteins or peptides are stable and are clustered into the nanoclusters.

In some embodiments, the composition as disclosed hereinabove includes one or more crowders selected from the group consisting of a glycerol, an erythritol, an arabinose, a xylose, a ribose, an inositol, a fructose, a galactose, a maltose, a glucose, a mannose, a trehalose, a sucrose, a poly(ethylene glycol), a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, organic co-solvents selected from ethanol, N-methyl-2-pyrrolidone (NMP), PEG 300, PEG 400, PEG 200, PEG 3350, Propylene Glycol, N,N Dimethylacetamide, a dimethyl sulfoxide, a solketal, a tetahydrofurfuryl alcohol, a diglyme, an ethyl lactate, a salt, a buffer or a combination thereof. In some embodiments, the nanocluster includes two or more different peptides or proteins. In some embodiments, the dispersion is a mixture of a first and a second dispersion of nanoclusters, wherein the first and second nanoclusters each having a different protein or peptide. In some embodiments, the dispersion includes nanoclusters that each have two or more different peptides or proteins. In some embodiments, the dispersion medium is at or near the isoelectric point of the proteins or peptides. In some embodiments, the dispersion medium is within 2.5, 2.0, 1.5, 1.0, 0.8, 0.75, 0.5, 0.3, 0.2, 0.1, 0.05 pH units of the isoelectric point of the protein or peptides. In some embodiments, the composition is sterilized by filtration. In some embodiments, the composition is an extended release composition.

In some embodiments, the proteins in the nanoclusters become a biologically stable monomer upon a decrease in protein concentration, the crowder or both. In some embodiments, the total concentration of peptides and proteins in the low viscosity high concentration dispersion is 25, 50, 100, 150, 200, 250, 300, 350, 400, 500 mg/mL or greater. In some embodiments, the low viscosity high concentration dispersion has a viscosity of less than 100, 90, 80, 70, 60, 50, 40, 30, 20, or 10 centipoise. In some embodiments, the nanocluster has a diameter of approximately 20, 30, 40, 50, 75, 100, 150, 250, 300, 400, 600, 800, or 1000 nm. In some embodiments, the nanocluster diameter is a hydrodynamic diameter.

In some embodiments, the one or more proteins or peptides are selected from an antibody, an antibody fragment (e.g. Fab, Fc, Fv, Fab′), a pegylated protein, a lipidated protein, a growth factor or antagonist, a cytokine or antagonist, a receptor or receptor antagonist, an antigen, a vaccine, an anti-inflammatory agent, a therapeutic polypeptide or peptide, or a combination thereof. In some embodiments, the viscosity of the dispersion of the present invention is less than that of an equivalent concentration of the protein or peptide in solution. In some embodiments, the protein or peptide is stable at a concentration where the equivalent protein or peptide concentration in solution is unstable. In some embodiments, the nanocluster is a reversible cluster having primary protein particles that dissociate into stable monomeric proteins upon parenteral administration. In some embodiments, the proteins are self-crowded within the cluster to maintain a stable conformation. In some embodiments, the low viscosity high concentration dispersion is syringeable through a 21 to 27-gauge needle.

In another aspect the one or more proteins or peptides are made into micron or submicron sized particles by one or more techniques selected from the group consisting of milling, precipitation, dialysis, sieving, spray drying, lyophilization, spiral wound in situ freezing technology (SWIFT), spray freeze drying, spray freezing into liquids, thin film freezing, and freezing directly in a dosage container. In yet another aspect the low viscosity high concentration dispersion is made by dispersing the micron or sub micron sized particles in the dispersion medium. In some embodiments, the dispersion medium includes a pharmaceutically acceptable solvent including a pharmaceutically acceptable aqueous solvent, a pharmaceutically acceptable non-aqueous solvent, or a combination. In some embodiments, the pharmaceutically acceptable solvents that may be used herein include benzyl benzoate or benzyl benzoate plus one or more oils selected from safflower, sesame, castor, cottonseed, canola, saffron, olive, peanut, sunflower seed, a-tocopherol, Miglyol 812, and ethyl oleate. In some embodiments, the composition described hereinabove may include one or more additives selected from the group consisting of a stabilizer, a surfactant, an emulsifier, a salt, a buffer, an amino acid, a small peptide, a polypeptide, a protein, a polymer, a cosolvent, and combinations thereof. In some embodiments, the proteins or peptides are self-crowding. In some embodiments, at least half of the proteins or peptides are not in solution. In some embodiments, following dilution from the dispersion medium, the proteins or peptides in the nanoclusters revert into a monomeric form. In some embodiments, the proteins or peptides retain at least 95%, 96%, 97%, 98%, 99% and 100% activity upon dilution from the dispersion medium.

Stability of the protein in the composition disclosed hereinabove may be measured by size exclusion chromatography, analytical ultracentrifugation, CD spectroscopy, FTIR spectroscopy, dynamic light scattering, static light scattering, ELISA, native PAGE gel, or biological activity assays. In some embodiments, the composition (e.g. dispersion) exhibits substantially similar pharmacokinetic properties on injection when compared to an injectable solution of the protein or the peptide, wherein the pharmacokinetic properties include the maximum serum concentration (Cₘₐₓ), the time after injection that the maximum concentration is achieved tₘₐₓ, the maximum available dose as represented by the area under the curve (AUC), tissue distribution (t_{1/2} alpha) and elimination times (t_{1/2} beta), or combinations thereof. In some embodiments, the injected dosage is 0.1, 0.3, 0.5, 1, 2, 4, 6, 8, 10 mg/kg of body weight of a mammal. In some embodiments, the area under the curve (AUC)/dose in the blood is 50%, 70%, 80%, 90%, 100%, 120%, 150%, 200%, 300% of the value observed for an intravenous delivery for an ending time between 2 and 30 days. In some embodiments, the AUC/dose in the blood is 50%, 70%, 80%, 90%, 100%, 120%, 150%, 200%, 300% of the value observed for an intravenous delivery for an ending time between 2 and 14 days. In some embodiments, the total AUC for an ending time of 2, 5, 7, 10, 14, 21, 28 and 30 days is 1, 2, 5, 6, 8, 10 times that of the total AUC for a subcutaneous (SQ) solution.

In some embodiments, the total AUC for 20 days is 1, 2, 5, 6, 8, 10 times that of the total AUC for an SQ solution, the total AUC for 14 days is 1, 2, 5, 6, 8, 10 times that of the total AUC for an SQ solution, or the total AUC for 10 days is 1, 2, 5, 6, 8, 10 times that of the total AUC for an SQ solution. In some embodiments, the Cₘₐₓ of the composition reaches 0.5, 0.7, 0.9, 1.5, 2, 4, 6, 8 times the Cₘₐₓ for a SQ solution injection. In some embodiments, the tₘₐₓ is delayed by 1.2, 1.4, 1.6, 1.8, 2.0 times the tₘₐₓ for an intravenous, oral, parenteral, or SQ solution. In some embodiments, the proteins or peptides retain at least 95%, 96%, 97%, 98%, 99%, and 100% activity upon dilution from the dispersion medium.

In some embodiments, half of the total AUC is observed in the blood over 1, 2, 3, 5, 10, 20, or 30 days. In some embodiments, the therapeutic protein retains full biological activity in the serum over 1, 2, 3, 5, 10, 20, of 30 days. In some embodiments, the c/cₘₐₓ is >/=0.5 at 2, 5, 10, 20, or 30 days, the c/cₘₐₓ is >/=0.3 at 2, 5, 10, 20, or 30 days, or the c/cₘₐₓ is >/=0.1 at 2, 5, 10, 20, or 30 days. In some embodiments, the composition is adapted for intravenous, subcutaneous, parenteral, or oral administration.

In another aspect is provided a method for treating a mammal including the step of administering a therapeutically effective amount of the composition (e.g. dispersion) as described hereinabove to the mammal, wherein the mammal has a disorder requiring treatment with the protein in the formulation. In some embodiments, the mammal is a human.

In another aspect is provided a method of treating pertussis by administrating to a patient in need thereof a therapeutically effective amount of a formulation as described herein.

In another aspect is provided a pertussis treatment method of administration to a patient in need thereof a therapeutically effective amount of a formulation as described above with co-administration of antibiotics.

In another aspect is provided a method of making a composition (e.g. dispersion) including: forming a high concentration dispersion of nanoclusters in a dispersion medium, wherein the nanoclusters include proteins or peptides and have an average diameter between 20 and 1,000 nanometers and the proteins or peptides are stable and the composition is a substantially transparent, high concentration, low viscosity protein or peptide dispersion. In some embodiments of the method, the nanoparticles are further processed by one or more techniques selected from the group consisting of milling, precipitation, dialysis, sieving, spray drying, lyophilization, spiral wound in situ freezing technology (SWIFT), spray freeze drying, spray freezing into liquids, thin film freezing, and freezing directly in a dosage container. In some embodiments, the nanoparticles are further processed by adding one or more additives to the one or more sub-micron or micron-sized particles. In some embodiments, the nanoparticles are further processed by adding one or more additives to the one or more sub-micron or micron-sized particles in an aqueous media. In some embodiments of the method hereinabove, adjusting a size of the nanoparticle to a desired hydrodynamic diameter is done by adding a predetermined crowder concentration and adjusting the size upon mild mixing in situ. In some embodiments of the method hereinabove, adjusting a size of the nanoparticle to a desired hydrodynamic diameter is done by adding a predetermined crowder concentration and adjusting the size upon mild mixing in situ with an aqueous media. In some embodiments, the nanoparticles are made and stored in the same vial, the composition is sterilized by filtration or the proteins or the peptides are stable and self-crowded.

In some embodiments, the crowders employed in the method of the present invention include a glycerol, an erythritol, an arabinose, a xylose, a ribose, an inositol, a fructose, a galactose, a maltose, a glucose, a mannose, a trehalose, a sucrose, a poly(ethylene glycol), a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt, a buffer, proteins, peptides, amino acids, conjugated proteins, non-conjugated proteins, or a combination thereof. In another embodiment, each nanocluster includes two or more different peptides or proteins. In another embodiment of the method the dispersion includes nanoclusters that each have two or more different peptides or proteins. In another embodiment, the method further includes the step of adjusting a pH of the dispersion medium to at or near the isoelectric point of the individual protein or peptide to assist in a formation of the one or more nanoclusters. In another embodiment, the dispersion medium is at or near the isoelectric point of the protein or peptides. In another embodiment, the dispersion medium is within 2.5, 2.0, 1.5, 1.0, 0.8, 0.75, 0.5, 0.3, 0.2, 0.1, 0.05 pH units the isoelectric point of the protein or peptides.

In another embodiment, the dispersion medium of the method of the present invention includes a pharmaceutically acceptable solvent including a pharmaceutically acceptable aqueous solvent, a pharmaceutically acceptable non-aqueous solvent, or a combination. In some embodiments, the pharmaceutically acceptable solvent includes benzyl benzoate or benzyl benzoate plus one or more oils selected from safflower, sesame, castor, cottonseed, canola, saffron, olive, peanut, sunflower seed, a-tocopherol, Miglyol 812, and ethyl oleate. In some embodiments, the composition may include one or more additives selected from the group consisting of a stabilizer, a surfactant, an emulsifier, a salt, an amino acid, a small peptide, a polypeptide, a protein, a polymer, a cosolvent, and combinations thereof. In some embodiments, the proteins in the nanoclusters become biologically stable monomers upon a decrease in protein concentration or the crowder. In some embodiments, the concentration of the low viscosity high concentration dispersion is 25, 50, 100, 150, 200, 250, 300, 350, 400, 500 mg/mL or greater. In some embodiments, the viscosity of the low viscosity high concentration dispersion is less than 100, 90, 80, 70, 60, 50, 40, 30, 20, or 10 centipoise. In some embodiments, the nanocluster has a hydrodynamic diameter of approximately 20, 30, 40, 50, 75, 100, 150, 250, 300, 400, 600, 800, or 1000 nm. In some embodiments, the nanocluster has a diameter of approximately 20, 30, 40, 50, 75, 100, 150, 250, 300, 400, 600, 800, or 1000 nm.

In some embodiments, the one or more proteins or peptides used in the method of the present invention are selected from an antibody, an antibody fragment (e.g. Fab, Fc, Fv, Fab′), a pegylated protein, a lipidated protein, a growth factor or antagonist, a cytokine or antagonist, a receptor or receptor antagonist, an antigen, a vaccine, an anti-inflammatory agent, a therapeutic polypeptide or peptide, or a combination thereof. In some embodiments, the viscosity of the dispersion is less than that of an equivalent concentration of the protein or peptide in solution. In some embodiments, the protein or peptide is stable at a concentration where the equivalent protein or peptide concentration in solution is unstable. In some embodiments, the nanocluster is a reversible cluster including primary proteins that dissociate into stable monomeric proteins upon parenteral administration. In some embodiments, the low viscosity high concentration dispersion is syringeable through a 21 to 27-gauge needle.

In some embodiments of the method disclosed herein the one or more micron or submicron sized particles of the protein or the peptide is formed by tray lyophilization. In some embodiments of the method disclosed herein the one or more micron or submicron sized particles of the protein or the peptide is formed by SWIFT. In some embodiments, the step of forming one or more micron or submicron sized particles of the protein or the peptide by SWIFT includes the steps of: (i) providing a concentrated and purified protein or peptide solution in a buffer, wherein the buffer is selected to maintain an integrity, a stability, and activity of the protein or the peptide during freezing, (ii) adding a cryoprotectant to the purified protein or peptide solution, (iii) sterilizing the protein or the peptide solution by a membrane filtration, (iv) transferring a fixed volume of the sterilized protein or peptide solution to a sterile freezing vial, (v) rotating the freezing vial on its side while contacting the vial base with liquid nitrogen or any other suitable cryogenic agent, (vi) freezing the entire volume of the protein or the peptide solution to form a powder, wherein the freezing results in a formation of an initial thin film of the frozen protein or the peptide solution on the inner side of the vial and one or more subsequent films in a spiral orientation towards the center of the vial, and (vii) performing one or more lyophilization cycles on the frozen powder.

In another embodiment, the method includes the step of assessing protein or peptide activity after reconstitution of the frozen powder in a buffer. In some embodiments of the method, the proteins or peptides are self-crowding. In some embodiments of the method, the proteins or peptides are not in solution. In some embodiments of the method, the proteins or peptides revert into a monomeric form upon dilution from the dispersion medium. In some embodiments of the method, the proteins or peptides retain at least 95%, 96%, 97%, 98%, 99%, and 100% activity upon dilution from the dispersion medium. In some embodiments of the method, the composition exhibits substantially similar pharmacokinetic properties on injection when compared to an injectable solution of the protein or the peptide, wherein the pharmacokinetic properties include the maximum serum concentration (Cₘₐₓ), the time after injection that the maximum concentration is achieved (tₘₐₓ), the maximum available dose as represented by the area under the curve (AUC), tissue distribution (t_{1/2} alpha) and elimination times (t_{1/2} beta), or combinations thereof. In another embodiment, the composition is formulated to provide an injected dosage of 0.1, 0.3, 0.5, 1, 2, 4, 6, 8, 10 mg/kg of body weight of a mammal. In another embodiment, the composition provides an area under the curve (AUC)/dose in the blood of 50%, 70%, 80%, 90%, 100%, 120%, 150%, 200%, 300% of the value observed for an intravenous delivery with an ending time between 2 and 30 days.

In some embodiments of the method, the composition provides an AUC/dose in the blood of 50%, 70%, 80%, 90%, 100%, 120%, 150%, 200%, 300% of the value observed for an intravenous delivery with an ending time between 2 and 14 days. In some embodiments of the method, the composition provides a total AUC for 2, 5, 7, 10, 14, 21, 28, and 30 days is 1, 2, 5, 6, 8, 10 times that of the total AUC for a subcutaneous (SQ) solution. In some embodiments of the method, the composition provides a total AUC for: (i) 20 days of 1, 2, 5, 6, 8, 10 times that of the total AUC for an SQ solution, (ii) 14 days of 1, 2, 5, 6, 8, 10 times that of the total AUC for an SQ solution, or (iii) 10 days of 1, 2, 5, 6, 8, 10 times that of the total AUC 10 for an SQ solution. In some embodiments of the method, the composition provides a Cₘₐₓ that reaches 0.5, 0.7, 0.9, 1.5, 2, 4, 6, 8 times the Cₘₐₓ for a SQ solution injection. In some embodiments of the method, the composition provides a tₘₐₓ that is delayed by 1.2, 1.4, 1.6, 1.8, 2.0 times the tₘₐₓ for an intravenous, oral, parenteral or subcutaneous solution.

In some embodiments of the method, upon dilution from the dispersion medium the proteins or peptides retain at least 95%, 96%, 97%, 98%, 99%, and 100% activity. In some embodiments of the method the composition provides one-half of the total AUC observed in the blood over 1, 2, 3, 5, 10, 20, or 30 days. In some embodiments of the method, the protein or peptide is a therapeutic protein or peptide that retains full biological activity in the serum over 1, 2, 3, 5, 10, 20, or 30 days. In some embodiments of the method, the composition provides a c/cₘₐₓ that is: (i) >/=0.5 at 2, 5, 10, 20, or 30 days, (ii) >/=0.3 at 2, 5, 10, 20, or 30 days, or (iii) >/=0.1 at 2, 5, 10, 20, or 30 days. In some embodiments of the method, the composition is adapted for intravenous, subcutaneous, parenteral or oral administration. In some embodiments of the method, the protein retains native conformation and activity within the dispersion and after dilution as measured by intrinsic tryptophan fluorescence, FTIR (fourier transmission infra-red spectroscopy), SEC, AUC, HPLC, light scattering, mass spectrometry, SEC, DLS, gel electrophoresis, antigen-specific or polyclonal ELISA, and specific in vitro activity assay. In some embodiments of the method, the composition may be made by the methods described hereinabove.

In another aspect is provided a method for administering a protein or peptide nanocluster composition for an application that requires one or more selected pharmacokinetic properties wherein the pharmacokinetic property is measured in a given medium and administered by a given route. The method includes the steps of: (i) providing the protein or peptide molecules that have an identifiable value for the one or more selected pharmacokinetic properties within a medium and in soluble form, (ii) forming the protein or peptide nanocluster composition having one or more protein or peptide nanoclusters and zero, one or more crowders in a dispersion medium, wherein the nanoclusters include proteins or peptides and have an average diameter between 20 and 1,000 nanometers, wherein the proteins or peptides are stable and are clustered into the nanoclusters, and (iii) administering the protein or peptide nanoparticle composition to a subject, wherein the nanoparticle composition has a value of the selected pharmacokinetic property that is substantially the same as the identifiable value when measured in the medium and when administered by the given route.

In another aspect is provided a method for administering protein or peptide nanoparticle compositions for an application that requires one or more selected pharmacokinetic property wherein the pharmacokinetic property is measured in a given medium and administered by a given route including the steps of: (i) providing the protein or peptide molecules that have an identifiable value for the one or more selected pharmacokinetic properties within a medium and in soluble form, (ii) forming the protein or peptide nanoparticle composition including one or more self-crowding protein or peptide nanoparticles including between 80 to 250 proteins or peptides per nanoparticle in a dispersion medium, wherein the nanoparticles are not in solution, wherein the proteins or the peptides are stable, self-crowded and are clustered when at or near their individual isoelectric points, wherein the composition provides at least 200 mg/mL of the protein or peptide on injection, and (iii) administering the protein or peptide nanoparticle composition to a subject, wherein the nanoparticle composition has a value of the selected pharmacokinetic property that is substantially the same as the identifiable value when measured in the medium and when administered by the given route.

In another aspect is provided a sterile nanocluster dispersion made by the process of forming one or more nanoparticles of the protein or the peptide in a dispersion medium, which optionally includes a crowder, under conditions that form protein or peptide nanoclusters having an average hydrodynamic diameter between 20 and 1,000 nanometers and the proteins or peptides are stable and the composition is a substantially transparent, high concentration, low viscosity protein or peptide dispersion.

### Example I

Polyclonal sheep IgG (Product No. 15131) was purchased from Sigma-Aldrich, Inc. (St. Louis, Mo.) and further purified by size-exclusion, fast protein liquid chromatography (FPLC). α-α trehalose, polyethylene glycol with an average molecular weight of 300 (PEG 300), n-methyl 2-pyrrolidone (NMP), and all other chemicals were purchased from Fisher Chemicals (Fairlawn, N.J.).

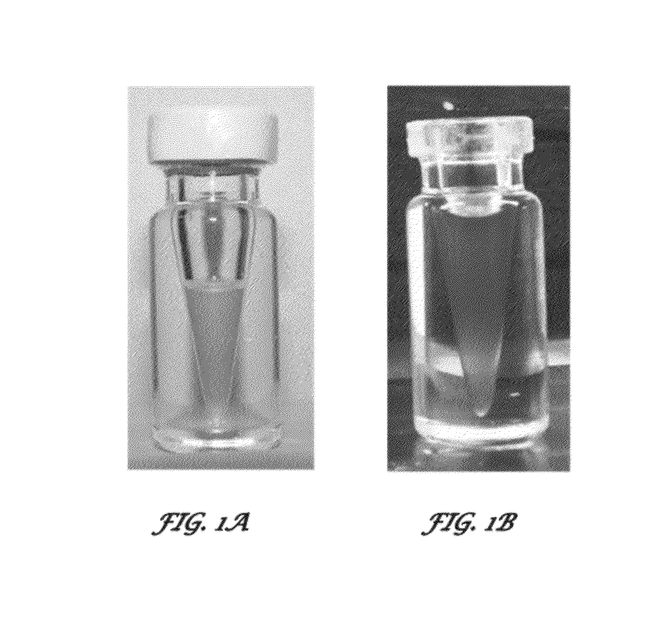

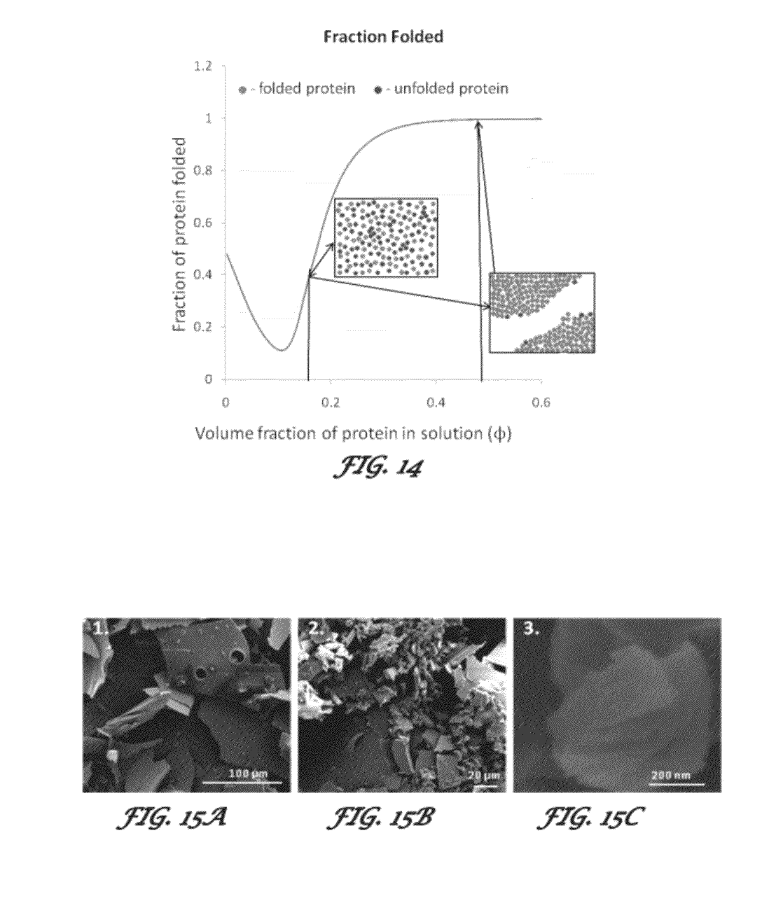

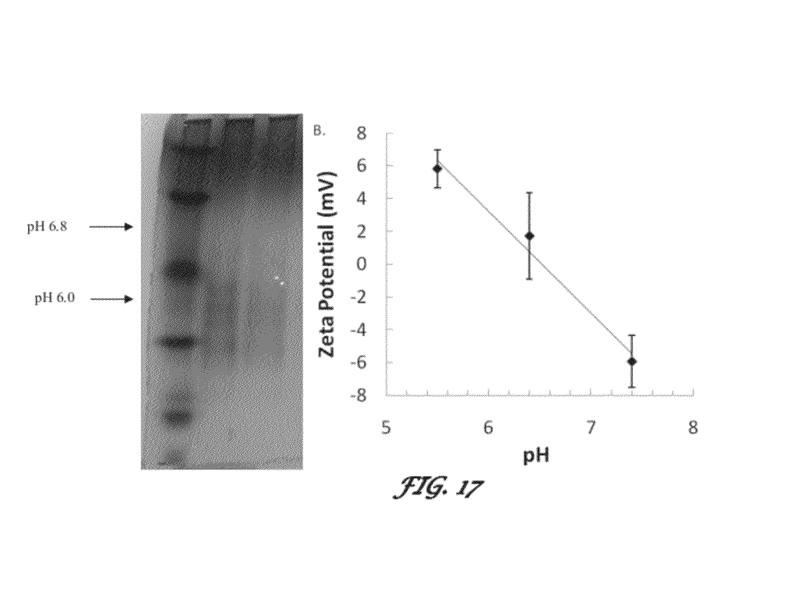

Powder and dispersion formation: The pI of the protein was determined to be 6.4 from the zeta potential in 20 mM histidine buffer at a pH of 5.5, 6.4 and 7.4 and confirmed by isoelectric focusing gel electrophoresis (FIG. 17). The IgG solution, purified by FPLC, at an initial concentration of 20 mg/ml in histidine buffer, pH 5.5, with 1:1 wt ratio of α-α trehalose, was slowly frozen over 6 hours in 8 ml vials on a pre-cooled lyophilizer tray at −40° C. (VirTis Advantage Plus Benchtop Freeze Dryer). The sample was then lyophilized to form a dry powder at 100 mTorr with 12 hours of primary drying at −40° C. followed by a 6 hour ramp to 25° C. and an additional 6 hours of secondary drying at 25° C. Scanning electron microscopy images of the powders formed upon lyophilization are shown in (FIGS. 15A-15C). Between 0.039 and 0.08 g 0.0005 g of powder were compacted with a spatula into a 0.1 ml conical vial (Wheaton Science Products No. 986211). 100+1 μl of an aqueous-based buffer were added to the conical vial with a 20-200 μl micropipette to yield a total dispersion volume of ˜0.1 ml. NaCl was added to 50 mM pH 6.4 phosphate buffer (the pI of sheep IgG38) to yield a total ionic strength of 154 mM. The mixture of powder and buffer was stirred gently, at low shear, with the tip of the 25 g needle to remove air pockets and form a transparent dispersion without the appearance of any visible inhomogeneities (FIGS. 1A and 1B) using the naked eye. The highly soluble trehalose in the powder dissolved and became an extrinsic crowding agent in the dispersion. In certain studies, the aqueous buffer contained a known volume of PEG300 as an additional crowder, or mixture of PEG300 and NMP. The total volumes of the various components in the concentrated dispersions are given in Table 1, based on known masses and densities (from partial molar volumes) of IgG and trehalose and known added volumes of the other (liquid) components. The volume fractions of the components (φ=φ, I for IgG, T for trehalose, P for PEG300, and N for NMP) are given in Table 1. This basic procedure was also used to determine volume fractions throughout the study.

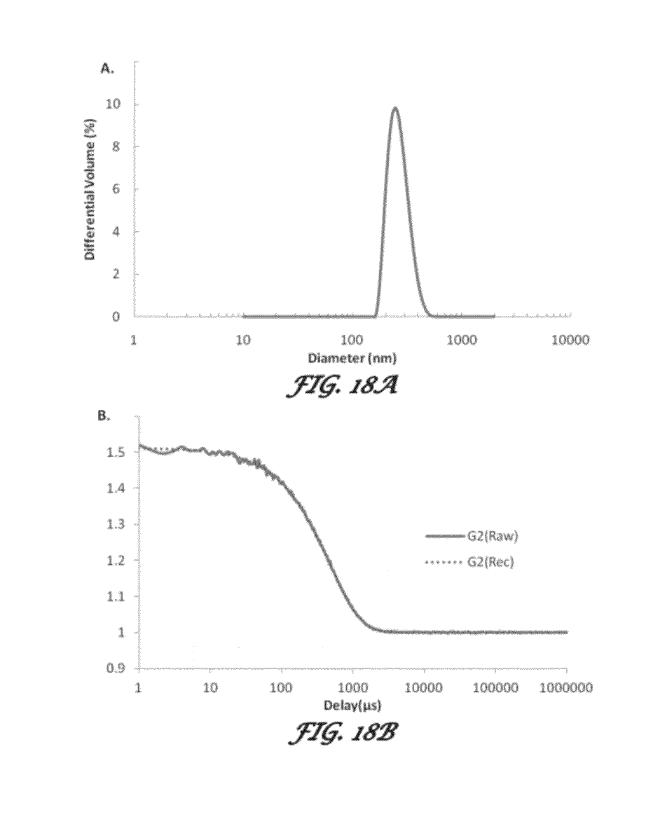

Hydrodynamic diameters, Dₕ=D_{c}, of nanoclusters and/or protein monomer in the aqueous crowder solutions were measured by dynamic light scattering (DLS) at various concentrations on a custom-built (Brookhaven) apparatus with a 632.8 nm laser, a fiber optic detector and an avalanche photodiode at various scattering angles and a temperature of ˜23° C., unless otherwise specified. The measurements at high cp ranging from 0.12 to 0.21 were made at 160-165° scattering angle to minimize multiple scattering (Horn 2000) with a specialized ˜60 μl sample cell (Beckman Coulter Part #A54094) to minimize the amount of protein required. To ensure that multiple scattering was minimized for the concentrated dispersion, additional measurements at a second scattering angle of 135° were conducted and found to give a Dₕ within 10% of the measurement at 160°. Data analysis was performed with CONTIN using a digital autocorrelator (Brookhaven BI-9000AT). DLS measurements in Table 1 were performed in triplicate. Reported average diameters corresponded to the DV50, or diameter at which the cumulative sample volume was under 50%. All samples contained one peak with a narrow distribution resulting in a relative standard deviation in peak width of less than 20% (see Table 6 for detailed analysis). The particular samples containing PEG300 without NMP (lines 3 and 4 in Table 1) were measured in the same cell, but with a Delsa Nano Particle Size Analyzer (Beckman Coulter, Fullerton, Calif.) at a scattering angle of 165°. The technique was validated with a polystyrene standard (φ=0.3) as shown in FIGS. 18A and 18B. A variety of studies were performed with much lower concentrations of protein (1 mg/ml) in 2 ml ampoules (Wheaton Scientific product #176776) at a scattering angle from 30° to 90° as previously reported. In addition, the average count rate for the larger volume, low concentration dispersions was recorded as the measured intensity for static light scattering (SLS) to determine the porosity and second osmotic virial coefficient.

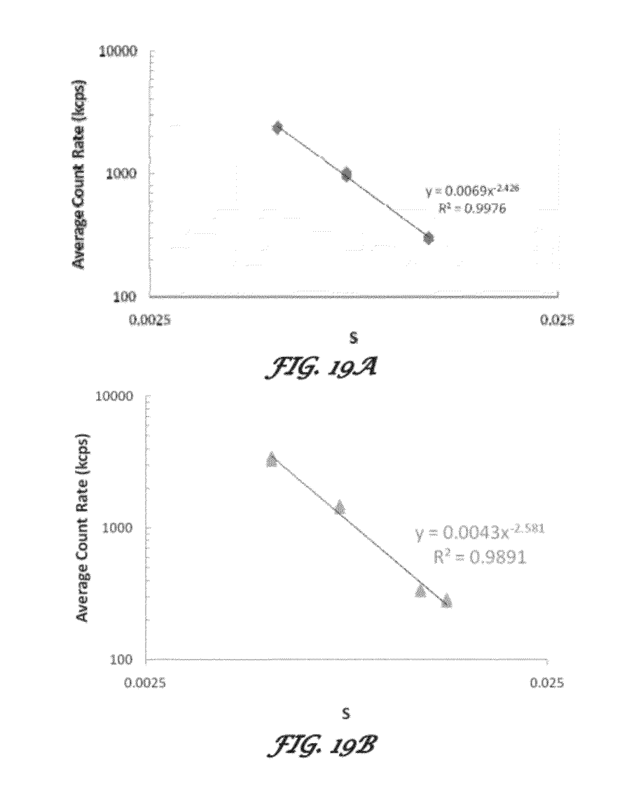

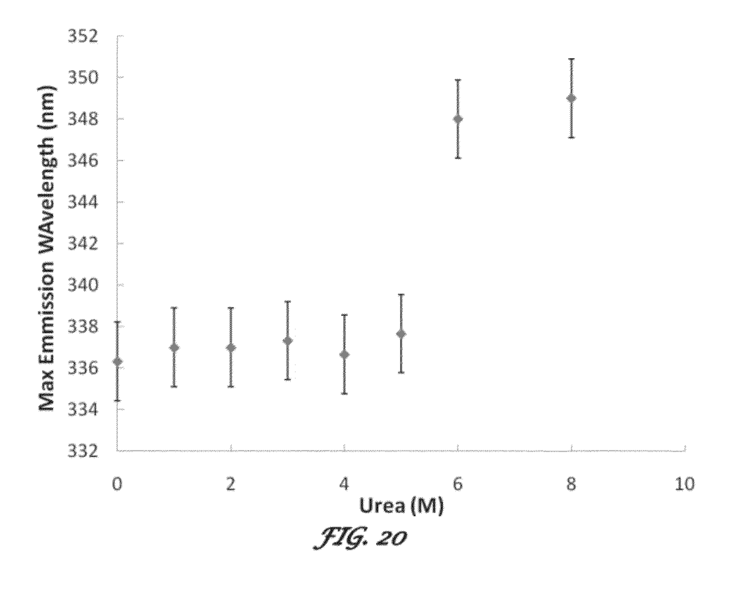

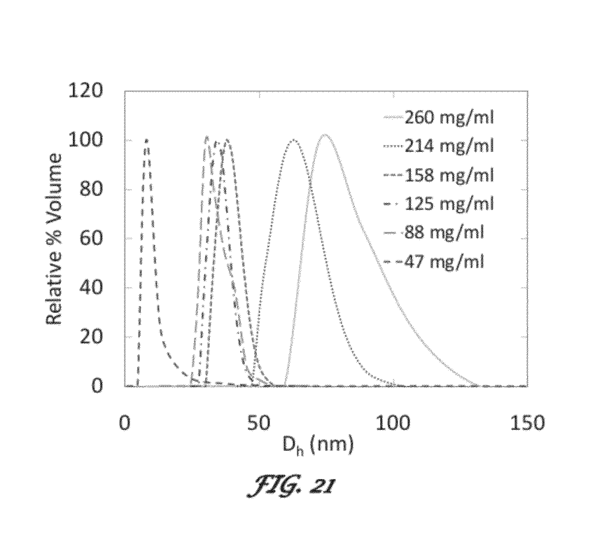

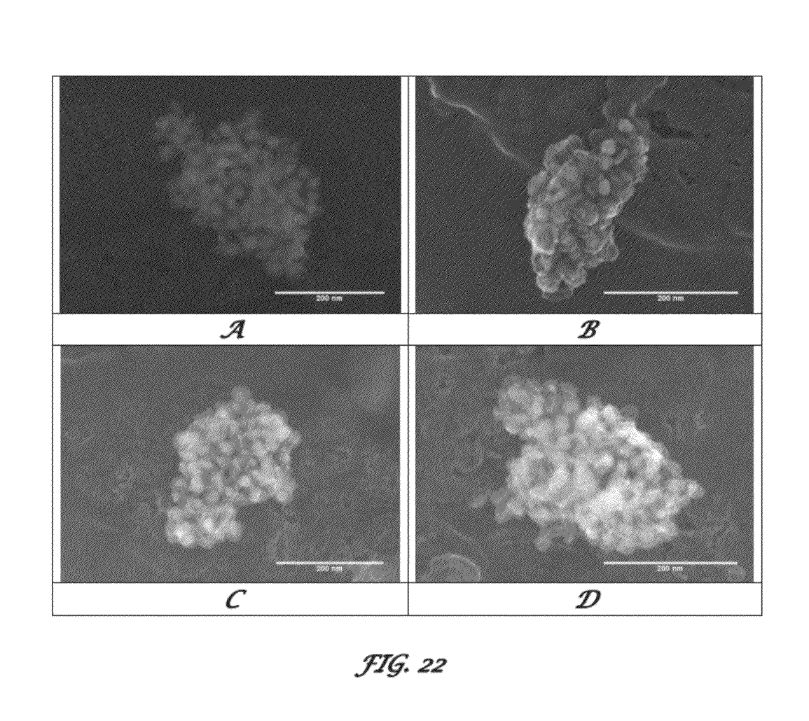

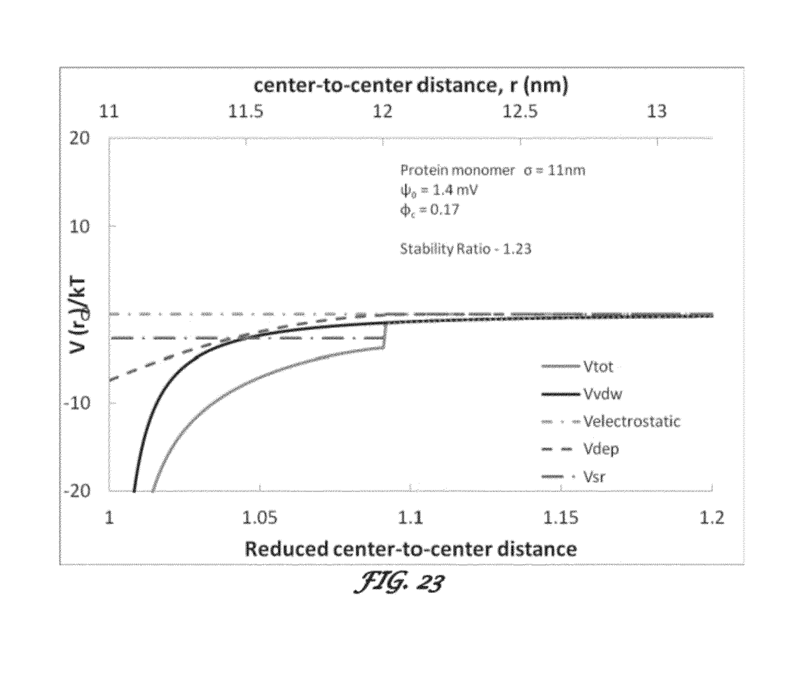

To further characterize particle morphology by scanning electron microscopy (SEM) and scanning transmission electron microscopy (STEM) the aqueous dispersions were diluted to 40 mg/ml and lyophilized. The degree of folding of the IgG within the concentrated dispersed particles was monitored from the λₘₐₓ fluorescence of the tryptophan residues in the fully unfolded protein (350 nm) versus the folded protein (336 nm). 25 A standard curve of the sheep IgG unfolding versus the concentration of a denaturant, urea, is shown in FIG. 20. The protein activity was characterized by a polyclonal capture enzyme-linked immunosorbent assay (ELISA), after 10 μl of the dispersion was diluted to 1 mg/ml in a phosphate buffer. These samples were also measured by DLS at 30° to characterize the protein monomer peak and to identify the presence of any irreversible aggregates. The monomeric peak obtained by DLS was also verified by size exclusion chromatography (SEC), described herein and Table 5.

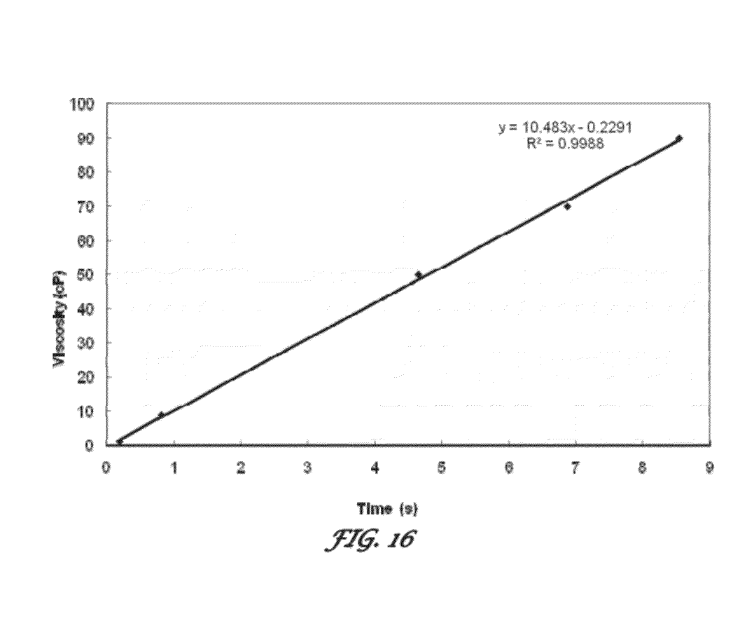

Viscosity Measurement: The apparent viscosity of the IgG nanocluster dispersions was measured in triplicate with 10% relative standard deviation using a 25 gauge (ID=0.1 mm) 1.5″ long needle attached to a 1 ml tuberculin slip tip syringe, according to the Hagen-Pouiselle equation. The velocity through the needle was determined with a video camera (Image J software) on the basis of the time to draw the dispersion from a height 0.4″ from the bottom of the cone to a height 0.1″ (−50 μl). The time was measured to within 0.05 seconds at least 3 times and averaged, while maintaining a nearly constant suction force by holding the end of the plunger at the 1 ml mark. A maximum volume of 10% of the cavity in the syringe was filled with dispersion to minimize variation in the pressure drop. A linear correlation between the time to draw 0.05 ml from the conical vial and the viscosity of various calibration fluids is shown in FIG. 16. (Miller 2011) The mixed aqueous-based solvent mixture viscosity (without protein) was measured using a Cannon-Fenske calibrated viscometer tube (Fisherbrand Catalog No. 13-617B) at least 3 times and averaged.

Formation of Highly Concentrated Nanocluster Dispersions: A transparent dispersion was formed upon gentle stirring of high concentrations of the lyophilized IgG:trehalose (1:1) particles in aqueous pH 6.4 phosphate buffer (FIGS. 1A and 1B). This transparent appearance is a consequence of the unusually low difference in refractive indices between the protein (˜1.42) and the aqueous solvent (˜1.33-1.37), despite the high protein concentration, cᵢ=c, of 150-275 mg/ml. The low turbidity enables visual observation that macroscopic particles were not present in all cases, which would be an important heuristic for the use of these dispersions for parenteral therapy.

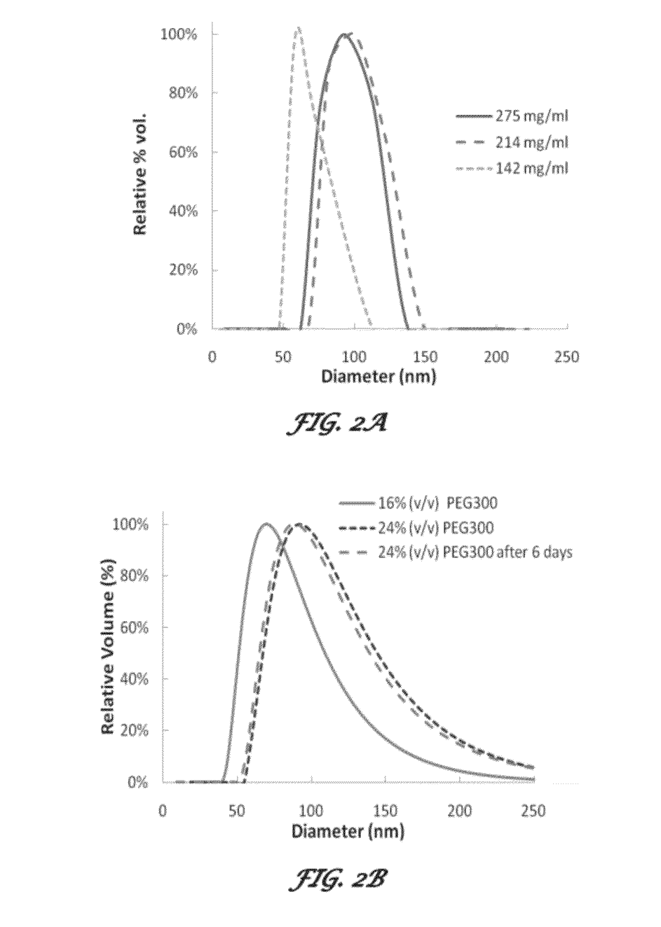

The DLS results are first presented for the highly concentrated dispersions in Table 1, followed by more specialized studies to determine the “DLS solubility” of the IgG and to vary pathways to prove that the clusters reached equilibrium. At a scattering angle of 160°, the Dₕ of the protein nanoclusters was approximately an order of magnitude larger than the value of 10 nm for an individual IgG molecule (Table 1). For the simplest cases in the first two rows with trehalose as the only extrinsic crowder, Dₕ-85-88 nm for cᵢ of 214 and 275 mg/ml (FIG. 2A and Table 1). The ability to accurately measure Dₕ at a scattering angle of 160° was determined by additional measurements at angles of 145° and 135° confirming the particle size within 10%. In FIG. 2A for one representative DLS run, no larger aggregates are observed in the size distribution and the peak with had a relative standard deviation of less than 10%. All Dₕ values reported in Table 1 are the average and standard deviation of 3 or more individual runs. For a lower concentration of trehalose as a crowder and a cᵢ of 142 mg/ml (φ_{T}=0.09), smaller 58 nm clusters were formed (FIG. 2A). When PEG300 was added (φ_{N}=0.16-0.24) to raise the total extrinsic crowder volume fraction, φ_{E}, to 0.25 and 0.34, the Dₕ increased modestly to 111 nm (FIG. 2B). Even larger 258 nm clusters were observed with a mixture of φ_{P}=0.08 and φ_{N}=0.16, despite a similar total φ_{E} of 0.33 as for the case without NMP.

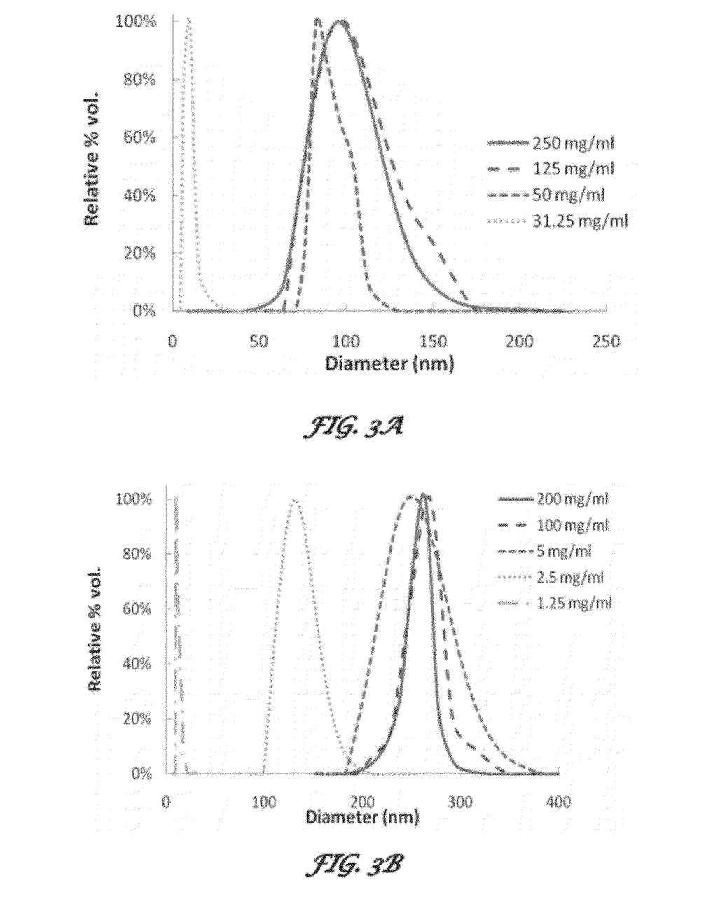

The IgG concentration, cᵢ, in the dispersion was diluted at constant compositions of all extrinsic crowders to define a “DLS solubility”, as shown in FIGS. 3A and 3B. The cᵢ where the Dₕ shifted from greater than ˜50 nm, to the hydrodynamic radius of the IgG, 11 nm was defined to be the solubility of the IgG in the extrinsic crowder solution. By this DLS solubility technique, the IgG solubility at the pI (pH 6.4) with 250 mg/ml trehalose (φ_{T}=0.15) was between a cᵢ of 31 and 50 mg/ml as the large clusters were still visible at 50 mg/ml, however only the soluble monomer was visible at 31 mg/ml (FIG. 3A). When 0.16 φ_{N} and 0.08 φ_{P} with 200 mg/ml trehalose are used in combination as crowders (φ_{E}=0.34), the IgG solubility decreased by 1 order of magnitude, to between 1.25 and 2.5 mg/ml (FIG. 3B). The DLS solubility at other crowder conditions, including the extrinsic crowder combination of PEG300 and trehalose, is also investigated (Table 1). Using the DLS IgG dilution method, the solubility was detected to be less than 1 mg/ml for an added 0.24 φ_{P} (Table 1). Solubilities of less than 1 mg/ml could not be detected by the DLS as the intensity of the scattered laser light was too weak.

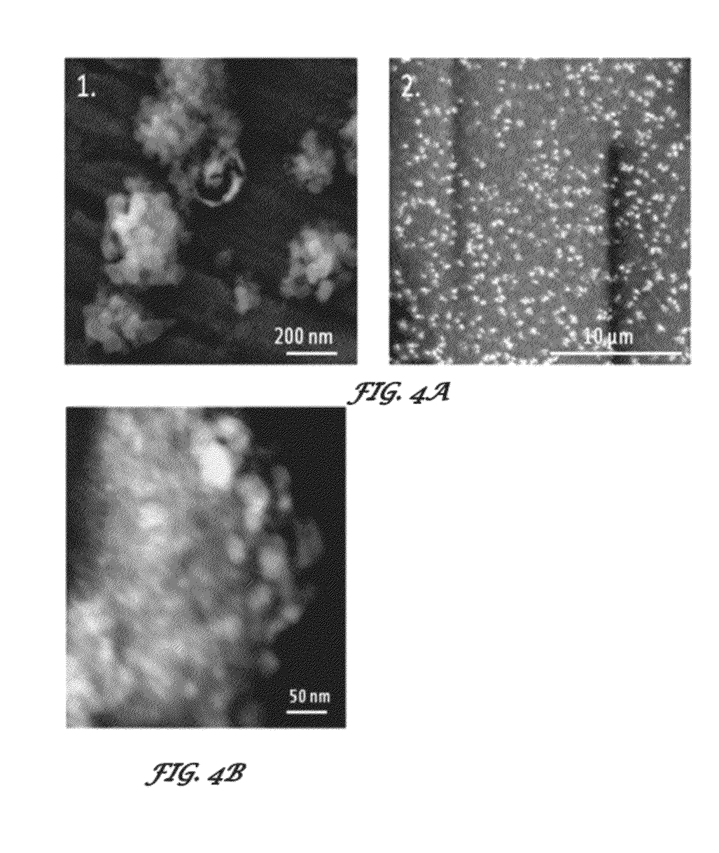

Rapidly frozen and lyophilized SEM and STEM images of the concentrated nanocluster dispersions confirm the particle size and show the morphology of the clusters formed with added extrinsic crowders 0.16 φ_{N} and 0.08 φ_{P} (φ_{E}=0.33, FIGS. 4A and 4B). As seen by both SEM (FIG. 4A1) and STEM (FIG. 4B), the particles are clusters formed of ˜50 nm and below primary particles. From SLS measured concurrently with the DLS at various scattering angles (30°, 45°, 75° and 90°), the fractal dimension of the nanoclusters can be determined as the exponent from a log-log plot of the scattering vector and the SLS intensity. The fractal dimension of a cluster, δ_{f}, characterizes the structure of a flocculated particle by relating the volume fraction of solid in the particle, φ₁ to the primary particle diameter, D, and the cluster diameter, D_{c}.

$${φ}_{E} = {( \frac{{D}_{o}}{D} )}^{{δ}_{f} - 3} \qquad ( 1 )$$

For a cluster composed of densely packed particles, δ_{f} approaches 3. For the nanoclusters formed with 250 mg/ml trehalose φ_{T}=0.15), a δ_{f} of 2.4 was measured experimentally (FIG. 19A) resulting in a φ₁ of individual proteins of 0.29. For the clusters formed with 0.16φ_{N} and 0.08φ_{P} (φ_{E}=0.34), a φ₁ of 0.29 was calculated using the measured δ_{f} of 2.6 (FIG. 19B).

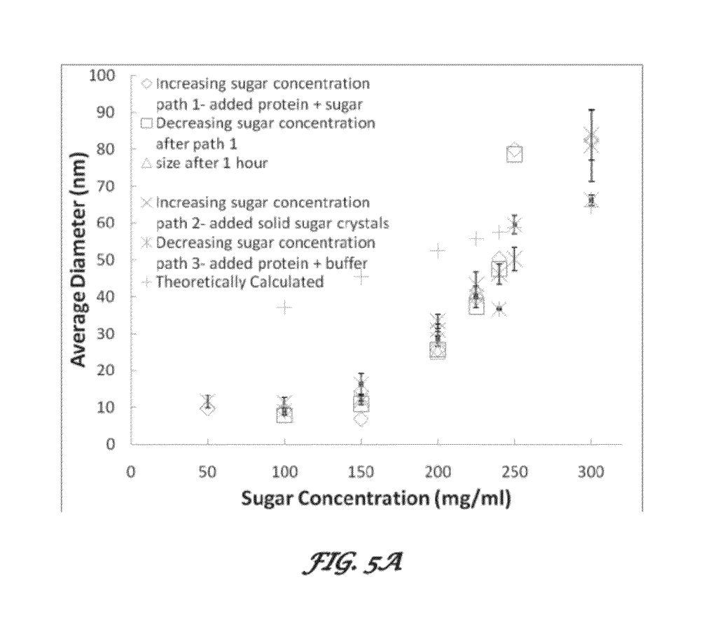

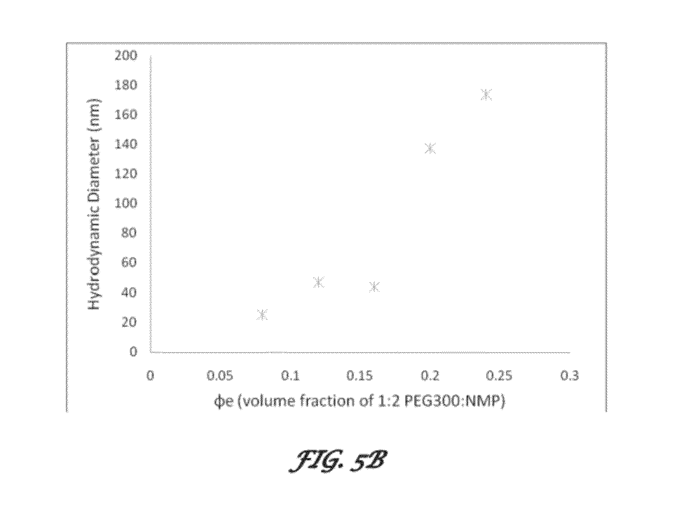

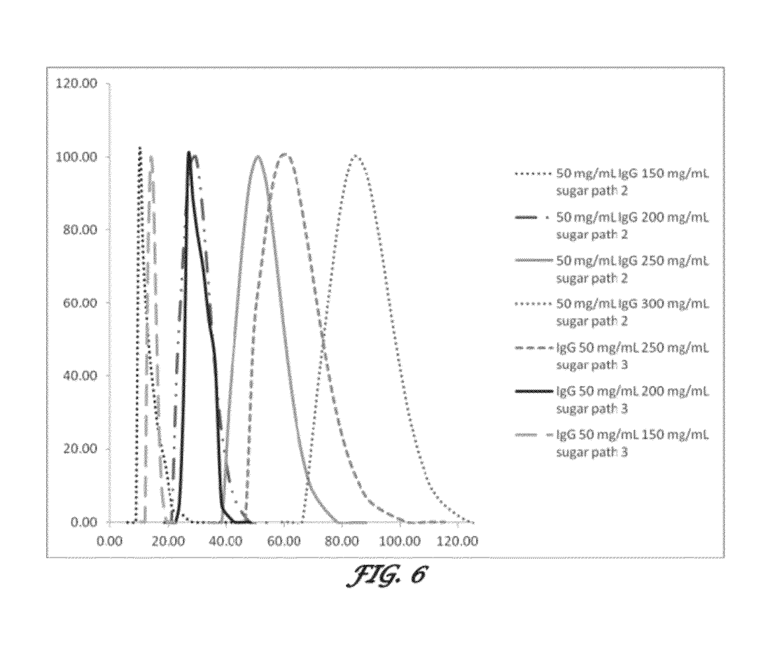

Effect of Crowder Concentration on Equilibrium Nanocluster Diameter: The effect of the total extrinsic crowder volume fraction (φ_{E}) on the size of the protein nanoclusters was determined by increasing and decreasing φ_{E} by a variety of paths at a constant cᵢ (FIG. 5A). In FIG. 5A path 1, φ_{T} was increased from 50 mg/ml up to 300 mg/ml by adding trehalose from a concentrated solution of trehalose (500 mg/ml trehalose). At each point, the cᵢ was maintained at 50 mg/ml by simultaneously adding a small volume of a 200 mg/ml concentrated protein dispersion with 200 mg/ml trehalose prior to measuring the Dₕ. By DLS, the protein at a concentration of 50 mg/ml trehalose was present as a monomer, as seen from the Dₕ of ˜10 nm, up to a trehalose concentration of 150 mg/ml. Above 150 mg/ml of trehalose, the protein formed clusters as shown by the increasing Dₕs. The protein cluster diameter increased linearly with trehalose concentration and reached ˜80 nm at a trehalose concentration of 300 mg/ml (FIG. 6). In FIG. 7, the mass of the trehalose was converted to φ_{T} by using the mass density of trehalose (1.64 g/ml). To verify the reproducibility of the cluster size by a separate pathway, the trehalose concentration starting from 300 mg/ml was decreased by adding a pure buffer solution. Again, the cᵢ was maintained at 50 mg/ml by adding small amounts of the concentrated protein dispersion mentioned above. The experimentally measured cluster size decreased at the same rate, based on φ_{T}, as it had increased while adding trehalose (FIG. 5A decreasing sugar concentration after path 1). In a separate study (FIG. 5A path 2), an alternate method was used to increase the trehalose concentration. Trehalose crystals were dissolved directly in the protein solution at 50 mg/ml IgG and 50 mg/ml trehalose to increase the trehalose concentration. At each trehalose concentration, the size of the protein clusters produced by both methods, whether increasing or decreasing trehalose concentration, closely agreed. In addition, path 3 was tried in which, the study was started with a trehalose concentration of 300 mg/ml and a constant IgG concentration of 50 mg/ml which was diluted to 100 mg/ml IgG by using pH 6.4 phosphate buffer with the requisite small amounts of a 200 mg/ml IgG dispersion to maintain the IgG concentration at 50 mg/ml. All these different paths yield sizes that agree well with each other at the different trehalose concentrations that were tried and seem to fall on the same straight line.

A second crowder composition, a 1:2 by volume solution of PEG300 and NMP, was also used to determine particle size at various total φ_{E}. In this case, a measured volume of the 1:2 volume solution of PEG300 and NMP was added to increase total φ_{E}, while constant protein and trehalose concentrations of 30 mg/ml were maintained by the addition of a small amount of the 1:1 wt ratio protein to trehalose lyophilized powder. Cluster growth was observed as the φ_{E} of PEG300 and NMP was increased to 0.15 and higher. The largest particles of ˜180 nm were seen at a φ_{E} of PEG300 and NMP of 0.3.

Actual hydrodynamic diameter distributions for some selected samples in FIG. 5A obtained by DLS are shown in FIG. 6. As can be seen the distributions are fairly narrow with a relative standard deviation of less than 10% over the mean. Also it can be seen that not only do the cluster sizes for different paths match up well as is shown in FIG. 5A but the distributions also match up well as can be seen in FIG. 6. The size of the protein clusters formed for both the trehalose crowder only and the 1:2 PEG 300: NMP crowder system was plotted against the total extrinsic crowder volume fraction in FIG. 7. Both types of crowder systems give very comparable linear growth of the protein cluster size as shown in FIG. 7. In fact both the crowder systems nearly fall on the same line.

Properties of Nanocluster Dispersions (Low Viscosity and High Molecular Stability): Syringable viscosities (e.g. <50 cP) were obtained for all of conditions in Table 1, except the final row with 204 mg/ml IgG (0.08 φ_{P}/0.16 φ_{N}). The viscosities for the samples with φ_{P} between 0.16-0.24 were modestly higher than those for the NMP-PEG mixtures. Even higher volume fractions of PEG300, 0.50, increased the viscosity to the point where it was not syringeable at 150 mg/ml IgG. When only trehalose was used as an extrinsic crowding agent, the viscosity of the protein dispersion at 214 mg/ml, was 37 cP. Viscosities this low have rarely been reported, if reported at all, for therapeutic proteins at such a high concentrations. Furthermore, solutions often cannot be formed at 200-300 mg/ml as the protein solubilities are not this high.

The apparent dispersion viscosity is commonly described as a function of the intrinsic viscosity, [n], maximum volume fraction of particles, φₘₐₓ, and the solvent viscosity, η₀, using the Krieger-Dougherty equation (Eq. 2).

$$\frac{η}{{η}_{o}} = {[ 1 - ( \frac{{φ}_{l}}{{φ}_{max}} ) ]}^{- [ η ] {φ}_{max}} \qquad ( 2 )$$

The η may be reduced by lowering η₀ or [η], which is a minimum of 2.5 for hard sphere colloids, and increasing φₘₐₓ. In Table 1, φ₁ is the volume fraction of protein and φₘₐₓ=0.55. Because we have an equal mass of trehalose as protein present in the solvent, the solvent viscosity is increased to account for the soluble sugar. For each of the PEG-NMP formulations, [η] was fairly low, between 13 and 16. At a concentration of 275 mg/ml IgG with only sugar as a crowder, the [η] for the protein dispersion is around the same value, 14. For the three studies with only PEG as an added crowder, the [η] values are a little larger (19-20) than for the NMP-PEG samples but still smaller than for many reported proteins with intrinsic viscosities as high as 100.

Given that the dispersions offer low viscosities at high concentrations, the protein stability within the dispersion and upon dilution is examined. At a cᵢ of 100 mg/ml, diluted from the concentrated dispersions with the crowders present, a fluorescence assay was utilized to show protein folding in the concentrated dispersion. Isolated protein amino acid side chains, tryptophan and to a lesser extent tyrosine, excited at 295 nm, will emit a maximum signal at 350 nm. Due to the local environment within a fully folded protein, the maximum emission wavelength (λₘₐₓ) will shift to 336 nm for the sheep IgG (FIG. 20). Upon full unfolding of the protein, the local environment of the amino acid residues will change and λₘₐₓ will increase to 350 nm. Thus a scan of the emission at wavelengths between 336 and 350 where the dispersed particles are excited at 295 nm and the maximum emission wavelength is recorded will indicate the folding of the protein within the nanoclusters. For both dispersions with pure sugar crowder and with NMP and PEG, the λₘₐₓ of 336 nm indicates the fully folded state of the protein (Table 2). High retention of monomeric protein and antibody activity for the protein diluted and dissolved to 1 mg/ml in a pH 7.0 phosphate buffer from the aqueous dispersion, is shown in Table 2. As the sheep IgG used in these initial studies does not bind a single target, a polyclonal anti-sheep IgG capture ELISA was used to monitor loss of conformational epitopes due to denaturation. The relative EC₅₀˜1.1 indicates similar binding to the standard IgG, within error of the experiment (Table 2). The relative EC₅₀ values close to 1 indicate a negligible change in activity. According to DLS measurements for the dissolved IgG from nanoclusters at 1 mg/ml, the protein dissolves to a Dₕ of ˜10 nm, the Dₕ of the sheep IgG (Table 2). Additional size exclusion chromatography to quantify the % monomer of the protein upon dilution into a pH 7.0 buffer is found herein (Table 5).

Protein Nanocluster Interactions: Interparticle interactions of the nanoclusters in dispersion were quantified by measuring the second virial coefficient, B₂, by static light scattering (SLS). Since it was necessary to dilute the concentrated dispersion to remove multiple scattering, only the NMP-PEG system was utilized. A plot of KcP(θ)/R_{θ} versus c was used to determine B₂ with the relationship

$$\frac{Kc}{{R}_{θ}} = \frac{1}{P ( θ )} ( \frac{1}{{M}_{w}} + 2 {B}_{2} c ) \qquad ( 3 )$$

where, K is an optical constant

$$K = \frac{4 {π}^{2} {{n}_{o}^{2} ( \frac{d n}{d c} )}^{2}}{{N}_{A} {λ}^{4}} \qquad ( 4 )$$

Here, nₒ is the refractive index of the solvent and λ is the wavelength of the incident beam. The refractive index increment (dn/dc) for the nanoclusters was taken to be the same as that for protein aqueous solutions (0.185 ml/g), as additional crowders and the formation of the nanoclusters are not anticipated to affect the value of do/dc. In the nanoclusters, intraparticle interference influences the measured intensity. To reduce this effect, an additional factor, P(θ), was added to Eq. 3 to account for the change from pure Rayleigh scattering to Debye scattering (Eq. 5).

TABLE 1
Hydrodynamic diameter of clusters, protein monomer solubility by
DLS dilution, and viscosities in pH 6.4 50 mM phosphate buffer.

| IgG (cᵢ) or | | | | | φ_{E} | | DLS | Dispersion | Solvent | |
|---|---|---|---|---|---|---|---|---|---|---|
| Trehalose | | | | | (Total Extrinsic | Hydrodynamic | Solubility | Viscosity | Viscosity | Intrinsic |
| Conc. (mg/ml) | φ_{I} | φ_{T} | φ_{P} | φ_{N} | Crowders) | Diameter | (mg/ml) | (cP) | (cP) | Viscosity |
| 214 | 0.16 | 0.13 | 0 | 0 | 0.13 | 85 ± 25 | 31-50 | 37 | 1.5 | 17 |
| 275 | 0.21 | 0.17 | 0 | 0 | 0.17 | 88 ± 9 | 31-50 | 63 | 2.1 | 14 |
| 157 | 0.12 | 0.09 | 0.16 | 0 | 0.25 | 111 ± 10 | ND | 51 ± 7 | 3.4 | 20 |
| 162 | 0.12 | 0.10 | 0.24 | 0 | 0.34 | 111 ± 10 | <1 | 48 ± 1 | 3.5 | 19 |
| 157 | 0.12 | 0.10 | 0.08 | 0.16 | 0.33 | 258 ± 24 | 1.25-2.5 | 25 ± 5 | 4.3 | 13 |
| 204 | 0.15 | 0.12 | 0.07 | 0.14 | 0.34 | ND | 1.25-2.5 | 102 | 5.6 | 16 |

TABLE 2
Characterization of protein stability.

| | φ protein | φ_{E} | | | DLS - |
|---|---|---|---|---|---|
| | within | (ex- | | ELISA | Hydro- |
| Dispersion | nanocluster | trin- | TRP | Relative | dynamic |
| name | from SLS | sic) | λₘₐₓ | EC₅₀* | Diameter |
| 275 mg/ml | 0.29 | 0.17 | 336 ± 1 | 1.1 ± 0.1 | 9 ± 2 |
| 157 mg/ml - | 0.29 | 0.33 | 336 ± 1 | 1.1 ± 0.2 | 10 ± 1 |
| 0.08 φ_{P}/0.16 φ_{N} | | | | | |

Maximum emission wavelength (λₘₐₓ) for concentrated protein dispersions from tryptophan fluorescence. λₘₐₓ for fully folded protein is 336 nm and for fully unfolded protein is 349 nm. For ELISA and DLS, dispersions were diluted in pH 7.0 buffer to 1 mg/ml.
*Relative EC₅₀ was calculated as the difference between the EC₅₀ of the reconstituted dry powder to the orginal purified solution prior to processing.

$$\frac{1}{P ( θ )} = 1 + \frac{16 {π}^{2} \overline{{R}_{g}^{2}}}{3 {λ}^{2}} {sin}^{2} ( \frac{θ}{2} ) \qquad ( 5 )$$

A low scattering angle, 30°, was chosen to reduce the second term of the 1/P(θ) equation (Eq. 5). The slope in FIG. 7 indicates a positive B₂ of 6.6*10⁻⁵ mol*ml/g² thus signifying that the nanocluster interparticle interactions are slightly repulsive. The repulsive nature of the nanocluster interparticle interactions is supported by other indirect characterization techniques. If the nanocluster interparticle interactions were attractive, we may not have seen discrete individual particles by SEM or DLS.

Assembly of non-gelling dispersions of monodisperse protein nanoclusters relies on properly balancing hierarchical, multi-scale interactions. Protein molecules should attract one another (favoring cluster formation), individual proteins should interact neutrally with the clusters (Groenewold and Kegel 2001) (limiting cluster size), and nanoclusters should repel one another (avoiding gelation).

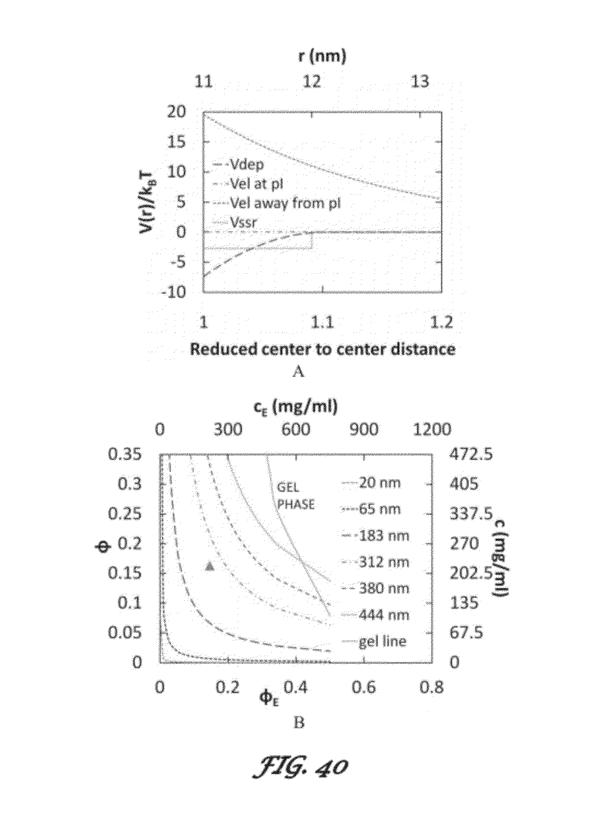

We begin by examining the potential of mean force between two proteins at the molecular level before discussing the nanoclusters. FIG. 40a shows estimates for the contributions to the potential of mean force V(r) for two 1B7 molecules (the parameters used in this case are given in Table 14). For pH 3 units away from pI, Vₑₗ(r) is strongly repulsive. At these conditions, as should be expected, only very small clusters have been observed, as seen for lysozyme. (Stradner, Sedgwick et al. 2004) Near the pI, Vₑₗ becomes very weak and thus with a strong V_{dep} for c_{E}=220 mg/ml, V(r) is attractive. This attraction may now be shown to drive formation of clusters, as described by the equilibrium free energy model.

To understand the cluster formation mechanism, consider an aqueous solution of protein and relatively concentrated crowder molecules at conditions near the protein's pI. Two protein molecules in this system will strongly attract one another because the magnitude of the electrostatic repulsion between the weakly charged monomers is vanishingly small compared to the short-range depletion attraction (FIGS. 36A and 40A). However, the interaction between a protein monomer and a cluster of proteins is more complex because the monomer feels, in addition to the short-range depletion attraction, the net effect of many weak, longer-ranged repulsions from the charged protein within the cluster. This interaction can be attractive or repulsive depending on the size of the cluster. (Groenewold and Kegel 2001; Groenewold and Kegel 2004) If the cluster is sufficiently large, then these repulsions balance the depletion attraction, limiting further cluster growth (FIG. 36A). The equilibrium cluster size increases with increasing strength of depletion interactions between the protein monomers (e.g., with increasing crowder concentration) and decreases with the increasing strength of the repulsive interactions (e.g., with number pH units away from the pI). Because of their collective electrostatic repulsions, it will be shown that fully grown clusters in solution do not attract one another.

The contours for protein cluster diameters, D_{c}, shown in FIG. 40B were computed from an extension of a simple equilibrium free energy model (Groenewold and Kegel 2001; Groenewold and Kegel 2004) which has previously been applied to understand clustering of polymeric colloids in organic solvents. (Sedgwick, Egelhaaf et al. 2004) In that model, D_{c} is determined by a balance between short-range interparticle attractions and weak, longer-range electrostatic repulsions.

To understand the equilibrium model, consider n_{c} proteins of radius R that form a cluster of radius R_{c} in solution, as shown in FIG. 36A. In our analysis, the only attraction we explicitly consider is the crowder-mediated depletion interactions, which (as explained above) is the dominant attractive interaction under strong clustering conditions. If the depletion interaction between two proteins is −∈ and each protein has C nearest neighbors in the cluster interior, then the effective depletion contribution to the free energy per protein molecule in the cluster interior will be −∈C/2. The “missing” depletion interactions for proteins on the cluster surface are accounted for by adding an effective surface energy term (4πR_{c}²γ), where the surface tension is approximated as γ=∈/4πR². In other words, the depletion attractions contribute the following to the cluster free energy,

$${F}_{att} = - \frac{ɛ {Cn}_{c}}{2} + 4 π {R}_{c}^{2} γ \qquad ( 6 )$$

Assuming that the charges are negligibly screened within the cluster (as discussed elsewhere herein), their repulsive self-energy can be approximated by that of a uniform distribution of point charges in a spherical volume with the cluster radius R_{c}, i.e.,

$${F}_{rep} = \frac{3 λ {k}_{B} {Tn}_{c}^{2} {q}^{2}}{5 {R}_{c}} \qquad ( 7 )$$

where λ is the Bjerrum length (λ=e²/4π∈ᵣ∈₀k_{B}T), ∈ᵣ is the relative permittivity of the medium, and q is the charge per protein. The minimization of the F=Fₐₜₜ+Fᵣₑₚ with respect to R_{c} (or n_{c}) gives

$${n}_{c} = \frac{10 πγ {R}^{3}}{3 {k}_{B} T λ {q}^{2}} \qquad ( 8 )$$

This simple result illustrates that the equilibrium n_{c} increases with attraction and decreases with electrostatic repulsion.

To further understand the cluster free energy in terms of the translational and combinatorial entropy of the counterions dissociating from the protein molecules, it is instructive to write (Groenewold and Kegel 2001)

F_{sntropic}=2η_{c}q[ln(q/q₀)−1] (9)

The quantity q₀ represents the charge per protein q that minimizes the overall cluster free energy (see also Eq. 10 below) for conditions corresponding to very low values of ζ potential (where λ/R_{c}→0; i.e., the weakly charged systems of interest here). It can be expressed as

q₀=(4πn_{d}/3φ)^{1/2}(R/b)^{3/2}e^{-λ/(2b-1)} (10)

where n_{d} is the number of dissociable sites on a protein surface, b is the distance of closest approach between a counterion and a charge on the protein surface, and φ is the volume fraction of proteins in solution. As discussed extensively elsewhere, (Groenewold and Kegel 2001) higher values φ generally result in lower q₀ because, with more proteins present in the system, fewer counterions per protein need to dissociate to achieve the same increase in counterion translational entropy. Combining terms, the free energy per protein of a cluster given by (Groenewold and Kegel 2004)

$$\frac{{f}_{c}}{{k}_{B} T} = \frac{ɛ C}{2 {k}_{B} T} + \frac{4 π {R}_{c}^{2} γ}{{k}_{B} {Tn}_{c}} + \frac{3 λ {n}_{c} {q}^{2}}{5 {R}_{c}} + 2 q [ ln ( \frac{q}{{q}_{0}} ) - 1 ] \qquad ( 11 )$$

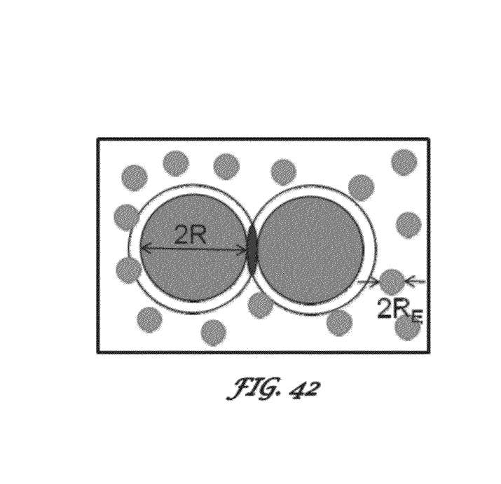

To take into account the porosity of the protein cluster, we modify the original model by expressing the cluster radius as

$${R}_{c} = {( \frac{{n}_{c}}{k} )}^{\frac{1}{{δ}_{f}}} R \qquad ( 12 )$$

where δ_{f} is the fractal dimension (2.6 from FIG. 44). The resulting modified free energy equation is

$$\frac{{f}_{c}}{{k}_{B} T} = \frac{ɛ C}{2 {k}_{B} T} + \frac{ɛ {n}_{c}^{( \frac{2}{{δ}_{f}} - 1 )}}{{k}_{B} T {k}^{2 / {δ}_{f}}} + \frac{3 λ {n}_{c}^{( 1 - \frac{1}{{δ}_{f}} )} {q}^{2}}{5 {R}_{c} {k}^{1 / {δ}_{f}}} + 2 q [ ln ( \frac{q}{{q}_{0}} ) - 1 ] \qquad ( 13 )$$

Minimizing f_{c} with respect to n_{c} at q=q₀ gives the following estimate for the equilibrium aggregation number (n*)

$${n}^{c} = {k}^{\frac{3}{3 - 2 {δ}_{f}}} {\{ \frac{5 ( {δ}_{f} - 2 ) ɛ R}{3 ( {δ}_{f} - 1 ) {k}_{B} T λ {q}_{0}^{2}} \}}^{\frac{{δ}_{f}}{2 {δ}_{f} - 3}} \qquad ( 14 )$$

As should be expected, the cluster size increases with increased strength of the attraction, E. Since we are interested here in cases where Vₛₛᵣ (hydrogen-bonding, hydrophobic interactions, etc.) is smaller in magnitude than the crowder-mediated depletion attraction (FIG. 40A); we approximate ∈ as the contact value of the depletion potential in Eq. 16 [−∈(φ_{E},R/R_{E})=V_{dep}(r=2R)]. In the limit of solid clusters with δ_{f}=3, Eq. 14 becomes Eq. 8 which is essentially the same as Eq. 23 in Groenewold and Kegel. (Groenewold and Kegel 2001) The only difference is in the coefficient which is explained elsewhere. (Arfken and Weber 1995)
Table 15 summarizes our input variables for the model to determine the R_{c} contours in FIG. 40B. The R_{c} is determined from setting n* from Eq. 9 into Eq. 7. The total number of dissociable sites on the protein monomer at a given pH, n_{d}, was chosen as 50 based on previous estimates. (Chari, Jerath et al. 2009) The fractal dimension is chosen as 2.6 based on the SEM images and SLS measurements (FIG. 44). The c, inside the clusters was chosen as 25 as explained in detail in elsewhere herein. The distance between opposite charges in an ionic bond is taken to be ˜0.1 nm and the protein diameter is 11 nm (Table 15). (Harn, Spitznagel et al. 2010)

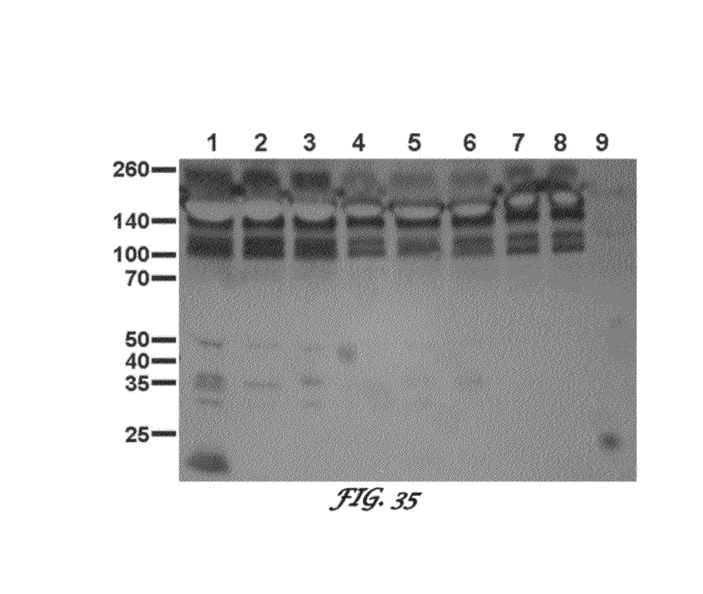

The effects of φ and φ_{E} on R_{c} are illustrated in FIG. 40B, from the equilibrium model for clustering of colloids, (Groenewold and Kegel 2001; Groenewold and Kegel 2004) which has been extended to account for the fractal dimension of the cluster (see Eq. 14). We assumed based on FIG. 40A that short-range attractive interactions between proteins are dominated by depletion attractions (Eq. 16) at high values of φ_{E} as is evident at contact. This attraction is balanced by weak long-ranged repulsions with negligible electrostatic screening within the dense clusters (described elsewhere herein). On a horizontal pathway in FIG. 40B, increasing φ_{E} at fixed φ strengthens V_{dep} (crowding) and hence increases R_{c}. This pathway raises the depletion attraction between protein monomers (higher E) and therefore the numerator in Eq. 14 (and likewise Eq. 8) which increases R_{c}. The predictions of the model are in reasonable agreement with experimental data as shown in FIG. 37B and FIG. 37D where the cluster size increases with an increase in the c_{E}. In addition, on a vertical pathway increasing φ at fixed φ_{E} lowers the charge per protein in the cluster, because fewer counterions per protein must dissociate to obtain the same balance between entropy and energy in the system, (Groenewold and Kegel 2001) which also increases R_{c}. For the combined change whereby φ and φ_{E} decrease upon dilution along a diagonal slant, R_{c} decreases (FIG. 40B). Here both the decrease in depletion attraction and the lower φ and its effect on charge produce a decrease in R_{c}. Again this prediction is in agreement with the experimental data as shown in FIGS. 37A-D. Our new model, as well as the one it is based on, (Groenewold and Kegel 2001; Groenewold and Kegel 2004) is only meant to provide qualitative predictions. The model does not consider intracluster charge screening, differences in ∈ᵣ inside and outside the cluster, and variations in the attractive interaction with r. However, the simple equilibrium model substantiates the novel experimental discovery of reversible equilibrium nanoclusters and qualitatively predicts the experimental trends in D_{c}.

In contrast to the predominantly attractive interactions between individual proteins near their pI in FIG. 40A, the resulting nanocluster interactions are highly repulsive (FIG. 40C). The dominance of intercluster repulsions is due to the large number of weakly charged proteins per cluster (>1000 proteins/cluster and ˜1 elementary charge/protein) and the longer range of Vₑₗ (Eq. 19) which scales as R_{c}. In contrast, the range of V_{dep} and Vₛₛᵣ (Eqs. 16 and 17) is <1 nm, and thus almost negligible versus the intercluster spacing (FIG. 40C inset).

Under conditions for which the electrostatic repulsion is insufficient to balance the attractive forces (i.e., very high crowder or protein concentrations), the protein can also form a gel. (Lu, Zaccarelli et al. 2008) The spinodal instability associated with this transition in the context of the clustering model (Groenewold and Kegel 2004) can be defined as the locus of points where d²f_{cl}/dq²=0 (see gray line in FIG. 40B). Note that equilibrium clusters with various sizes may be formed before the gel phase boundary, according to the experimental data and the theoretical cluster size contours.

### Potential of Mean Force Between Two Proteins in the Presence of the Surrounding Media

The potential of mean force V(r) between two protein particles, whether protein monomers or nanoclusters, in the presence of the other molecules in the media, provides a basis for understanding the relevant multiscale interactions. It can be modeled as a sum of components, which typically include depletion (dep) interactions, specific short-ranged (ssr) interactions, and van der Waals (vdw) interactions, as well as electrostatic (el) interaction, i.e.,

V(r)=V_{dep}(r)+Vₛₛᵣ(r)+V(r)_{vdw}+Vₑₗ(r) (15)

Where r is the separation between particle centers. The depletion attraction (Asakura and Oosawa 1958; Minton 2007; Zhou 2008; Zhou, Rivas et al. 2008) (commonly referred to as “crowding”) is an effective (osmotic) interaction that particles experience due to the presence of smaller cosolutes or “extrinsic crowders” (here, trehalose molecules) in solution. It arises because entropy favors microstates where protein particles are close to one another; i.e., configurations which make more of the volume available to the smaller crowders (FIG. 42). In FIG. 42, because of the proximity of the proteins, the actual three dimensional volume represented by the area shaded dark gray between the two large gray circles (in the two dimensional figure) becomes available to the trehalose molecules.

The depletion attraction is often described by the Asakura-Oosawa potential (S. Asakura 1954; Asakura and Oosawa 1958)

$$\frac{{V}_{dep}}{{k}_{B} T} = - \frac{{φ}_{E}}{2} {( 1 - \frac{r - 2 R}{2 {R}_{E}} )}^{2} ( 2 + \frac{3 R}{{R}_{E}} + \frac{r - 2 R}{2 {R}_{E}} ) \qquad ( 16 )$$

where R is the protein particle radius and φ_{E} and R_{E} represent the volume fraction and radius of the extrinisic crowder, respectively. (Tuinier, Rieger et al. 2003) Since the strength of the depletion attraction is proportional to φ_{E}, it can be tuned experimentally by modifying the crowder concentration. The range of this attraction scales with R_{E}(˜0.5 nm for trehalose), and so it is considerably smaller than R (˜5.5 nm for the protein monomer).

What we term the specific short-ranged attraction between protein particles represents a combination of molecular-scale interactions including hydrogen bonding, hydrophobic interactions between exposed apolar protein patches, and fluctuating charge dipoles. (ten Wolde and Frenkel 1997; Curtis, Prausnitz et al. 1998; Rosenbaum, Kulkarni et al. 1999; Kulkarni, Dixit et al. 2003) For simplicity, it is often modeled as asquare-well potential—

$${V}_{ssr} / {k}_{B} T = \{ \begin{matrix}∞ & r < 2 R \\ - {V}_{0} / {k}_{B} T & 2 R \leq r \leq 2 R ( 1 + Δ ) \\ 0 & r > 2 R ( 1 + Δ )\end{matrix} \qquad ( 17 )$$

where V₀/k_{B}T is the well depth (V₀/k_{B}T˜2.7 for a monoclonal antibody (Bajaj, Sharma et al. 2007)) and the width (2RΔ) is ˜1 nm. (ten Wolde and Frenkel; Curtis, Prausnitz et al.; Rosenbaum, Kulkarni et al.; Kulkarni, Dixit et al.; Stradner, Sedgwick et al. 2004) It is reasonable to assume that the range of the specific short-ranged interactions (2RΔ) is constant and thus independent of particle size (R); i.e., Δ˜R⁻¹. (Curtis, Prausnitz et al.; Kanai, Liu et al.; Yadav, Liu et al.) Thus, the range of influence of a 1 nm ssr interaction becomes negligible for a 100 nm protein colloid relative to a 10 nm protein molecule, which will be shown to play a key role for the low viscosity of the nanocluster dispersions.

The van der Waals attraction between two particles can be expressed in terms of a Hamaker constant between two proteins through water A_{pwp} as (Hiemenz and Rajagopalan 1997)—

$$\frac{{V}_{vdw}}{{k}_{B} T} = \frac{- {A}_{pwp}}{6 {k}_{B} T} [ \frac{2 {R}^{2}}{( r - 2 R ) ( r + 2 R )} + \frac{2 {R}^{2}}{{r}^{2}} + ln [ \frac{( r - 2 R ) ( r + 2 R )}{{r}^{2}} ] ] \qquad ( 18 )$$

It is relatively weak compared to the other interactions considered in this study (A_{pwp}/k_{B}T in water is only ˜0.04), and hence it is not considered explicitly in our analysis.

The electrostatic repulsion between particles is given by (Hiemenz and Rajagopalan 1997)

$$\frac{{V}_{EL}}{{k}_{B} T} = \frac{64 π R {Γ}_{0}^{2} {η}_{∞}}{{κ}^{2}} exp ( - κ ⌈ r - 2 R ⌉ ) \qquad ( 19 )$$

where Γₒ is a function of ψ₀ the particle surface potential (Hiemenz and Rajagopalan 1997), η_{∞} is the bulk ion concentration (50 mM), κ is the inverse Debye length (κ⁻¹=0.7 nm for the bulk buffer solution). Note that the magnitude of the electrostatic repulsion depends on both the charge and the size of the particles.

### Surface Potential and Zeta Potential of IgG Clusters

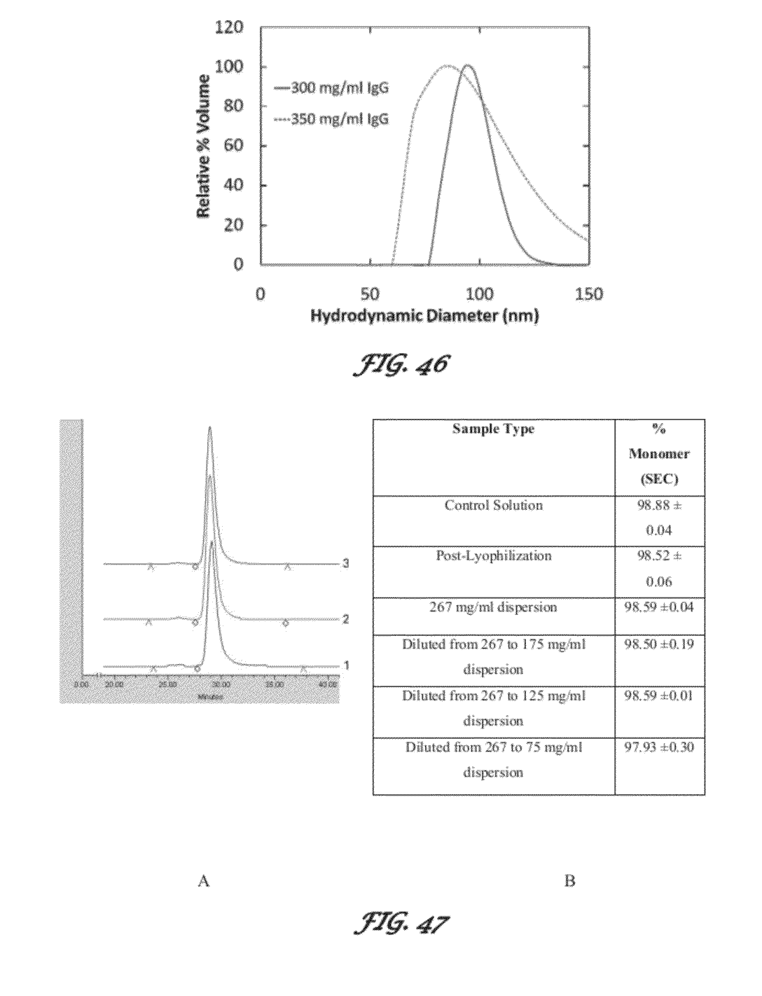

The zeta potential for 50 nm clusters of polyclonal sheep IgG, produced from dispersing the same powder as in FIG. 47 with c=50 mg/ml and c_{E}=270 mg/ml was measured to be 3.9±0.75 mV at a pH of 6.4 near the pI of 6.4. From this value and a Debye length of 2 nm, we estimated about 1-2 effective charges on the surface of each protein molecule from the relation

$$ζ = \frac{Q}{4 πε {R}_{s} ( 1 + κ {R}_{s} )} \qquad ( 20 )$$

where Q is the surface charge on a particle, Rₛ the radius for the particle at the shear plane, which was approximated as equal to the radius of the particle (R_{c}), and κ is the inverse Debye length. Based on this Q, a surface potential of 64 mV was calculated for the IgG nanoclusters

$${ψ}_{0} = \frac{Q}{4 πε {R}_{c}} \qquad ( 21 )$$

This surface potential gives a potential barrier of about 15 k_{B}T in the potential of mean force for two protein clusters as shown in FIG. 4c which stabilizes the clusters against aggregation. Given the large quantities of protein required to measure ζ, it was not feasible to perform these measurements for 1B7. However, given the similar molecular weights for the proteins and similar results for n_{c} and the other properties, we believe the a similar large surface potential would stabilize the 1B7 clusters.
Low Effective Dielectric Constant within the Clusters

The concept of equilibrium cluster formation assumes long ranged electrostatic forces in the cluster, which is favored by a low dielectric constant. (Groenewold and Kegel 2001) The dielectric constant of water within the clusters will be influenced by confinement between the protein surfaces. Analogously, the heterogeneous environment within each dense protein cluster is very different from that of bulk water. As stated in the main text, we have estimated that the φᵢₙₜ in the clusters is ˜0.60 based on the SEM images and SLS measurements on IgG clusters

$${φ}_{int} = {( \frac{{D}_{c}}{{D}_{m}} )}^{( {δ}_{f} - 3 )} \qquad ( 22 )$$

where Dₘ is the diameter of a protein monomer. The dielectric constant in the cluster according to effective medium theory is given as (Bottcher 1945; Reynolds and Hough 1957)

$$\frac{ε - {ε}_{2}}{3 ε} = {φ}_{1} \frac{{ε}_{1} - {ε}_{2}}{{ε}_{1} + 2 ε} \qquad ( 23 )$$

where ∈₁ is the protein dielectric constant (5), ∈₂ is the dielectric constant of water (80) and φ₁ is the volume fraction of protein in the medium. The calculated ∈ is 20 for φ₁ of 0.6. This value is similar to the choice of 25 in Table 17.

Assuming uniformly-spaced spherical proteins of R=5.5 nm at φᵢₙₜ=0.6 implies from simple geometry (if we assume each spherical protein to be contained in a cube and the cubes when put together side to side form the cluster of proteins where Vₛₚₕₑᵣₑ=0.6 V_{cube}) that that water inside the cluster is confined to channels on the order of 1 nm or less (see, (Rintoul and Torquato 1998)) between protein surfaces. At this level of confinement, (Paddison 2003; Biswas, Rohman et al. 2008), (Senapati and Chandra 2001; Wang and Pan 2007; Ahmad, Gu et al. 2011), the effective dielectric constant of water is reduced to ˜40. (Ahmad, Gu et al. 2011) Also only the ions that dissociate from the proteins and few of the extraneous ions tend to be present in these extremely confined spaces. (Kralchevsky, Danov et al. 2011) This low ion concentration and low ∈ within the clusters will produce less Debye screening as compared to bulk water buffer solutions. This low screening level would further enhance the longer-ranged electrostatic repulsion that influences the cluster size.

### Cluster Dissolution Time

The dissolution time for protein in the nanocluster is of interest for understanding in vitro dilution experiments, and more importantly, cluster dissociation upon in vivo subcutaneous injection. The dissolution time t_{F} of a 300 nm cluster was calculated from a shrinking sphere model, assuming a solid sphere of protein (McCabe, Smith et al. 1985):

$${t}_{F} = \frac{ρ}{4 {D}_{v} ( {c}_{sat} - {c}_{bulk} )} * \frac{1}{2} {D}_{c}^{2} \qquad ( 24 )$$

Where ρ is the density of the protein (1.34 g/ml), Dᵥ is the diffusion coefficient of a single protein in water (4.5×10⁻⁷ cm²/s, calculated using the Stokes-Einstein equation), cₛₐₜ is the concentration of a saturated protein solution (assumed to be 50 mg/ml), c_{bulk}˜0 mg/ml. The dissolution time was found to be 7 ms for a 300 nm diameter cluster. The rapid dissolution to protein monomer is favorable for rapid pharmacokinetics for high bioavailability. It may also be beneficial for minimizing time concentrated protein is exposed to fluids where protein denaturation may possibly take place.

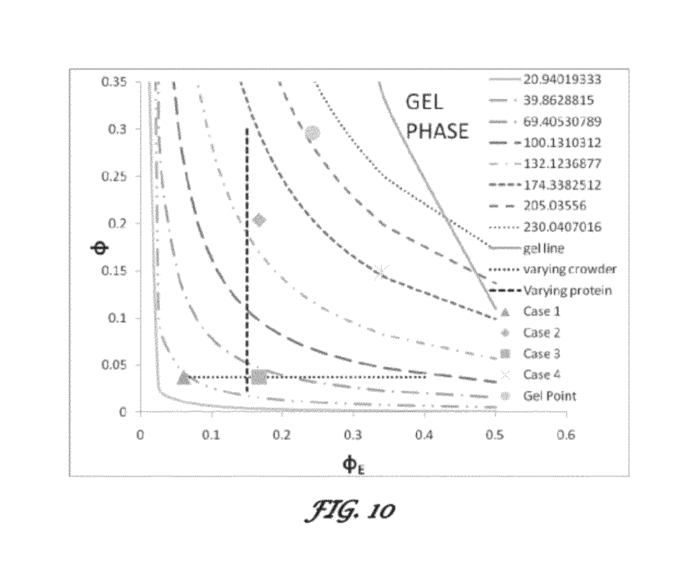

FIG. 10 presents a set of contours for cluster sizes ranging from 20 to 230 nm, for a given φₚ, as a function of φ_{c}. The calculated z₀ (=q₀), which depends upon φₚ, as shown in the supplementary section, is on the order of one charge/protein molecule near the pI. (Groenewold and Kegel 2001; Chad, Jerath et al. 2009). Given the uncertainty in ∈ᵣ and zₒ (=q₀), the value of ∈ was approximated as the contact value for the depletion potential in Eq. 7, which depends upon φ_{c}, while the other attractive terms were neglected as they are not influenced by crowding. Horizontal and vertical pathways for varying either φₚ or φ_{c}, which were also used in the experimental studies, are shown explicitly in FIG. 10. For a given φₚ, an increase in φ_{c} raises the depletion attraction between protein monomer and hence increases the cluster diameter, D, in reasonable agreement with the data shown in FIG. 5A. As shown by the cluster sizes in FIG. 5A, the sizes predicted from theory shown in FIG. 10 are in reasonable agreement with experiment given the simplicity of the model. For instance, the model does not consider charge screening, differences in ∈ᵣ inside and outside the cluster, and variations in the attractive interaction with H. Similarly, at a given φ_{c} when φₚ increases, D increases as shown by the vertical dashed black line, corresponding to the data in FIGS. 3A and 3B. Here larger clusters are the result of a decrease in z₀ (=qₒ) with φₚ, thus a decrease in the Coulombic repulsion. A few specific cases are also denoted. For Case 1 at 100 mg/ml sugar and a φₚ of 0.037 (FIG. 10 and Table 3), the predicted σ is ˜35 nm while the experimentally value was 10 nm. For 275 mg/ml sugar and a φₚ of 0.037, the predicted and measured values of σ were ˜60 nm. For Case 3 (row 2 Table 1, 275 mg/ml sugar and protein) σ˜140 nm compared to the experimentally measured size of 88 nm.

TABLE 3
Input and output variables for the proposed model.¹

| | Case 1 | Case 2 | |
|---|---|---|---|
| Quantity | FIG. 5A | FIG. 3A | Kegel Case |
| δ_{f} | 2.5 | 2.5 | 33 |
| Dielectric constant (∈ᵣ) | 15 | 15 | 10.72 |
| Bjerrum Length (λ) | 3.733 | 3.733 | 5.22 |
| No. of dissociable sites per | 0.2 | 0.2 | 0.15 |
| unit area of colloid surface | | | |
| (σ) (nm⁻²) | | | |
| Distance between opposite | 0.2 | 0.2 | 0.134 |
| charges in an ionic bond (b) | | | |
| Radius of primary particle | 5.5 | 5.5 | Disc (75 nm |
| (R) | | | length, 15 |
| | | | nm diameter) |
| Φ_{E} (if applicable) | 0.061 | 0.167 | |
| Attractive energy (∈/kT) | 2.63 | 7.26 | 7.5 |
| Surface Tension (γ) | 0.006919 | 0.01910 | 0.002122 |
| Φₚ | 0.037 | 0.204 | 0.3 |
| q₀ | 0.435 | 0.185 | 0.0004 |
| Q | 9 | 116 | 80 |
| Aggregation number (n_{c}) | 21 | 627 | 200000 |

TABLE 4
Material balance to determine volume fractions in the dispersions

| | Actual weight of | Actual volume | Composition of actual | Vol. of | Vol. of | Vol. of | Volume of | Volume | Total Volume |
|---|---|---|---|---|---|---|---|---|---|
| Name of | 1:1 IgG:trehalose | of total solvent | solvent added (excluding | IgG | trehalose | pure buff- | PEG300 | of NMP | of all com- |
| sample | powder (mg) | added (mL) | dissolved sugar) | (μl) | (μl) | er (μl) | (μl) | (ml) | ponents (μl) |
| 214 mg/ml | 60.2 | 0.100 | 50 mM phosphate buffer | 23 | 18 | 100 | 0 | 0 | 141 |
| 275 mg/ml | 79.3 | 0.090 | 50 mM phosphate buffer | 30 | 24 | 90 | 0 | 0 | 144 |
| 157 mg/ml - | 40.4 | 0.100 | 20%(v/v) PEG300 in | 15 | 12 | 80 | 20 | 0 | 127 |
| 0.16 φ_{P} | | | 50 mM phosphate buffer | | | | | | |
| 162 mg/ml - | 41.4 | 0.100 | 30%(v/v) PEG300 in | 15 | 13 | 70 | 30 | 0 | 128 |
| 0.24 φ_{P} | | | 50 mM phosphate buffer | | | | | | |
| 157 mg/ml - | 39.8 | 0.100 | 20%(v/v) NMP, | 15 | 12 | 70 | 10 | 20 | 127 |
| 0.08 φ_{P}/ | | | 10%(v/v) PEG300 in | | | | | | |
| 0.16 φ_{N} | | | 50 mM phosphate buffer | | | | | | |
| 204 mg/ml - | 56.5 | 0.100 | 20%(v/v) NMP, | 21 | 17 | 70 | 10 | 20 | 138 |
| 0.08 φ_{P}/ | | | 10%(v/v) PEG 300 in | | | | | | |
| 0.16 φ_{N} | | | 50 mM phosphate buffer | | | | | | |

TABLE 5
Comparison of % monomer

| | % monomer after | % monomer after |
|---|---|---|
| Sample name | purification | dilution |
| 157 mg/ml - 0.08 φ PEG, | 96.2 ± 0.6 | 97.0 ± 0.1 |
| 0.16 φ NMP | | |

TABLE 6
Summary of various individual DLS samples
run for average colloid sizes in Table 1:

| IgG or Trehalose | | | | Percentage Stan- |
|---|---|---|---|---|
| Concentration | Additional | | Mean diam- | dard Deviation |
| (mg/ml) | Crowders | Run | eter (nm) | over mean |
| 214 | — | Run 1 | 103 | 9 |
| | | Run 2 | 68 | 21 |
| 275 | — | Run 1 | 92 | 8 |
| | | Run 2 | 78 | 8 |
| | | Run 3 | 94 | 9 |
| 157 | 0.16 φ_{P} | Run 1 | 95 | 9 |
| | | Run 2 | 93 | 9 |
| | | Run 3 | 140 | 23 |
| 162 | 0.24 φ_{P} | Run 1 | 118 | 10 |
| | | Run 2 | 103 | 16 |
| 157 | 0.08 φ_{P} | Run 1 | 280 | 8 |
| | 0.16 φ_{N} | Run 2 | 259 | 12 |
| | | Run 3 | 256 | 8 |
| | | Run 4 | 220 | 5 |
| | | Run 5 | 275 | 6 |

The large clusters may be contrasted with small clusters of highly charged lysozyme monomer at a pH of about 8, far from the isoelectric point, with aggregation numbers<5 and lifetimes of ˜25 ns. (Porcar, Falus et al. 2010) The small size and short lifetime are consistent with the dominance of the large repulsion for the highly charged particles relative to the attractive forces. In Table 3, results are shown for clusters of Boehmite rods in ortho-dichlorobenzene (∈ᵣ=29) where several of the parameters are similar to the protein clusters. (Groenewold and Kegel 2001) However, the low z₀ (=q₀) results in massive clusters with an aggregation number of 200,000 as observed experimentally, as described theoretically. (Groenewold and Kegel 2001) The nanoclusters in the current study have a charge intermediate between these two cases, which results in long lived equilibrium clusters with n ˜10² to 10³. In addition, the size could be manipulated by varying φ_{E} at a given φ_{P}.

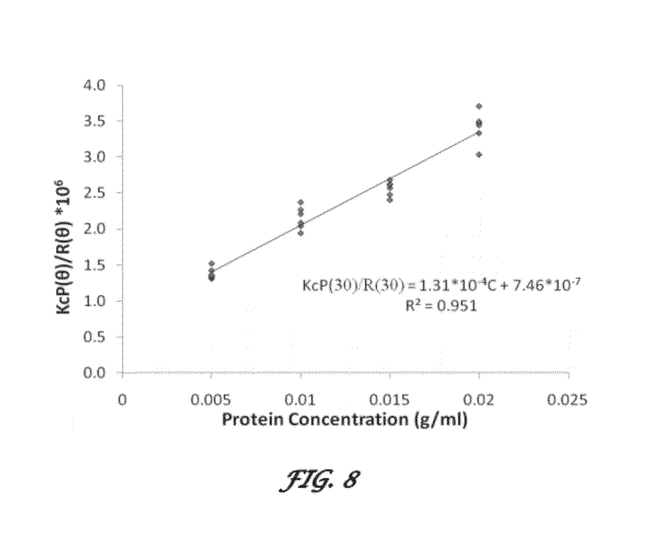

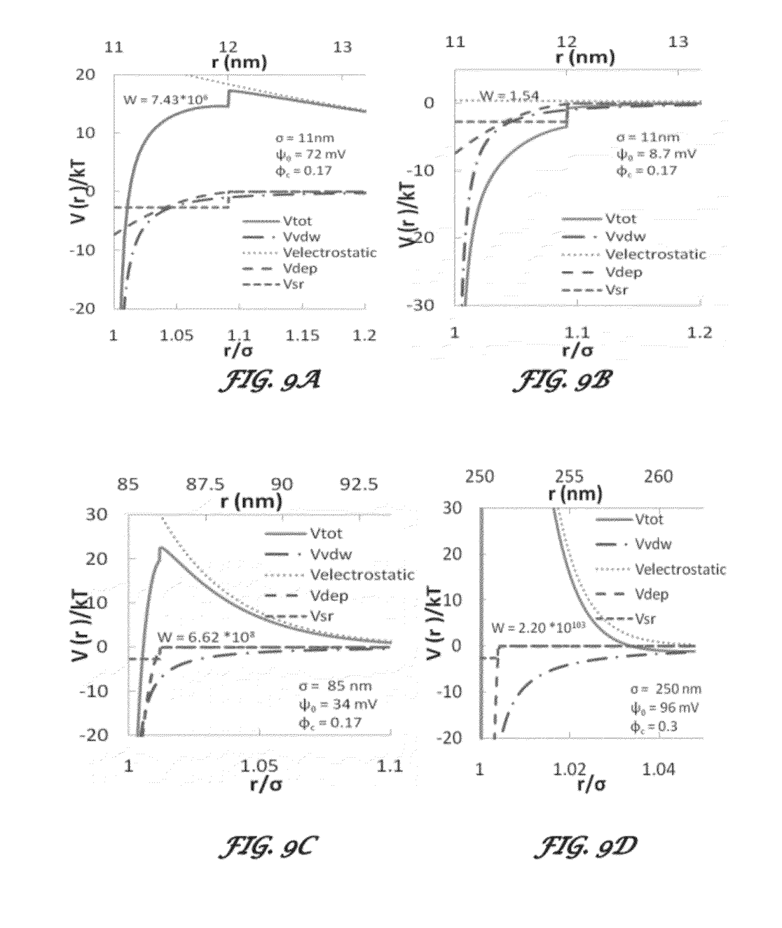

Intercluster interactions: Since the number of protein monomers is well defined for an equilibrium nanocluster well below the gel point, the nanocluster may be viewed as an individual colloidal particle. A sufficient repulsive Vₜₒₜ between two nanoclusters is required to prevent aggregation of the clusters and to maintain a low viscosity. As shown in FIG. 9C, a large repulsive barrier is present for an 85 nm protein nanocluster near the pI, consistent with the stable nanocluster dispersions for the case of φ_{c}=0.17 in Table 1. In contrast, the protein monomer dispersion was unstable in FIG. 9B, where the following parameters were held constant: 1 charge/protein monomer, Δσ=1 nm, and φ_{c}=0.17. For ˜277 protein molecules within the cluster, the large charge of ˜277 produces a surface potential of ˜24 mV well above the value for the monomer of only 0.71 mV. Thus, V_{EL} is substantial for the nanocluster, despite the proximity to the pI, and simultaneously, negligible for the monomer. Furthermore, V_{EL} scales as Rₚ such that the range of repulsion is much longer than for V_{DEP} (<1 nm) in and V_{SSR}. (Δσ˜1 nm). Since the range of these attractive forces is not influenced significantly by Rₚ, it is similar for the protein monomer and the nanoclusters. Thus, the reduced range, r/σ, of these attractive interactions of <˜1.01 for the nanocluster is far below that of ˜1.1 for the protein monomer, In contrast, V_{EL} versus r/σ is relatively insensitive to Rₚ. Since V_{EL} is dominant for the nanoclusters, they do not aggregate and remain colloidally stable with a W of 4.63*10⁷.

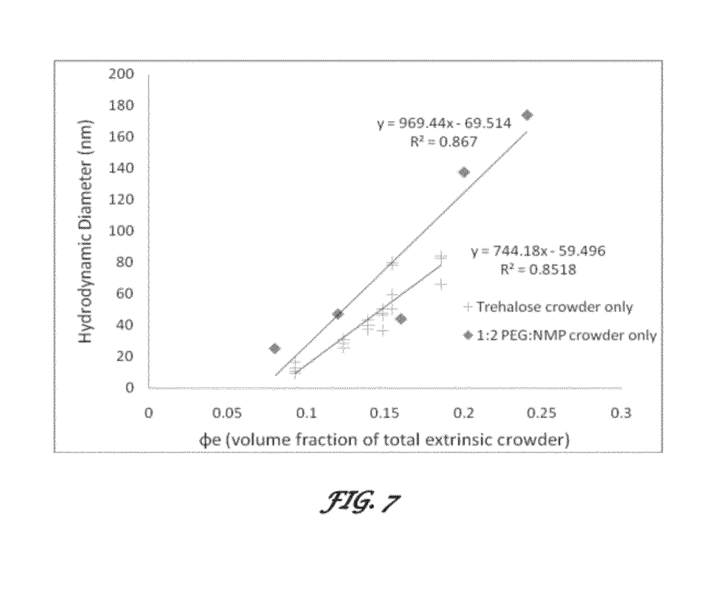

Of the various attractive interactions for the nanoclusters, the range of the VDW interaction is the longest as it scales with σ. However, since the nanocluster are porous, as shown in the SEM and STEM images (FIGS. 4A and 4B), the Hamaker constant is reduced. In FIGS. 9C and 9D, the Hamaker constant is reduced two fold to 2.5 kT. Even when including the VDW attractions, the electrostatic interactions were much stronger and longer-ranged than the total attractive interactions.⁸ For a 250 nm protein nanocluster formed with a higher crowder volume fraction of 0.3 and greater charge (similar to the experimental conditions in row 5 Table 1), electrostatic repulsion was even more dominant as shown in FIG. 9D. With ˜5870 protein monomers, the larger charge corresponded to a higher surface potential of ˜32 mV, and W reached 6*10¹⁶. Repulsive interactions between the nanoclusters were measured by SLS, with a B₂ of 6.6×10⁻⁵ mol*ml/g² (FIG. 8).

In this case, the theoretical determination of B₂ from Vₜₒₜ is poorly defined as the contribution from V_{DEP} diverges at very small r. A direct comparison of the potentials in FIGS. 9B and 9C reveals a novel concept of hierarchical interactions where Vₜₒₜ is strongly attractive for protein monomer and simultaneously, highly repulsive for the nanoclusters, consistent with the experimental data. This difference is due primarily to the increase in the range and strength of V_{EL} with an increase in Q and Rₚ, relative to a negligible change in the range of interaction for V_{SSR} and V_{DEP}. The strongly attractive intracluster interactions at r/σ=1.1 help generate the nanoclusters, whereas the weak intercluster attraction at the same r/σ prevents aggregation. The ability to control the hierarchical colloidal interactions may be expected to be universal and applicable to a wide variety of peptides and proteins.

Decreased Viscosity in nanocluster dispersions versus protein solutions: Eventually, at the point where the electrostatic repulsion becomes insufficient to balance the attractive forces, the protein forms a gel. The gel point may be defined by the spinodal curve, where the second derivative of f_{charge} with respect to q is zero. FIG. 10 shows the calculated spinodal curve and an experimental condition which results in a gel of equilibrium nanoclusters (gel point). The location of the gel curve relative to the experimental point is quite reasonable given the simplicity of the model and complexity of the electrostatic interactions with the cluster.

Protein stability section: For therapeutic proteins to retain activity without inducing adverse immunogenic reactions, it is important to maintain the native three-dimensional conformation during recovery and formulation. (Saluja and Kalonia) Currently, antibodies are challenging to formulate at high concentrations as solutions, since the high-level of protein mobility facilitates protein denaturation and exposure of internal hydrophobic patches, leading to reversible intermolecular association and, eventually, irreversible aggregation. However, excluded volume interactions from added crowding agents thermodynamically increase the stability of the native protein state. As the φ_{c} increases, the protein molecule will entropically favor the reduced volume of the natively folded state over the unfolded state. (Zhou, Rivas et al. 2008) For sugar as a crowding agent, the increase in the stability of a protein solution has been observed in terms of the negative preferential binding parameter between the protein and sugar. Thus, the extrinsically crowded solution environment of the protein nanoclusters will prevent or reduce unfolding of the protein molecules.

The low levels of protein denaturation observed are helped by stabilization of the protein native state during sample processing. Lyophilization is widely used in biopharmaceutical processing and has been shown to stabilize the protein native state by kinetically trapping protein molecules in an amorphous solid, thus reducing protein mobility which can lead to aggregation. Addition of the crowder, trehalose, during lyophilization further stabilizes the protein native state in solution by excluded volume and upon dehydration by forming hydrogen bonds with protein. Within the protein nanoclusters, the solid state is maintained, restricting protein mobility both in the particles and on the particle surface, relative to a solution.

Particle dissolution upon dilution occurs rapidly (˜1 second), given the high particle surface area and solubility of the protein monomer in physiological buffers (upon dilution of the crowding agents). The presence of the diluted crowders such as trehalose and PEG300 in the dissolution buffer further prefers the native protein state by entropically favoring it by excluded volume.

### Example II

Murine IgG2a monoclonal antibody 1B7, which binds and neutralizes the pertussis toxin (PTx) associated with whooping cough infection (Sutherland and Maynard). The amorphous protein particles were generated via a new freezing method, spiral-wound in situ freezing technique (SWIFT). In contrast, previous protein nanocluster studies used tray freezing to produce the protein particles. (Miller, 2011) As opposed to traditional tray freezing lyophilization, the much more rapid SWIFT freezing may offer advantages for achieving high protein stability, as has been shown for spray freeze drying, spray freezing into liquids and thin film freezing. Unlike the other rapid freezing processes, in SWIFT, the particles are produced in the actual dosage vial to simplifying processing. The amorphous particles were gently dispersed in a dispersion buffer comprised of histidine buffer adjusted to the approximate 1B7 pI augmented with three pharmaceutically acceptable crowding agents, water-soluble organic n-methyl-2-pyrrolidone (NMP), polyethylene glycol (PEG), and trehalose to confer low viscosity and limit 1B7 solubility to prevent particle dissolution. Under these conditions, the transparent dispersion exhibits a low viscosity even at high antibody concentrations (<50 cP at 200 mg/ml), with ˜200 nm 1B7 particles in equilibrium with 2.5-5 mg/ml dissolved 1B7, as measured by DLS. Importantly, the protein native structure is preserved, as seen by comparing the activity of the diluted dispersions and the untreated 1B7 by SDS-PAGE and ELISA analysis.

Furthermore, an in vivo murine pharmacokinetic study was performed to compare the bioavailability of three different subcutaneously administered dispersions with traditional IV and SQ administration of antibody solutions. To compare the dispersion and solution controls at similar dosages, we administered a standard dosage (˜5 mg/kg) in a large 100 μl volume. The 1B7 distribution and elimination half-lives were very similar for these three groups, while the time to peak serum concentration (tₘₐₓ) was delayed for the SQ injections, consistent with the expected slower diffusion kinetics from this injection site. The dispersion was then prepared at high concentration (200 mg/ml) to compare the pharmacokinetics resulting from a small volume (1 μl), standard dose injection and a large volume (100 μl), high-dose (52 mg/kg) injection. Again, the pharmacokinetic profiles were remarkably similar for all SQ groups, although the small volume injection had a slightly shorter tₘₐₓ, indicating that the more rapid diffusion kinetics of a small injection may impact the overall pharmacokinetics, consistent with results for the single crowder, trehalose, system (Miller, 2011). The analysis of terminal serum samples for total 1B7 protein and specific PTx binding activity by ELISA, as well as an in vitro PTx neutralization test, were unable to detect a loss in 1B7 activity or development of anti-1B7 immune responses over a 14-day period. The high protein stability during injection, residence in the subcutaneous tissue and transport into the bloodstream is shown to be consistent with in vitro data previously reported (Miller, 2011), whereby folded protein molecules rapidly diffuse away from the surface of the equilibrium nanoclusters. The ability to achieve high stabilities for the severe test of extremely high dosages may offer new opportunities for more modest increases in dosages in subcutaneous injection. Dispersions are a promising approach to highly concentrated, low viscosity protein formulations that preserve activity and confer favorable pharmacokinetics. Moreover, dispersions can achieve dosages at least 10-fold higher than can be attained via solutions and can be formulated with a variety of pharmaceutically acceptable agents.

Antibody expression, purification and biotinylation: Murine hybridoma cells producing the IgG2a antibody 1B7 were grown in T-flasks in Hybridoma-SFM serum-free media at 37° C. with 5% CO₂ until cell death, as reported previously (Sutherland and Maynard 2009; Miller 2011) Briefly purification of the antibody consisted of centrifugation at 3000 rpm for 20 minutes, followed by filter sterilization using a 0.45 μm filter, dilution 1:1 with binding buffer (20 mM pH 7.0 sodium phosphate) and loading with binding buffer onto a pre-equilibrated Protein-A column (GE Healthcare). After baseline stabilization, 1B7 was eluted into collection tubes containing 1 M Tris pH 8.0 using an elution buffer (0.1M glycine pH 2.7). Protein concentration was measured with micro-bicinchonoinic acid (BCA) assay (Pierce, Rockford, Ill.), while non-reducing SDS-PAGE verified protein preparation homogeneity and purity. Purified 1B7 was labeled with biotin using EZ-link® Sulfo-NHS-LC-Biotin (Pierce, Rockford, Ill.). A 5 mM solution of the biotin reagent was added at a 5:1 molar ratio to a 1 mg/ml solution of the 1B7 in PBS at room temperature and allowed to react for 30 minutes. Excess biotin was removed by buffer exchange using 50,000 MWCO Centricon concentrators with PBS.

Particle formation by spiral wound in-situ freezing technology (SWIFT): Purified and biotinylated 1B7 was buffer exchanged into 20 mM pH 5.5 histidine buffer using Centricon filters, as above. The protein concentration was measured again, solid α-α trehalose was added to a 1:1 wt ratio as a cryoprotectant and gently mixed to dissolve. The resulting solution was filter sterilized (0.22 μm), diluted to 20 mg/ml protein and transferred to a sterile 8 ml (1.9 cm×4.8 cm) glass vial for SWIFT freezing. During SWIFT, the base of the vial was contacted with liquid nitrogen while rotating the vial on its side (˜1 revolution/second), resulting in a thin film of frozen solution on the inside edge of the vial, with subsequent thin films freezing in a spiral towards the center of the vial. After the entire volume was frozen (˜10-40 seconds), the samples were placed upright on a pre-cooled lyophilizer shelf at −40° C. The samples were then lyophilized for 12 hours at −40° C. at 100 mTorr, followed by a 6 hour ramp to 25° C. at 50 mTorr, and maintained for secondary drying at 25° C. at 50 mTorr for at least an additional 6 hours. To assess protein activity after freezing, powder was reconstituted at 5 mg/ml in PBS for analysis by dynamic light scattering (DLS) and enzyme-linked immunosorbent assay (ELISA) as described below. Samples of the dry powders after lyophilization for scanning electron microscopy (SEM) analysis were placed on adhesive carbon tape to fix the sample to the SEM stub. Each sample for SEM was platinum-palladium sputter coated using a Cressington 208 bench top sputter coater to a thickness of 10 nm. Micrographs were taken using a Zeiss Supra 40 VP scanning electron microscope with an accelerating voltage of 5 kV.

Dispersion formation: To form the dispersion, SWIFT frozen and lyophilized 1B7 protein powder was compacted into 0.1 ml conical vials (Wheaton Science Products No. 986211) such that the total powder weight was 0.04±0.001 g. An aqueous-based solvent dispersion buffer, containing 10% (v/v) PEG300 and 20% (v/v) n-methyl-2-pyrrolidone (NMP) in a 50 mM phosphate buffer with the pH adjusted to match the measured antibody pI (pH 7.2, see FIG. 26), was added to the lyophilized protein. Gentle stirring with the tip of a needle removed air pockets, to yield a uniform, optically clear dispersion with a final 1B7 concentration of 160-200 mg/ml. Neither sonication nor violent mixing was necessary to form a uniform transparent dispersion.

Viscosity measurement: The apparent viscosity of the 1B7 dispersion was measured as the time to draw 50 μl of the dispersion into a 25 gauge 1.5″ long needle attached to a 1 ml tuberculin slip tip syringe, as reported previously for sheep IgG dispersions. Briefly, videos of the conical vial containing the dispersion were taken and the time to draw from a height 0.4″ from the bottom of the cone to a height 0.1″ from the bottom of the cone was measured using Image J software. A standard curve using known solutions with various viscosities provided a linear correlation between the time to draw 0.05 ml from the conical vial to the viscosity with an r² value greater than 0.99. These results are consistent with previous work with suspensions of model proteins and protein solutions which found that the time to draw up a specified amount of the sample in a syringe was correlated linearly to viscosity.

Colloidal size determination/characterization: Dynamic light scattering (DLS) was used to measure the sizes of particles present in the purified 1B7 preparation, concentrated 1B7 dispersion and dilutions of the dispersion using a custom-built DLS apparatus modified to include backscattering angles up to 165°. (Miller, 2011) Particle sizes in the concentrated dispersion were measured with a small volume cell (60 μl, Beckman Coulter #A54094) at −23° C. and a 160° scattering angle, while all other measurements were made in a standard 1 ml cell at ˜23° C. and scattering angles optimized to detect the relevant particle size. To estimate the solubility of 1B7 in the dispersion buffer, the 200 mg/ml dispersion was diluted 1:40, 1:80 and 1:160 in dispersion buffer and particle sizes measured at a 90° scattering angle. The concentration of 1B7 at which the protein monomer peak is observed by DLS is defined as the solubility. (Miller 2011) To mimic the effects of dilution on particle size and detect formation of aggregates, the dispersion was diluted 1:40 in PBS to give a final 5 mg/ml 1B7 concentration and the resulting particle sizes measured at a scattering angle of 30°. The size of purified 1B7 monomeric antibody in PBS was measured at 5 mg/ml and a scattering angle of 30°.

In vitro antibody activity and aggregation assays: The 1B7 tertiary structure within the dispersed particles was assessed by intrinsic tryptophan fluorescence, as the emission maximum shifts based on the local environment of the tryptophan side chain. A SpectraMax M5 spectrophotometer (Molecular Devices) was used to fluoresce protein samples at a wavelength of 295 nm, with the emission spectrum recorded at 1 nm increments between 310-380 nm. For 1B7, a shift in the emission maximum is observed from 342 nm for folded protein to 350 nm for fully unfolded protein. This approach was used to qualitatively look for evidence of an altered tertiary protein structure of the protein dispersed in 200 nm particles at 200 mg/ml. All subsequent assays of protein activity and structural stability were conducted on 1B7 dispersion diluted to 1 mg/ml in PBS. Controls included lyophilized 1B7 (reconstituted in PBS at 1 mg/ml) and purified 1B7 with no further processing (1 mg/ml in PBS). First, the formation of insoluble and di-sulfide linked aggregates was monitored by non-reducing SDS-PAGE. Briefly, 3 μg 1B7 sample was combined with loading buffer and separated on a 4-20% precast linear gradient polyacrylamide gel (Bio-Rad) and stained with Gel-Code Blue (Bio-Rad).

To monitor ligand-binding activity, an indirect PTx ELISA was employed as reported previously. (Sutherland and Maynard 2009; Miller 2011) High-binding ELISA plates (Costar) were coated with 50 μl pertussis toxin (PTx, List Biological Laboratories) at 0.75 μg/ml in PBS and incubated at 4° C. overnight. Wells were blocked with assay buffer (PBS-1% milk) for 1 hr, prior to addition of 1B7 samples in a √10 serial dilution scheme from 50 μg/ml in assay buffer. After one hour incubation at room temperature and triplicate washes with PBS-0.05% Tween 20, 50 μl goat anti-mouse IgG-horseradish peroxidase conjugate (1:2000 dilution in assay buffer, Sigma) was incubated for one hour at room temperature. Plates were washed in triplicate and signal developed with tetramethylbenzidine dihydrochloride (TMB) substrate (Pierce), quenched with 1N HCl and the resulting absorbance at 450 nm recorded using a SpectraMax M5 instrument. The EC₅₀ value was calculated from the linear range of the dose-response curve as the antibody concentration corresponding to 50% of the maximum absorbance (Eq. 25).

$${EC}_{50} = \frac{C ( {A}_{450 , max} ) - C ( {A}_{450 , min} )}{2} \qquad ( 25 )$$

For comparison between samples, the relative EC₅₀ was calculated as the ratio of the sample EC₅₀ to unprocessed control antibody EC₅₀. All samples were run in triplicate.

In vivo bioavailability in BALB/c mice: An in vivo pharmacokinetic study of the 1B7 dispersion and control solution was performed over a 14 day period using four to six healthy 24-27 g, female BALB/c mice per group. Mice were administered a single 1 or 100 μl subcutaneous (SQ) injection of 1B7 at low (4.6-7.3 mg 1B7/kg body weight) or high (51.6 mg/kg) doses. The five sample groups compared in this study included two groups (1) IV and (2) SQ injections of 100 μl of a 1B7 solution (1.4 mg/ml solution for a final 5.6 mg/kg dose) reported previously (Miller, 2011), as well as (3,4) SQ injections of 100 μl antibody dispersion at low (4.6 mg/kg) and high (51.6 mg/kg) doses; and (5) SQ injection of 1 μl at a low (7.3 mg/kg) dose of the antibody dispersion in the dispersion buffer (see Table 7). The previously reported solution samples (groups 1 and 2) were prepared from a 20 mg/ml 1B7 solution in PBS diluted to 1.4 mg/ml in PBS (Miller, 2011) while the dispersion samples were diluted in the dispersion buffer from a 200 mg/ml 1B7 dispersion to a concentration of 1.2 mg/ml for group 3, 12.9 mg/ml for group 4, and 182 mg/ml for group 5 immediately prior to injection.

Prior to the injection and at eight additional timepoints between 12 and 336 hours, mice were weighed and a blood sample (˜20 μl) collected from the tail vein. After collection, the samples were allowed to clot, centrifuged at 5000 rpm for 10 minutes and serum transferred to a new tube. At the terminal timepoint (336 hours), mice were anaesthetized and between 0.2 and 1 ml serum collected by cardiac puncture. These samples were used in ELISA assays, to measure the total and active concentrations of 1B7 in the serum and, for the terminal time point, to measure antibody activity via an in vitro neutralization assays and to provide an initial estimate of mouse anti-1B7 responses. This study was performed with approval by the Institutional Animal Care and Use Committee at the University of Texas at Austin (protocol #AUP-2010-5 00070) in compliance of guidelines from the Office of Laboratory Animal Welfare.

TABLE 7
Animal study data comparing various administration methods:

| | | | | AUC_{0-14 day}/dose | | | | CHO neutralization |
|---|---|---|---|---|---|---|---|---|
| Type of | | Dosage | Cₘₐₓ/dose | (μg*hr/ml)/ | tₘₐₓ | t_{1/2}, _{α} | t_{1/2}, _{β} | assay titer |
| injection | N | (mg/kg) | (μg/ml)/(mg/kg) | (mg/kg) | (hrs.) | (hrs.) | (hrs.) | (ml/μg) |
| IV solution | 6 | 5.6 | 24.3 | 2073 | 12 ± 0 | 45.7 ± 22.8 | 227.1 ± 24.9 | 500 |
| injection | | | | | | | | |
| SQ solution | 4 | 5.6 | 18.2 | 1630 | 21 ± 6 | 43.4 ± 17.3* | 210.0 ± 17.4 | 400 |
| injection | | | | | | | | |
| SQ 1 μl | 6 | 9.4 | 13.8 | 2214 | 24 ± 0 | 42.1 ± 24.8* | 243.2 ± 35.5 | TBD |
| injection | | | | | | | | |
| dispersion | | | | | | | | |
| trehalose | | | | | | | | |

Measurement of 1B7 in serum samples: To determine the concentration of active 1B7 in serum samples, a standard ELISA approach was used with the following modifications as previously reported. (Miller, 2011) ELISA plates were coated with PTx at 1.5 μg/ml in PBS. The assay buffer used as diluent in all steps consists of 4% bovine serum albumin, 4% fetal bovine serum (FBS), 0.05% Tween 20, in PBS, pH 7.4. After blocking with assay buffer, 2.3 μl serum sample was serially 1:√10 diluted in 50 μl per well assay buffer. Each plate included mouse serum (Sigma) as a negative control and a 1B7 standard curve diluted to an initial concentration of 100 μg/ml in mouse serum. Additional samples were analyzed for total protein detected using a streptavidin coating on the ELISA plates to detect the biotinylated 1B7.

After measurement of the resulting absorbances, SoftMax Pro v5 was used to calculate EC₅₀ values based on the serum dilution using a 4 parameter logistic (4PL) model for each individual curve. Concentrations of active 1B7 in each serum sample were calculated from a linear correlation between the log [(sample EC₅₀)/(standard EC₅₀)] versus the log of the known 1B7 concentration in the standard curve. A linear correlation with a fit>0.95 from at least 5 independent standard curves was determined (FIG. 27).

Measurement of active antibody by CHO cell neutralization assay: As an orthogonal activity measurement to determine the concentration of serum 1B7 able to neutralize PTx activity in vitro, we employed a CHO cell neutralization assay. (Sutherland and Maynard 2009) The concentration of neutralizing antibody was measured as the sera dilution that completely inhibited PTx-induced CHO cell clustering relative to a standard curve of purified 1B7 with known concentration. Briefly, 50 μl of 1.5 ng/ml pertussis in Dulbecco's Modified Eagle Medium (DMEM) with 10% FBS was added directly to each well of a sterile 96 well tissue culture plate. Terminal serum samples (2.3 μl) were serially diluted using a 1:√10 dilution scheme to maintain a constant PTx concentration. After incubation for 30 minutes at 37° C. and 5% carbon dioxide, 100 μl/well of freshly trypsinized CHO cells at 10⁵ cells/ml were seeded in each well. After 24 h of incubation at 37° C. and 5% CO₂, wells were scored for CHO clustering using 0-3 scale, with 0 as elongated (non-clustered) and 3 as completely clustered.

Stable Protein Particles made by SWIFT freezing: As a first step in the preparation of concentrated aqueous dispersions, a dried powder of protein particles was formed. The choice of freezing method is critical to both protect antibody structure and activity during freezing, as well as to produce particles of the appropriate size and morphology to yield a colloidally stable dispersion. To address these concerns, a novel freezing technique, SWIFT, was developed which rapidly freezes an antibody solution directly in the final packaging vial prior to lyophilization (FIG. 25). The rationale in developing this technique is that two major sources of protein denaturation during freezing are exposure to liquid-gas interfaces during spray-freeze drying and the slow rate of freezing in larger volumes which can result in freeze concentration and subsequent concentration-dependent aggregation. By rotating the vial of protein solution while in contact with liquid nitrogen, each concentric layer freezes in less than a second. The remaining liquid is gently mixed due to rotation, normalizing any concentration gradients.

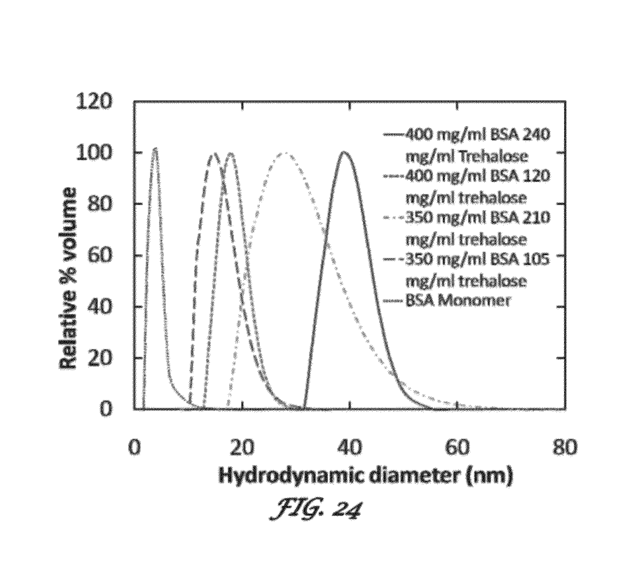

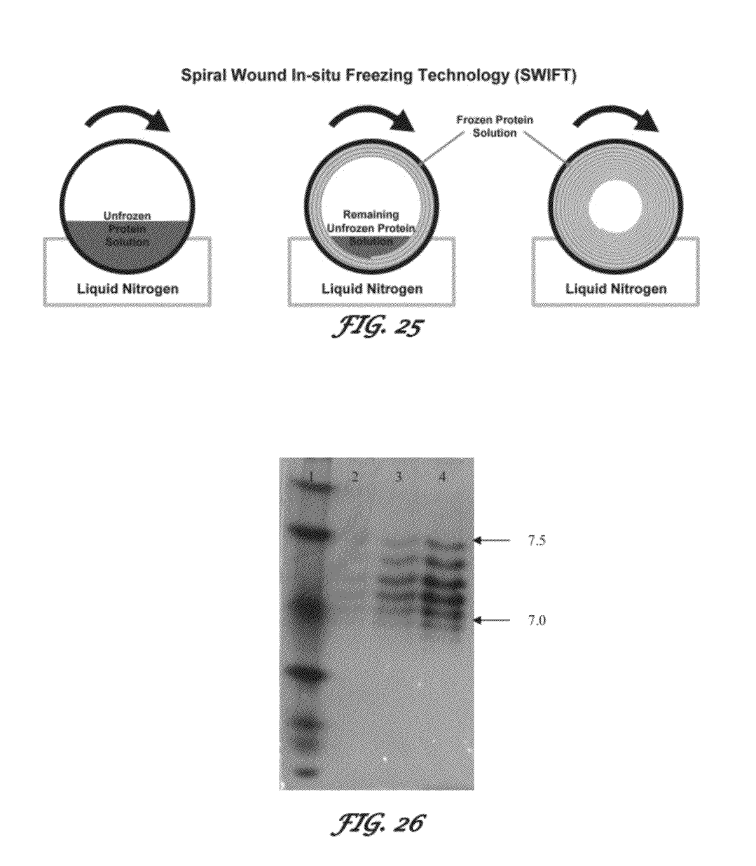

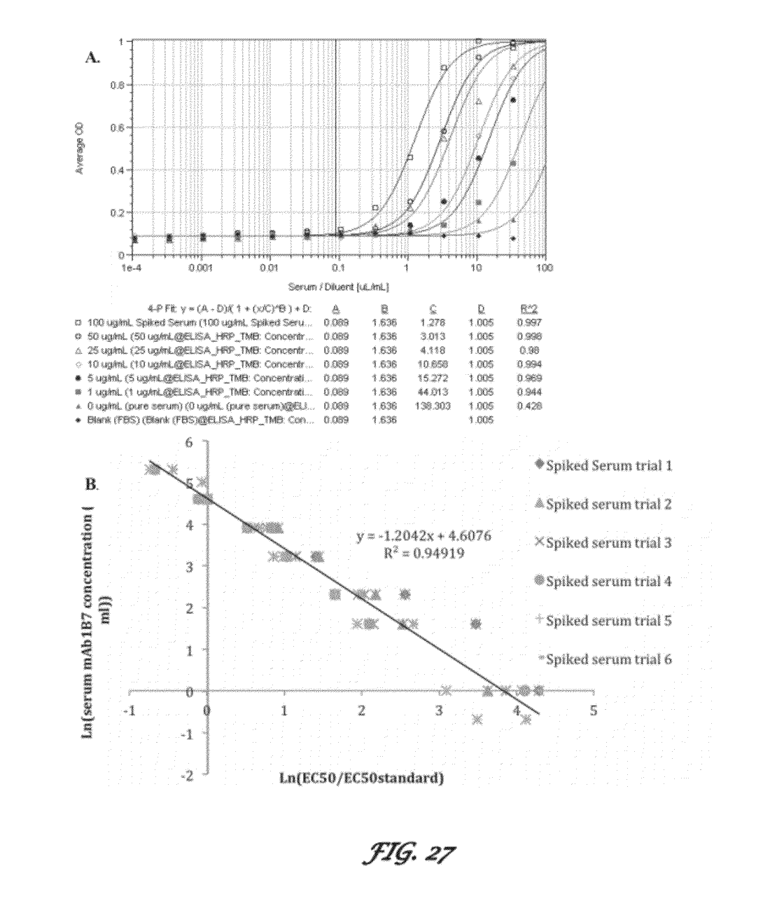

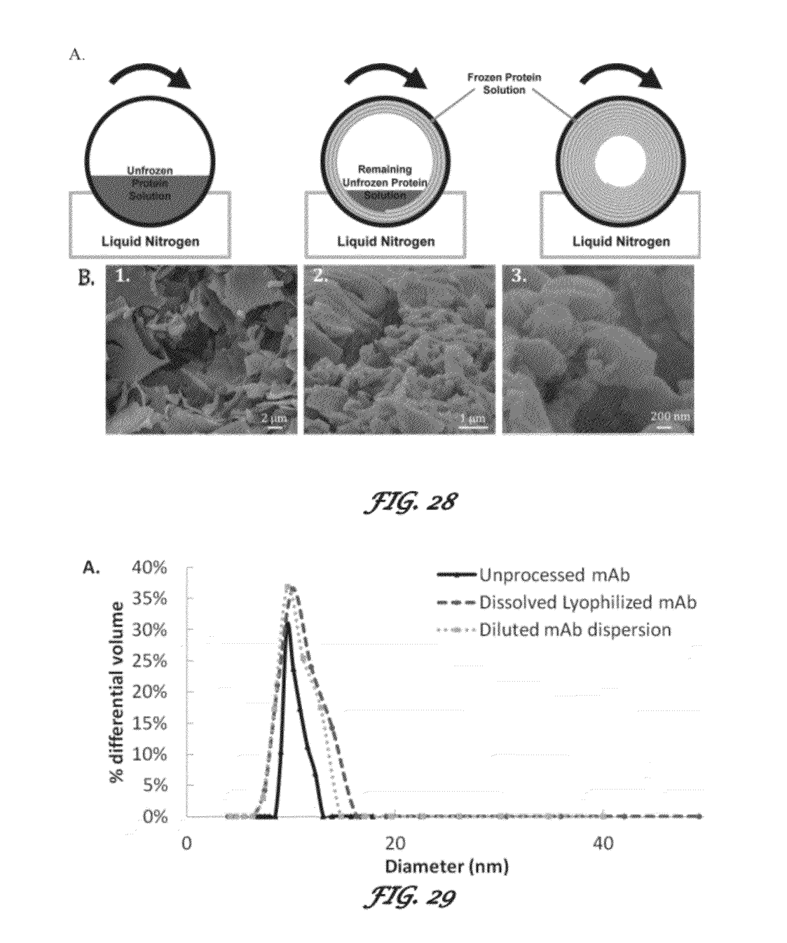

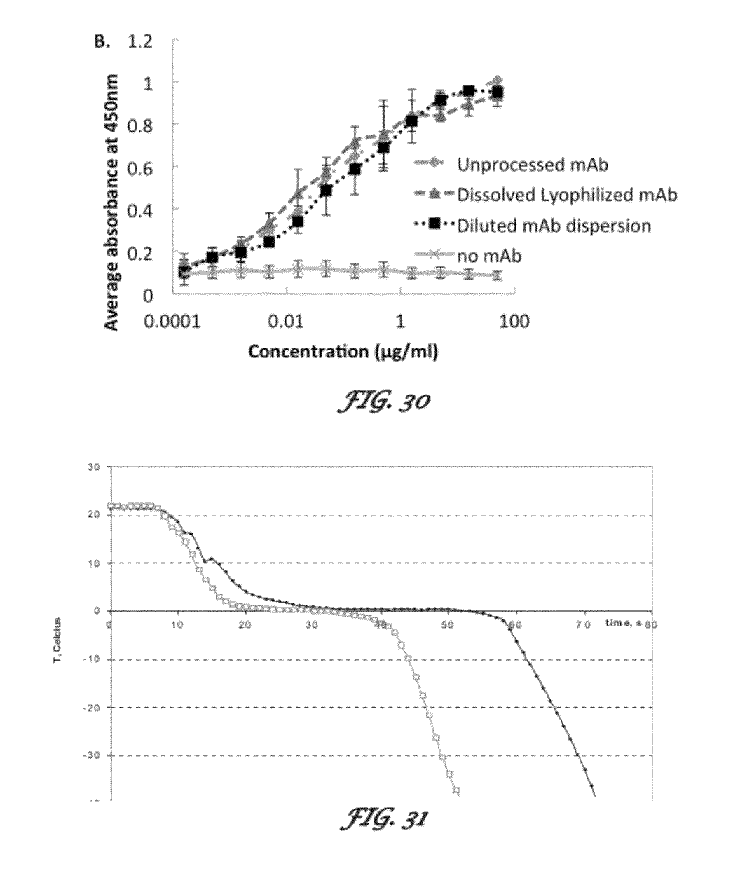

SWIFT was used followed by lyophilization to form sub-micron particles of the 1B7 antibody used in the dispersions. To prevent protein aggregation during freezing, the protein solution was adjusted to contain a 1:1 weight ratio of trehalose as a cryoprotectant. The buffer selected, 20 mM histidine pH 5.5, is commonly used during lyophilization steps. An SEM analysis of the frozen and lyophilized 1B7 clearly indicates the presence of sub-micron particles, similar to the size desired in the final dispersion (FIG. 28). Importantly, antibody processed in this manner retains native conformation and activity upon reconstitution with PBS at 5 mg/ml. At this concentration, DLS detected a single species with a ˜10 nm hydrodynamic diameter, as expected for an antibody monomer (FIG. 29). The absence of larger particles indicates that the antibody did not form irreversible aggregates during SWIFT and lyophilization. In addition, an ELISA to monitor the specific PTx-binding activity of the reconstituted antibody revealed no significant change in activity due to these processing steps versus the untreated control (FIG. 30).

The SWIFT process was designed to produce particles of the desired morphology while protecting protein structure and activity. This is achieved via rapid freezing with minimal liquid-air interface, goals inspired by related process, thin film freezing (TFF). In SWIFT, each film layer, corresponding to a single vial revolution, is ˜200 nm thick. Indirect contact with liquid nitrogen as a heat sink confers cooling rates of ˜10² K/s. In TFF, a small volume of protein solution is deposited on a cryogenically cooled surface, where it spreads to ˜210 nm thickness, freezing within a single second. Scaling-up to compare freezing times for equal volumes, TFF freezes at a rate of ˜5.1 seconds per ml of protein solution, while SWIFT results in a similar rate, ˜7.5 seconds/ml (FIG. 31).

As a result of the similarities in freezing rates and film thicknesses, TFF and SWIFT processing of similar protein solutions yields dry particles with similar morphologies (FIG. 28). For TFF, and by extension, SWIFT, the rapid cooling and freezing rates generate a large number of ice nuclei, which exclude solute molecules due to freezing point depression effects. The remaining liquid present in thin channels between ice nuclei, becomes supersaturated with dissolved crowder molecules and protein. Rapid vitrification of these liquid channels due to rapid freezing decreases the collision rate between the protein and sugar molecules/particles. As these precipitate due to supersaturation, the coagulation of small particles generates larger particles. In addition, as the concentrations of dissolved solutes rise in the unfrozen liquid, the associated viscosity increase will further reduce the mobility of the growing particle nuclei. In contrast, traditional tray freezing lyophilization using a slower freezing rate contains much larger liquid channels and larger final particles after drying. Thus smaller submicron protein particles, as shown in FIG. 28 are formed during SWIFT freezing versus standard tray freezing lyophilization.

With both SWIFT and traditional tray lyophilization, low levels of protein denaturation and aggregation are achieved due to the kinetic and thermodynamic stabilization of the native protein structure during freezing and lyophilization. The native protein state is stabilized during lyophilization by kinetically trapping protein molecules in an amorphous solid, thereby reducing protein mobility which can lead to aggregation. Addition of the lyoprotectant trehalose during lyophilization further thermodynamically stabilizes the protein native state during freezing by entropically favoring the native folded state and during dehydration by forming hydrogen bonds with proteins. However, processes to form submicron protein particles such as spray freeze drying (SFD), have been shown to increase protein aggregation versus standard tray freezing lyophilization due to the large gas-liquid interface in the spraying step. The large area/volume of the gas-liquid interface of ˜6000 cm⁻¹ in SFD for 10 μm sprayed droplets can lead to protein adsorption at the interface, denaturation and aggregation. In the case of SWIFT, the gas-liquid interface is minimized as the only exposure of the liquid protein solution to the air is the liquid interface inside the glass vial. As a result, the estimated gas-liquid interface decreases 3 orders of magnitude when compared to SFD to ˜4 cm⁻¹. Thus the 1B7 was anticipated and found to remain stable upon reconstitution to monomer from the dry powder form after SWIFT freezing and lyophilization.

One practical advantage of SWIFT freezing is the ability to freeze directly in the final dosage vial when compared to other rapid freezing techniques such as TFF and SFD. This approach avoids the need for costly, solid transfer steps while maintaining aseptic conditions. In this case, if a dosage of 80 mg of the protein is required at a concentration of 20 mg/ml, the 8 ml vial used in the study can serve as both the freezing and reconstitution vial. However, since the cooling rate SWIFT freezing is governed by the liquid cryogen used and the thickness of the glass vial, as well as the heat transfer coefficients of the materials used, the vial can be readily scaled-up or down to meet dosage requirements. In addition, by removing the transfer step to the final vial, all of the protein can be recovered after lyophilization and utilized in the formation of the final dosage.

Colloidal Characterization of 1B7 particles in dispersion: To form the colloidally stable, transparent dispersion, the dry, sub-micron particles of antibody and trehalose produced via SWIFT were combined with a specially formulated dispersion buffer. To reduce protein solubility, this includes a 50 mM phosphate buffer adjusted to the antibody pI (pH 7.2) and two additional crowding agents: 20% n-methyl-2-pyrrolidone (NMP) and 10% polyethylene glycol 300 (PEG300) by volume. After combining the SWIFT particles and dispersion buffer, the trehalose contained in the dry powder will dissolve. A fraction of the trehalose will diffuse into the solution, increasing the volume fraction of crowding agents as observed previously for sheep IgG. (Miller 2011) Sufficient dispersion buffer was added to the dry powder to yield a final antibody concentration of 160-200 mg/ml with a final volume fraction (φ) of crowding agents of 0.34.

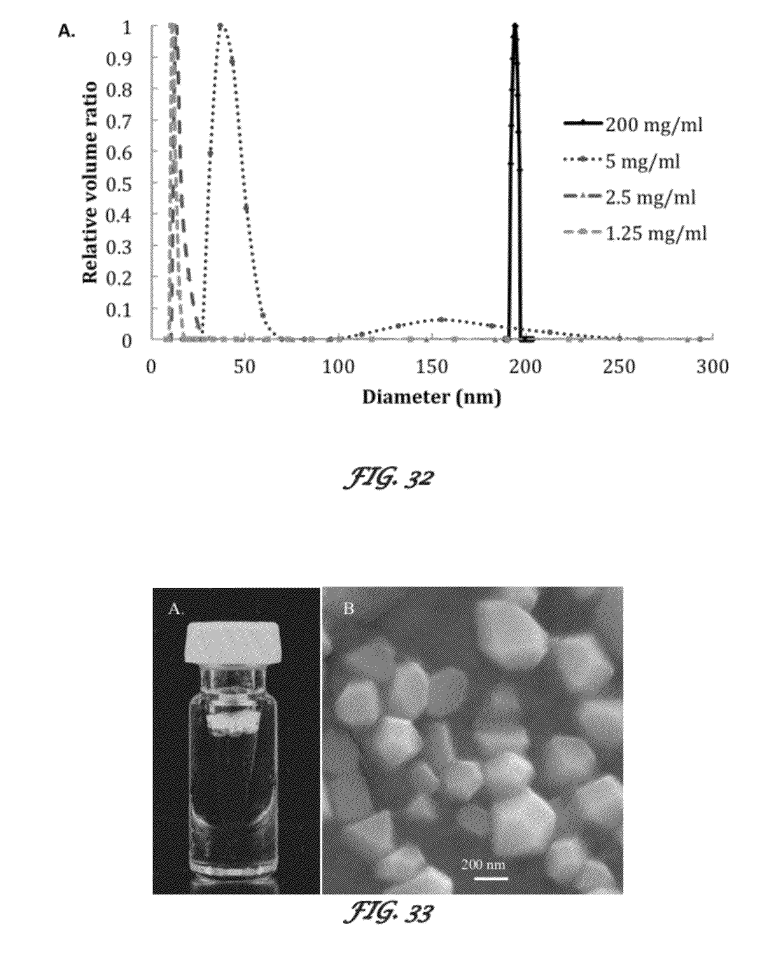

Under these conditions, DLS analysis of the dispersion using a low volume (60 μl) cell identifies a single population of particles with a ˜200+/−14 nm diameter. This colloid size was reproduced in three separate studies, measured each time in triplicate, with a representative curve shown in FIG. 32. This particle size was further confirmed by SEM images of the dispersion after dilution to 100 mg/ml in the dispersion buffer, rapid freezing and lyophilization onto an SEM stage (FIG. 33). FIG. 33 shows nanoparticles of a size consistent with DLS measurements, but a different shape due to coating with crystallized trehalose. Previously, SEM and STEM images of dispersed sheep IgG and 1B7 particles at lower trehalose concentrations, visualized the dispersed particles as clusters of smaller particles (Miller 2011). Similar images were obtained for the current formulation after adding dispersion buffer to reduce the trehalose concentration, simultaneously reducing the 1B7 concentration to 40 mg/ml (FIG. 15C). To confirm that these results are not affected by dissolution of the 1B7 nanoparticles, the 1B7 solubility in dispersion buffer was measured, using methods reported previously (Miller 2011). Starting with the 200 mg/ml dispersion, added dispersion buffer was progressively added to reduce the protein concentration and measured the resulting particle sizes by DLS (FIG. 32). A single peak at ˜200 nm was observable until the protein concentration was reduced to 2.5 mg/ml or less. At this concentration, only a single ˜10 nm peak at is present, corresponding to the hydrodynamic diameter of a single monoclonal antibody molecule. From these data, it can be concluded that the solubility of 1B7 this dispersion buffer is ˜2.5 mg/ml and that 1B7 nanoparticles formed with trehalose, PEG and NMP are fully reversible.

The dispersed particles were formed and exhibited colloidal stability due to a balancing of the intermolecular attractive and repulsive interactions at the protein molecular and colloidal levels, respectively (Miller, 2011). Briefly, individual protein molecules are subject to highly attractive depletion and specific short-ranged interactions such as hydrophobic interactions, hydrogen bonding and charge-dipole interactions resulting in low protein solubility. (Miller 2011) Near the 1B7 pI, electrostatic repulsion is relatively weak and thus the attraction force dominates between individual protein molecules. However, once these molecules assemble into nanoclusters, the interactions between particles are slightly repulsive, stabilizing the dominant size. (Miller 2011) Each protein monomer on the cluster surface will have a small number of charges; summed over all the monomers on a particle surface, the repulsive interactions become significant. (Miller 2011). Attractive specific and short-range depletion-attraction interactions between clusters are minimized as the average distance between clusters increases as the colloid size increases, but the distance over which these forces act is fixed. To exert the same force, an interaction with a range of 1 nm on a 10 nm protein monomer would need to act over 20 nm on a 200 colloid. Between clusters, short-range attractive interactions are negligible relative to electrostatic repulsion resulting in a colloidally-stable dispersion of protein nanoclusters, as has been previously shown for sheep IgG. (Miller 2011) To formulate a stable antibody dispersion and balance repulsive forces, the depletion attraction forces need to be adjusted by varying the concentrations of the crowding agents. (Miller, 2011) As observed herein and previously (Miller 2011), an increase in crowder concentrations dramatically reduces 1B7 solubility due to depletion-attraction interactions. While 1B7 and the sheep IgG dispersions could both be formulated with a single crowding agent, trehalose, the ternary crowder system as used herein provides additional flexibility to tune solubility and formulate dispersions with highly soluble proteins or to further control the nanocluster size, protein stability, dispersion viscosity and nanocluster degradation during delivery.

The low apparent viscosity, 24 cP, of the ˜190 mg/ml 1B7 dispersion was measured as the viscosity through a 25 gauge 1.5 inch needle. This viscosity measurement was previously characterized for subcutaneous injections of highly concentrated solutions of monoclonal antibodies and non-aqueous suspensions of lyosyzme. The apparent dispersion viscosity is commonly described as a function of the intrinsic viscosity, [η], maximum volume fraction of particles, φₘₐₓ, and the solvent viscosity, ηₒ, using the Krieger-Dougherty equation (Eq. 26).

$$\frac{η}{{η}_{o}} = {[ 1 - ( \frac{φ}{{φ}_{max}} ) ]}^{- [ η ] {φ}_{max}} \qquad ( 26 )$$

The η may be reduced by lowering ηₒ, or [η], which has a minimum of 2.5 for hard sphere colloids, and increasing φₘₐₓ. For protein molecules in solution at high concentrations, for example φ=0.1 to 0.3, strong short-range specific attractive interactions, often produce viscosities 5 to 100 times the hard sphere value. For monoclonal antibody solutions with concentrations of 150 mg/mL, viscosities greater than 100 cP have been attributed to reversible self-association of protein molecules, on the basis of measurements by analytical ultracentrifugation. In contrast, the low viscosities observed in the present study for the nanocluster dispersions may be consistent with the weak interactions between the nanoclusters, as reported previously (Miller 2011).

TABLE 8
Examples of protein dispersions made.

| | Protein Conc. | | | | |
|---|---|---|---|---|---|
| Protein | (mg/ml) | Trehalose Conc. | φ | φ_{E} | Dh |
| MAb 1B7 | 220 | Trehalose | 0.16 | 0.14 | 322 |
| Sheep IgG | 300 | Trehalose | 0.21 | 0.19 | 85 |
| BSA | 350 | Trehalose | 0.26 | 0.14 | 39 |

Sterile filtration of dispersions: 160-170 mg/mL dispersions of Bovine Serum Albumin (a 66 kDa protein) were formed using trehalose as the crowder. The dispersions were found by DLS to have a hydrodynamic radius of approximately 60 nm (see Table 9 for details). These dispersions were then filtered through a 0.22 micron Millex-GV syringe filter (Durapore PVDF membrane, 13 mm in diameter). Concentration of the filtered was less than 5% different than the original dispersion, and nanoclusters could be observed via DLS.

TABLE 9
Sterile Filtration Experiment.

| | Pre Filtra- | Post Filtra- | | |
|---|---|---|---|---|
| | tion Con- | tion Con- | Pre Filtra- | Post Filtra- |
| | centration | centration | tion Cluster | tion Cluster |
| Dispersion | (mg/mL) | (mg/ML) | Size (nm) | Size (nm) |
| 200 mg/mL 1:1 | 160 | 161 | 57.7 | 31.9 |
| BSA:Tre, 20% | | | | |
| NMP, 10% PEG | | | | |
| 250 mg/mL 1:1 | 174 | 168 | 54.5 | 31.9 |
| BSA:Tre | | | | |

In vitro molecular stability of 1B7 in dispersion: The conformation of antibody contained within the dispersion and after dilution was assessed using multiple techniques. Tryptophan fluorescence assay, as reported previously for sheep IgG (Miller 2011), no change in the maximum emission wavelength solvent exposed tryptophans was observed, measured by a tryptophan fluorescence assay, suggesting preservation of the active protein structure within the dispersed particles. After 10-fold dilution from the 200 mg/ml dispersion into PBS, DLS measured a single species with a ˜10 nm hydrodynamic diameter, as expected for a single antibody monomer (FIG. 32). As with the 1B7 reconstituted from the SWIFT frozen and lyophilized powder, a lack of larger particles suggests that the antibody does not form irreversible aggregates upon dispersion and can recover its individual monomer size upon rapid dilution. This is further confirmed by non-reducing SDS-PAGE (FIG. 34A), in which a single band is observed, with a molecular weight corresponding to that of an antibody monomer, ˜150 kDa, indicating an absence of irreversible thiol-linked and SDS-resistant aggregates. Finally; an ELISA to monitor the specific PTx-binding activity of the antibody reveals no significant change in activity due to the formation or dilution of the dispersion versus untreated control (FIG. 34B).

To maintain therapeutic efficacy without inducing an adverse immunogenic response upon in vivo injection (Saluja and Kalonia), the conformational stability of the antibody must be maintained through every processing and delivery step: from creation of the dry, lyophilized powder to dispersing of the powder in the dispersion buffer. While instability of protein molecules includes both chemical degradation as well as physical denaturation, the higher order dependence on protein concentration of physical denaturation is expected to be a more severe challenge for the successful development of stable high protein concentration formulations and thus is examined in further detail. (Saluja and Kalonia) As discussed hereinabove, the protein powder formed by SWIFT freezing and lyophilization did not lose activity or developed aggregates after reconstitution in buffer. Stability of the protein within the dispersed particles is maintained due to the high volume fraction of protein within each particle. A high protein volume fraction allows protein self-crowding effects to result in the thermodynamic favoring of the natively-folded lowest surface area conformation of the protein. (Shen, Cheung et al. 2006; Miller 2011) The concept of self-crowding to increase the fraction of natively folded proteins is similar to the idea that within cells proteins are stabilized by a high concentration of molecular crowding agents. In the case of self-crowding, the only difference is that the protein acts as its own molecular crowding agent. (Shen, Cheung et al. 2006) As a result when compared to a solid protein crystal, the difference between the amorphous protein particles formed herein and crystalline protein particles does not lead to a reduction in the stability of the native conformation of the protein. The entropic stabilization of the native protein state from self-crowding has been shown previously in theoretical arguments (Shen, Cheung et al. 2006), however, as protein solutions cannot achieve the high (>0.15) volume fractions necessary, it has only been realized recently for protein dispersions. (Miller 2011) In addition, unfolding and aggregation of the protein molecules in the dispersion are also reduced by decreased protein mobility of the solid state versus the solution state. Kinetically, the protein molecules on the outside of the particles in the dispersion are also stabilized by the reduction in collisions which could lead to the formation of aggregates. (Miller 2011)

The retention of active protein and lack of detectable aggregates of the protein upon dilution from the concentrated dispersion is an important indication of potential in vivo protein stability. The predicted dissolution time in PBS using the Noyes-Whitney equation for high surface/volume 200 nm particles with a solubility of greater than 50 mg/ml is less than 1 second. In a previous study, a misfolded protein refolded during the slow dissolution process. (Webb 2002) In the present study, the protein starts out in the folded state and has little time to unfold during the rapid dissolution. In addition, the molecular crowders present in the dispersion formulation will also be present simultaneously in the boundary layer surrounding the protein particles and help preserve the folded state. (Zhou, Rivas et al. 2008) For 1B7, high protein stability was observed upon diluting the protein at a constant crowder concentration by DLS and ELISA measurements, consistent with this rapid dissolution/crowding mechanism. (Miller 2011) The fact that the clusters are natively-folded and reversible is highly beneficial for maintaining protein stability during dissolution of the clusters. With sheep IgG, at a constant protein concentration, a steady decrease in nanoparticle size was achieved upon diluting a single low molecular weight crowder, trehalose, to weaken the attractive forces. (Miller 2011) At each step the protein in the cluster was found to be folded and full activity of the protein upon dissolution of the cluster was confirmed by ELISA. (Miller 2011) The protein molecules on the cluster surface are crowded by interior protein molecules and on the exterior by sugar molecules. As the folded molecules rapidly diffuse off the cluster surface into the PBS media, they remain folded as shown by the DLS and ELISA studies.

In vivo bioavailability of stable 1B7 from dispersions: No reliable in vitro models exist to mimic in vivo dissolution of the rapidly dissolving (<1 second predicted dissolution time) dispersion after subcutaneous injection. Thus, a mouse model was used to measure the pharmacokinetic parameters as well as the specific activity of in vivo dissolved antibody material. The five treatment groups included three control groups to allow direct PK comparison, a low volume, high concentration and large volume, high concentration dispersion test groups. The control groups received a standard antibody dose (4.6-5.6 mg/kg delivered in 100 μl) to allow for a direct comparison of pharmacokinetics resulting from a subcutaneous dispersion injection and intravenous and sub-cutaneous delivery of an antibody solution. The fourth group was designed to assess the combined effects of dispersion concentration and delivered volume on in vivo dissolution rates and the resulting pharmacokinetics. These mice received a standard dose (7.3 mg/kg) administered as a high concentration dispersion (182 mg/ml) in a small 1 μl volume. The fifth group was designed to administer an ultra-high dose, which can only be achieved with high concentration, low viscosity formulations such as dispersions. These mice received a ten-fold higher dose than the other groups (51.6 mg/kg in 100 μl). For all groups, serum samples were collected from the tail vein over 14 days, with the concentrations of total and active 1B7 antibody in each sample measured by streptavidin and PTx capture ELISAs, respectively. The efficacy of antibody present at the terminal time point was also assessed using an in vitro activity assay, based on antibody-mediated inhibition of toxin activity.

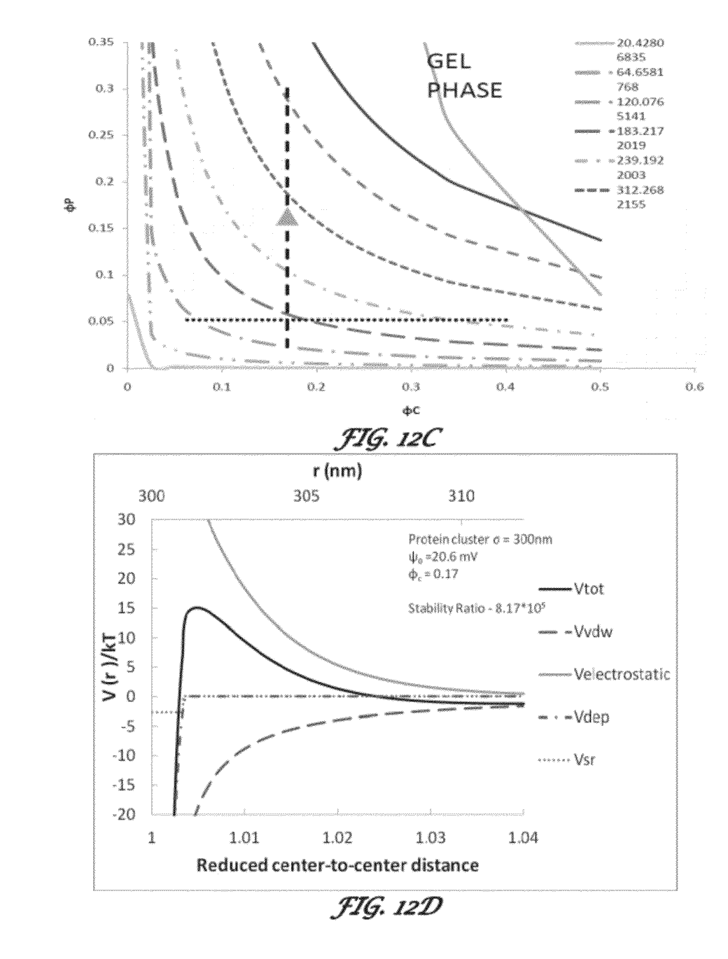

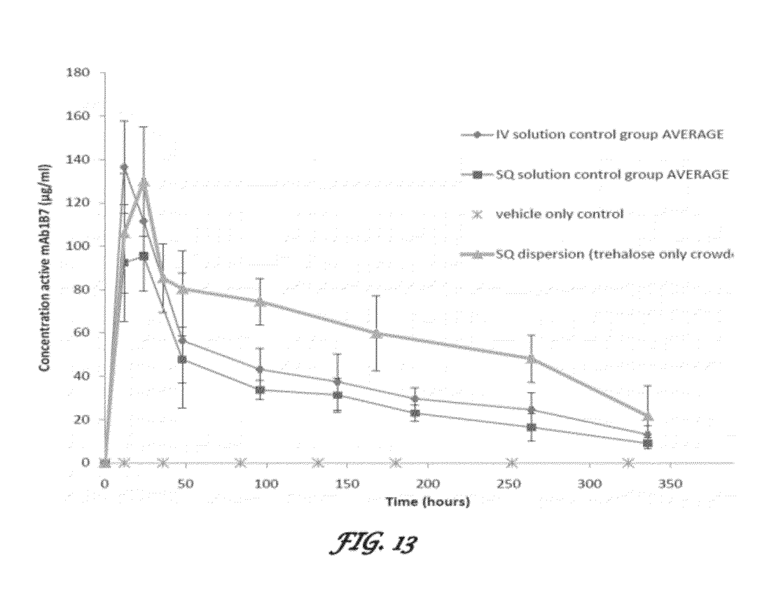

Overall, the 1B7 pharmacokinetic profile is quite similar for all groups, with nearly identical distribution and elimination kinetics. The primary differences result from the injection site and injection volume, affecting the time to reach the maximum concentration a (tₘₐₓ) and the value of the maximum concentration (Cₘₐₓ/dose). Looking first at the three control groups, delivery via subcutaneous dispersion resulfed in a reduced burst phase (lower Cₘₐₓ/dose and delayed tₘₐₓ) as compared to IV and SQ delivery of solutions (Table 7; FIG. 13). The IV solution group reached a maximum serum concentration at the first measured time point (12 hours), followed by a rapid decrease as the antibody is distributed throughout the tissues. (Miller 2011) In comparison, the SQ solution group displayed a slightly reduced Cₘₐₓ/dose (24 versus 18 μg/ml/mg/kg) and statistically significant delayed tₘₐₓ (12 versus 21 hrs; p<0.05). While IV-administered material is instantly diluted in the blood volume, material administered SQ must diffuse from the injection site through interstitial fluid to reach the lymphatic and blood vessels before distribution in the blood volume, delaying these PK parameters (FIG. 15A). (Miller 2011) The SQ dispersion injections exhibit similar trends as the SQ solution but with a lower Cₘₐₓ and delayed tₘₐₓ when compared to the SQ solution. This may reflect the effects of the dispersion buffer on mixing and antibody diffusion, as the effect is minimized with SQ dispersion 2, which was injected as a 1 μl volume instead of a 100 μl volume. For this sample, the tₘₐₓ was identical (within error) to that of the SQ solution.

Once the maximum serum concentration is attained, all groups show similar 1B7 pharmacokinetics. As seen in FIG. 13, these data fit a biphasic exponential profile, with a distribution and (3 elimination time constants that are within experimental error for all groups, based on 1B7 concentrations measured by the PTx ELISA (Table 7). The 13 elimination half-life was also within error for all groups when measured using a total protein ELISA assay (results not shown). The distribution phase represents passive antibody diffusion from the well-mixed blood volume into other tissues, driven by the 1B7 concentration gradient and the elevated vascular pressure, while antibody elimination rates are controlled by interactions with specific receptors such as the FcRn. Notably, both mechanisms require a monomeric, properly folded antibody molecule. A soluble aggregate will have a larger size and consequently larger diffusion constant and slower t1/2a, while a misfolded monomer or soluble aggregate will exhibit different binding kinetics for the FcRn and a different t1/2 beta. The similar kinetics observed for all groups indicate that the antibody delivered as a SQ dispersion is able to dissociate from the nanocluster and diffuse away from the injection site while retaining an active, monomeric form, similar to our in vitro observation in which active 187 monomer is rapidly recovered upon dispersion dilution.

The studies described hereinabove were performed in mice, where the large allowed injection volume per body mass (100 μl/25 g) allows for direct comparisons between solutions and dispersions formulated at the same concentration. A similar comparison is not possible in humans, as SQ injections are restricted to ˜1.5 ml volume. To demonstrate that dispersions can achieve dosages relevant for humans, group 4 was prepared as a scaled-down version of a human dose. Here, a 1 μl volume of highly concentrated dispersion (182 mg/ml) was administered subcutaneously, for a final 7.6 mg/kg murine dosage. Scaling-up to calculate the human dosage, in which a 182 mg/ml dispersion could be administered in a L5 ml volume, this is equivalent to a 4.3 mg/kg human dose, exceeding current dosing guidelines (2 mg/kg). To evaluate the potential for dispersions to result in less-frequent administration of ultra-high antibody dosages, which are not currently achievable with solutions, group 5 mice received a large, 100 μl injection volume of highly concentrated dispersion (182 mg/ml), for a 51.6 mg/kg dose. This group also exhibited similar pharmacokinetics (similar tₘₐₓ, t_{1/2,α}, t_{1/2,β}) and 1B7 bioavailability indicating concentration and dose-independent pharmacokinetics.

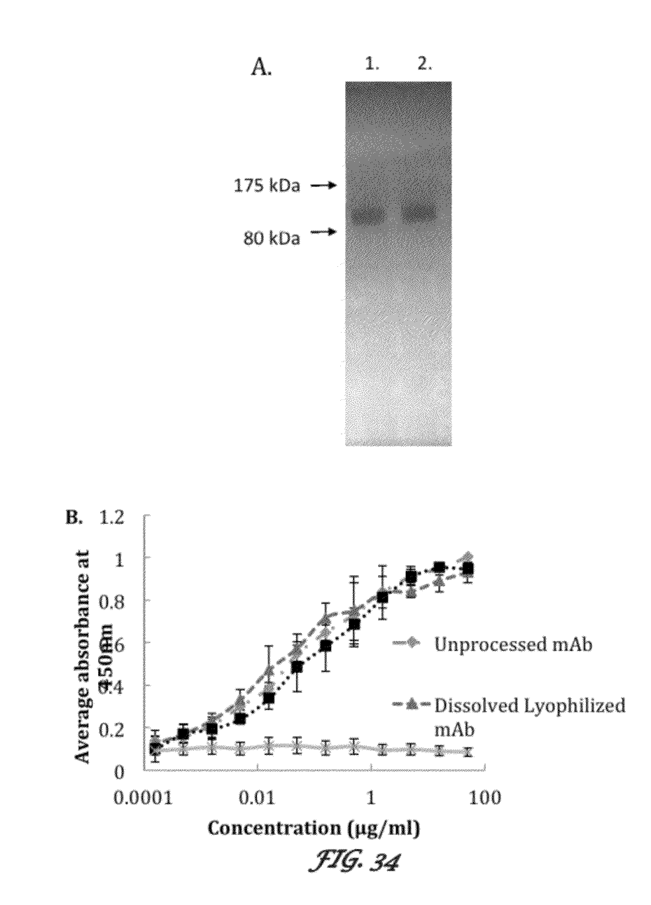

To provide an orthogonal measurement of antibody quality to complement antigen ELISA, we measured 1B7 biological activity with an in vitro CHO cell neutralization assay using sera from the terminal time point. Free PTx will bind cell-surface receptors, undergo receptor-mediated endocytosis and eventually ADP-ribosylation of G_{i/o} coupled receptors; phenotypically, this results in loss of contact inhibition and CHO cells grow in a clustered morphology. Antibody-mediated neutralization of PTx blocks toxin entry into cells, protecting normal growth phenotype. Sera were diluted in the presence of a fixed PTx concentration, CHO cells added and, after 24 hrs growth, scored for normal or clustered morphology. The highest sera dilution completely preventing CHO cell clustering was recorded and compared versus purified control 1B7 antibody. This assay resulted in no statistically significant differences between groups on titre per μg antibody basis. Based on this assay, there is no evidence for a loss in antibody efficacy as a result of injection site (SQ vs. IV) or formulation (Table 7). Western blot analysis was used to demonstrate the absence of gross physical changes in serum antibody due to formulation and administration route, such as formation of insoluble or disulfide bonded aggregates (FIG. 35).

As shown by both in vitro and in vivo data, the protein within the dispersion shows no detectable loss of native conformation during any processing step or after dissolution and systemic absorption. As described previously within the dispersion, the native conformation is maintained by protein self-crowding and the addition of crowding agents entropically stabilizing the native conformation. (Shen, Cheung et al. 2009) For aggregation to occur upon dissolution, the native protein must reversibly unfold to an aggregation-prone intermediate and collide with another aggregation-prone protein molecule, which leads to irreversible inactivation of the protein. As in the in vitro dilution experiments, initially in vivo the crowders within the dispersion (trehalose, PEG, and NMP) are still present as the particles dissolve and thus entropically prevent the protein from unfolding. Due to the fast dissolution time, the nearby crowders from the dispersion will help prevent unfolding of the protein. In vivo, as the crowders from the dispersion are diluted, additional crowders in the extracellular environment around the SQ injection or in the blood stream will help maintain the protein stability. Thus during the in vivo particle dissolution and distribution of 1B7 near the injection site, stabilizing crowders, either from the injection or naturally occurring within the body, reduce the number of aggregation-prone protein intermediates. Furthermore the fast dissolution kinetics will dilute the therapeutic protein and decrease the number of collisions that lead to the formation of aggregates. As the dissolved crowder is diluted upon injection, if individual protein molecules diffuse away from the nanocluster surface, as in the in vitro studies, stabilization by self-crowding will still be present in the remaining protein molecules in the nanocluster. Preliminary immunogenicity studies support this conclusion as no propensity for the generation of anti-drug antibodies is detected in any sample. Therefore, with the result from the CHO assay, the protein particles from dispersion likely retain the native antibody state even after in vivo injection.

The nanocluster dispersions as described herein allow formulation of a monoclonal antibody at high concentration and low viscosity, with no detectable loss in antibody structure or activity in vitro or in vivo and similar pharmacokinetics when administered subcutaneously to mice. Highly concentrated ˜200 mg/ml aqueous-based dispersions of a therapeutically relevant antibody, 1B7 (Sutherland and Maynard 2009), were formed from stable, submicron protein particles (e.g. nanoclusters) containing a 1:1 weight ratio of trehalose in an aqueous buffer with multiple crowding agents, including trehalose, PEG and NMP. These particles were produced by rapid freezing in a dosage vial using spiral-wound in-situ film technology (SWIFT) to minimize protein denaturation and aggregation. The solubility of 1B7 was lowered in the aqueous-based solvent by adding pharmaceutically acceptable crowding agents, PEG300 and NMP, along with the trehalose from the dry powder to facilitate formation of the dispersion.

The protein particles described herein retained their native conformation in the dispersion as shown by fluorescence of the tryptophan residues on the protein. Additional analyses, ELISA, DLS and SDS-PAGE upon dilution of the dispersion into a pure buffer, indicate that the protein rapidly recovers monomeric form with full activity. Similar in vivo distribution and elimination half-lives were measured from the dispersion and solution formulations at similar doses, while the time to peak serum concentration (tₘₐₓ) was delayed for the SQ injections, consistent with the expected slower diffusion kinetics from this injection site. Specific PTx binding activity by ELISA, as well as an in vitro PTx neutralization test, were unable to detect a loss in 1B7 activity or development of anti-1B7 immune responses. The ability to form stable, highly concentrated dispersions of a protein therapeutic with low viscosities and favorable bioavailability as described in the present invention will increase the potential use of subcutaneous injection, possibly for treatment of many chronic diseases.

### Example III

The crowded macromolecular environment within cells (˜400 mg/ml) is known to favor the compact native state of proteins over unfolded conformations. (Hartl and Hayer-Hartl 2002; Zhou, Rivas et al. 2008) As described with scaled particle theory, (Davis-Searles, Saunders et al. 2001; Zhou, Rivas et al. 2008) simulation, (Hall and Minton 2003; Cheung and Truskett 2005; Shen, Cheung et al. 2006) and experiment, (Kendrick, Carpenter et al. 1998; Krishnan, Chi et al. 2002; Cheung, Klimov et al. 2005; Zhou, Rivas et al. 2008; Dhar, Samiotakis et al. 2010) not only in cells, but also in vitro, (Hall and Minton 2003; Cheung and Truskett 2005; Cheung, Klimov et al. 2005; Oconnor, Debenedetti et al. 2007; Zhou, Rivas et al. 2008; Dhar, Samiotakis et al. 2010; Pielak and Miklos 2010) proteins are stabilized against unfolding by the presence of other macromolecules (volume fraction φ˜0.3 to 0.4), which effectively “crowd out” (i.e., entropically penalize) more expanded, non-native protein conformations. Simulation and theory with coarse-grain models (Cheung and Truskett 2005; Shen, Cheung et al. 2006) also predict that high concentrations (c>400 mg/ml) of a single type of protein in solution favor the compact folded state via a mutual or self crowding mechanism. However, stable protein solutions at these ultrahigh concentrations have not been realized experimentally since proteins are rarely soluble and tend to gel at substantially lower concentrations in part due to specific short-ranged attractive interactions, especially hydrogen bonding and hydrophobic interactions. (Rosenbaum, Zamora et al. 1996; ten Wolde and Frenkel 1997; Shire, Shahrokh et al. 2004; Zaccarelli 2007; Zhou, Rivas et al. 2008; Scherer, Liu et al. 2010) In fact, at concentrations of 100-200 mg/ml, proteins in solution commonly undergo irreversible aggregation, (Fields, Alonso et al. 1992; Zhou, Rivas et al. 2008; Young and Roberts 2009; Scherer, Liu et al. 2010) gelation and precipitation. (Rosenbaum, Zamora et al. 1996; ten Wolde and Frenkel 1997; Shire, Shahrokh et al. 2004; Zaccarelli 2007) Therefore to avoid gelation, while simultaneously attaining “local” protein concentrations high enough to stabilize the native conformation via self-crowding, novel types of stable and reversible protein assemblies (e.g., nanoclusters) are needed.

Insights into nanocluster formation and phase behavior of protein solutions may be obtained from considering model polymeric colloid suspensions. (Gast, Hall et al. 1983; Rosenbaum, Zamora et al. 1996; Sedgwick, Egelhaaf et al. 2004; Stradner, Sedgwick et al. 2004; Lu, Conrad et al. 2006; Zaccarelli 2007; Lu, Zaccarelli et al. 2008) In the latter, tunable short-range colloidal attractions (e.g., cosolute-induced depletion interactions) are often present. (Sedgwick, Egelhaaf et al. 2004; Lu, Zaccarelli et al. 2008) Strengthening such attractions (e.g., by increasing cosolute concentration) causes highly polydisperse particle assemblies to form, which percolate and then gel near the colloid phase separation boundary. (Lu, Zaccarelli et al. 2008) (Sedgwick, Egelhaaf et al. 2004; Pan, Vekilov et al. 2010) Whereas phase separation and gelation result from strong attractions between uncharged colloids at high concentrations, (Lu, Conrad et al. 2006; Zaccarelli 2007; Lu, Zaccarelli et al. 2008) the physics change qualitatively when weak, longer-range electrostatic repulsion between particles is also present. (Sedgwick, Egelhaaf et al. 2004; Zaccarelli 2007) In such cases, as predicted with an equilibrium model, (Groenewold and Kegel 2001; Groenewold and Kegel 2004) long-lived and very large clusters of primary colloidal particles (i.e., cluster/to particle diameter ratio of 5-10 with low cluster-size polydispersity) have been observed in single-phase organic solvents (FIG. 36a). (Groenewold and Kegel 2001; Groenewold and Kegel 2004; Sedgwick, Egelhaaf et al. 2004; Stradner, Sedgwick et al. 2004; Zaccarelli 2007) These clusters form due to the presence of short and long-ranged interactions at the monomer scale which, in turn, produce diverse multi-scale (monomer-monomer, monomer-cluster, and cluster-cluster) interactions that affect both self-assembly and transport properties of the particle dispersions.

Clusters of proteins observed to date in water have been small (Stradner, Sedgwick et al. 2004; Porcar, Falus et al. 2010) (N˜10, cluster/particle diameter ratio of 2.5), dilute, (Pan, Vekilov et al. 2010) and short-lived. (Porcar, Falus et al. 2010) Recently, reversible clusters of Au particles in water have been assembled with diameters from 30 to 100 nm (cluster/particle diameter ratios from 6 to 20) by tuning the charge on the Au particles with a weakly adsorbing non-electrolyte. (Tam, Murthy et al. 2010; Tam, Tam et al. 2010) More recently, nanoclusters have been reported for CdSe. (Xia, Nguyen et al. 2011) It remains a challenge to properly balance the attractive and repulsive interactions to form large clusters of proteins.

In analogy with the model colloid systems discussed above, the strength of effective protein-protein attractions in solution can also be tuned through the presence of cosolutes. For example, even cosolutes that interact weakly with the proteins still produce protein-protein depletion attraction. (Davis-Searles, Saunders et al. 2001; Ping, Yang et al. 2006; Oconnor, Debenedetti et al. 2007) These depletion attractions reflect the osmotic pressure imbalance that occurs when the surfaces of two protein molecules approach close enough to exclude cosolutes from the intervening gap (FIG. 42). They are known to strongly influence the equilibrium behavior (Minton 1999; Zhou, Rivas et al. 2008) and rates (del Alamo, Rivas et al. 2005; Zhou, Rivas et al. 2008) of association of proteins into dimers or small oligomers. However, this behavior has received far less attention than other related crowding (i.e., excluded volume) effects that low (Lee and Timasheff 1981; Kendrick, Carpenter et al. 1998; Davis-Searles, Saunders et al. 2001; Krishnan, Chi et al. 2002; Chi, Krishnan et al. 2003; Oconnor, Debenedetti et al. 2007) and high (Hall and Minton 2003; Zhou, Rivas et al. 2008; Dhar, Samiotakis et al. 2010; Pielak and Miklos 2010) molecular weight cosolutes (crowders) have on protein folding and/or site binding. The potential of mean force for depletion attraction between proteins, V_{dep}(r) is proportional to the volume fraction of the cosolute (extrinsic crowder) φ_{E}, as described with scaled particle theory (Davis-Searles, Saunders et al. 2001; Oconnor, Debenedetti et al. 2007) or by the Asakura-Oosawa model. (Asakura and Oosawa 1958; Vrij 1976; Gast, Hall et al. 1983; Sharma and Walz 1996; Sedgwick, Egelhaaf et al. 2004; Ping, Yang et al. 2006; Mutch, van Duijneveldt et al. 2007; Zaccarelli 2007). For model monomeric and oligomeric cosolutes at a fixed high concentration, V_{dep} can produce a strongly attractive osmotic second virial coefficient for a wide range in diameter (ratio of extrinsic crowder to that of protein monomer) from 0.02 to 1. (Asakura and Oosawa 1958; Vrij 1976; Tuinier, Vliegenthart et al. 2000; Lu, Conrad et al. 2006; Lu, Zaccarelli et al. 2008) An example of a diameter ratio of 0.1 would be a 10 nm protein molecule and a 1 nm disaccharide. Thus, similar to the behavior of model colloids, depletion attractions due to small crowders—such as trehalose at high concentrations—could potentially be utilized to provide sufficient attraction to balance weak electrostatic interactions and form large protein clusters.

Herein we assemble ˜100 nm equilibrium clusters of proteins (mAb 1B7, polyclonal sheep IgG and BSA), which dissociate into stable protein monomer upon diluflon in buffer. The nanoclusters are formed simply by gently mixing lyophilized protein powder containing trehalose, and buffer solution with protein concentrations up to 267 mg/ml for mAb 1B7, 350 mg/ml for IgG and 400 mg/ml for BSA. To drive formation of large clusters in water, we (1) minimize the net protein charge with a buffer pH near the pI to weaken electrostatic repulsion, and (2) add high concentrations of a cosolute (extrinsic crowder), trehalose, to provide strong depletion attraction. The size of the clusters is either increased or decreased reversibly over a continuum by varying the concentration of cosolute (crowder), as shown by dynamic light scattering (DLS). The cluster size is predicted qualitatively by an extension of an earlier free energy model to account for the fractal dimension (δ_{f}) of the cluster. By adjusting φ_{E} and the pH, we balance hierarchical (protein-protein, protein-cluster, and cluster-cluster) interactions in such a way that promotes assembly of fluid dispersions of nearly monodisperse, weakly-interacting protein nanoclusters with ultra-high internal volume fractions (φ>0.5 or c>˜700 mg/ml). The high internal c stabilizes proteins in their folded state via self-crowding, as shown theoretically. (Cheung and Truskett 2005; Shen, Cheung et al. 2006).

The stability of the protein after delivery from the clusters is of interest in protein therapeutics. After diluting the nanoclusters in buffer, the protein nanoclusters are shown to dissociate to protein monomers by dynamic light scattering (DLS), (Horn 2000) size exclusion chromatography (SEC), and sodium dodecyl sulfate polyacrylamide gel electrophoresis (SDS-PAGE). The protein is demonstrated to be folded by circular dichroism (CD), thermodynamically stable by determination of the apparent melting temperature (Tₘ), (Lavinder, Hari et al. 2009) and biologically active by an enzyme-linked immunosorbent assay (ELISA). (Sutherland and Maynard 2009) Finally, the low viscosity of 40 cP, resulting from weak intercluster interactions, allows subcutaneous injection of the concentrated clusters at concentrations including up to 267 mg/ml. As an indication of the ability of these dispersions to dissociate and deliver active protein, an in vivo bioavailability study is performed with mice. The pharmacokinetic profile of the dispersed protein nanocluster dose is compared to both subcutaneous and intravenous doses of dilute antibody solution, with activity of protein in the bloodstream quantified by both ELISA and an in vitro antibody neutralization assay. (Sutherland, Chang et al. 2011)

### Nanocluster Morphology and Tunability With Trehalose and Dilution in Buffer

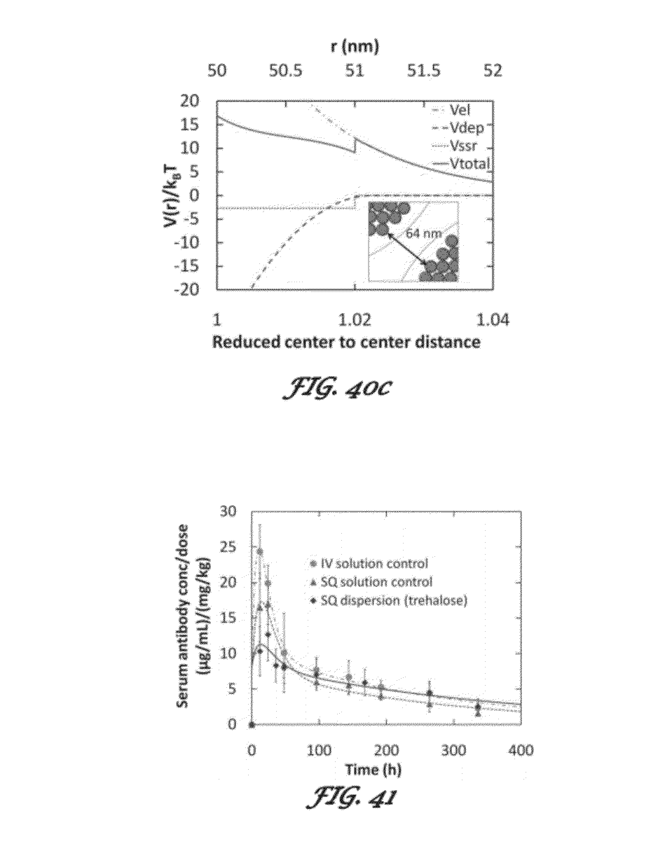

FIG. 36b shows a colloidally-stable, transparent dispersion of the monoclonal antibody 1B7 (Sutherland and Maynard 2009) that formed immediately upon gentle stirring of lyophilized protein powder (with a 1:1 mass ratio of trehalose to protein) in phosphate buffer solution at the pI (pH 7.2). The concentrations of protein, c, and extrinsic crowder, trehalose, c_{E}, were each 220 mg/ml. The low turbidity is a consequence of the small D_{c} and small difference in refractive indices of the porous cluster and solvent. The SEM images of the dispersions after cryo-preparation revealed ˜300 nm nanoclusters composed of primary particles about the size of protein monomer, ˜11 nm (FIG. 36c and FIG. 43), as shown with the help of a graphic visualizing these clusters in dispersion in FIG. 36d. The “halos” about the primary particle the nanoclusters are a result of trehalose deposition during SEM sample preparation, and thus of minor interest. For c=c_{E}=220 mg/ml, the average hydrodynamic diameter, D_{c}, of the clusters from dynamic light scattering (DLS) was 315 nm (std. dev. in peak width of 6% over the mean) in agreement with the SEM images (FIG. 37a). For the porous clusters, the volume fraction of protein within a cluster φᵢₙₜ, was measured to be 0.6 with static light scattering (SLS, FIG. 44), as a function of the fractal dimension (δ_{f}) (Eq. 22). The δ_{f} is the slope in the log-log plot of the intensity against the scattering vector. The fractal dimension in the case of 80 nm IgG clusters was found to be 2.6 versus 3, 2 and 1 for completely space filled spheres, disks and long thin rods respectively, which suggests that the protein has a high volume fraction inside the nanoclusters.

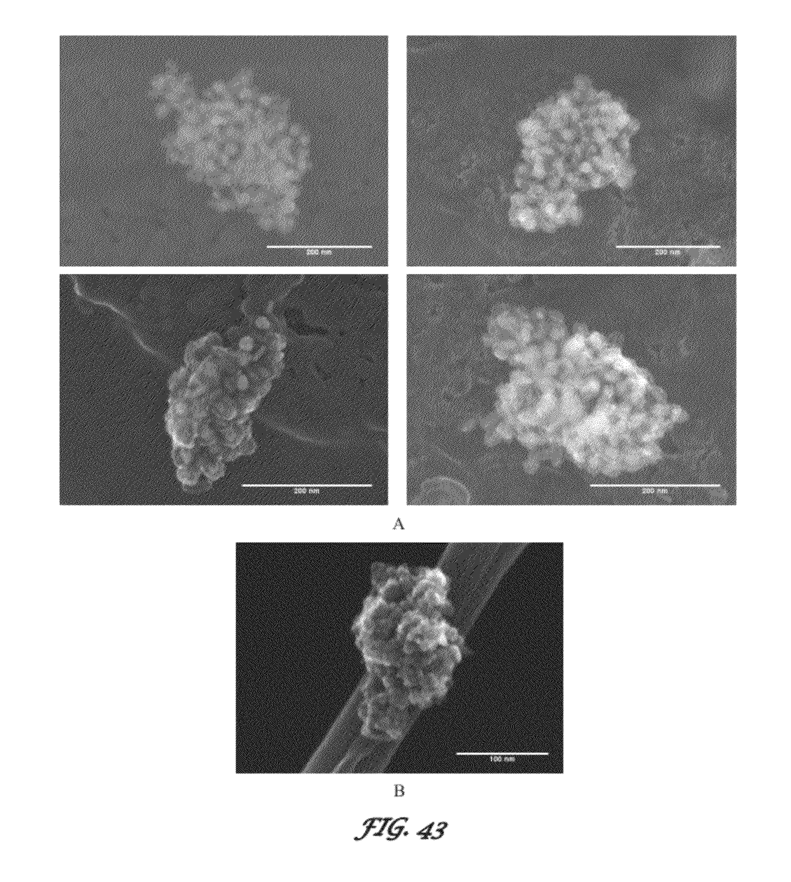

Upon successive dilutions of the 220 mg/ml 1B7 dispersion in phosphate buffer to maintain a constant c/c_{E} ratio, D_{c} decreased over a continuum as protein molecules left the cluster surface (FIGS. 37a and 37b). D_{c} then reached a plateau at ˜12.3 nm for c=c_{E}=75 mg/ml, the expected size of an antibody monomer. Similarly, dilution of c_{E} from 270 to 150 mg/ml with c fixed at 70 mg/ml 1B7 was used to tune the cluster size until reaching a c_{E} below which only ˜10 nm species, presumably antibody monomers, were observed (FIGS. 37b and 37c). The trehalose concentration was decreased using pH 7.2 phosphate buffer along with small amounts of dispersion with c=c_{E}=100 mg/ml to maintain a constant c. Upon subsequently increasing c_{E} back to 270 mg/ml, the original D_{c} values of ˜300 nm were recovered. Similar experiments with a polyclonal sheep IgG mixture (FIG. 37d and FIGS. 43b and 45) resulted in the same trends. FIG. 43b shows a nancocluster of sheep IgG from a dispersion at c=c_{E}=260 mg/ml, which was diluted down to 50 mg/ml followed by cryo-preparation. The IgG nanocluster size decreased from ˜80 nm at c_{E}=270 mg/ml to ˜10 nm (monomeric protein) for c_{E}=150 mg/ml at a constant c=50 mg/ml (FIG. 37d). (When increasing c_{E} a 500 mg/ml trehalose solution in pH 6.4 phosphate buffer (pI of IgG) was used along with small amounts of dispersion with c=c_{E}=200 mg/ml to maintain a constant c). Very similar values of D_{c} were observed upon either increasing or decreasing the trehalose concentration. This reversibility in the nanocluster size suggests the nanoclusters were in an equilibrium state, as further explained below with the predictions from the free energy model. The cluster size for the sheep IgG also decreases from 80 nm to 11 nm (monomeric protein) when the dispersion was sequentially diluted in pH 6.4 phosphate buffer from c=c_{E}=260 mg/ml to c=c_{E}=47 mg/ml as shown in FIG. 45. Taken together, these data demonstrate a novel type of long-lived (tested for several hours) well-defined nanocluster in aqueous media, with reversible equilibrium behavior, which was unexpected. (Stradner, Sedgwick et al. 2004; Pan, Vekilov et al. 2010; Porcar, Falus et al. 2010)

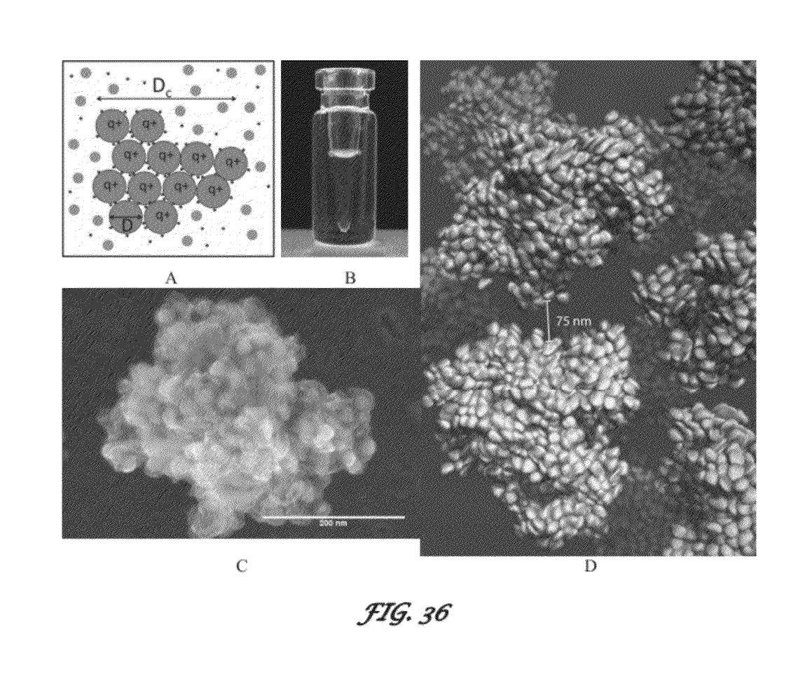

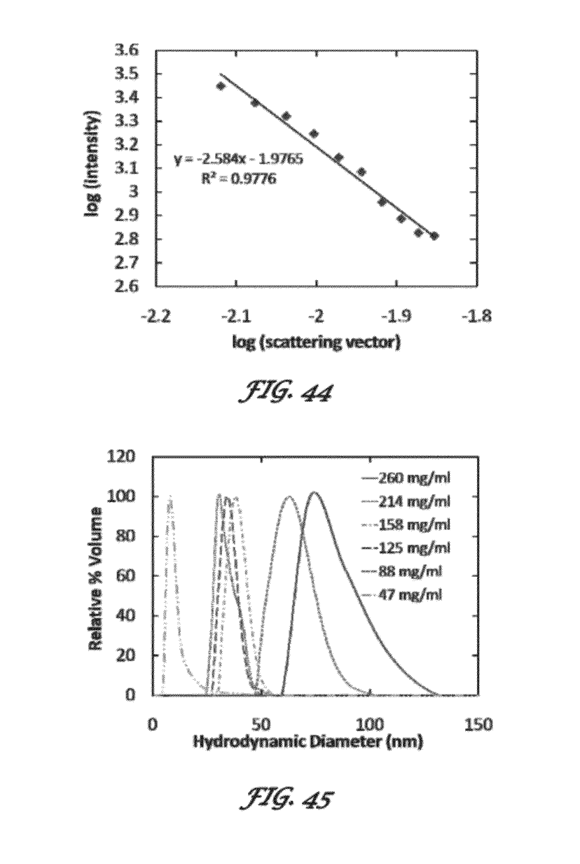

To demonstrate further the generality of the technique, clusters were also formed with macromolecular crowders including PEG (M.W. 300), N-methylpyrrolidone (NMP) and dextran (M.W. 10,000). With sheep IgG at a concentration of 162 mg/ml with 162 mg/ml trehalose and 20% (v/v) PEG-300, the cluster diameter was 110 nm. For sheep IgG at a concentration of 157 mg/ml with 157 mg/ml trehalose, 10% (v/v) PEG-300 and 20% by volume NMP, the clusters were ˜250 nm in diameter. Also 315 mg/ml BSA with 5% (v/v) PEG300 and 20% (v/v) ethanol yielded clusters of size 30 nm (BSA monomer is 4-5 nm). These examples with macromolecular crowders, illustrate the generality of the technique. Apart from that, in order to demonstrate the possibility of using this technique at higher concentrations of protein as a proof of concept, higher concentration dispersions of proteins were prepared. FIG. 38 shows nanoclusters of BSA at a very high c of 400 mg/ml and c_{E}=240 mg/ml which have a D_{c}=40 nm. The number of protein monomers, about 1000, in the cluster is of the same order as the clusters formed from mAb 1B7 and sheep IgG. Highly concentrated dispersions are also shown for sheep IgG in FIG. 46 where nanoclusters with D_{c} of ˜100 nm were observed for c=300 and 350 mg/ml and c/c_{E}=1:0.5 where trehalose was the extrinsic crowder.

Protein Stability after Dilution of the Nanoclusters

A major concern for protein formulations at high concentrations is the potential for individual protein monomers to misfold and form irreversible aggregates. These events may result from the dynamic nature of a protein molecule: at any given moment, a system of identical molecules will present an ensemble of related three-dimensional structures, some of which transiently expose normally buried hydrophobic patches. At low concentrations, the protein will frequently recover its native conformation, but at high concentrations the probability of two proteins with exposed hydrophobic patches colliding and associating irreversibly is high. (Kendrick, Carpenter et al. 1998) These misfolded and irreversibly aggregated proteins do not present the native structure and therefore exhibit reduced potency and, due to their modified apparent size and exposed surface charges, altered pharmacokinetics. Moreover, the presentation of these non-native surfaces to the immune system can induce a response against the therapeutic protein, which will in itself change biological activity and pharmacokinetics. (Tabrizi, Tseng et al. 2006)

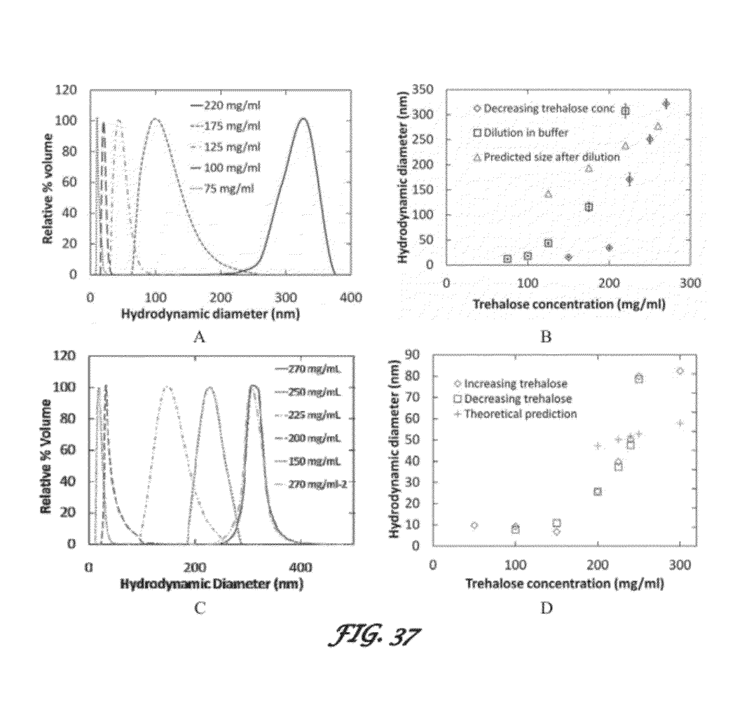

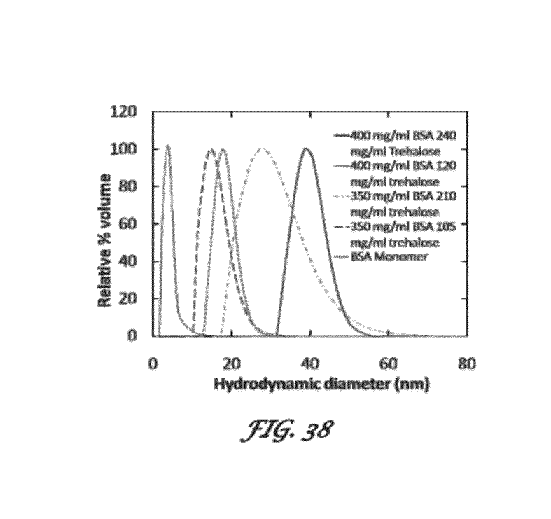

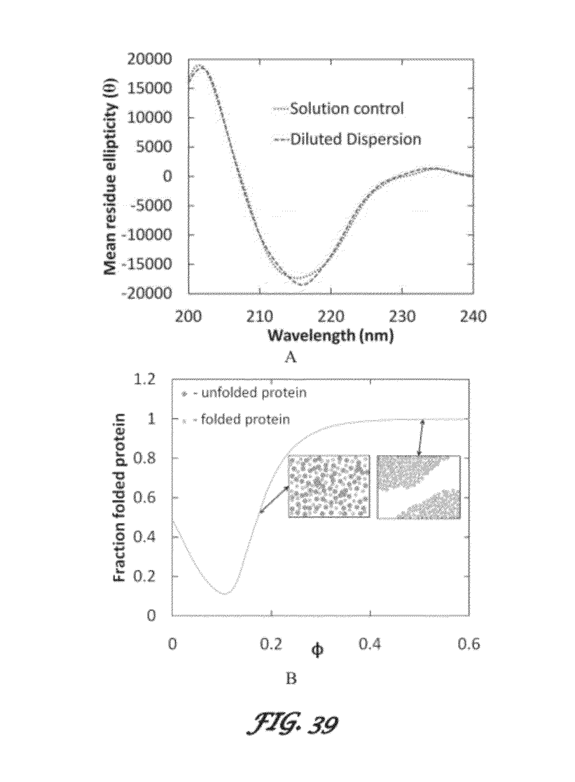

As discussed below, simulation results of earlier studies (Cheung and Truskett 2005; Shen, Cheung et al. 2006; Zhou, Rivas et al. 2008) suggest that the folded state is strongly favored for model proteins at high concentrations (i.e., values comparable to the local protein concentration within the nanoclusters). To investigate this hypothesis, experimental studies on actual antibodies are needed to determine whether proteins in the nanoclusters are in the folded state upon dissociation of the nanoclusters to protein monomer. To determine whether irreversible protein aggregates are present in our 1B7 nanocluster dispersions at 267 mg/ml, we performed a battery of biophysical and biochemical tests. The dispersions were diluted several hours after formulation, as long term storage stability is outside the scope of this work. (In practical applications, the dispersions could be formed and then injected into patients shortly thereafter.) However, the protein within the dispersion was stressed through viscosity testing earlier, as it was drawn through a 25 gauge needle, subjecting it to significant shear forces with a shear rate estimated to be as high as 9500 s⁻¹ assuming a Newtonian fluid. Remarkably, after dilution to 1 mg/ml in PBS, we were unable to detect a change in protein conformation or activity relative to the control antibody in solution (Table 10). Prior to dispersion, analysis of a control 1B7 antibody solution in PBS exhibited a stability typical of monoclonal antibodies, (Garber and Demarest 2007) with an apparent thermal unfolding transition temperature (Tₘ) of 68° C. (Table 10) and an unfolding midpoint at 6.2 M urea. After dilution of the dispersion, the Tₘ was again measured to be 68° C. (Table 10). Since a Tₘ change of two-to-three degrees indicates a change in conformational stability, this data demonstrates that the average 1B7 thermal stability was not altered. (Kumar, Sharma et al. 2009) Circular dichroism (CD) was used to monitor the presence of secondary structure elements in the protein as a function of absorption of polarized light at particular wavelengths. Both the control solution and diluted dispersion retained the same strong negative signal at 217 nm, indicative of the folded β sheet structure characteristic of antibodies (FIG. 39a and Table 11). (Chari, Jerath et al. 2009) Table 11 shows the secondary structure as estimated by Dichroweb, using the CDSSTR fitting algorithm. It is generally accepted that a normalized root mean square deviation (NRMSD) of <0.1 indicates a good fit. (Wallace, Janes et al. 2009) As shown in Table 11 the calculated percent β-sheet structure (the predominant secondary structure in antibodies) does not differ between the 1B7 control solution and the diluted dispersion.

Finally, two additional sizing methods were used to directly assess whether or not a small population of misfolded and larger molecular weight aggregates was present. As opposed to analysis of high concentration antibody solutions, (Scherer, Liu et al. 2010) HPLC size exclusion chromatography (HPLC-SEC) and SDS-PAGE analyses of the diluted dispersions show a negligible increase in higher molecular weight aggregates, when compared with the initial solution control (Table 10 and FIGS. 47 and 48). The presence of aggregates was also not apparent by DLS in the sharp monomer peaks (FIGS. 37a and 37c). HPLC size exclusion chromatography is able to discriminate antibody monomers from non-covalent and covalent aggregates, while non-reducing SDS-PAGE detects covalent multimers. FIG. 47 also shows the HPLC-SEC data for the intermediate steps in the dilution experiment for the 1B7 dispersion that are shown by DLS in FIGS. 37a and 37c. In all cases, there was not an increase in aggregates over the initial solution control.

TABLE 10
1B7 stability and activity in nanocluster dispersion samples with c =
c_{E} = 267 mg/ml diluted to 1 mg/ml in PBS prior to analysis.

| Sample | Tₘ (° C.)^{†} | % monomer (SEC) | EC₅₀ (ELISA) |
|---|---|---|---|
| Control solution | 67.7 ± 0.3 | 98.88 ± 0.04 | 1.00 ± 0.24 |
| Diluted dispersion | 68.3 ± 0.3 | 98.59 ± 0.04 | 1.03 ± 0.20 |
| (from 267 mg/ml) | | | |

Error indicated is ± s.d

TABLE 11
Estimation of 1B7 Secondary Structure from Circular Dichroism

| | | | % Turn and | |
|---|---|---|---|---|
| Sample | % α-helix | % β-strand | Unordered | NRMSD* |
| Control solution | 0 | 39 | 63 | 0.006 |
| Diluted dispersion | 1 | 40 | 60 | 0.006 |
| (from 267 mg/mL) | | | | |

*NRMSD is the normalized root mean square deviations between the calculated and experimental CD spectra. The program CDSSTR was used for all secondary structure estimates via the Dichroweb online analysis.

Although these biophysical tests (SEC, DLS, CD and SDS-PAGE) did not detect protein structural perturbations or aggregation, it is possible that the dispersed samples may have folded monomeric protein that does not retain biological activity. Thus, sensitive biological assays were used for determining activity that may be applied for protein concentrations<10 ng/ml. To monitor ligand binding activity, indirect ELISAs using pertussis toxin as a capture molecule measured the 1B7 activity in terms of the relative 50% effective concentration (EC_{50,disp}/EC_{50,control}). This ratio is the concentration of antibody resulting in 50% of the maximum ELISA response for the dispersion (after dilution to 1 mg/mL) versus that for an unmodified control solution. Here, the diluted dispersion yielded a relative activity of 1.03±0.20, which is indistinguishable from measurements made with the solution control (Table 10). (Crowther and Editor 1995) This result demonstrates that antigen binding ability, a powerful measure of protein activity, is identical for antibody recovered upon diluting a dispersion and a solution control.

The experimentally demonstrated stability of the native protein state in the large self-crowded nanoclusters may be anticipated from coarse-grain globular protein models (Cheung and Truskett 2005; Shen, Cheung et al. 2006) (FIG. 39b). Specifically, for ultrahigh volume fractions of proteins within the nanoclusters (φᵢₙₜ˜0.6), the fraction of folded protein approaches unity. This reflects the entropic self-crowding (inset in FIGS. 39b, 36c and 36d) penalty for unfolding to more expanded non-native conformations, which overwhelms other factors (e.g., the increase in both chain conformational entropy and favorable hydrophobic protein-protein interactions upon unfolding) that can otherwise destabilize the native state in less crowded environments. Importantly, the high φᵢₙₜ within the clusters (>400 mg/ml) strongly favors the native state via self-crowding, even for overall φ values where proteins aggregate and unfold when in solutions without clusters.

Regarding protein stability and conformation, upon dilution the proteins were clearly active, stable, and monomeric. Thus irreversible aggregates appear to not have been present within the nanoclusters, despite the high protein concentrations. As discussed above, within the nanoclusters, the native conformation would be expected to be entropically stabilized by protein self-crowding. In addition, the relatively low mobility of the proteins in the clusters, given the high intracluster concentrations of ˜700 mg/ml, may kinetically frustrate protein conformational changes that could otherwise lead to contact between hydrophobic patches and stabilize non-native complexes and aggregated states.

During these in vitro dilution experiments, the rapid dissolution (estimated at <1 msec) also lowers the probability of protein collisions that may otherwise produce irreversible aggregates. Immediately upon dilution, concentration and solubility gradients will result in release of antibody molecules from the nanocluster surface, while molecules buried within the cluster remain self-crowded, thus favoring stable folded protein within the cluster. This hypothesis is supported by the lack of an increase in the aggregates based on HPLC-SEC data and SDS-PAGE data upon dilution of the clusters, which decreases D_{c}, as is shown in FIGS. 47 and 48. Finally, the trehalose within the dispersion is present as the nanoclusters dissolve and thus favors folded protein.

### Viscosity of Nanocluster Dispersions

The very weak attraction between clusters led to a viscosity of the dispersion of 1B7 at c=267 mg/ml of only 40 cP which is a syringeable value (Table 12). Similarly, it was 63 cP for polyclonal sheep IgG at c=275 mg/ml. However, at c=300 mg/ml for c_{E}=0 where the protein did not form nanoclusters, the viscosity of the IgG solution was found to be not measureable as the solution was in the form of a gel that did not flow. This gelation was a manifestation of the attraction between the protein molecules with small spacings. In contrast, the nanocluster dispersions did flow at this c with c=c_{E′} with a viscosity of 250 cP. In the future, the viscosity may be further lowered by optimizing the composition of the extrinsic crowder.

TABLE 12
Viscosity and hydrodynamic diameter for monoclonal
1B7 antibody and polyclonal sheep IgG dispersions.

| Protein | Trehalose | | Hydro- | Hydro. |
|---|---|---|---|---|
| concentration | concentration | Viscosity | dynamic | Diam. |
| (c, mg/ml) | (c_{E}, mg/ml) | (η, cP) | diameter | St. Dev. |
| 267 (1B7) | 270 | 40 | 315 | 17 |
| 275 (IgG) | 275 | 63 | 88.0 | 9.0 |

### In Vivo Study of Protein Stability and Pharmacokinetics in Mice

To test the potential for drug delivery of protein nanocluster dispersions, we performed an in vivo pharmacokinetics (PK) study in mice. Control groups received 100 μl of dilute antibody solution via intraveneous or subcutaneous injection to provide a baseline defined as full bioavailability. Using a highly concentrated 235 mg/ml nanocluster dispersion, 1 μl was injected subcutaneously at pH 7.2 (Table 13). The viscosity of this dispersion was well below 40 cP (see Table 12), which is below the typical limit of 50 cP for subcutaneous injection. Remarkably, the resulting PK parameters, including normalized bioavailability (AUC/dose), C_{max/dose}, tₘₐₓ and elimination kinetics were statistically indistinguishable from those of the two subcutaneous groups (FIG. 41). The similar bioavailabilities suggest that the antibody molecules in the nanoclusters readily dissociated (the predicted time in buffer is 7 ms, Eq. 24), were transported from the injection site and entered the blood stream, while identical alpha and beta rates indicates the presence of predominantly monomeric antibody in the blood. If the antibodies were to aggregate or misfold during dissolution, the molecular weight and surface properties would change, in turn affecting renal and hepatic clearance rates. (Tabrizi, Tseng et al. 2006) Finally, analysis of antibody activity in the terminal blood samples with an in vitro toxin neutralization test showed similar activities versus control antibody, indicating that, in addition to antibody conformation, activity was unaffected. It is likely this nanocluster drug delivery concept could be extended to even higher dosages, given that dispersion concentrations up to 400 mg/ml for BSA and 350 mg/ml for polyclonal IgG, were attained (FIGS. 38 and 46). The dispersions could be formed by mixing powder and buffer and then injected soon thereafter to avoid the need for long term storage stability. For proteins with an isoelectric point more than 2 units away from physiological pH, this approach may require even greater concentrations of crowder to overcome electrostatic repulsion.

TABLE 13
Pharmacokinetic parameters for curves shown in FIG. 41.

| | | AUC_{0-∞}/dose | | | | |
|---|---|---|---|---|---|---|
| | Cₘₐₓ/dose | (μg · hr/ml)/ | tₘₐₓ | t_{1/2, α} | t_{1/2, β} | Relative |
| Formulation | (μg/ml)/(mg/kg) | (mg/kg) | (hrs) | (hrs) | (hrs) | neutralization titer |
| IV solution | 25.5 ± 3.8 | 3582 ± 990 | 15.1 ± 0.7 | 45.7 ± 22.8 | 227.1 ± 24.9 | 2.3 ± 1.7 |
| SQ solution | 18.8 ± 4.4 | 2699 ± 583 | 18.9 ± 3.1 | 43.4 ± 17.3 | 210.0 ± 17.4 | 1.0 ± 1.8 |
| SQ | 14.3 ± 3.1 | 3269 ± 291 | 21.4 ± 2.9 | 42.1 ± 24.8 | 243.2 ± 35.5 | 1.3 ± 0.5 |
| nanocluster | | | | | | |
| dispersion | | | | | | |

Error is ± s.d.

Low viscosity dispersions of concentrated protein in monodisperse equilibrium nanoclusters, with high conformational stability in vitro and high biological activity in vivo upon dilution, have been formed simply by mixing lyophilized protein, an extrinsic crowder and buffer. The high degree of self-crowding of the protein within the nanoclusters at an unusually high concentration of 700 mg/ml is shown theoretically to favor folding, as confirmed experimentally upon dilution of 1B7 nanoclusters. The size of the nanoclusters is tunable by adjusting the protein and extrinsic crowder concentrations near the pI, as shown both experimentally and with a free energy model. The ability to simultaneously achieve self-crowded clusters and low viscosities results from a general concept of tuning the multi-scale interactions with: attraction dominant at the protein monomer level, repulsion at the intercluster level and a neutral balance of the two for the monomer-cluster interaction. The intercluster repulsion favors colloidal stability and low viscosity without gelation. Remarkably, an analysis with a variety of physical, chemical and biological assays indicated conformationally stable protein monomer without any loss of protein activity after dilution of the nanocluster dispersions. In vivo sub-cutaneous administration of dispersed antibody resulted in indistinguishable pharmacokinetics and activity compared to control antibody solutions. This general approach for formulating dispersions of protein nanoclusters with crowding agents and a pH near the isoelectric point, offers the potential of subcutaneous administration of a variety of therapeutic biologics, which would otherwise gel when formulated as solutions.

### Formation of Nanocluster Dispersion

The murine IgG2a antibody 1B7 was expressed, purified and characterized as previously reported (Sutherland and Maynard 2009) and the pI determined via silver stained isoelectric focusing gel. Prior to lyophilization, the 1B7 solution was buffer exchanged into a 20 mM histidine buffer (pH 5.5) using a 50,000 molecular weight cutoff (MWCO) Centricon filter and solid α,α-trehalose added to a 1:1 protein:trehalose weight ratio as a cryoprotectant. The solution was filter-sterilized (0.22 μm), diluted to 20 mg/ml protein with 20 mM histidine buffer (pH 5.5), and transferred to a sterile 8 ml glass vial. It was frozen over 6 hours on a pre-cooled lyophilizer tray at −40° C. (VirTis Advantage Plus Benchtop Freeze Dryer) and then lyophilized at 150 mTorr with 12 hours of primary drying at −40° C. followed by a 6 hour ramp to 25° C. and an additional 6 hours of secondary drying at 25° C. To create a dispersion, typically 28 mg±0.02 mg of lyophilized protein was compacted into a tared 0.1 ml conical vial (Wheaton Science Products). After addition of 50 mM sodium phosphate buffer (pH 7.2) the resulting dispersion was stirred gently with the tip of a 25 gauge needle. The total volume and volume fractions of the components were calculated assuming ideal mixing based on known masses, and hypothetical pure liquid protein (1.35 g/cm³) and trehalose (1.64 g/cm³) densities, from their partial molar volumes at infinite dilution (Pilz, Puchwein et al. 1970; Miller, dePablo et al. 1997) and a known buffer volume. The final protein concentration was verified using a BCA assay or light absorbance at 280 nm with a mass extinction coefficient of 1.37 L/g·cm (Nanodrop, Thermo Scientific) to be within experimental error of the predicted value.

### Characterization of the Nanocluster Dispersions

The hydrodynamic diameters of protein monomers and nanoclusters were measured by dynamic light scattering (DLS) with a 632.8 nm (red) laser and an avalanche photodiode at ˜23° C. using CONTIN (Brookhaven B1-9000AT). The scattering angles ranged from 135° to 165° to minimize multiple scattering (Horn 2000) with the use of a 60 μl sample cell (Beckman Coulter). In order to verify the accuracy of this technique, the hydrodynamic diameter of a 298 nm polystyrene standard was measured at φ˜0.1 and found to be within 5% of the actual size. The scattering measurements for each sample of protein monomer or nanocluster were done at two separate angles consisting of 135°, 150° or 165° and the size was found to be within 5-10% for the two angles. According to a study of DLS and rheology of concentrated colloids, the calculation of the hydrodynamic diameter from the Stokes-Einstein equation based on the solvent viscosity is relatively accurate at our highest φ of 0.25. (Horn 2000) At higher φs, interactions between particles during the time scale of the measurement may produce much larger deviations from the Stokes-Einstein equation. To avoid these complexities, the particle size may be determined from small angle X Ray scattering. (Roosen-Runge, Hennig et al. 2011) For determining the fractal dimension of the IgG nanoclusters (FIG. 44), the scattered laser light intensity was measured at scattering angles every 5° between 45° and 90° using a cylindrical 2 ml capacity ampoule.

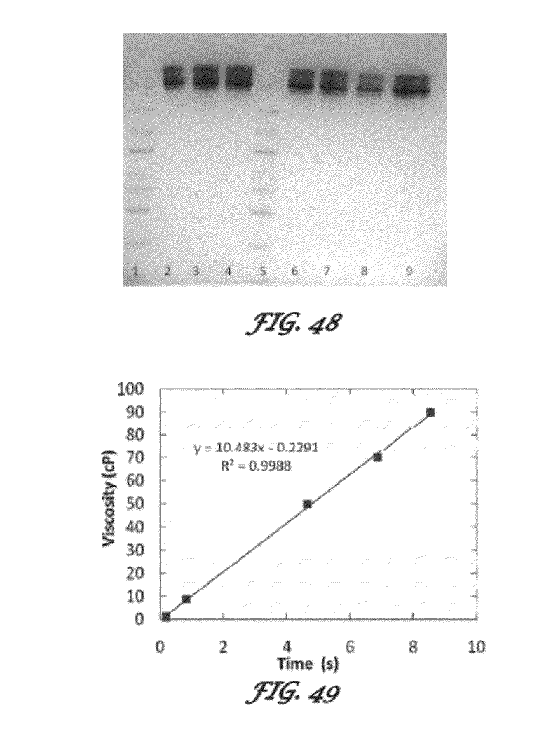

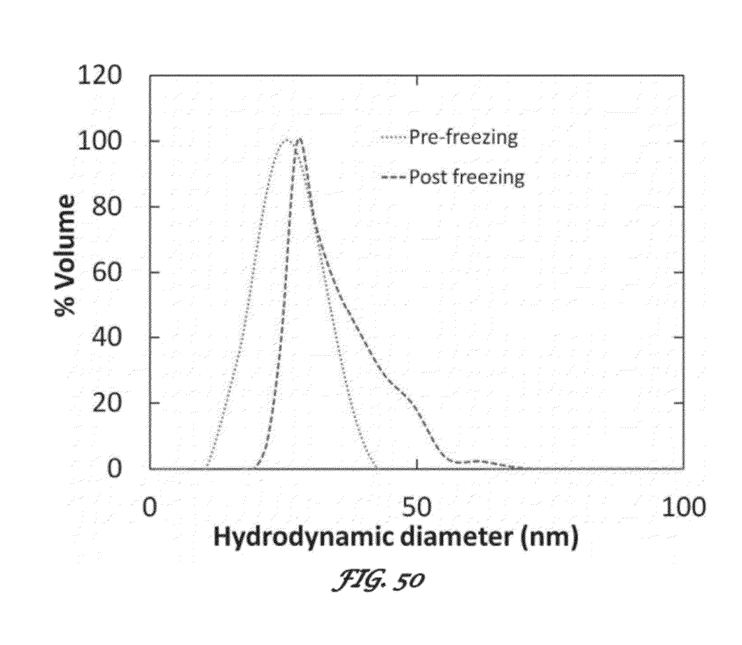

To prepare samples for scanning electron microscopy (SEM, Hitachi S-5500 at 30 KV), the dispersions were diluted to 40 mg/ml at a constant crowder volume fraction of 0.18 (corresponding to original dispersion at 220 mg/ml) using PEG 300 as a crowder, placed on a copper TEM grid with a carbon film coated with formvar, blotted to remove the excess liquid, rapidly frozen by immersion in liquid nitrogen and lyophilized. The viscosity of the nanocluster dispersions were measured in triplicate using a 25 gauge (ID=0.1 mm) 1.5″ long needle attached to a 1 ml syringe, according to the Hagen-Pouiselle equation. The time to draw the dispersion from a height from the bottom of the cone from 0.4″ to 0.1″, corresponding to a volume of ˜50 μL, was determined from analysis of a digital video. (Miller, Engstrom et al. 2010) A linear correlation between the time to draw 0.05 ml from the conical vial and the viscosity of various calibration fluids is shown in FIG. 49. (Liu, Nguyen et al. 2005; Miller, Engstrom et al. 2010) The shear rate varied decreased with the viscosity and was 1000 s⁻¹ at a viscosity of 50 cp. The viscosities of trehalose solutions were calculated from Uchida et al. (Uchida, Nagayama et al. 2009)

### Characterization of the Protein Structure and Activity

To monitor antibody structure and ligand-binding activity, lyophilized and dispersed protein were diluted to 1 mg/ml in PBS, prior to analysis by a battery of biophysical and biochemical assays versus solution control antibody. Typically, the dilution was performed within ˜4-6 hours of the formation of the dispersion. Circular dichroism (CD) measurements were collected from 260 to 185 nm in 0.1 nm steps using a Jasco J-815 CD Spectrometer. The formation of insoluble and di-sulfide linked aggregates was monitored by analysis of 5 μg samples of dilute protein on a 4-20% non-reducing SDS-PAGE gel. Formation of non-covalent aggregates were monitored by SEC, with 20 μg of diluted dispersion analyzed with a Waters Breeze HPLC. To analyze ligand-binding activity, an indirect PTx ELISA was performed as previously described (Sutherland, Chang et al. 2011) and reported as the ratio of 50% effective concentration values (EC₅₀) for the sample versus solution control. The thermal melting temperature (Tₘ) was quantified with using a 7900HT thermocycler from Applied Biosystems and SYPRO Orange Protein Gel Stain (Sigma-Aldrich). (Lavinder, Hari et al. 2009)

### In Vivo Bioavailability in BALB/c Mice

An in vivo pharmacokinetic study of the 1B7 dispersion and a control solution was performed over a 14 day period using 24-27 g, female BALB/c mice. The three sample groups included (1) intravenous (IV) and (2) subcutaneous (SQ) control injections of 100 μl of a dilute 1B7 solution and (3) a test condition, SQ injection of an antibody dispersion (235 mg/ml in a 1 μl volume to yield a 9.4 mg/kg dose). Prior to injection and at eight additional time-points between 12 and 336 hours, serum samples (˜20 μl) were collected from the tail vein. At the terminal time-point, mice were anaesthetized and serum collected by cardiac puncture. This study was performed with approval by the Institutional Animal Care and Use Committee at the University of Texas at Austin (protocol #AUP-2010-00070) in compliance of guidelines from the Office of Laboratory Animal Welfare. To determine the concentration of active 1B7 in each serum sample, an indirect PTx ELISA was performed as previously described. (Sutherland and Maynard 2009) Each plate included mouse serum (Sigma) as a negative control and a 1B7 standard curve diluted in mouse serum. SoftMax Pro v5 was used to calculate EC₅₀ values based on the serum dilution using a 4 parameter logistic (4PL) model and total concentrations of active 1B7 present in serum samples calculated from the standard curve. An orthogonal antibody activity assay, based on in vitro CHO cell neutralization of PTx, was performed using serum from the terminal time point. (Sutherland and Maynard 2009).

TABLE 14
Parameters used FIGS. 40a and 40c to
determine the potential of mean force

| | Monomer at pI | Monomer 3 pH units | Cluster |
|---|---|---|---|
| Quantity | (FIG. 39a) | from pI (FIG. 39a) | (FIG. 39c) |
| Charge per protein | 1 | 25 | 0.6 |
| Debye Length (κ⁻¹) | 0.7 | 0.7 | 0.7 |
| Γ₀ | 0.036 | 0.72 | 0.76 |
| φ_{E} | 0.17 | 0.17 | 0.17 |

TABLE 15
General parameters for calculating cluster
diameter contours in FIG. 40b

| | Value for 1B7 | Value for IgG |
|---|---|---|
| Quantity | (FIGS. 37b and 39b) | (FIG. 37c) |
| Fractal dimension (δ_{f}) | 2.6 | 2.6 |
| Dielectric constant (∈ᵣ) | 25 | 15 |
| No. of dissociable sites per unit | 0.2 | 0.2 |
| area of particle surface (σ, nm⁻²) | | |
| Distance between opposite charges | 0.1 | 0.2 |
| in an ionic bond (b, nm) | | |
| Radius of primary particle (R, nm) | 5.5 | 5.5 |

TABLE 16
Particular parameters for calculating cluster
diameters for specific case in FIG. 40b

| Quantity | Case 1 |
|---|---|
| Concentration of extrinsic crowder (c_{E}, mg/ml) | 220 |
| Attractive energy (∈/k_{B}T) | 6.52 |
| Protein volume fraction (φ) | 0.16 |
| Concentration of protein (c, mg/ml) | 220 |
| Charge on a protein monomer (q₀) | 0.09 |
| Aggregation number (n_{c}) | 4500 |
| Predicted diameter of the cluster (D_{c}, nm) | 280 |
| Actual diameter of the cluster (D_{c}, nm) | 320 |

### Example IV

A solution (typically 50 mg/mL) of polyclonal sheep IgG (abbreviated IgG, Sigma Aldrich or Rockland Immunochemicals) was prepared in the desired dispersion buffer; the buffer was formulated at 150 mM ionic strength and with pH in the range of the pI of the protein, typically within one or two pH units. The concentration was verified using absorbance at 280 nm with a mass extinction coefficient of 1.37 L/g·cm (Nanodrop, Thermo Scientific). In some cases, the protein was purified by FPLC as indicated below.

### Formation of Nanoclusters by Removal of Aqueous Media

Tare weights were taken of the centrifugal filter assembly (Millipore Microcon, Ultracel YM-50 membrane, 50 kD nominal molecular weight limit, diameter of filter, 0.25″ diameter). The required amount of crowder (e.g. trehalose, sucrose, small molecular weight polyethylene glycol, dextran or amino acid) was weighed into the retentate chamber of the filter assembly. The desired volume of protein solution was pipetted into the retentate chamber, and the solution was mixed with the pipet tip to ensure dissolution of the crowder. The filter assembly was then centrifuged (Eppendorf Centrifuge 5415D) at 10,000 rcf for a measured amount of time typically less than one hour. Post-centrifugation, the amount of flow through the filter was measured either by weighing the permeate tube or by computer-aided image analysis of the meniscus height in the permeate tube or in the filter capsule itself. The centrifugation was repeated to obtain the desired level of concentration.

Once sufficient aqueous solution had been removed to reach the desired concentration, the dispersed protein in the retentate was recovered by inverting the filter assembly into a retentate recovery tube, and centrifuging it for 3-4 minutes at 1,000 rcf. The resulting dispersion was transferred to a 0.1 mL conical vial (V-Vial, Wheaton), and the concentration was verified spectrophotometically using absorbance at 280 nm. The concentration of the crowder will be indicated as the starting concentration.

### Viscosity Measurement

The viscosity of the nanocluster dispersions were measured in triplicate using a 25 gauge (ID=0.1 mm) 1.5″ long needle (Becton Dickinson & Co. Precision Glide Needle) attached to a 1 ml syringe (Becton Dickinson & Co. 1 mL syringe with Luer-Lok™ tip), according to the Hagen-Pouiselle equation. The time to draw the dispersion (in a 0.1 mL conical vial) from a height from the bottom of the cone from 0.4″ to 0.1″, corresponding to a volume of 48 ΞL was determined from digital video. This time was correlated to viscosity from a calibration curve derived from a set of standards of known viscosities as shown in Table 19.

### Measurement of Hydrodynamic Diameter

The hydrodynamic diameters of protein monomers and nanoclusters were measured by dynamic light scattering (DLS) at an angle of 135° with a 632.8 nm laser and an avalanche photodiode at ˜23° C. using the CONTIN algorithm (Brookhaven B1-9000AT). The samples were placed in a 60 μl sample cell (Beckman Coulter).

For analysis of non-covalent aggregates, the sample was diluted in mobile phase (100 mM sodium phosphate, 300 mM sodium chloride, pH 7) to 1 mg/mL. A volume of diluted dispersion containing 20 μg of protein was analyzed with a Waters Breeze HPLC, using TOSOH Biosciences TSKgel3000SWXL and TSKgel2000SW columns in series, with eluate monitored by absorbance at 214 nm.

### Tonicity

One of the challenges in injectable administration is that the material should be isotonic. A media is isotonic with another if it has the same effective osmotic pressure as the liquid inside the cell across the membrane of a given type of cell. This tonicity is a function of the permeability of the cell membrane to the particular solute molecule and therefore varies depending on the type of cells involved and the identity of the solute molecule. An isotonic solution (compared to blood) is generally defined as a solution having the same colligative properties as a solution of sodium chloride containing 0.9 g NaCl per 100 ml of the solution. The osmolality of a given formulation with its excipients was calculated relative to the osmolality at isotonic conditions based on equivalents of sodium chloride tabulated as a function of the relative permeability through biological membranes in the Merck Index (Twelfth ed.) and Sinko 2006. The osmolality was assumed to be linearly additive for the individual components.

Crowding agents such as polysaccharides which may be used to form protein nanoclusters dispersions for subcutaneous administration will influence the tonicity of the solution. For injection into the body, in some embodiments, formulation should be as close to isotonic in order to avoid pain due to the injection. For hypotonic fluids, membrane transport will influence the size of cells adjacent to the fluid. These changes could potentially influence immunogenicity. Therefore the crowding agents may be optimized for tonicity and for controlling the cluster size, without raising the viscosity above 50 cp or 100 cp or 150 cp. Finally, in some embodiments, the formulation does not cause immunogenicity upon administration and the protein has the desired pharmacokinetics and biological activity.

Formation of Hypertonic Protein Dispersions by Removing Water with 100 mg/ml or More Trehalose

The dispersions at the protein concentrations listed in Table 20 formulated with the corresponding amount of trehalose were prepared by the centrifugation method described earlier and their viscosity was measured using a syringe. The viscosities were observed to be lower and the intrinsic viscosities given in Table 20 in the range of 5-6 in the first three rows. The intrinsic viscosities were higher in the case of row numbers 4 and 5 where sheep IgG from a different supplier (Rockland Immunochemicals) with a higher amount of aggregates in it was used, which might explain the higher viscosities. These low intrinsic and solution viscosities were the result of the new technique of generating the dispersion. Lower amounts of trehalose in the solution lead to a lower solvent viscosity. This method may provide advantages over methods based on dispersing protein powders made by lyophilization. The ability to avoid the step of dissolution of lyophilized powder may avoid potential complex colloidal gel states that may result in the viscosity being higher. This new technique also puts less stress on the protein by avoiding the lyophilization step and by increasing the protein concentration gradually. The new technique also allows flexibility in formulation as no cryoprotectant molecules are required.

The hydrodynamic diameter is observed (Table 20) to be around 30-35 nm for these nanoclusters which is much smaller than the ˜80 nm diameter observed for the sheep IgG nanoclusters formed as described herein. The lower size may be the result of the lower levels of trehalose present in the dispersion which leads to a lower magnitude of the attractive depletion attractions which drive cluster formation resulting in smaller cluster sizes. Smaller clusters are beneficial in terms of the clusters passing through a sterilizing filter more easily leading to easier sterilization of the dispersion. In addition, SEC data provides evidence that there is little irreversible aggregation among the protein molecules. Unmodified polyclonal sheep IgG as provided by Sigma Aldrich is 92.63% monomer and as can be seen in Table 20, the process of cluster formation through centrifugation and the subsequent shearing through the needle of the syringe to measure viscosity do not lead to a significant decrease in the amount of monomeric protein in the solution. Therefore, the clustering process does not lead to irreversible aggregation of the protein and the protein dissociates back into monomer upon dilution in vitro.

High or Moderate Concentration Dispersions with Hypotonic or Isotonic Low Concentrations of Crowding Agents Formed by Lyophilization: Comparison with Solution and with Larger Clusters.

The nanoclusters were made as described herein from lyophilized powders of protein and a cryoprotectant, trehalose for rows 1-3 in Table 21. The lyophilized powder from Sigma Aldrich sheep IgG was directly dispersed in the buffer containing dissolved trehalose in order to make a dispersion of protein nanoclusters at 350 mg/ml as shown in rows 1, 2 and 3 in Table 21. The concentrations of these dispersions were determined by weight and volume. The nanoclusters formed stable colloidal dispersions in rows 2 and 3. In these experiments we did not measure the conformational stability of the protein or formation of protein aggregates. For row 1, no trehalose or other crowding agent was added, as a result of which, the protein was in solution and formed a highly viscous gel that was not syringeable. Nanoclusters were not formed. Here the attractive forces between protein molecules in solution led to gelation. However with the addition of trehalose as shown in Table 21, rows 2 and 3, nanoclusters formed and the dispersion was syringeable and had a much lower and measureable viscosity. The lower viscosity for the nanoclusters is expected on the basis of the colloidal forces. The trehalose crowds the protein into clusters as is evidenced by the cluster hydrodynamic diameter measured by DLS as shown in Table 21 rows 2 and 3. In these two cases the osmolality/osmolality at isotonic conditions was below unity.

Typically the ratio of cryoprotectant to protein is 1:1, weight by weight for particles produced by the lyophilization process. For a protein at 200 mg/ml, this ratio corresponds to 200 mg/ml trehalose, well over the limit of ˜100 mg/ml for isotonic conditions. (For disaccharides like sucrose and lactose, the concentrations of an isotonic solution are 92.5 mg/ml and 97.5 mg/ml, respectively (Sinko 2006, Merck Index). The tonicity problem becomes even more severe for larger protein concentrations. One method to avoid this high tonicity would be to use less cryoprotectant (or lyoprotectant) during lyophilization. However, with less cryoprotectant, the protein may undergo denaturation during the lyophilization process. In rows 1-3 in Table 21, no cryoprotectant was used in the lyophilization process

High or Moderate Concentration Hypotonic or Isotonic Dispersions with Low Concentrations of Crowding Agents Formed by Removal of Water.

The high viscosities of these dispersions in rows 2-3 formed from powders from lyophilization in Table 21 indicate that alternative processes would be beneficial to make the nanoclusters. Another method to overcome the need for large amounts of cryoprotectant to make protein powders, which are then to form nanoclusters by mixing, would be to avoid lyophilization or other freeze drying processes. In this example, nanoclusters as dispersions in aqueous media were formed from monomeric protein solutions by removing water. We demonstrate this by using centrifugal filtration which allows the passage of water and small molecules including trehalose, thus increasing the protein concentration. As the protein is concentrated, nanoclusters were formed as a function of the amount of crowding agent in the solution and the protein concentration. In this type of process, which does not require lyoprotectants to make powder by lyophilization, the ratio of crowding agent to protein may be much smaller, that is, from 1:2 to even 1:5 weight by weight or less. Then if the protein is stable upon dilution of the nanoclusters, the need for lyoprotectants in powder formation processes by freezing or lyophilization may be circumvented.

Lower amount of cryoprotectant in the solution leads to a lower solvent viscosity. A lower solvent viscosity ηₒ will produce a lower effective dispersion viscosities based on equation 25. Also in this method it becomes possible to avoid the step of lyophilization and dispersion of lyophilized powder to form nanoclusters. During dispersion of powder, the protein starts out in highly concentrated powder form. As it mixes with the aqueous solvent, complex colloidal gel states may be formed which have the potential to raise the viscosity (see rows 1-3 of Table 21).

In the new process for removal of aqueous media to concentrate the protein, the mass transfer pathway is the opposite than in the case of mixing power with aqueous media. Rather than adding the aqueous solvent, it is removed, by centrifugation as described above. Upon removal of the aqueous solvent, the protein concentration never goes above the protein concentration in the final dispersion. This new technique also puts less stress on the protein by avoiding the lyophilization step and by increasing the protein concentration gradually.

The formation of the nanoclusters depends upon the relevant colloidal interactions as shown experimentally and with a free energy model as described herein. Suppose the final protein concentration is 300 mg/ml. Then a crowder concentration of only 100 mg/ml or even lower will often still lead to the formation of nanoclusters as shown by actual experimental examples in Table 21 in rows 4 to 11 and colloid theory presented earlier herein In these experiments the crowder concentration is lower than in many earlier examples described herein.

In row 4, the osmolality/osmolality at isotonic conditions ratio was not far above unity while in row 5 it was essentially unity and in the subsequent rows 6 through 11, the ratio was below unity. The dispersions from row 5 through 8 were found to have clusters of hydrodynamic diameters ranging from 28 to 45 nm by DLS which evidences that the protein was present in the form of nanoclusters in all these cases, as the protein monomer is only 11 nm in diameter. Thus the colloidally stable nanocluster dispersions in rows 5 to 11 were isotonic or below isotonic while still at an extremely high protein concentration. For the hypotonic conditions, it would be straightforward to add more buffer or salt to make the dispersion isotonic.

The dispersions in row 6 and 7 were made in a buffer with a lower total salt concentration 20 mM versus 50 mM in the earlier cases throughout these examples of the sodium monophosphate and sodium biphosphate buffer. This lower concentration of buffer enables the use of higher crowder concentrations while still maintaining the dispersion as isotonic. Furthermore, the use of lower crowder concentrations in Table 21 relative to Table 20 lowers the tonicity.

The concentrations of protein are above 290 mg/ml in rows 4-5, 7 and 9-10. In rows 9 and 10 the relatively low crowder concentration produces a relatively low solvent viscosity. Furthermore, the intrinsic viscosity is below 8. These factors contribute to the fact that the solution viscosities are below 120 cp despite the very high protein concentrations.

The concentration of crowder was decreased to 50 mg/ml (see row 12 of table 21). This dispersion was highly viscous and scattered light poorly, indicating that it did not form uniform nanoclusters. However, a dispersion at a similar concentration with a slightly higher concentration of crowder (row 4 of table 21) had approximately 50% lower viscosity and formed uniform clusters. Thus, a sufficient amount of crowder is required to form the dispersion of nanoclusters for a favorable viscosity. At lower crowder concentrations, the morphology is of proteins gelled from solution with uncontrolled morphologies as is well known for colloidal gels in the literature.

Morphology of clusters, colloidal properties and protein stability after freezing and thawing or freezing, storing, and thawing with implications for storage stability. In order to be acceptable for clinical use, it would be necessary for these high-concentration formulations to be stable from the time they are prepared until when they are injected. Here the formulations are stabilized by storage in a freezer. The dispersion (row 6 in table 21) was frozen and stored at −40° C. for a week in a glass conical vial that was sealed for airtightness. After passage of the week, the dispersion was thawed gradually in a refrigerator at 4° C. over the course of a few hours and then the thawed dispersion was characterized. The vials were not stirred or shaken. The nanoclusters were found in the dispersions without any need for agitation. The viscosity, hydrodynamic diameter and the concentration of the dispersion were quantified (see table 22) and were found to be the same as those measured before freezing the dispersion for the first example. For the second two examples, the size is only shown post freezing and found to be well below 100 nm. This demonstrates that the dispersion is stable upon freezing, storage and thawing. It is conceivable this approach may be used whereby the storage time is months to even a year given knowledge of the state of the art for storing proteins in the frozen state. Furthermore, the storage of the proteins as frozen nanoclusters has the potential to provide even greater stability than when storing proteins as solutions.

### Effect of Smaller Cluster Size and Higher Packing Fraction Effect on Viscosity and Other Properties.

The next experiments relate the dispersion viscosity to the morphology of the nanoclusters. In rows 8 and 9 in Table 21, the concentration of trehalose was only 70 mg/ml leading to an isotonic dispersion with a low viscosity in each case, despite the high protein concentrations. The hydrodynamic diameter is observed to be 40 nm in row 8 in Table 21. This size is much smaller than the ˜80 nm diameter observed for the sheep IgG nanoclusters as described herein, where the trehalose concentration was much higher. The smaller size of the nanocluster in Table 21 was expected given the lower amount of the extrinsic crowding agent trehalose, as described by the free energy model herein. The intrinsic viscosity for the nanoclusters shown in Table 20 and rows 8 to 10 in Table 21 and the dispersion viscosity values in these cases and in row 11 in Table 21 were lower than those achieved at comparable protein concentrations by the lyophilization/mixing process.

Physical arguments may be used to explain how the cluster size and packing fraction of protein within a cluster will influence the viscosity for a dispersion of nanoclusters. The viscosity depends upon the value of the nanocluster volume fraction φ relative to φₘₐₓ as shown in Table 19 as well as other factors. For a given protein concentration in a dispersion, where the protein is in the form of nanoclusters, the effective volume fraction φ_{eff} is now shown to depend upon the density or packing fraction of each nanocluster. The packing fraction increase is a result of the proteins not occupying the entire space in the cluster. Volume unoccupied by protein is present within the clusters. Water is present in the unoccupied volume. The packing fraction depends upon the cluster morphology, which depends upon the shape of the protein molecules and the composition of the dispersions. The internal volume fraction of protein within a single nanocluster φᵢₙₜ is given by

$${φ}_{int} = {( \frac{{D}_{c}}{{D}_{m}} )}^{( {δ}_{f} - 3 )} \qquad ( 27 )$$

where D_{c} is the cluster diameter, Dm is the protein monomer diameter and δf is the fractal dimension of the cluster. An effective value the cluster volume fraction φ_{eff} may be defined as φ/φᵢₙₜ. The viscosity in eq. 26 is a function of φ_{eff} rather than φ□ since it depends upon the volume fraction occupied by the colloidal particles, which in this case are nanoclusters.

An increase in φᵢₙₜ may be used to decrease φ_{eff} for a given overall protein concentration in a nanocluster dispersion. A decrease in φ_{eff} would favor a lower dispersion viscosity according to eq 26. Furthermore, a decrease in φ_{eff} will correspond to a greater spacing between clusters. A larger spacing will favor weaker interactions and thus a weaker intrinsic viscosity, which would also lower the dispersion viscosity. In summary, the ability to raise may be expected from a theoretical point of view to favor a lower dispersion viscosity according to eq. 26.

The value of effective cluster volume fraction φeff was defined as φ/φᵢₙₜ, on the basis of the φint from SLS, ˜0.6 which was obtained as described herein. It may also be measured by small angle x-ray scattering and by small-angle neutron scattering. For a given protein and δf, as the cluster size, Dc increases, φₙ. For a fractal object, the packing fraction decrease from the center to the outside of the object. Thus φᵢₙₜ may be expected to be lower for small clusters. Therefore the use of smaller amounts of crowding agents that form small nanocluster will favor lower viscosities as long as short ranged attraction between clusters is weak enough. Eventually when the amount of crowding agent becomes too small the dispersion gels as shown in row 1 of table 21. Thus the concentration of crowding agent, as well as the composition of the crowding agent, may be optimized to achieve the lowest dispersion viscosity. For example, for a cluster size of around 30 to 40 nm as the intrinsic viscosity is lower (6-7, rows 1-3 in Table 20 and rows 8-11 in Table 21) when compared with the relatively higher values of ˜8 as described herein for certain clusters of size 80 nm

Balancing crowding for nanocluster size with tonicity by varying the crowding agent molecular weight. The formation of high concentration and low viscosity dispersions is highly dependent upon the crowding agent concentration and molecular weight based on the free energy model. The effect of crowder size on the depletion attraction is described by equation 6. An increase in crowder size increases the range of the depletion attraction, which may further increase the overall attraction between protein monomers. An increase in depletion attraction increases the cluster size. The molecular weight and composition of the crowder also influences the tonicity as well as the solvent viscosity.

A nanocluster dispersion was formed using 100 mg/ml 1kD molecular weight dextran as the crowder as shown in row 10 in Table 21. The tonicity of the dispersion was lower than most of the other cases in Tables 20 and 21, indicating a benefit of the higher molecular weight of the crowder. The intrinsic viscosity is seen to be in the same range as dispersions made with Table 20. Therefore it is possible to use other crowding agents in solution for forming clusters in the solution. The polysaccharide dextran also acts as a crowding agent. The dextran causes the formation of clusters by causing depletion attraction between the protein molecules as shown in the free energy model. The solution viscosity for a dextran solution is higher for the same mass concentration as for trehalose solution. However, at a given mass concentration, the dextran will contribute to a lower extent to the osmolality of the solution due to its higher molecular weight, and thus smaller number of particles. Thus, the concentration of extrinsic crowder may be increased while maintaining isotonic conditions by raising the molecular weight. Eventually, as the molecular weight becomes too large, the solvent viscosity will be prohibitive large. Thus, the crowding agent molecular weight must be optimized to satisfy the constraints of tonicity, cluster size, solvent viscosity, and dispersion viscosity. It will also influence protein stability.

Demonstration of lowering viscosity by raising crowder concentration to form clusters relative to solutions. Use of surfactant as an excipient to lower viscosity and stabilize dispersion. In row 11 of Table 21, the dispersion included trehalose as a crowder also had 1% polysorbate 80 (PS80) as an excipient. Protein molecules have patches on their surface that are hydrophobic, hydrophilic or charged. Interactions between the hydrophobic patches are short-ranged and attractive and cause increased attractions between the proteins or clusters in solution leading to higher intrinsic viscosity and hence increased effective viscosity (Equation 26). Surfactants like PS 80 will coat the hydrophobic patches and convert them to hydrophilic patches. The masking of hydrophobicity may result in lowered attraction and hence lowered dispersion viscosity. In addition to this due to the lowered specific short ranged attractions, surfactant usage will reduce the tendency of aggregation for free protein monomers in the dispersion. The lowered specific short-ranged attractions between the protein monomers are also useful for decreasing the solution viscosity as the specific short-ranged forces will not bridge between clusters through hydrophobic patch interactions causing increased intrinsic viscosities. These concepts agree with the extremely low viscosity of ˜150 cP measured for a ˜350 mg/ml dispersion of protein nanoclusters as shown in row 11 in Table 21. In row 13, ethanol was added in addition to trehalose, with the thought of achieving a similar effect.

TABLE 17
Nanoclusters of Sheep IgG at pH 6 formed by mixing lyophilized
powders with buffer with two different orders of mixing

| | Final | Treha- | Vis- | | |
|---|---|---|---|---|---|
| | conc IgG | lose | cosity | Intrinsic | |
| No. | mg/ml | mg/ml | cp | viscosity | Method |
| 1 | 400 | 200 | 598 | 6.41 | Buffer added to powder |
| 2 | 350 | 175 | 269 | 7.36 | Buffer added to powder |
| 3 | 350 | 175 | 248 | 7.24 | Buffer added to powder |
| 4 | 300 | 300 | 304 | 9.17 | Buffer added to powder |
| 5 | 275 | 275 | 63.0 | 7.42 | Buffer added to powder |
| 6 | 214 | 214 | 37.0 | 9.47 | Buffer added to powder |
| 7 | 334 | 150 | 604 | 9.25 | Powder added to buffer |
| 8 | 306 | 175 | 321 | 9.55 | Powder added to buffer |
| 9 | 310 | 125 | 94 | 7.83 | Powder added to buffer |
| 10 | 190 | 125 | 25 | 10.5 | Powder added to buffer |

The data in Table 17 are from dispersions manufactured in a manner similar to Example I. The IgG is lyophilized with the desired amount of trehalose (either a mass ratio of 1:1 protein to trehalose or 1 to 0.5 protein to trehalose) as a cryoprotectant as shown by the concentrations in the table. Once lyophilized, the protein-trehalose powder is weighed into a vial, and a buffer solution (at the pH described above) is added to create the dispersion. Rows 2 and 3 demonstrate that these dispersions are reproducible with regards to viscosity measurement. In a second method, powder is added to a buffer solution. In both methods, it was possible to achieve an intrinsic viscosity below 10.

### Example V

Number of iterations in the concentration process, which may be more specifically a filtration process, wherein the protein-crowder liquid combination is concentrated. Concentration processes include: centrifugal filtration, mechanical filtration, tangential flow filtration, and dialysis. The concentration (e.g. filtration) process may be performed in a single iteration. Or it may be performed in multiple iterations. A variety of strategies may be performed during the concentration (e.g. filtration) process to control the properties of the dispersion produced by this process. These variations will influence the mass transfer pathways during the concentration (e.g. filtration) process. Various agents may be added to the feed (e.g. to the filter during the filtration process). The agents may be added continuously or in increments. The concentration (e.g. filtration) may be performed in one iteration. Or it may be performed in multiple interations. If it is performed in multiple iterations, agent (e.g. crowder) may be added between iterations or during iterations, or both.

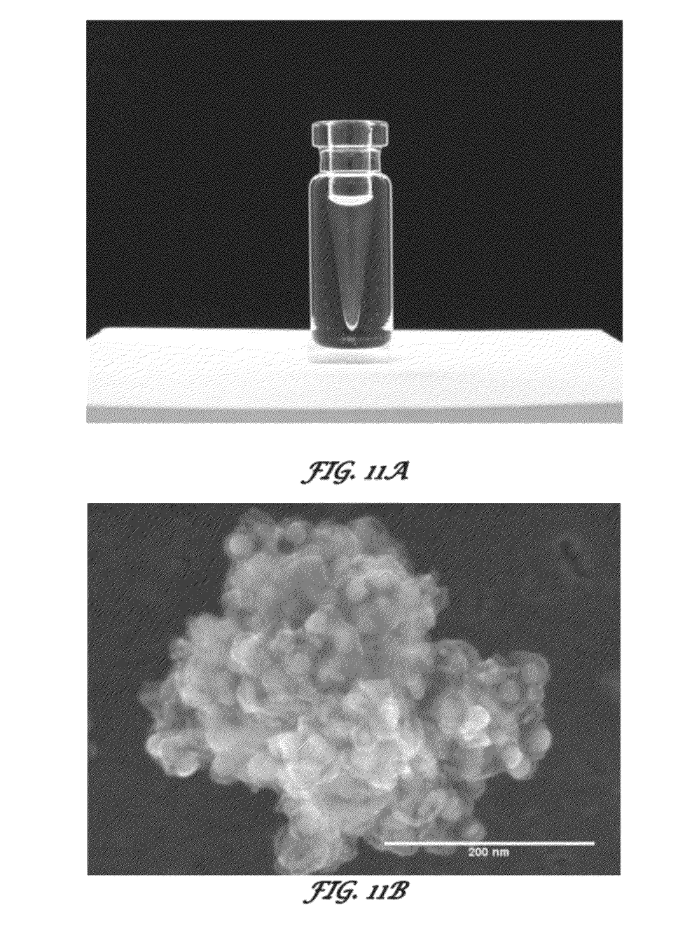

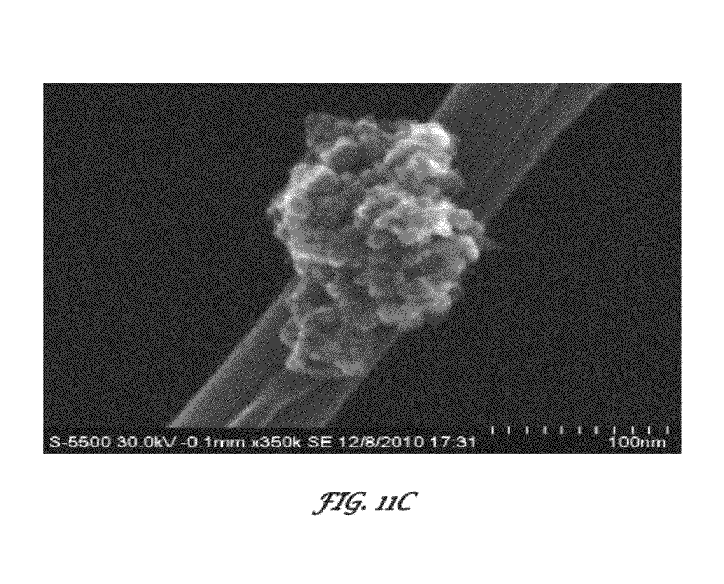

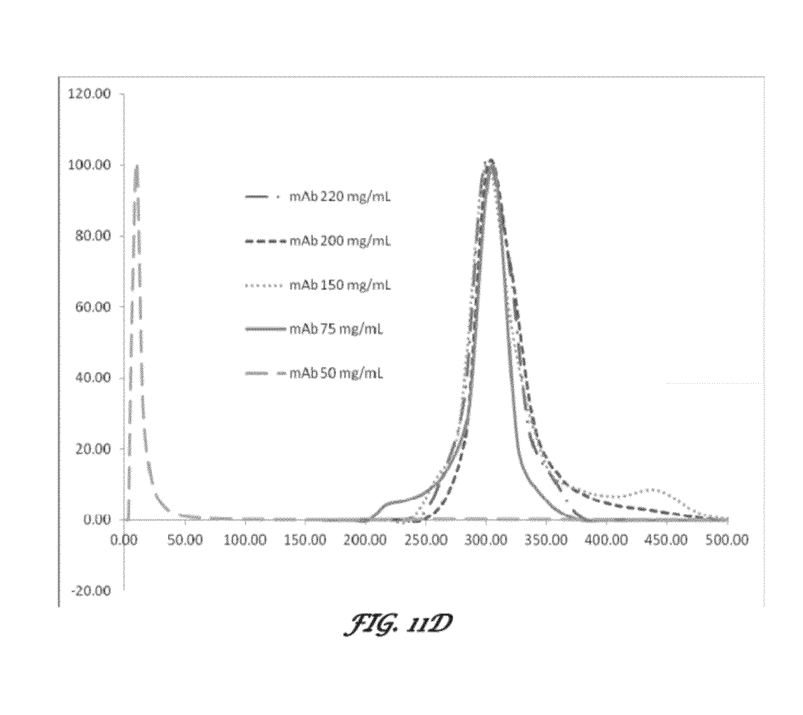

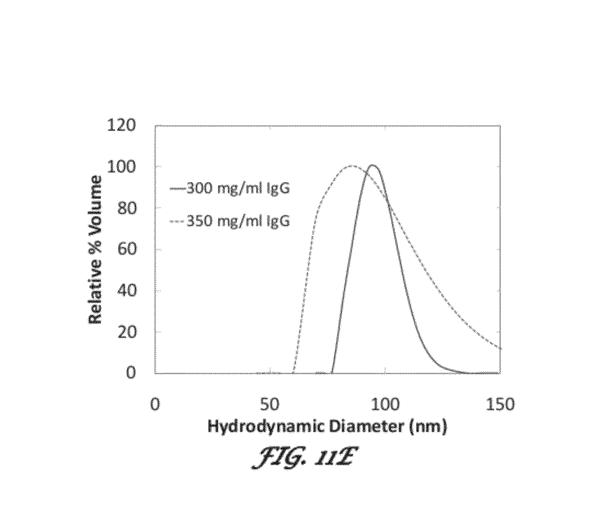

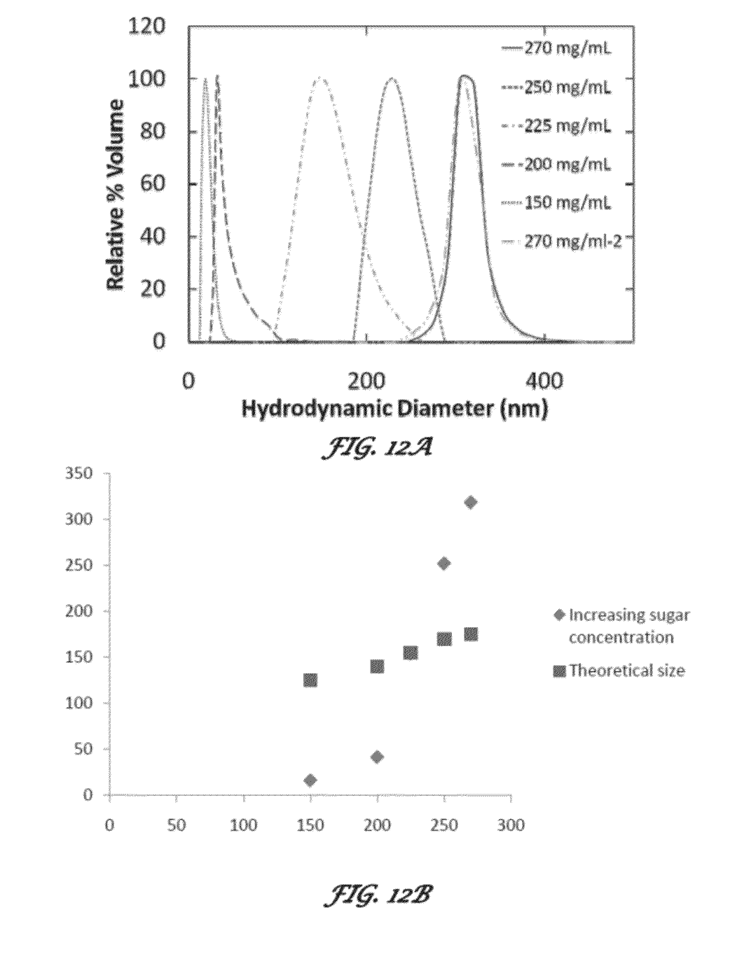

The agent added during concentration (e.g. filtration) may be a crowder to influence nanocluster morphology. Or it may be a concentration (e.g. filtration) aid (e.g. to minimize fouling of the filter by the protein for filtration). The tuning of the cluster size with the addition of crowder may be designed based on the concept of FIG. 12 where φ is plotted versus φ_{E}. An increase in either of these quantities raises the nanocluster size. They will also influence the packing fraction of the cluster. The changes in cluster size and packing fraction during the concentration (e.g. filtration) process will influence the final cluster size and packing fraction. These morphological aspects will also influence the protein folding, which is influenced by the extrinsic crowder and self crowder.

The components in the dispersion may be designed to influence their retention or reduction during the concentration (e.g. filtration) process (e.g. permeation through the filter for filtration). The extrinsic crowding agent, including polysaccharides and amino acids and peptides and proteins may be designed such that they are retained or not retained during the concentration (e.g. filtration) process (e.g. permeate through the filter with the buffer during filtration). The agent may also be a nonsolvent for the protein such as an alcohol, my or another organic solvent or a salt. It may be an agent to change pH. The agent may also be higher molecular dextrans or polyethylene glycol or other polymeric crowders including peptides and proteins and natural polymers such as alginates and chitosan that do not get removed or decreased during the concentration (e.g. filtration) process (e.g. do not pass through the filter in the case of filtration).

For the crowders that are reduced during the concentration (e.g. filtration) process (e.g. pass through the filter for filtration), the increase in the φ raises the nanocluster size. For these the concentration of the crowder decreases during the concentration (e.g. filtration) process as the overall volume fraction of protein increases. For the crowders that are retained during the concentration (e.g. filtration) process (e.g. do not pass through the filter), their concentration will build up as the buffer permeates. Thus, the nanocluster size will increase more in this non-permeating crowder case as would be evident from the concepts in FIG. 12. It would be possible to make a formulation of a mixture of crowders, where one is reduced and one is retained (e.g one permeates and one does not, respectively, for filtration).

Starting and Ending Concentration of Protein in the Concentration (e.g. Filtration) Process where Starting Material is a Protein Solution of Monomer

The starting and ending protein concentration φ will influence the nanocluster properties. At a given φ_{E}, at a small φ, the protein may start as a monomer. During a concentration process, such as filtration, as φ increases, clusters will be formed as in FIG. 12. An increase in either φ or φ_{E} or both will raise the cluster size. The difference between the starting φ and the final φ and the change in the pathway in φ during filtration will influence the nanocluster morphology and protein stability.

Starting and Ending Concentration of Protein in the Filtration Process where the Starting Material is Already a Nanocluster Dispersion

The starting material may already be a nanocluster dispersion. In this case an increase in φ or in φ_{E} during concentration, for example filtration, will influence the nanocluster morphology and raise the nanocluster size.

### Reduction of Nanocluster Size by Dilution

Buffer (e.g. aqueous buffer) may be added to lower protein and/or extrinsic crowder concentrations to lower the nanocluster size from a starting size.

### Example VI

TABLE 18
Theoretical shear rate as a function of viscosity for a Newtonian
fluid in a 0.1 mm (25 gauge) syringe with a length of 1.5″
where the viscosity and flow rate was determined from Table 19.

| Viscosity of | | Shear rate in the |
|---|---|---|
| dispersion (cP) | Flow rate (μL/s) | syringe needle (s⁻¹) |
| 50 | 9.602 | 906 |
| 100 | 4.833 | 456 |
| 300 | 1.614 | 152 |

The principle of the syringe viscometer is to have a relatively small variation in the pressure drop in the needle by displacing the piston by a set amount cause flow through the needle of a known diameter. As the dispersion volume generally takes <5% of the 1 mL syringe volume upon flow, the pressure drop changes only a small amount. Since the syringe plunger is displaced the same amount each experiment, the pressure drop is constant. The flow rate and the viscosity are related through the calibration as described herein, the data for which is given in table 18. For the viscosity, listed in table 18 (with viscosity standards listed in table 19), the flow rate is determined through the calibration. The needle is a cylinder and so assuming that the equation for flow through a pipe with no slip at the walls holds, the shear rate at the wall is calculated based on the flow rate that was calculated at that viscosity. The shear rate is a function of the fluid's flow velocity and hence fluids which are more viscous have more resistance to flow as a result of which they flow slower and hence undergo a lower shear rate in the needle. Even for fluids with deviations from Newtonian behavior, an approximate shear rate is given in Table 18.

TABLE 19
Standards for Syringe Viscometer Method

| | | Average Time | Average Viscosity |
|---|---|---|---|
| | Known | to Draw 48 μL | as Calculated by |
| Sample | Viscosity (cP) | (s, ±, rage | Equation (cP ± s.d.) |
| Deionized Water | 1 | 0.20 ± 0.04 | 1.8 ± 0.4 |
| Benzyl Benzoate | 8.8 | 0.81 ± 0.11 | 8.3 ± 1.1 |
| PEG 200 | 50 | 4.65 ± 0.21 | 48.5 ± 2.3 |
| PEG 300 | 70 | 6.88 ± 0.66 | 71.8 ± 6.9 |
| PEG 400 | 90 | 8.55 ± 0.45 | 89.4 ± 4.7 |

TABLE 20
Dispersion properties for nanoclusters.

| | Initial | Initial | Time of | | | Osmolality/ | | | | |
|---|---|---|---|---|---|---|---|---|---|---|
| | conc of | volume of | centri- | Final conc | Conc of | osmolality | | | Hydrodynamic | |
| | protein | protein | fugation | of protein | trehalose | at isotonic | Viscosity | Intrinsic | diameter by | % monomer |
| No. | (mg/ml) | solution (μl) | (min) | (mg/ml) | (mg/ml) | conc | (cP) | viscosity | DLS (nm) | by SEC |
| 1 | 33 | 498 | 80 | 220 | 100 | 1.19 | 12.70 | 6.63 | 33.50 | 91.0 |
| 2 | 51 | 499 | 80 | 260 | 125 | 1.41 | 13.16 | 5.25 | 30.07 | 92.3 |
| 3 | 67 | 250 | 40 | 268 | 125 | 1.41 | 20.73 | 6.00 | 32.27 | 92.6 |
| 4 | 42 | 353 | 60 | 310 | 125 | 1.41 | 94.28 | 7.83 | — | — |
| 5 | 40 | 370 | 82 | 300 | 100 | 1.19 | 193.8 | 9.06 | — | — |

Dispersion viscosities were determined by using the syringe viscosity method described herein. Dynamic light scattering (DLS) was done to determine the hydrodynamic diameter of the clusters and size exclusion chromatography (SEC) to check for the presence of irreversible aggregates. Samples for SEC were diluted from dispersion down to 1 mg/mL solution in 100 mM sodium phosphate buffer with 300 mM sodium chloride prior to analysis.

TABLE 21
Table 21. Isotonic dispersions of nanoclusters of sheep IgG.

| | Initial | Initial | Time of | | | | Osmolality/ | | | Hydro- | % monomer |
|---|---|---|---|---|---|---|---|---|---|---|---|
| | conc of | volume of | centri- | Total Conc | Final conc | Conc of | osmolality | | | dynamic | of diluted |
| | protein | protein | fugation | of buffer | of protein | trehalose | at isolonic | Viscosity | Intrinsic | diameter | sample |
| No. | (mg/ml) | solution (μl) | (min) | (mM) | (mg/ml) | (mg/ml) | conc | (cP) | viscosity | (nm) | by SEC |
| 1 | Lyophilized protein powder | | | 50 | 350 | 0 | 0.30 | Gel | — | — | — |
| 2 | directly dispersed into solu- | | | 50 | 350 | 35 | 0.61 | 591.13 | 8.92 | 21 | — |
| 3 | tion of trehalose in a buffer. | | | 50 | 350 | 70 | 0.92 | 873.27 | 9.24 | 24 | — |
| 4 | 189 | 210 | 80 | 50 | 331 | 100 | 1.19 | 368.44 | 8.75 | — | — |
| 5 | 67 | 517 | 202 | 50 | 335 | 80 | 1.01 | 397.73 | 8.79 | 45 | — |
| 6 | 34 | 678 | 25 | 20 | 215 | 100 | 1 | 28.07 | 8.38 | 28 | — |
| 7 | 36 | 525 | 40 | 20 | 296 | 100 | 1 | 227.19 | 9.45 | 42 | 98.2 |
| 8 | 90 | 190 | 35 | 50 | 276 | 70 | 0.92 | 42.93 | 7.48 | 40 | 93.4* |
| 9 | 37 | 530 | 35 | 50 | 317 | 70 | 0.92 | 91.80 | 7.26 | 22** | 99.9 |
| 10 | 49 | 500 | 45 | 50 | 292 | 100 | 0.33 | 110.48 | 7.38 | 48** | 98.0 |
| | | | | | | (1 kD | | | | | |
| | | | | | | Dextran) | | | | | |
| 11 | 37 | 486 | 55 | 50 | 348 | 70 | 0.94 | 146.88 | — | — | 99.5 |
| | | | | | | (+1% | | | | | |
| | | | | | | PS80) | | | | | |
| 12 | 36 | — | 50 | 50 | 333 | 50 | 0.74 | 552.50 | 9.51 | — | 99.7 |
| 13 | 40 | 528 | 65 | 20 | 326 | 70 | 2.19 | 608.59 | 9.80 | — | 98.8 |
| | | | | | | (2% | | | | | |
| | | | | | | ethanol) | | | | | |
| 14 | 88 | 234 | 40 | 50 | 324 | 70 | 0.94 | 228.81 | 8.32 | — | — |

Dispersion viscosities were determined by using a syringe as described herein. Dynamic light scattering (DLS) was done to determine the hydrodynamic diameter of the clusters and size exclusion chromatography (SEC) to check for the presence of irreversible aggregates. In rows 1-3 dispersions were formed by lyophilization. In all the other rows, dispersion were formed by centrifugation to remove water. For comparison, all SEC samples except starred value were using purified IgG with monomer content of 99.9%.
*Sigma Aldrich unpurified protein used - 92.6% monomer prior to any handling
**measured after freezing, storing, and thawing Rows 6-14 are purified by fplc and from Rockland. All others in this document are unpurified. Rows 4 and 5 are from Rockland unpurified. All of the others in Table 20 and 21 besides these are from sigma without fplc.

TABLE 22
A description of the important dispersion characteristics
before and after a dispersion was frozen for ~1 week.
% monomer can be compared with 99.9% monomer for
aliquot of purified IgG from same lot.

| | | | Hydro- | % Monomer |
|---|---|---|---|---|
| | Vis- | Intrinsic | dynamic | of Diluted |
| State of Dispersion | cosity | Viscosity | Diameter | Sample |
| 215 mg/mL, 100 mg/mL | 28.07 | 8.38 | 27.57 | 98.7 |
| Trehalose - pre-freezing | | | | |
| row 6 Table 21 | | | | |
| 215 mg/mL, 100 mg/mL | 25.37 | 8.84 | 25.8 | 99.0 |
| Trehalose - post-freezing | | | | |
| row 6 table 21 | | | | |
| Row 9 post freezing | | | 24 | — |
| Row 10 post freezing | | | 48 | — |

### Example VII

### Sheep IgG Nanoclusters with Amino Acid as Crowding Agent and Sterile Filtration for Nanoclusters with Trehalose as Crowding Agent

Use of Amino Acid as Crowding Agent:

In row 1 of Table 23, a dispersion of 298 mg/mL IgG was made using 100 mg/mL Arginine as the crowding agent. Amino acids and peptides are often used in protein formulations. In addition, arginine has been shown to have a stabilizing effect on protein solutions {Timasheff, 2006, Biophys Chem}, and may have the same effect on protein clusters. The viscosity was only 73 cp despite the very high protein concentration of 298 mg/ml. This concept may be utilized with a wide variety of amino acids, dipeptides, tripeptides, and oligopeptides as crowders. At a given amino acid or peptides mass concentration in unit of mg/ml, the tonicty will decreases with the molecular weight of the peptide.

Sterile Filtration of Nanocluster Dispersions:

In rows 2a and 2b of Table 23, a dispersion of 223 mg/mL IgG was made using 70 mg/mL trehalose as the crowding agent. The size of the nanoclusters may be estimated to be smaller than 40 nm based on similar nanocluster formation conditions in Table. 21. The dispersion was then filtered through a Millex PVDF 0.22 μm syringe filter (4 mm diameter) into a vessel, and the permeate concentration was measured. The receiving vessel contained a small amount of water, which accounted for the very small decrease in concentration. This experiment indicates that these dispersions may be produced in a non-sterile environment, then sterile-filtered (e.g. as part of the filling process), which may decrease the cost of manufacturing. The final concentration after going through the filter was 199 mg/ml, indicating only a small amount of protein was lost in the filter.

Charged Crowding Molecule.

In row 3 of Table 23, a dispersion of 238 mg/mL IgG was made using 100 mg/mL sodium citrate as the crowding agent. Sodium citrate is a charged molecule similar in size to Trehalose that can act as a crowder, proving that a dispersion of nanoclusters can be formed with both charged and uncharged molecules.

TABLE 23
Sheep IgG Nanoclusters formed with arginine and nanocluster filtered for sterile filtration.

| | | | Time of | | Final conc | Conc of | Osmolality/ | | |
|---|---|---|---|---|---|---|---|---|---|
| | Initial conc of | Initial volume of | centrifugation | Total Conc of | of protein | trehalose | osmolality at | Viscosity | Intrinsic |
| No. | protein (mg/ml) | protein solution (μl) | (min) | buffer (mM) | (mg/ml) | (mg/ml) | isotonic conc | (cP) | viscosity |
| 1 | 52 | 503 | 20 | 20 | 298 | 100 | 1.89 | 73.36 | 7.21 |
| | | | | | | (arginine) | | | |
| 2a (pre | 5 | 25.84 mL | 40* | 50 | 217 | 70 | 0.92 | 46.89 | 10.39 |
| Filtration) | | | | | | | | | |
| 2b (post | 5 | 25.84 mL | 40* | 50 | 200 | 70 | 0.92 | 26.27 | 9.91 |
| Filtration) | | | | | | | | | |
| 3 | 44 | 609 | 66 | 20 | 238 | 100 | 3.56 | 391 | 11.7 |
| | | | | | | (sodium | | | |
| | | | | | | citrate) | | | |

*at 4500 rcf, due to larger sample volume required

### Example VII

These experiments were performed similar to those in Example IV and VI unless indicated otherwise. All samples used Polyclonal Sheep IgG (manufactured by Rockland Immunochemicals), which was purified via FPLC prior to use (99.9% monomer). The concentration after centrifugation was measured as follows: 2.00 μl of dispersion was diluted into 998 μl in either 20 mM or 50 mM phosphate buffer (pH 6.4), corresponding to the salt concentration of the buffered solution the dispersion was formed in. The sample was mixed well by manual shaking and then transferred to a 1 mL Hellma QS 10 mm cell. The absorbance at 280 nm was measured on a Cary 3E UV/Vis spectrophotometer, and then converted to concentration using Beer's law with an extinction coefficient (E^{0.1%}) of 1.43, as provided by Rockland Immunochemicals.

The E^{0.1%} extinction coefficient is a commonly used molar extinction coefficient with a reference state of a 1 mg/mL protein solution. This normalization is manifested as a change in units from M⁻¹ cm⁻¹ for the molar extinction coefficient, ∈, to (mg/mL)⁻¹cm⁻¹ for the E^{0.1%} coefficient. These two quantities are related by the expression ∈=(E^{0.1%}) MW. Use of the E^{0.1%} extinction coefficient in Beers' law gives protein concentration directly in mg/mL, while the molar extinction coefficient c yields molar concentrations. The E^{0.1%} extinction coefficient is more practical for direct mass concentration measurements, particularly when molecular weight is not known accurately.

Nanoclusters of sheep IgG were formed by centrifugal filtration-concentration with results shown in Table 24 and Table 25 with trehalose as the crowder. Dispersion viscosities were determined by using a syringe as previously described. Dynamic light scattering (DLS) was done to determine the hydrodynamic diameter of the clusters and size exclusion chromatography (SEC) to check for the presence of irreversible aggregates. The dispersions were slightly hypotonic in each case with the exception of row 3 in Table 25, which was isotonic. As indicated, longer centrifugation times coordinated accordingly to more concentrated dispersions and increased viscosities. The viscosities and hydrodynamic diameters for the dispersions are reported.

TABLE 24
Dispersion properties for nanoclusters of sheep IgG formed by the centrifugal filtration concentration method.

| | Initial conc | Initial volume | Time of | Conc of | Osmolality/ | Final conc | Solvent | | | Hydrodynamic |
|---|---|---|---|---|---|---|---|---|---|---|
| | of protein | of protein | centrifuga- | trehalose | osmolality at | of protein | viscosity | Viscosity | Intrinsic | diameter by |
| No. | (mg/ml) | solution (μl) | tion (min) | (mg/ml) | isotonic conc | (mg/ml) | (cP) | (cP) | viscosity | DLS (nm) |
| 1 | 43 | 473 | 22 | 70 | 0.92 | 233 | 1.13 | 33 | 9 | 30 |

TABLE 25
Isotonic dispersions of nanoclusters of sheep IgG formed by the centrifugal filtration concentration method.

| | Initial conc | Initial volume | Time of | Conc of | Osmolality/ | Final conc | Solvent | | | |
|---|---|---|---|---|---|---|---|---|---|---|
| | of protein | of protein | centrifuga- | trehalose | osmolality at | of protein | viscosity | Viscosity | Intrinsic | Hydrodynamic |
| No. | (mg/ml) | solution (μl) | tion (min) | (mg/ml) | isotonic conc | (mg/ml) | (cP) | (cP) | viscosity | diameter (nm) |
| 1 | 46 | 547 | 46 | 70 | 0.92 | 312 | 1.13 | 393 | 10 | 30 |
| 2 | 48 | 568 | 46 | 70 | 0.92 | 321 | 1.13 | 389 | 9 | 31 |
| 3 | 48 | 598 | 37 | 100 | 1.0 | 254 | 1.22 | 128 | 11 | 40 |

Using arginine as a crowder was also tested as arginine has been shown to have a stabilizing effect on protein solutions (Timasheff, 2006, Biophys Chem). As shown in Table 26, nanoclusters of sheep IgG from Rockland chemical were formed by centrifugal filtration with arginine as a crowder. The count rate in DLS was on the order of 1000 counts per second (versus tens of thousands of counts per second for the dispersions containing trehalose), and thus too small to produce accurate autocorrelation functions. For a final protein concentration of 296 mg/ml, the viscosity was only 73 cp with an intrinsic viscosity of only 7. Using arginine and other amino acids as crowders has advantages because at a given amino acid or peptides mass concentration in unit of mg/ml, the tonicity will decreases with the molecular weight of the peptide.

A 296 mg/mL IgG dispersion consisting of 100 mg/mL arginine crowder in pH 6.4 20 mM sodium phosphate buffer (row 3 in Table 26) was analyzed by scanning electron microscopy (SEM). To prepare the arginine sample for SEM (using a Hitachi S-5500 scanning electron microscope at 30 KV), the dispersion was diluted to about 75 mg/ml (a fourth of the original protein concentration) at a constant crowder volume fraction of 0.077 (corresponding to the volume of fraction of crowder in the original dispersion at 296 mg/ml) using NMP as a crowder, dropped on a copper TEM grid with a lacey carbon film, blotted to remove the excess liquid, rapidly frozen by immersion in liquid nitrogen and then lyophilized. The images of individual nanoclusters can be seen in FIG. 52. Each image contains a single nanoparticle on top of a lacey carbon gride. The nanoclusters are between 50-100 nm in diameter.

TABLE 26
Dispersions of nanoclusters of sheep IgG formed by the centrifugal filtration concentration
method with arginine as the crowder at a concentration of 100 mg/ml.

| | | Conc of | Osmolality/ | | Initial volume of | Time of | | | | |
|---|---|---|---|---|---|---|---|---|---|---|
| | Crowder | crowder | osmolality at | Initial conc of | protein solution | centrifugation | Final conc of | Solvent | Viscosity | Intrinsic |
| No. | used | (mg/ml) | isotonic conc | protein (mg/ml) | (μl) | (min) | protein (mg/ml) | viscosity | (cP) | viscosity |
| 1 | Arginine | 100 | 1.89 | 69 | 550 | 25 | 298 | 1.36 | 166 | 8 |
| 2 | Arginine | 100 | 1.89 | 69 | 550 | 25 | 323 | 1.36 | 207 | 8 |
| 3 | Arginine | 100 | 1.89 | 58 | 595 | 21 | 296 | 1.36 | 73 | 7 |
| 4 | Arginine | 100 | 1.89 | 47 | 555 | 17 | 218 | 1.36 | 24 | 9 |

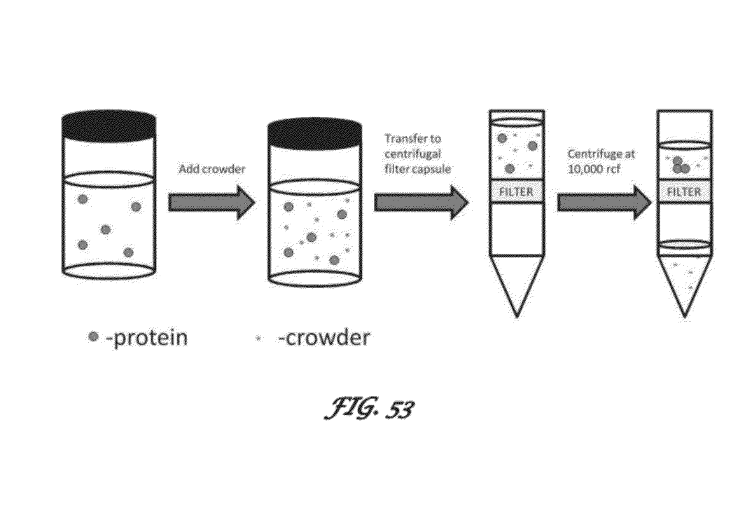

Storage stability. The storage stability for the dispersions prepared using trehalose as the extrinsic crowder was analyzed and is reported in Table 27 and FIG. 50. A 217 mg/mL IgG, 70 mg/mL trehalose dispersion formed by the centrifugal filtration concentration method as shown in FIG. 53, in a 50 mM phosphate buffer (Osmolality/osmolality at isotonic concentration of 0.92) was frozen for ˜1 month. After one month of storage in the frozen state at −40° C., the sample was thawed in a fridge at 4° C. and then analyzed. The viscosity and hydrodynamic diameter changed very little upon storage.

TABLE 27
Dispersion characteristics before and after freezing.

| | | Initial volume of | Time of | | | |
|---|---|---|---|---|---|---|
| State of | Initial conc of | protein solution | centrifugation | Viscosity | Intrinsic | Hydrodynamic |
| Dispersion | protein (mg/ml) | (ml) | (min) | (cP) | Viscosity | Diameter (nm) |
| Pre-freezing | 5 | 25.84 | 35 | 36 ± 9 | 9 | 30* |
| Post-freezing | 5 | 25.84 | 35 | 35 | 9 | 26* |

Note:
All sizes were obtained post thawing. In addition, the large initial volume is due to the use of a Millipore Centricon filter in order to gain the large volumes required for the sterile filtration experiment described in Table 28.

Forming sterile dispersions can be beneficial for industrial applications to reduce overall costs of manufacture. Therefore, use of sterile filtration was analyzed. The results for sterile filtration are given in Table 28 for samples also shown in Table 27. The sterile filtration experiment was done after forming the dispersions. A 217 mg/mL IgG, 70 mg/mL trehalose dispersion in a 50 mM phosphate buffer (Osmolality/osmolality at isotonic conc of 0.92) was initially prepared and passed through a 0.22 μm filter. The dispersions were then frozen, stored and thawed according to the conditions shown in Table 27. Subsequently, the hydrodynamic diameters were measured. The similar size, pre- and post-filtration, indicate the nanoclusters passed through the filter.

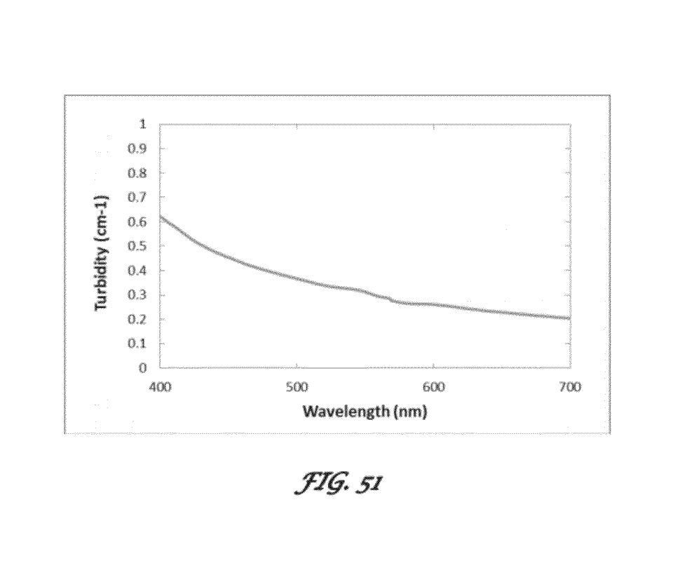

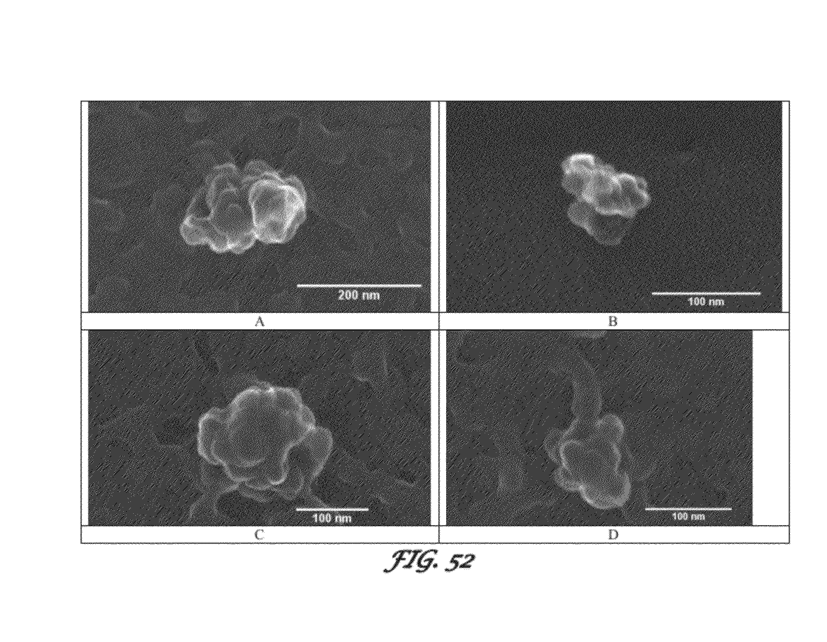

Turbidity of dispersions was measured on Cary 3E UV/Vis spectrophotometer and is reported in Table 296. FIG. 51 demonstrates that the turbidity for the pre-filtration is very low at varying wavelengths thus reinforcing the optical clarity of the dispersions.

TABLE 28
Sterile filtration.

| State of | Initial conc of | Initial volume of | Time of | Final conc of | Viscosity | Intrinsic | Hydrodynamic |
|---|---|---|---|---|---|---|---|
| dispersion | protein (mg/ml) | protein solution (ml) | centrifugation (min) | protein (mg/ml) | (cP) | viscosity | diameter (nm) |
| Pre- | 5 | 25.84 | 35 | 217 | 36 ± 9 | 9 | 30 |
| filtration | | | | | | | |
| Post- | 5 | 25.84 | 35 | 200 | 26 | 10 | 26 |
| filtration | | | | | | | |

Dispersion characteristics before and after freezing.
Note:
All sizes were obtained after samples were frozen and thawed. In addition, the large initial volume is due to the use of a Millipore Centricon filter in order to gain the large volumes required for the sterile filtration experiment.

TABLE 29
Optical properties of dispersion of nanoclusters in FIG. 2

| | Absor- | Turbidity | Absorbance | Turbidity |
|---|---|---|---|---|
| | bance at | at 600 | average | average 400- |
| Dispersion | 600 nm | nm (1/cm) | 400-700 nm | 700 nm (1/cm) |
| 2l7 mg/ml | 0.113 | 0.261 | 0.145 | 0.335 |
| IgG + 70 mg/ml | | | | |
| trehalose | | | | |

It is contemplated that any embodiment discussed in this specification can be implemented with respect to any method, kit, reagent or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

U.S. Pat. No. 7,374,782: Production of Microspheres.
U.S. Patent Application 20100158899: Protein Formulation.
U.S. Patent Application No. 20060024379: Protein Microspheres having Injectable Properties at High Concentrations.
Chari, R., K. Jerath, et al. (2009). “Long- and Short-Range Electrostatic Interactions Affect the Rheology of Highly Concentrated Antibody Solutions.” Pharm. Res. 26(12): 2607-2618.
Groenewold, J. and W. K. Kegel (2001). “Anomalously large equilibrium clusters of colloids.” Journal of Physical Chemistry B 105(47): 11702-11709.
Horn, F. (2000). “Hydrodynamic and Colloidal Interactions in Concentrated Charge-Stabilized Polymer Dispersions.” Journal of Colloid and Interface Science 225(1): 166-178.
Kulkarni, A. M., N. M. Dixit, et al. (2003). “Ergodic and non-ergodic phase transitions in globular protein suspensions.” Farady Discuss. 123: 37-50.
Maynard, J. A., C. B. M. Maassen, et al. (2002). “Protection against anthrax toxin by recombinant antibody fragments correlates with antigen affinity.” Nature Biotechnology 20: 597-601.
Miller, M. A. (2011). Ph. D. Thesis. Department of Chemical Engineering. Austin, University of Texas at Austin. Ph. D.
Porcar, L., P. Falus, et al. (2010). “Formation of the Dynamic Clusters in Concentrated Lyoszyme Protein Solutions.” J. Phys. Chem. Lett. 1: 126-129.
Saluja, A. and D. S. Kalonia (2008). “Nature and consequences of protein-protein interactions in high protein concentration solutions.” Int. J. Pharm. 358:1-15.
Shen, V. K., J. K. Cheung, et al. (2006). “Coarse-Grained Strategy for Modeling Protein Stability in Concentrated Solutions. II: Phase Behavior.” Biophysical Journal 90(6): 1949-1960.
Shen, V. K., J. K. Cheung, et al. (2009). “Insights into Crowding Effects on Protein Stability from Coarse-Grained Model.” Journal of Biomechanical Engineering 131: 071002 (071007 pg).
Sinko, Patrick J., and Alfred N. Martin. Martin's Physical Pharmacy and Pharmaceutical Sciences Physical Chemical and Biopharmaceutical Principles in the Pharmaceutical Sciences. Philadelphia: Lippincott Williams & Wilkins, 2006.
Sutherland, J. N. and J. A. Maynard (2009). “Characterization of a Key Neutralizing Epitope on Pertussis Toxin Recognized by Monoclonal Antibody 1B7.” Biochemistry 48: 11982-11993.
Zhou, H.-X., G. Rivas, et al. (2008). “Macromolecular Crowding and Confinement: Biochemical, Biophysical, and Potential Physiological Consequences*.” Annual Review of Biophysics 37(1): 375-397.
Arfken, G. B. and H. J. Weber (1995). Mathematical Methods for Physicists. San Diego, Academic Press.
Asakura, S, and F. Oosawa (1958). “Interaction between particles suspended in solutions of macromolecules.” Journal of Polymer Science 33(126): 183-192.
Chari, R., K. Jerath, et al. (2009). “Long- and short-range electrostatic interactions affect the rheology of highly concentrated antibody solutions.” Pharmaceutical Research 26(12): 2607-2618.
Cheung, J. K. and T. M. Truskett (2005). “Coarse-grained strategy for modeling protein stability in concentrated solutions.” Biophysical Journal 89(4): 2372-2384.
Cheung, M. S., D. Klimov, et al. (2005). “Molecular crowding enhances native state stability and refolding rates of globular proteins.” Proceedings of the National Academy of Sciences of the United States of America 102(13): 4753-4758.
Chi. E. Y., S. Krishnan, et al. (2003). “Roles of conformational stability and colloidal stability in the aggregation of recombinant human granulocyte colony-stimulating factor.” Protein Science 12(5): 903-913.
Crowther, J. R. and Editor (1995). ELISA: Theory and Practice. [In: Methods Mol. Biol. (Totowa, N.J.), 1995; 42], Humana.
Davis-Searles, P. R., A. J. Saunders, et al. (2001). “Interpreting the effects of small uncharged solutes on protein-folding equilibria.” Annual Review of Biophysics and Biomolecular Structure 30: 271-306.
del Alamo, M., G. Rivas, et al. (2005). “Effect of Macromolecular Crowding Agents on Human Immunodeficiency Virus Type 1 Capsid Protein Assembly In Vitro.” Journal of Virology 79(22): 14271-14281.
Dhar, A., A. Samiotakis, et al. (2010). “Structure, function, and folding of phosphoglycerate kinase are strongly perturbed by macromolecular crowding.” Proceedings of the National Academy of Sciences of the United States of America 107(41): 17586-17591.
Fields, G. B., D. O. V. Alonso, et al. (1992). “Theory for the aggregation of proteins and copolymers.” Journal of Physical Chemistry 96(10): 3974-3981.
Garber, E. and S. J. Demarest (2007). “A broad range of Fab stabilities within a host of therapeutic IgGs.” Biochemical and Biophysical Research Communications 355(3): 751-757.
Gast, A. P., C. K. Hall, et al. (1983). “Polymer-induced phase separations in nonaqueous colloidal suspensions.” J Colloid Interface Sci 96(1): 251-267.
Groenewold, J. and W. K. Kegel (2001). “Anomalously large equilibrium clusters of colloids.” Journal of Physical Chemistry B 105(47): 11702-11709.
Groenewold, J. and W. K. Kegel (2004). “Colloidal cluster phases, gelation and nuclear matter.” Journal of Physics-Condensed Matter 16(42): S4877-S4886.
Hall, D. and A. P. Minton (2003). “Macromolecular crowding: qualitative and semiquantitative successes, quantitative challenges.” Biochimica et Biophysica Acta (BBA)-Proteins & Proteomics 1649(2): 127-139.
Harn, N., T. Spitznagel, et al. (2010). Biophysical Signatures of Monoclonal Antibodies. Current Trends in Monoclonal Antibody Development and Manufacturing. S. J. Shire. New York, Springer: 229-246.
Hartl. F. U. and M. Hayer-Hartl (2002). “Protein folding-Molecular chaperones in the cytosol: from nascent chain to folded protein.” Science 295(5561): 1852-1858.
Hiemenz, P. C. and R. Rajagopalan (1997). Principles of Colloid and Surface Chemistry. New York, Marcel Dekker, Inc.
Horn, F. (2000). “Hydrodynamic and colloidal interactions in concentrated charge-stabilized polymer dispersions.” Journal of Colloid and Interface Science 225(1): 166-178.
Kendrick, B. S., J. F. Carpenter, et al. (1998). “A transient expansion of the native state precedes aggregation of recombinant human interferon-γ.” PNAS 95: 14142-14146.
Krishnan, S., E. Y. Chi, et al. (2002). “Aggregation of Granulocyte Colony Stimulating Factor under Physiological Conditions: Characterization and Thermodynamic Inhibition.” Biochemistry 41: 6422-6431.
Kumar, V., V. K. Sharma, et al. (2009). “In Situ Precipitation and Vacuum Drying of Interferon Alpha-2a: Development of a Single-Step Process for Obtaining Dry, Stable Protein Formulation.” International Journal of Pharmaceutics 366(1-2): 88-98.
Lavinder, J. J., S. B. Hari, et al. (2009). “High-throughput thermal scanning: a general, rapid dye-binding thermal shift screen for protein engineering.” Journal of the American Chemical Society 131(11): 3794-3795.
Lee, J. C. and S, N. Timasheff (1981). “The stabilization of proteins by sucrose.” Journal of Biological Chemistry 256(14): 7193-7201.
Liu, J., M. D. H. Nguyen, et al. (2005). “Reversible Self-Association Increases the Viscosity of a Concentrated Monoclonal Antibody in Aqueous Solution.” Journal of Pharmaceutical Sciences 94(9): 1928-1940.
Lu, P. J., J. C. Conrad, et al. (2006). “Fluids of Clusters in Attractive Colloids.” Physical Review Letters 96(2).
Lu, P. J., E. Zaccarelli, et al. (2008). “Gelation of particles with short-range attraction.” Nature 453(7194): 499-503.
Miller, D. P., J. J. dePablo, et al. (1997). “Thermophysical Properties of Trehalose and its Concentrated Aqueous Solutions.” Pharmaceutical Research 14(5): 578-590.
Miller, M. A., J. D. Engstrom, et al. (2010). “Low Viscosity Highly Concentrated Injectable Nonaqueous Suspensions of Lysozyme Microparticles.” Langmuir 26(2): 1067-1074.
Minton, A. P. (1999). “Adsorption of globular proteins on locally planar surfaces. II. Models for the effect of multiple adsorbate conformations on adsorption equilibria and kinetics.” Biophysical Journal 76(1): 176-187.
Mutch, K. J., J. S. van Duijneveldt, et al. (2007). “Colloid-Polymer Mixtures in the Protein Limit.” Soft Matter 3(2): 155.
Oconnor, T., P. Debenedetti, et al. (2007). “Stability of proteins in the presence of carbohydrates; experiments and modeling using scaled particle theory.” Biophysical Chemistry 127(1-2): 51-63.
Pan, W. C., P. G. Vekilov, et al. (2010). “Origin of anomalous mesoscopic phases in protein solutions.” Journal of Physical Chemistry B 114(22): 7620-7630.
Pielak, G. J. and A. C. Miklos (2010). “Crowding and function reunite.” Proceedings of the National Academy of Sciences 107(41): 17457-17458.
Pilz, I., G. Puchwein, et al. (1970). “Small Angle X-Ray Scattering of a Homogeneous GammaG1 Immunoglobin.” Biochemistry 9(2): 211-219.
Ping, G., G. Yang, et al. (2006). “Depletion force from macromolecular crowding enhances mechanical stability of protein molecules.” Polymer 47(7): 2564-2570.
Porcar, L., P. Falus, et al. (2010). “Formation of the dynamic clusters in concentrated lyoszyme protein solutions.” J. Phys. Chem. Lett. 1: 126-129.
Roosen-Runge, F., M. Hennig, et al. (2011). “Protein self-diffusion in crowded solutions.” Proceedings of the National Academy of Sciences 108(29): 11815-11820.
Rosenbaum, D. F., P. C. Zamora, et al. (1996). “Phase behavior of small attractive colloidal particles.” Phys Rev Let 76(1): 150-153.
Scherer, T. M., J. Liu, et al. (2010). “Intermolecular interactions of IgG1 monoclonal antibodies at high concentrations characterized by light scattering.” J Phys Chem B 114: 12948-12957.
Sedgwick, H., S. U. Egelhaaf, et al. (2004). “Clusters and gels in systems of sticky particles.” Journal of Physics: Condensed Matter 16(42): S4913-S4922.
Sharma, A. and J. Y. Walz (1996). “Direct Measurement of the Depletion Interaction in a Charged Colloidal Dispersion.” Journal of the Chemical Society, Faraday Transactions 92(24): 4997.
Shen, V. K., J. K. Cheung, et al. (2006). “Coarse-grained strategy for modeling protein stability in concentrated solutions 11: Phase behavior.” Biophysical Journal 90: 1949-1960.
Shire, S. J., Z. Shahrokh, et al. (2004). “Challenges in the development of high protein concentration formulations.” J. Pharm. Sci. 93(6): 1390-1402.
Stradner, A., H. Sedgwick, et al. (2004). “Equilibrium cluster formation in concentrated protein solutions and colloids.” Nature 432(7016): 492-495.
Sutherland, J. N., C. Chang, et al. (2011). “Antibodies recognizing protective Pertussis toxin epitopes are preferentially elicited by natural infection versus acellular immunization.” Clinical and Vaccine Immunology 18(6): 954-962.
Sutherland, J. N. and J. A. Maynard (2009). “Characterization of a key neutralizing epitope on Pertussis toxin recognized by monoclonal antibody 1B7.” Biochemistry 48: 11982-11993.
Tabrizi, M. A., C. M. L. Tseng, et al. (2006). “Elimination mechanisms of therapeutic monoclonal antibodies.” Drug Discovery Today 11(1-2): 81-88.
Tam, J. M., A. K. Murthy, et al. (2010). “Kinetic Assembly of Near-IR-Active Gold Nanoclusters Using Weakly Adsorbing Polymers to Control the Size.” Langmuir 26(11): 8988-8999.
Tam, J. M., J. O. Tam, et al. (2010). “Controlled Assembly of Biodegradable Plasmonic Nanoclusters for Near-Infrared Imaging and Therapeutic Applications.” Acs Nano 4(4): 2178-2184.
ten Wolde, P. R. and D. Frenkel (1997). “Enhancement of protein crystal nucleation by critical density fluctuations.” Science 277: 1975-1978.
Torquato, S., T. M. Truskett, et al. (2000). “Is Random Close Packing of Spheres Well Defined?” Physical Review Letters 84(10): 2064-2067.
Tuinier, R., G. A. Vliegenthart, et al. (2000). “Depletion interaction between spheres immersed in a solution of ideal polymer chains.” Journal of Chemical Physics 113(23): 10768-10775.
Uchida, T., M. Nagayama, et al. (2009). “Trehalose Solution Viscosity at Low Temperatures Measured by Dynamic Light Scattering Method: Trehalose Depresses Molecular Transportation for Ice Crystal Growth.” Journal of Crystal Growth 311(23-24): 4747-4752.
Vrij, A. (1976). “Polymer at interfaces and interactions in colloidal dispersions.” Pure and Applied Chemistry 48(4): 471-483.
Wallace, B. A., R. W. Janes, et al. (2009). Modern Techniques for Circular Dichroism and Synchrotron Radiation Circular Dichroism Spectroscopy. [In: Adv. Biomed. Spectrosc., 2009; 1], IOS Press.
Xia, Y. S., T. D. Nguyen, et al. (2011). “Self-Assembly of Self-Limiting Monodisperse Supraparticles from Polydisperse Nanoparticles.” Nature Nanotechnology 6(9): 580-587.
Yadav, S., J. Liu, et al. (2010). “Specific interactions in high concentration antibody solutions resulting in high viscosity.” J Pharm Sci 99(3): 1152-1168.
Young, T. M. and C. J. Roberts (2009). “Structure and Thermodynamics of Colloidal Protein Cluster Formation: Comparison of Square-Well and Simple Dipolar Models.” The Journal of Chemical Physics 131(12): 125104.
Zaccarelli, E. (2007). “Colloidal gels: equilibrium and non-equilibrium routes.” J Phys: Condens. Matter 19: 323101.
Zhou, H. X., G. Rivas, et al. (2008). “Macromolecular crowding and confinement: biochemical, biophysical, and potential physiological consequences.” Annual Review of Biophysics 37(1): 375-397.
Ahmad, M., W. Gu, et al. (2011). “Adhesive water networks facilitate binding of protein interfaces.” Nature Communications 2: 261.
Asakura, S, and F. Oosawa (1958). “Interaction between particles suspended in solutions of macromolecules.” Journal of Polymer Science 33(126): 183-192.
Bajaj, H., V. K. Sharma, et al. (2007). “A high-throughput method for detection of protein self-association and second virial coefficient using size-exclusion chromatography through simultaneous measurement of concentration and scattered light intensity.” Pharm. Res. 24(11): 2071-2083.
Biswas, R., N. Rohman, et al. (2008). “Intramolecular charge transfer reaction, polarity, and dielectric relaxation in AOT/water/heptane reverse micelles: pool size dependence.” The Journal of Physical Chemistry B112(31): 9379-9388.
Bottcher, C. J. F. (1945). “The dielectric constant of crystalline powders.” Recl. Tray. Chim. Pays-Bas Belg. 64 (Copyright (C) 2011 American Chemical Society (ACS). All Rights Reserved.): 47-51.
Curtis, R. A., J. M. Prausnitz, et al. (1998). “Protein-protein and protein-salt interactions in aqueous protein solutions containing concentrated electrolytes.” Biotechnology and Bioengineering 57(1): 11-21.
Groenewold, J. and W. K. Kegel (2001). “Anomalously large equilibrium clusters of colloids.” Journal of Physical Chemistry B105(47): 11702-11709.
Hiemenz, P. C. and R. Rajagopalan (1997). Principles of Colloid and Surface Chemistry. New York, Marcel Dekker, Inc.
Kanai, S., J. Liu, et al. (2008). “Reversible self-association of a concentrated monoclonal antibody solution mediated by Fab-Fab interaction that impacts solution viscosity.” J Pharm Sci 97(10): 4219-4227.
Kralchevsky, P. A., K. D. Danov, et al. (2011). “Hydration force due to the reduced screening of the electrostatic repulsion in few-nanometer-thick films.” Current Opinion in Colloid & Interface Science.
Kulkarni, A. M., N. M. Dixit, et al. (2003). “Ergodic and non-ergodic phase transitions in globular protein suspensions.” Farady Discuss. 123: 37-50.
McCabe, W. J., J. C. Smith, et al. (1985). Unit Operations of Chemical Engineering. 4th Ed, Macgraw Hill.
Minton, A. P. (2007). “The effective hard particle model provides a simple, robust, and broadly applicable description of nonideal behavior in concentrated solutions of bovine serum albumin and other nonassociating proteins.” Journal of Pharmaceutical Sciences 96(12): 3466-3469.
Paddison, S. J. (2003). “Proton conduction mechanisms at low degrees of hydration in sulfonic acid-based polymer electrolyte membrane.” Annu. Rev. Mater. Res. 33: 289-319.
Reynolds, J. A. and J. M. Hough (1957). “Formulas for dielectric constant of mixtures.” Proc. Phys. Soc., London70B (Copyright (C) 2011 American Chemical Society (ACS). All Rights Reserved.): 769-775.
Rintoul, M. D. and S. Torquato (1998). “Hard-sphere statistics along the metastable amorphous branch.” Physical Review E58(1): 532.
Rosenbaum, D. F., A. M. Kulkarni, et al. (1999). “Protein interactions and phase behavior: Sensitivity to the form of the pair potential.” J Chem Phys 111(21): 9882-9890.
S. Asakura, F. O. (1954). J. Chem. Phys. 22.
Senapati, S, and A. Chandra (2001). “Dielectric constant of water confined in a nanocavity.” The Journal of Physical Chemistry B 105(22): 5106-5109.
Stradner, A., H. Sedgwick, et al. (2004). “Equilibrium cluster formation in concentrated protein solutions and colloids.” Nature 432(7016): 492-495.
ten Wolde, P. R. and D. Frenkel (1997). “Enhancement of protein crystal nucleation by critical density fluctuations.” Science 277: 1975-1978.
Tuinier, R., J. Rieger, et al. (2003). “Depletion-induced phase separation in colloid-polymer mixtures.” Advances in Colloid and Interface Science 103: 1-31.
Wang, M. and N. Pan (2007). “Numerical analyses of effective dielectric constant of multiphase microporous media.” Journal of Applied Physics 101(11): 114102.
Yadav, S., J. Liu, et al. (2010). “Specific interactions in high concentration antibody solutions resulting in high viscosity.” J Pharm Sci 99(3): 1152-1168.
Zhou, H. X. (2008). “Effect of mixed macromolecular crowding agents on protein folding.” Proteins: Structure, Function, and Bioinformatics 72(4): 1109-1113.
Zhou, H. X., G. Rivas, et al. (2008). “Macromolecular crowding and confinement: biochemical, biophysical, and potential physiological consequences.” Annual Review of Biophysics 37(1): 375-397.
Chu, T. C. “Aptamer:Toxin Conjugates That Specifically Target Prostate Tumor Cells.” Cancer Research 66.12 (2006): 5989-992.
Hamblett, K. J. “Effects of Drug Loading on the Antitumor Activity of a Monoclonal Antibody Drug Conjugate.” Clinical Cancer Research 10.20 (2004): 7063-070.
Krop, I. E., M. Beeram, S. Modi, S. F. Jones, S, N. Holden, W. Yu, S. Girish, J. Tibbitts, J.-H. Yi, M. X. Sliwkowski, F. Jacobson, S. G. Lutzker, and H. A. Burris. “Phase Study of Trastuzumab-DM1, an HER2 Antibody-Drug Conjugate, Given Every 3 Weeks to Patients With HER2-Positive Metastatic Breast Cancer.” Journal of Clinical Oncology 28.16 (2010): 2698-704.

What is claimed is:

1. A transparent, low viscosity, high protein concentration dispersion, wherein said dispersion comprises a plurality of nanoclusters, wherein each of said plurality of nanoclusters comprises a plurality of proteins, wherein each of said plurality of proteins shares amino acid sequence identity.

2. The dispersion of claim 1, wherein said dispersion is syringeable and wherein an aqueous solution of the plurality of proteins at an identical concentration is not syringeable.

3. The dispersion of claim 1, comprising between about 200 mg/mL and about 400 mg/mL of the protein.

4. The dispersion of claim 1, comprising a crowder.

5. The dispersion of claim 1, comprising a crowder selected from the group consisting of a trehalose, a poly(ethylene glycol), ethanol, N-methyl-2-pyrrolidone (NMP), a buffer, or a combination thereof.

6. The dispersion of claim 1, comprising about a 1:1 weight ratio of protein to a crowder.

7. The dispersion of claim 1, comprising about a 2:1 weight ratio of protein to a crowder.

8. The dispersion of claim 1, wherein said dispersion is isotonic with human blood.

9. The dispersion of claim 1, wherein said plurality of proteins is a plurality of conjugates, wherein each of said conjugates is a protein bonded to a low molecular weight compound, wherein said low molecular weight compound is a diagnostic agent, a pharmaceutical agent, a contrast agent, a fluorophore, a radioisotope, a toxin, a paramagnetic agent, or an aptamer.

10. A pharmaceutical composition comprising the dispersion of claim 1, wherein said plurality of proteins is a plurality of pharmaceutically active proteins.

11. A method of making a transparent, low viscosity, high protein dispersion of protein nanoclusters comprising concentrating a protein-crowder liquid combination and thereby forming said dispersion, wherein said dispersion comprises a plurality of nanoclusters, wherein each of said plurality of nanoclusters comprises a plurality of proteins, wherein each of said plurality of proteins shares amino acid sequence identity; wherein said dispersion is a transparent, low viscosity, dispersion; wherein said dispersion comprises a concentration of said protein of greater than about 200 mg/mL, and wherein said dispersion comprises a plurality of a crowder.

12. The method of claim 11 wherein the crowder is a glycerol, an erythritol, an arabinose, a xylose, a ribose, an inositol, a fructose, a galactose, a maltose, a glucose, a mannose, a trehalose, a sucrose, a polyethylene glycol), an amino acid, peptide, a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt, a buffer or a combination thereof.

13. A method of making a transparent, low viscosity, high protein dispersion of protein nanoclusters comprising the step of combining a protein in powder form with a crowder and a dispersion liquid thereby forming a dispersion comprising a plurality of nanoclusters comprising a plurality of said protein, wherein each of said plurality of proteins shares amino acid sequence identity; wherein said dispersion is a transparent, low viscosity, dispersion; wherein said dispersion comprises a concentration of said protein of greater than about 200 mg/mL.

14. The method of claim 13, comprising, prior to said combining, applying spiral wound in situ freezing technology (SWIFT) to a protein-crowder mixture thereby forming said protein in powder form.

15. The method of claim 14, wherein applying SWIFT comprises the steps of:
a. rotating a vial, containing said mixture, while contacting the vial with a cryogenic agent;
b. freezing all of said mixture, wherein the freezing results in a thin film of the frozen mixture on the inner side of the vial and one or more subsequent films in a spiral orientation towards the center of the vial, and
c. lyophilizing said frozen mixture.

16. A method of making a transparent, low viscosity, high protein dispersion of protein nanoclusters comprising the step of combining a protein in powder form with a dispersion liquid thereby forming a dispersion comprising a plurality of nanoclusters comprising a plurality of said protein, wherein each of said plurality of proteins shares amino acid sequence identity; wherein said dispersion is a transparent, low viscosity, dispersion; wherein said dispersion comprises a concentration of said protein of greater than about 200 mg/mL.

17. A method of treating a disease in a patient in need of such treatment, said method comprising administering an effective amount of the dispersion of claim 1 to said patient.

18. A method of modifying the average protein nanocluster diameter of a transparent, low viscosity, high protein dispersion of protein nanoclusters comprising increasing or decreasing the concentration of a crowder or said protein in said dispersion, wherein said dispersion comprises a plurality of nanoclusters, wherein each of said plurality of nanoclusters comprises a plurality of proteins, wherein each of said plurality of proteins shares amino acid sequence identity; wherein said dispersion is a transparent, low viscosity, dispersion; and wherein said dispersion comprises a concentration of said protein of greater than about 200 mg/mL.

19. A kit, wherein the kit comprises the dispersion of claim 1 or the pharmaceutical composition of claim 10.

20. A kit, wherein the kit comprises a protein in powder form or a protein-crowder mixture in powder form, and a dispersion liquid.
